(19) United States
(12) Patent Application Publication
(10) Pub. No.: US 2024/0122895 A1
(43) Pub. Date: Apr. 18, 2024

(54) ENTACTOGEN-ASSISTED THERAPY FOR ALCOHOL USE DISORDER AND ADDICTION-RELATED CONDITIONS AND COMORBIDITIES

(71) Applicant: Awakn LS Europe Holdings Limited, Dublin (IE)

(72) Inventors: Ben Sessa, Bristol (GB); Laurie Higbed, Bristol (GB); David Nutt, London (GB)

(21) Appl. No.: 18/219,057

(22) Filed: Jul. 6, 2023

Related U.S. Application Data
(63) Continuation-in-part of application No. PCT/US22/11511, filed on Jan. 6, 2022.
(60) Provisional application No. 63/137,615, filed on Jan. 14, 2021.
(60) Provisional application No. 63/134,582, filed on Jan. 6, 2021.
(60) Provisional application No. 63/389,364, filed on Jul. 14, 2022.

(51) Int. Cl. A61K 31/357; A61K 45/06; A61P 25/32

(52) U.S. Cl. CPC A61K 31/357; A61K 45/06; A61P 25/32

(57) ABSTRACT

Provided are methods of administering an entactogen in conjunction with psychotherapy to treat a condition and/or to treat one or more comorbidities in a subject. In other aspects, provided are compositions, such as pharmaceutical compositions, comprising an entactogen for use in an entactogen-assisted psychotherapy (EAP) regimen. In some embodiments, the entactogen is MDMA, for use in MDMA-assisted therapy. In some aspects, the provided EAP methods are used to treat a substance use disorder (SUD) or a behavioral addiction. In some embodiments, the SUD is alcohol use disorder (AUD), including physically dependent (“dependent use”) AUD or non-physically dependent (“harmful use”) AUD. In a further aspect, the provided EAP methods are used to treat one or more of a comorbid SUD, behavioral addiction, or mental health disorder.

### CROSS REFERENCE

This is a continuation-in-part application with priority to PCT/US2022/11511, filed Jan. 6, 2022, with priority under PCT Article 8(1) and Rule 4.10 to U.S. Provisional Application Nos. 63/134,582, filed Jan. 6, 2021, and 63/137,615, filed Jan. 14, 2021; priority is also claimed under § 119(e) to U.S. Provisional Application No. 63/389,364, filed Jul. 14, 2022; each of the above are incorporated by reference for all purposes as if fully set forth herein.

### FIELD OF THE INVENTION

This disclosure relates, in some aspects, to the use of entactogens such as MDMA, in conjunction with therapy, to treat patients with alcohol use disorder (AUD), substance use disorders (SUDs), and behavioral addictions, as well comorbidities in a patient diagnosed with a condition.

### BACKGROUND OF THE INVENTION

The cost of excessive alcohol use is high, both to the individual, in terms of physical and mental health, and to society in terms of social and medical care. This is a growing problem with increasing numbers of adults labeled as problem drinkers. Despite a wide variety of psychological and pharmacological treatment options, long term outcomes are poor, with high rates (up to 60% at 12 months and 80% at 3 years) of relapse after medical detoxification and dedicated treatment programs (Evren et al. 2010, Moos & Moos 2006).

The impact of alcohol misuse is widespread, encompassing alcohol-related illness and injuries as well as significant social impacts to family, friends, and the wider community. People with AUD broadly commonly present with high levels of depression, social anxiety, and social exclusion (Castillo-Carniglia et al. 2019). Additionally, in patients with physical dependence, studies indicate that repeated detoxifications are associated with more complications (Malcolm et al. 2000). This underscores the need for successful treatments to help patients remain abstinent after their first detoxification and avoid the complications that can occur with repeated detoxifications.

Although therefore recognized as an area of medicine that needs new approaches, none have been forthcoming. Indeed, AUD is one of the hardest diagnoses to treat in all of psychiatry.

Beyond AUD, other substance use disorders (SUDs) as well as behavioral addictions (BAs) further plague many millions of individuals worldwide. SUDs, for example, are associated with, among other things, impairments in judgment, attention, and perception that can lead to a loss of control, and may result in damage to personal relationships, harm to the individual, and harm to others. Current treatment options are generally ineffective for long-term abstinence from substance use, one reason for which may be the myriad factors that may cause substance use disorders, including environmental, social, and genetic factors. Behavioral addictions are similarly difficult to treat and can be equally destructive to an individual and the individual's relationships.

In many cases, those being treated for one condition may present with other conditions, and may for example have comorbid substance use disorders, behavioral addictions, and/or mental health disorders. In such cases, successful treatment can be even more challenging.

To address these and other issues, Applicant herein discloses novel methods of using entactogens in conjunction with therapy, such as MDMA-assisted therapy, to treat AUD, other SUDs, behavioral addictions, and comorbid SUDs and behavioral addictions.

### INCORPORATION BY REFERENCE

Each patent, publication, and non-patent literature cited in the application, or listed in the section entitled “References” below, is hereby incorporated by reference in its entirety as if each was incorporated by reference individually, and as if each is fully set forth herein. However, where such reference is made, and whether to patents, publications, non-patent literature, or other sources of information, it is for the general purpose of providing context for discussing features of the invention, and accordingly shall not be construed as an admission that the document or underlying information, in any jurisdiction, is prior art, or is part of the common general knowledge in the art.

### BRIEF SUMMARY OF THE INVENTION

In some aspects are disclosed compositions of entactogens, such as MDMA, and their use in methods of entactogen-assisted psychotherapy (equivalently herein, “EAP” or “entactogen-assisted therapy”), which will be understood to include MDMA-assisted psychotherapy (equivalently herein, “MDMA-assisted therapy”), to treat patients with AUD. In further aspects are disclosed compositions of entactogens, such as MDMA, and their use in methods of EAP to treat addiction-related conditions, such as SUDs and behavioral addictions, including when such addiction-related conditions are comorbid with another condition. Such compositions and methods have various advantages over current treatments, as further disclosed and discussed herein.

The following presents a simplified summary of some embodiments of the invention and some of the advantages of the claimed compositions and methods, in order to provide a basic understanding of the invention. This summary is not an extensive overview of the invention, nor intended to identify key or critical elements of the invention or to delineate the scope thereof. Its sole purpose is to present some embodiments of the invention in a simplified form as a prelude to the detailed description that follows.

In some aspects are disclosed methods of treating a substance use disorder (SUD) in a patient diagnosed therewith, comprising providing an entactogen-assisted psychotherapy (EAP) regimen, wherein the EAP regimen comprises one or more drug-assisted therapy sessions with an entactogen, and one or more non-drug therapy sessions.

In some embodiments, the SUD is any of alcohol use disorder (AUD), opioid use disorder, nicotine dependence or tobacco use disorder, sedative, hypnotic, and anxiolytic use disorder, and stimulant use disorder. In some embodiments, the SUD is AUD.

In some embodiments, the AUD is dependent use AUD. In some embodiments, the method further comprises providing an alcohol detoxification course prior to the EAP regimen. In some embodiments, the method further comprises providing a pre-detoxification motivational group therapy course prior to the alcohol detoxification course.

In some embodiments, the AUD is harmful use AUD. In some embodiments, the method further comprises providing a preparatory group therapy course prior to the EAP regimen.

In some embodiments, the method is effective to: (a) reduce use of the substance, (b) reduce cravings for the substance, (c) promote abstinence from the substance, (d) prevent relapse into use of the substance, (e) improve a symptom of the SUD, or (f) reduce the severity of a diagnostic criterion of the SUD. In some embodiments, the method is effective for at least 12 months after the patient has completed the EAP regimen. In some embodiments, the method is effective to reduce use of the substance for at least 12 months after the patient has completed the EAP regimen. In some embodiments, the SUD is AUD, and the method is effective to reduce use of alcohol for at least 12 months after the patient has completed the EAP regimen.

In some embodiments, the EAP regimen comprises at least two drug-assisted therapy sessions with an entactogen. In some embodiments, the entactogen is any of MDMA, BDB, MBDB, MDA, MDEA, 3-MMC, 4-MMC, methylone, ethylone, butylone, αET, aMT, 5-IAI, MDAI, 5-APDI, 6-APB, 5-APB, 5-MAPB, MDAI, MDOH, and 4-FA, including individual enantiomers and non-racemic mixtures thereof, and including pharmaceutically acceptable salts thereof. In some embodiments, the entactogen is MDMA, including individual enantiomers and non-racemic mixtures thereof, and including pharmaceutically acceptable salts thereof.

In some embodiments, the method further comprises administration of an additional active agent, wherein the additional active agent is any of an opioid antagonist, a CB1 antagonist, a CRH1 receptor antagonist, a NK1R antagonist, an OTR agonist, a GABA agent, a voltage-gated sodium channel inhibitor, a voltage-dependent calcium channel agonist, an α7 nicotinic acetylcholine receptor agonist, a 5-HT3 antagonist, a 5-HT1A receptor partial agonist, a 5-HT2A receptor antagonist, a 5-HT reuptake inhibitor, a SERT inhibitor, a serotonergic agent, an α1 adrenoreceptor antagonist, a glucocorticoid receptor antagonist, an α1 adrenoreceptor agonist, an AChE inhibitor, a dopamine D2 receptor antagonist, an α2 adrenoreceptor agonist, an NMDA modulator, an NMDA antagonist, an aldehyde dehydrogenase inhibitor, a phenethylamine, and a tryptamine, including pharmaceutically acceptable salts thereof.

In some aspects are disclosed methods of treating a behavioral addiction in a patient diagnosed therewith, comprising providing an entactogen-assisted psychotherapy (EAP) regimen, wherein the EAP regimen comprises one or more drug-assisted therapy sessions with an entactogen, and one or more non-drug therapy sessions. In some embodiments, the behavioral addiction is any of gambling disorder, gaming disorder, compulsive sexual behavioral disorder, compulsive buying-shopping disorder, technology addiction, kleptomania, internet addiction, pornography addiction, technology addiction, binge eating disorder, workaholism, perfectionism, pathological skin picking, exercise addiction, excessive tanning, tattoo addiction, masturbation addiction, sex addiction, and love addiction.

In some aspects are disclosed methods of treating a comorbidity in a patient diagnosed with a condition, comprising providing an entactogen-assisted psychotherapy (EAP) regimen, wherein the EAP regimen comprises one or more drug-assisted therapy sessions with an entactogen, and one or more non-drug therapy sessions. In some embodiments, the comorbidity is a comorbid mental health disorder in a patient diagnosed with AUD, and wherein the comorbid mental health disorder is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, a phobia disorder, a binge disorder, body dysmorphic disorder, a mood disorder related to another health condition, a disruptive behavior disorder, an eating disorders, an impulse control disorder, obsessive compulsive disorder (OCD), attention deficit hyperactivity disorder (ADHD), a personality disorder, an attachment disorder, a dissociative disorder, a substance-related disorder including substance use disorder (SUD), and a behavioral addiction. The foregoing has outlined broadly and in summary certain pertinent features of the disclosure so the detailed description of the invention may be better understood, and so the present contribution to the art more fully appreciated. Hence, this summary is to be considered as a brief and general synopsis of only some of the aspects and embodiments of the invention, is provided solely for the benefit and convenience of the reader, shall not limit in any manner the scope, or range of equivalents, to which the claims are lawfully entitled. Certain specific aspects and embodiments of the disclosed compositions and methods are described. It will be appreciated by those in the art that all such disclosed specific compositions and methods are only exemplary, and may be readily utilized as a basis for modifying or designing other compositions and methods for carrying out the same and similar purposes. Such equivalent compositions and methods will be appreciated to be also within the scope and spirit of the invention as claimed.

It will be appreciated that the headings within this document are utilized only to expedite its review by a reader. They should not be construed as limiting the invention in any manner.

### BRIEF SUMMARY OF THE DRAWINGS

To further clarify various aspects of the disclosure, a more particular description thereof is rendered by reference to certain exemplary aspects which are illustrated in the included Figures. It should be understood and appreciated that the Figures depict only illustrated aspects of certain exemplary embodiments of the invention and therefore are not to be considered limiting of its scope. They are simply provided as qualitative illustrations of the concepts of the present invention.

Certain aspects and embodiments of the invention are therefore further described and explained with additional specificity and detail, but still by way of example only, with reference to the accompanying Figures in which:

FIG. 1 illustrates drinking behavior outcomes for patients undergoing a community alcohol detoxification, as measured by Timeline Follow-Back (TLFB), up to nine months (reproduced from Sessa et al. 2020). Specifically, individual drinking behavior is displayed as units of alcohol consumed per week (n=12). In the case of additional detoxification TLFB results are expressed as an extrapolation of drinking behavior from the point of their initial relapse (dotted lines indicate the point). Incomplete lines indicate drop-out. This population of patients (“Outcomes patients”) did not receive MDMA-assisted therapy.

FIG. 2 illustrates drinking behavior outcomes for the Outcomes patients undergoing a community alcohol detoxification (as measured by TLFB) up to nine months (reproduced from Sessa et al. 2020). Specifically, the average weekly alcohol consumption for all 12 patients up to nine months post-detox as shown in FIG. 1 is illustrated.

FIG. 3 illustrates the results of the PHQ-9 assessment of outcome measures (see TABLE 2 and discussion below), in table and graph form, demonstrating high levels of depression among the population of Outcomes patients, levels of depression that remained significantly high throughout the nine months of the study (reproduced from Sessa et al. 2020).

FIG. 4 illustrates the results of the PHQ-9 assessment of outcome measures (see TABLE 2 and discussion below), in table and graph form, demonstrating high levels of anxiety among the population of Outcomes patients, levels of anxiety that remained significantly high throughout the nine months of the study (reproduced from Sessa et al. 2020).

FIG. 5 illustrates the results of the SF-36 outcome measure (see TABLE 2 and discussion below), in table and graph form, suggesting that for those who provided results to nine months, despite the majority of patients relapsing to high levels of drinking by three months (and continuing high rates of drinking, even if they had second or third detoxes) their overall level of psychosocial functioning appears to have improved over the course of nine months (although an incomplete dataset at nine months is noted) (reproduced from Sessa et al. 2020).

FIG. 6 illustrates the results of the Severity of Alcohol Dependence Questionnaire (SAD-Q) measure of alcohol dependency, from the patients who received MDMA-assisted therapy as in Example 8. A score of 31 or higher indicates severe alcohol dependence, a score of 16-30 indicates moderate dependence (middle band, between the two horizontal lines), anything lower than 16 indicates mild dependence (bottom band, below the first horizontal line). Also illustrated are the results of the Short Inventory of Problems for Alcohol (SIP), a 15 question measure of self-noted consequences of drinking. Both observed at screening. Four SAD-Q questions were unanswered, in which case, mean substitution was applied using the average row value for the relevant time period and participant. B05, B16, B20 and B21 had one question missing each.

FIG. 7 illustrates pooled data of Blood Pressure, Temperature, Heart Rate, Observed Drug Effects, and Subjective Units of Distress (SUDS) observed over the duration of the MDMA sessions in patients who underwent MDMA-assisted therapy in the study of Example 8. SUDS and Drug Effects observed over 8 hours, physiological data observed over 6 hours following dosing. Mean data for each session is used, except in the case of missing data, where available session data is applied. Error bars (where applied) indicate+/−SEM.

FIG. 8 illustrates the results of the TLFB, which assesses drinking behavior prior to, and following the study of Example 8. Data is collected daily by self-reporting and reviewed at one month prior to detox, immediately following detox and at 1, 3, 6, and 9 months follow-up. A full dataset was not available for three of the participants. One participant dropped out of the study at three months and two patients failed to provide data at the nine month follow-up. Two participants had a second detox since starting the study. For these participants, TLFB drinking behavior data was carried forward from the point of drinking levels pre-second detox.

FIG. 9 illustrates the results of the Profile of Mood States (POMS), individual composite scores of mood disturbance observed daily over a week following dosing, obtained from the patients in the study of Example 8. Mean data shown for both MDMA sessions. In the case of absent data for either session the available data for the remaining session is used.

FIG. 10 illustrates the results of General Anxiety Disorder 7 (GAD-7) and Patient Health Questionnaire 9 (PHQ-9) obtained from the patients in the study of Example 8. These are self-report scales for anxiety and depression, respectively (as shown for Outcomes patients in FIGS. 3 and 4). Recording was at screening, baseline, week 10, then 3, 6, and 9 months follow-up. Greater scores report indication of heightened anxiety/depression. Error bars indicate+/−SEM.

FIG. 11 illustrates TLFB showing the percentage of patients consuming more than the 14 recommended daily units of alcohol, and comparing the results of the patients in the study of Example 8 with the results of the Outcomes patients reported in Sessa et al. 2020.

FIG. 12 illustrates an embodiment of the invention comprising a therapy protocol useful in the treatment of AUD, such as non-physically dependent (harmful use) AUD, and other SUDs.

FIG. 13 illustrates an embodiment of the invention comprising a therapy protocol useful in the treatment of AUD, such as physically dependent (dependent use) AUD, and other SUDs.

FIG. 14 illustrates an exemplary detoxification course, according to some embodiments of the invention.

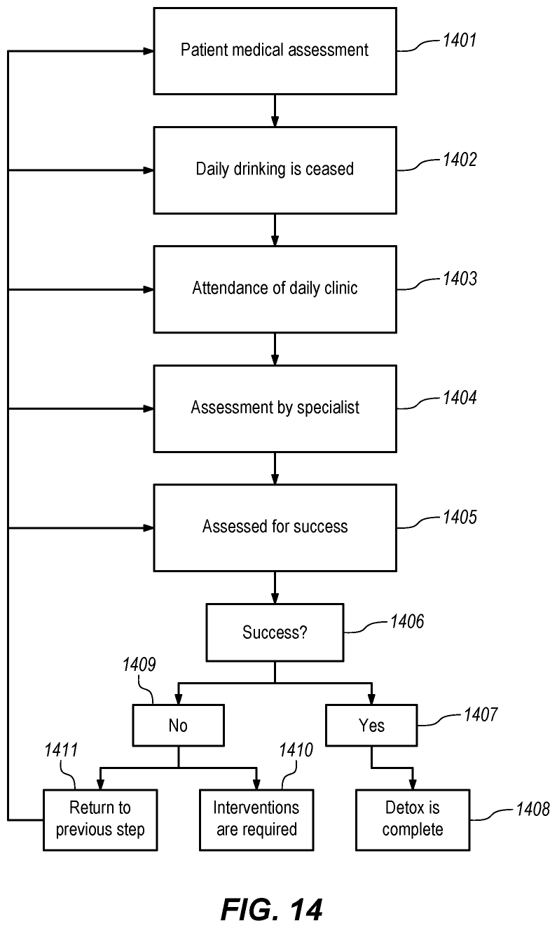

FIG. 15 illustrates an overview of an exemplary treatment protocol useful in treating a patient diagnosed with AUD or another SUD, according to some embodiments of the invention.

FIG. 16 illustrates an exemplary treatment protocol useful in treating a patient diagnosed with AUD or another SUD, according to some embodiments of the invention.

FIG. 17: illustrates the study procedure for the 13-week study course, including assessments completed, according to some embodiments of the invention.

### DETAILED DESCRIPTION OF THE INVENTION

Alcohol use disorder (AUD) (DSM-5, APA 2013), which includes what are sometimes referred to as alcohol abuse, alcohol dependence, alcohol addiction, and colloquially “alcoholism,” is a psychiatric disorder in which individuals present with a cluster of symptoms of varying severity, that cause psychological and physical harms and social dysfunction due to the use of alcohol. Although drinking alcohol is a widely socially acceptable behavior and many people consume alcohol without experiencing any problems, a growing number of people drink in a harmful manner. Approximately 24% of the adult population of England (where certain research underlying the present invention has taken place, and from where the inventors hail) consume alcohol in a way that is potentially or actually harmful, i.e., “harmful use” (McManus et al 2009).

Physical dependence to alcohol (“alcohol dependence” or “dependent use”) is a less common, more severe form of AUD. In the UK, the prevalence of physical alcohol dependence is about 4%, with 6% of men and 2% of women meeting criteria for this psychiatric disorder. Typically, alcohol dependence is characterized by: often serious withdrawal symptoms on cessation of alcohol, or drinking to avoid withdrawal symptoms, tolerance and the persistent desire to drink, and continuing drinking despite negative consequences (NICE Guidelines on AUD, 2011).

“Harmful use” of alcohol, by contrast, is defined in the World Health Organization (WHO)'s International Classification of Diseases, 10th Revision (The ICD-10 Classification of Mental and Behavioral Disorders) as: “[A] pattern of psychoactive substance use that is causing damage to health. The damage may be physical (e.g., hepatitis) or mental (e.g., depressive episodes secondary to heavy alcohol intake). Harmful use commonly, but not invariably, has adverse social consequences; social consequences in themselves, however, are not sufficient to justify a diagnosis of harmful use” (ICD-10; WHO, 1992).

From a clinical perspective, it can be helpful to separate this group of “harmful use” drinkers from “dependent use” drinkers with more severe and enduring physical dependence upon alcohol that may require medical support (e.g., detoxification with benzodiazepine assistance to avoid a severe physical withdrawal syndrome with associated risk of seizures).

In 2013, almost 200,000 items of medication were prescribed (in a primary care setting or NHS hospital) for the treatment of AUD and dispensed in the community. The Net Ingredient Cost (NIC) of these prescription items in 2013 was £3.13 million. In 2012, there were 6,490 alcohol-related deaths (HSCIC 2014). Taking into account alcohol related health disorders and disease, crime and antisocial behavior, accidents, loss of productivity in the workplace, and domestic problems, the Department of Health estimates that alcohol misuse is now costing around £20 billion a year in England alone (HM Government 2012).

It is difficult to find agreement about outcomes of alcohol treatments because there are many different sorts of treatments, there are overlaps between treatments, and there are large differences between the sorts of patients, severity of disease, presentation of patients, and multiple confounding psychosocial factors. Nonetheless, the outcomes of current treatments are generally considered relatively poor.

Licensed pharmacological options for treating AUD include acamprosate, disulfiram, naltrexone, and nalmefene. The U.S. Food and Drug Administration (FDA) has approved only disulfiram, naltrexone, and acamprosate for the treatment of AUD, and the European Medicines Agency (EMA) has approved, in addition, nalmefene. Acamprosate, a glutamate antagonist, reduces NMDA activity associated with alcohol withdrawal and reduces relapse rates (Rosner et al. 2010). Disulfiram is used as an agent to deter relapse by preventing the elimination of acetaldehyde, which results in an unpleasant physical reaction if alcohol is used together (Krampe et al. 2006). Naltrexone is a competitive opioid antagonist which decreases cravings from alcohol (Soyka and Rosner 2008). Nalmefene is an opioid antagonist, like naltrexone but with a longer half-life and lower risk of toxicity than naltrexone. This can be given to patients while still drinking in order to reduce cravings (Paille and Martini 2014). Benzodiazepines are commonly prescribed as a time-limited course as part of alcohol detoxification program (Lingford-Hughes et al. 2012).

The efficacy of each of these above medications is only modest. Thus, there is still a high medical need to find more effective treatments for AUD. Numerous other pharmacological agents have been tested, although none have shown marked promise alone. These include opioid antagonists (e.g., nalmefene), CB₁ antagonists (e.g., rimonabant), CRH₁ receptor antagonists (e.g., verucerfont, pexacerfont), NK_{1R} antagonists (e.g., tradipitant), OTR agonists (e.g., oxytocin), GABA agents (e.g., topiramate, baclofen, benzodiazepines), voltage-gated sodium channel inhibitors (e.g., oxacarbazepine, valproic acid, zonisamide), voltage-dependent calcium channel agonists (e.g., gabapentin, pregabalin), α₇ nicotinic acetylcholine receptor agonists (e.g., varenicline), 5-HT₃ antagonists (e.g., ondansetron), 5-HT_{1A} receptor partial agonists (e.g., aripiprazole), 5-HT_{2A} receptor antagonists (e.g., quetiapine, olanzapine, mirtazapine), 5-HT reuptake inhibitors (e.g., trazodone), SERT inhibitors (e.g., duloxetine), α₁ adrenoreceptor antagonists (e.g., doxazosin, prazosin), glucocorticoid receptor antagonists (e.g., mifepristone), α₁ adrenoreceptor agonists (e.g., guanfacine), AChE inhibitors (e.g., citicoline), a dopamine D₂ receptor antagonist (e.g., tiapride), and α₂ adrenoreceptor agonists (e.g., clonidine), among others.

In terms of psychosocial interventions, the best approaches are those that foster good relationships and are delivered by the most experienced, highly trained therapists. However, the efficacy of current available treatments is far from satisfactory, with high rates of relapse. A systematic review of 361 controlled studies of treatments for alcohol dependence in 2002 identified 46 possible interventions (Miller and Wilbourne 2002). Interventions were ranked according to rates of abstinence achieved. The brief intervention approach ranked highest and motivational enhancement therapy, a counselling approach that helps individuals resolve their ambivalence about engaging in treatment and stopping their drug use, ranked second. The motivational enhancement approach aims to evoke rapid and internally motivated change, rather than guide the patient stepwise through the recovery process. Pharmacotherapy with acamprosate and the opiate antagonist naltrexone ranked third and fourth respectively. The lowest ranked approaches were designed to educate, confront, shock, or foster insight regarding the nature of alcoholism.

A large three year follow-up study, project MATCH (Project MATCH Research Group 1997a), identified that 12-step facilitation (TSF), Motivational Enhancement Therapy (MET), and Cognitive-Behavioral Coping Skills Therapy (CBT) are in general equally effective. At three years, 36% of the TSF clients were abstinent, compared with 27% of MET and 24% of CBT clients. A more recent review, the COMBINE Project (Anton et al. 2006), was designed to evaluate the efficacy of two relapse prevention medications in various combinations with behavioral treatment. Results indicated that both naltrexone and acamprosate showed only minor positive effects in relapse prevention, and only when used in conjunction with psychosocial interventions. A similar UK-based study, ‘UKATT’ (UKATT Research Team 2005), identified that Social Behavior and Network Therapy (SBNT) and MET produced similar improvements in drinking and general functioning, with clients all reporting that total alcohol consumption had decreased by less than 50% at 12 months, with no significant differences in effectiveness between groups.

In recent years, mindfulness techniques, originally derived from Vipassana meditation, which encourage awareness and acceptance of thoughts, feelings, and bodily sensations, have been explored as a potential approach for treating AUD (Marcus and Zgierska 2009). Mindfulness promotes nonjudgmental acceptance of moment-to-moment thoughts. This helps the person in recovery interrupt the tendency to respond to stress with alcohol use and not to react automatically to cravings (Shapiro 2006, Hsu et al. 2008). Although mindfulness shows promise as an adjunctive treatment to help individuals endure craving, it has limited evidence of effectiveness, and many individuals need further cognitive and behavioral skills to cope with high risk situations for relapse.

3,4-Methylenedioxymethamphetamine (MDMA) was first synthesized in 1912 by chemists at the pharmaceutical company Merck in Germany, as an intermediate in the synthesis of a compound, hydrastinine, that had been developed as a regulator of bleeding. In 1914 Merck was issued German Patent Nos. DE274350 and DE279194 for that work. MDMA at the time was referred to as “methylsafrylamin.” MDMA however was not tested in humans until 1959. Starting in 1977, following its introduction by Alexander (“Sasha”) Shulgin to psychiatrist Leo Zeff (the “Secret Chief”) and subsequent widespread promotion by Zeff as an adjunct to psychotherapy (as “Adam,” because he believed it returned one to a state of primordial innocence), MDMA became used therapeutically across the United States. Starting around 1979, non-clinical use of MDMA, now often called “ecstasy,” began to grow, and came to the attention of U.S. authorities, including the Drug Enforcement Administration (DEA).

In 1985, because of this attention, MDMA was placed by emergency ruling in Schedule I of the Controlled Substances Act (CSA)—the most restrictive class of drugs, reserved for substances that “have no currently accepted medical use,” “a lack of accepted safety for use under medical supervision, and a high potential for abuse”—and was similarly scheduled in other countries around the world. This resulted in no research being conducted into the potential therapeutic applications of MDMA between 1985 and 2000.

However, starting in 1986, the Multidisciplinary Association for Psychedelic Studies (MAPS) began funding animal toxicity studies and human safety studies at Stanford University and Johns Hopkins University. In 1990, the first FDA approved, double-blind, placebo-controlled Phase I dose-response safety study of MDMA was published by Charles Grob, working with MAPS. In 2004, the first Phase 2 clinical trial of MDMA-assisted therapy for PTSD was approved by the FDA, sponsored by MAPS. By the time of the first priority filing of this application, MDMA had been included in over 5,000 academic articles and administered to over 1,100 human volunteers. Twelve clinical trials had been completed globally under sponsorship of MAPS, investigating MDMA-assisted therapy for PTSD, anxiety related to advanced-stage illness, social anxiety in autistic adults, and there was a study of healthy subjects via an MDMA therapist training program.

One Phase 1 study, “Open-Label Proof of Concept Feasibility Study to Explore the Safety, Tolerability and Potential Role of MDMA-assisted therapy for the Treatment of Detoxified Patients With Alcohol Use Disorder,” sponsored by Imperial College London, was the first to initiate an exploration that was expanded and built upon until disclosed herein (see also Sessa et al. 2021, which was still forthcoming at the time of the filing of the priority documents of which this disclosure claims the benefit). The concept was that of the inventors hereof.

Chemically, MDMA is a ring-substituted phenethylamine. It exerts its effects primarily through promoting raised levels of monoamine neurotransmitters in the brain, in particular serotonin, but also dopamine and noradrenaline. Increased activity at 5-HT_{1A} and 5-HT_{1B} receptors reduces feelings of depression and anxiety, reduces the amygdala fear response and increases levels of self-confidence (Brunner 1997). Furthermore, the effect of raised serotonin at 5-HT_{2A} receptors provides alterations in the perceptions of meanings and facilitates new ways of thinking about old experiences (Nash et al. 1994). The effect of increased dopamine and noradrenaline is to raise levels of arousal and awareness, which increases sense of readiness and improves recall of state-dependent memories of stressful events (Cozzi 1999). Alongside this increase in arousal there is also a paradoxical increase in relaxation mediated by the effects of MDMA at alpha-2 receptors (Lavelle 1999). MDMA also has been shown to facilitate the release of oxytocin, which is the hormone associated with bonding and increased levels of empathy and closeness (Thompson 2007).

MDMA produces elevated mood, increased sociability and feelings of closeness to others, slight alterations in perception that facilitate imagination and memory (Harris et al. 2002), greater compassion, feelings of sociability, closeness and increased empathy for others and themselves (Hysek et al. 2013). MDMA reduces activity in the amygdala (Gamma et al. 2000) which results in users experiencing a reduced fear response, and greater activity in the reward pathways in the Ventral Tegmental Area (VTA) in comparison to placebo, associated with less activity in the left amygdala after viewing angry faces, suggesting less reactivity to anger and improved prosocial behavior. Subjects receiving MDMA showed a better ability to detect positive facial expressions and a greater difficulty detecting negative facial expressions (Hysek et al. 2012a).

These effects of MDMA give rise to its description as an “empathogen” (meaning “generating a state of empathy,” and independently suggested in 1983-84 by the psychologist and psychopharmacologist Ralph Metzner and the Purdue University professor of pharmacology and medicinal chemistry David Nichols) and an “entactogen” (subsequently coined by Nichols in 1986, and meaning “to touch within”) (Holland 2001 at 182 n.2).

Taken together, these changes in social cognition, interpersonal closeness, communication, and brain activity may influence the outcome of psychotherapeutic treatments for psychological disorders (Jerome et al. 2013). Nevertheless, the potential of MDMA to enhance the psychotherapeutic processes in the treatment of AUD was not before tested in a controlled clinical setting until the work of the inventors hereof.

The use of MDMA-assisted therapy has been explored as a treatment for post-traumatic stress disorder (PTSD) since the 1980s (Greer 1986, Sessa 2012). Long-term follow-up data from the first completed trial of MDMA-assisted therapy for chronic, treatment-resistant PTSD found statistically and clinically-significant gains in symptom relief with no subjects reporting harm (Mithoefer et al. 2010; Mithoefer et al. 2013). Another study in 12 subjects (Oehen 2013) showed significant self-reported improvement, with an effect size of 1.1 similar to Mithoefer et al. 2010. Findings from a recent randomized, double-blind, placebo-controlled, multi-site phase 3 clinical trial demonstrated that, compared to therapy with inactive placebo, MDMA-assisted therapy is highly efficacious in individuals with severe PTSD, and treatment is safe and well-tolerated, even in those with comorbidities (Mitchell 2021). Studies have also demonstrated potential for MDMA to address other difficult-to-treat mental health conditions, including obsessive compulsive disorder (OCD), phobias, eating disorders, depression, end-of-life anxiety, and social anxiety.

How the primary actions of MDMA could treat AUD has not before been established or well-studied. In previously proposing a feasibility study, an inventor hereof postulated that MDMA-assisted therapy may influence the outcome of psychotherapeutic treatments for AUD (Sessa 2017). However, evidence that would support an expectation that MDMA-assisted therapy indeed would work for AUD did not then exist.

Since the earliest days of psychedelic research in the 1950s, AUD has been a target for psychedelic-assisted psychotherapy—the theory being that an intense, drug-induced spiritual or mystical peak experience could be used as a method of inducing sobriety (Sessa, 2017). LSD-assisted psychotherapy was explored with varying rates of success, and heterogeneity between studies. Early uncontrolled studies showed abstinence rates of 30-50% (McLean et al., 1961; Kurland et al., 1967; Ditman & Bailey, 1967; Rydzynski et al., 1968).

Drug-assisted psychotherapies with “classical” psychedelic compounds, such as LSD and psilocybin, utilize the induced subjective mystical and spiritual effects of the psychedelic experience. Researchers have found that the depth of this experience is strongly associated with maintained abstinence from substance use (Bogenschutz 2015, Johnson et al. 2014, Kruspitsky 1997). In contrast, use of MDMA is generally not associated with a mystical experience. LSD has been found to induce mystical experiences of significantly greater intensity relative to MDMA (Holze, 2019), as determined by the 5 Dimensions of Altered States of Consciousness scale (5D-ASC) (Dittrich, 1998) and the Mystical Experience Questionnaire (MEQ) (Barrett, 2015).

Additionally, not all patients are able to tolerate the classical psychedelic experience, or even the idea of a treatment involving these experiences, and compliance is a critical part of addiction therapy. MDMA is generally more easily tolerated than the classical psychedelics, with less perceptually disturbing effects compared to LSD and psilocybin. But whether this in fact offers an alternative opportunity for enhanced psychotherapy was not before demonstrated. Indeed, given that the depth of the mystical experience is strongly associated with maintained abstinence from substance use, it would have been logical to presume that MDMA-assisted therapy for substance use disorders, including AUD, would have less success in treating patients than LSD- or psilocybin-assisted therapy (cf McCulloch et al. 2021).

Applicant herein discloses compositions of entactogens, for example MDMA, and their use to treat AUD, including both “dependent use” AUD and “harmful use” AUD. Using the disclosed compositions and methods, the psychotherapeutic processes involved in the treatment of AUD are not only enhanced and intensified, but are improved in novel, unexpected, and surprising ways. As described below, the unique effects of entactogens such as MDMA, such as when given in a psychotherapeutic context disclosed and using the disclosed compositions and methods, reduce avoidance of emotionally distressing thoughts, images, or memories while increasing empathy for the self and others. Moreover, as taught herein, the disclosed compositions and methods can lead to reductions in alcohol cravings, problematic consumption, and rates of relapse.

The disclosed compositions and methods can also address symptoms of other conditions that are frequently comorbid with substance abuse. For instance, Applicant also discloses compositions of entactogens, such as MDMA, to treat comorbid disorders, including comorbid substance use disorders (SUDs) and comorbid behavioral addictions (BAs). Using the disclosed compositions and methods, the psychotherapeutic processes involved in the treatment of SUDs and BAs also are not only enhanced and intensified, but are improved in novel, unexpected, and surprising ways. As taught herein, the disclosed compositions and methods can lead to a reduction in substance use, a reduction in craving for a substance, a promotion of abstinence from substance use, a prevention of relapse into substance use, and/or an improvement of at least one symptom of substance use disorder; as well as a reduction in the frequency of a compulsive or problematic behavior, a reduction in drive or urge to engage in a compulsive or problematic behavior, prolonged abstinence from a compulsive or problematic behavior, a prevention of relapse into a compulsive or problematic behavior, a reduction in severity of symptoms of a compulsive or problematic behavior, a reduction in the time spent on a compulsive or problematic behavior; an increase in quality of life, including subjective sleep; and an increase in psychosocial functioning.

For these reasons and others, the invention represents a significant step toward solving the serious clinical, social, and personal problems caused by AUD and by comorbid SUDs and BAs.

Among the various aspects of the disclosure are compositions of entactogens, such as MDMA, and methods of their use in EAP and MDMA-assisted therapy for the treatment of patients with AUD, including physically dependent alcohol use disorder (“dependent use” or “alcohol addiction”) and non-physically dependent alcohol use disorder (“harmful use”); as well as for other SUDs, such as opioid use disorder, nicotine dependence and tobacco use disorder, sedative, hypnotic, and anxiolytic use disorder; and stimulant use disorder; and BAs, including gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction.

Entactogen-assisted psychotherapy (EAP), including MDMA-assisted therapy, as described in the disclosed methods, may involve the careful screening of patients, such as through specific inclusion and exclusion criteria on which the success of treatment may depend. The therapeutic program involves both non-entactogen and entactogen-assisted sessions which are all delivered by trained healthcare professionals, in a safe and supportive setting. Visits involve entactogen dosing, including MDMA which typically lasts 6-8 hours during which patients are carefully monitored. EAP using entactogens other than MDMA may last longer, or in some embodiments, preferably shorter, depending on the duration of action of the drug, or the effects of the drug. In such embodiments, where a drug-assisted psychotherapy session lasts longer or shorter than a session with MDMA, the exemplary treatment protocols will be modified accordingly, based on the teachings herein and the general knowledge in the art. In some embodiments (equivalently herein, and used simply as shorthand, “in embodiments”), a drug-assisted psychotherapy session with an entactogen other than MDMA lasts the same or substantially the same length of time as one with MDMA. In some embodiments, a drug-assisted psychotherapy session with an entactogen other than MDMA lasts a longer length of time as one with MDMA. In some embodiments, a drug-assisted psychotherapy session with an entactogen other than MDMA lasts a shorter length of time as one with MDMA. In some embodiments, a drug-assisted psychotherapy session with an entactogen other than MDMA lasts less than 1 hour, about 1 hour, about 2 hours, about 3 hours, about 4 hours, about 5 hours, about 6 hours, about 7 hours, about 8 hours, or greater than 8 hours.

Using the disclosed compositions and methods, EAP and MDMA-assisted therapy can be safely delivered, can be well tolerated, and can enhance and intensify the psychotherapeutic processes in the treatment of patients with AUD, other SUDs, BAs, and comorbid SUDs and BAs.

Enactogens, including MDMA, given in the psychotherapeutic context described by Applicant, can reduce avoidance of emotionally distressing thoughts, images, or memories of alcohol and substance misuse, while increasing empathy for the self and others. Entactogens like MDMA can also address symptoms of other conditions that are frequently comorbid with substance abuse, particularly those symptoms associated with a history of psychological trauma. Further, patients treated with the disclosed compositions and methods can reduce their alcohol consumption and substance use over the long term, resulting in reduced rates of relapse, and producing benefits not only to the individual patients treated, but to their friends, family, and society at large.

As disclosed by Applicant and detailed below, the disclosed methods of EAP and MDMA-assisted therapy are superior methods of treating patients with AUD, SUDs, and BAs, including comorbid SUDs and BAs, with numerous significant improvements over prior art methods. As shown below, EAP and MDMA-assisted therapy have significant advantages over standard psychotherapy or other existing treatments for AUD, SUDs, and BAs, offering patients a potential new approach to many devastating, chronic, and relapsing conditions.

While the invention is described in terms of particular embodiments and applications, it is not intended that these descriptions in any way limit its scope to any such embodiments and applications, and it will be understood that many modifications, substitutions, changes, and variations in the described embodiments, applications, and details of the invention illustrated herein can be made by those skilled in the art without departing from the spirit of the invention, or the scope of the invention as recited in the appended claims.

### A. DEFINITIONS AND TERMS

Terminology used herein is for describing embodiments and is not intended to be limiting.

As used herein, the singular forms “a,” “an,” “and,” “the,” and “said” are intended to mean that there are one or more of the elements, and hence include plural referents, unless the content and context clearly dictate otherwise. Thus, for example, a reference to “an excipient” may include a single such excipient, or a combination of two or more excipients.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

Unless otherwise indicated, all numbers expressing quantities used to describe and claim certain embodiments of the invention are to be understood as being modified in some instances by the term “about.” Accordingly, in some embodiments, the numerical parameters set forth in the description and claims are approximations that can vary depending upon the desired properties sought to be obtained by a particular embodiment. In some embodiments, “about” refers to plus or minus five percent (±5%) of the recited unit of measure. The term “substantially,” where it is applied to modify a parameter or characteristic herein, will be read in the context of the invention and in light of the knowledge in the art to provide certainty, e.g., by using a standard that is recognized in the art for measuring the meaning of “substantially” as a term of degree, or by ascertaining the scope as would one of skill.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless defined otherwise, all technical and scientific terms herein have the meaning as commonly understood by one of ordinary skill in the art to which this invention belongs. Further definitions that may assist a reader to understand the disclosed embodiments are throughout.

“Psychosocial or behavioral therapy” may refer to, but is not limited to, therapies other than the EAP or MDMA-assisted therapy of the invention, e.g., 12-step facilitation therapy (e.g., NIAAA, Project MATCH Monograph Series. Volume 1, NIH Publication No. 94-3722, (1995) reprinted 1999), cognitive behavioral therapy (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2): 354-59; or in Case Reports in Psychiatry, Vol. 2012, Article ID 731638), community reinforcement approach based therapy (e.g., as described in Drug Alcohol Depend 2004; 74:1-13), motivational interviewing based therapy (e.g., as described in J. Consul. Clin. Psychol. 2001; 69(5): 858-62), motivational enhancement based therapy (e.g., as described in Drug Alcohol Depend 2007, 91:97-101) or meditation based therapy, such as transcendental meditation based therapy (e.g., as described in Addiction 2004; 99(7):862-874 or J. Consul. Clin. Psychol. 2000; 68(3): 515-52).

“Standardized psychological treatment” or “standardized psychological support” refers to standardized sessions, which may be, e.g., once a week, twice a week, once every two weeks, once a month, and the like, and in particular may include psychological treatment, support, or other counseling focused on the SUD or BA being treated, such as for AUD, on alcohol consumption.

“Impulsivity” refers to a predisposition toward rapid, unplanned reactions to internal or external stimuli with diminished regard to the negative consequences of these reactions to the impulsive individual or others” (Moeller et al. Am J Psych. 2001. 158: 1783-93).

“Mental health disorder,” “mental illness,” or “psychiatric disorder” refer to a disease condition in a mammal, and preferably in a human, that generally involves negative changes in emotion, mood, thinking, and/or behavior. In some embodiments, “mental health disorder,” “mental illness,” or “psychiatric disorder” refer to a behavioral or mental pattern that causes distress or impairment of personal functioning (Bolton, 2008). Mental health disorders include post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, dissociative disorders, and substance-related disorders, including alcohol or drug abuse or dependence disorders like AUD and SUDs (see Merck Manual of Diagnosis & Therapy, 20th Ed. (2018)). Mental health disorders also may be referred to herein as “mental health conditions.”

“Behavioral addictions” refer to non-substance addictions that are characterized by an inability to resist an urge to perform a behavior resulting in actions that are harmful to oneself or others (Grant, Schreiber, & Odlaug, 2013). Although behavioral addictions share some at least surface similarities with alcohol and substance abuse, they are separate and distinct categories and classifications. In some embodiments, a behavioral addiction will be any of gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction. In some embodiments, a behavioral addiction will be any of pathological gambling, kleptomania, pyromania, compulsive buying, compulsive sexual behavior, Internet addiction, pornography, and binge eating disorder. In some embodiments, the behavioral addiction will be pathological gambling or gambling disorder.

“MDMA” refers to 3,4-methylenedioxymethamphetamine, i.e., 1-(1,3-benzodioxol-5-yl)-N-methylpropan-2-amine (IUPAC), whether as a racemate, as pure or substantially pure individual enantiomers, or as an enantiomerically enriched mixture in any amount greater than 0%, and whether in salt or freebase form, and including polymorphs. “MDMA” as used herein thus will be understood to encompass all salt forms of MDMA, such as MDMA hydrochloride salt (3,4-MDMA HCl, available from, e.g., Cayman Chemical Co., Ann Arbor, MI, with purity ≥98%, as Item No. 13971, NSC 168383, CAS No. 64057-70-1). “MDMA” will include the hydrochloride salt in all hydrated crystalline forms, such as quarter-hydrate, hemihydrate, three-quarter hydrate, and monohydrate (see, e.g., Shulgin 1986, and references cited). It includes MDMA HCl “forms I-III” (see Nair 2021). It includes MDMA base, generally a colourless oil insoluble in water. It will also include MDMA as a racemic mixture (i.e., S,R(±)-3,4-methylenedioxymethamphetamine) (CAS 69610-10-2), as an enantiomerically enriched mixture (of whatever proportions), or as individual enantiomers (i.e., pure or substantially pure R-MDMA or S-MDMA). Both enantiomers function as monoamine releasers (Hiramatsu and Cho, 1990; Johnson et al. 1986; Setola et al. 2003), with S-MDMA being a more potent dopamine releaser, and preclinical evidence suggesting that R-MDMA may provide an improved therapeutic index, maintaining the therapeutic effects of (±)-MDMA with a reduced side effect profile (see, e.g., Anderson et al. 1978; Curry 2018; Pitts 2018). “MDMA” thus may refer to racemic MDMA or to enantiomerically enriched compositions (e.g., to balance the individual therapeutic effects of S-MDMA and R-MDMA, or to minimize certain negative effects, such as the suggested greater neurotoxicity of the S-(+) enantiomer) or compositions of individual MDMA enantiomers. It also includes salt forms of individual enantiomers, e.g., S-MDMA HCl (CAS 69558-32-3) and R-MDMA HCl (CAS 69558-31-2), or mixtures in any portions thereof. In enantiomeric or enantiomerically enriched embodiments, the dosage may be adjusted accordingly based on the known differences in potency, and may be done so based on the teachings herein in combination with the general knowledge in the art.

Accordingly, all reference to “MDMA” (unless stated or necessarily implied otherwise, or where needed to provide technical effect) will be understood to include single enantiomers, enriched mixtures, and racemic mixtures, as well as all pharmaceutically acceptable salts, hydrates, solvates, co-crystals, polymorphs, prodrugs (e.g., as defined below), or metabolites thereof (e.g., 3,4-methylenedioxyamphetamine (MDA), 4-hydroxy-3-methoxymethamphetamine (HMMA), 4-hydroxy-3-methoxyamphetamine (HMA), 3,4-dihydroxyamphetamine (HHA), and 3,4-dihydroxymethamphetamine (HHMA)), and combinations thereof.

As used herein, the terms “subject,” “user,”. “patient,” and “individual” are used interchangeably, and refer to any mammal although preferably a human. In some embodiments, the terms “subject,” “user,” “patient,” and “individual” refer to any mammal, although preferably a human, who has an indication for which a compound, composition, or method described herein may be efficacious, or who otherwise may benefit by the invention. In general, all of the disclosed compounds, compositions, and methods will be appreciated to work for all individuals, although individual variation is to be expected, and will be understood. In embodiments, as used herein, the terms “subject,” “user,” “patient,” and “individual” includes one who has a mental health disorder, or a condition related to a mental health disorder for which similar treatment may be efficacious; in embodiments, unless otherwise specified, an “alcohol use disorder” shall refer to both “harmful use” and “dependent use” AUD. When a “subject,” “user,” “patient,” or “individual” is participating in a course of therapy as described, the term “participant” also may be used. These terms also shall refer to patients in need of treatment for such a disorder, persons predisposed to such a disorder, and subjects whether or not diagnosed with such a disorder. Moreover, these terms shall likewise refer to persons who have received treatment or therapy for a mental health disorder, are currently receiving therapy or treatment for a mental health disorder, or who may receive therapy or treatment for such a disorder in the future. In embodiments, the disclosed methods also can be used to improve mental health and improve psychological functioning in non-disease states, i.e., in an individual without a diagnosed mental health disorder, or specific symptoms thereof.

In general, all of the disclosed compositions and methods will be appreciated to work for all individuals, although individual variation is to be expected, and will be understood. Where there is variation between individuals, modification to the compositions and methods will be understood based on the teachings herein in combination with the general knowledge of the art. In some instances, certain personalized approaches (i.e., “personalized” or “precision” medicine) may be utilized, based on individual characteristics, including drug metabolism (e.g., CYP2D6 or CYP3A4) or individual genetic variation. The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations. In one embodiment, the genetic variation is a genetic variation in metabotropic glutamate receptor type 5 (mGluR5), which has been implicated in mood and anxiety symptoms in humans, as well as playing a direct role in the pathogenesis of SUDs, e.g., AUD, showing involvement in the development of behavioral sensitization towards ethanol in animal models. In another embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression, PTSD, and anxiety disorders (Yehuda 2016, Bierer 2020).

In one embodiment, the genetic variation is a genetic variation in one or more cytochrome P450 (CYP or CYP450) enzymes that affects drug metabolism, including metabolism of a composition of the invention, and including CYP1A2, CYP2C9, CYP2D6, CYP2C19, CYP3A4 and CYP3A5. In other embodiments, CYP enzymes include CYP1A1, CYP1B1, CYP2A6, CYP2A13, CYP2B6, CYP2C8, CYP2C9, CYP2C18, CYP2E1, CYP2G1, CYP2J2, CYP2R1, CYP2S1, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, a composition of the invention is taken together with a compound that is metabolized by the same CYP enzyme(s) as the composition of the invention, so as to permit a lower dose to be taken, increase the effective bioavailability of one or both, or otherwise affect drug metabolism or pharmacokinetics. In some embodiments, the dose of a composition of the invention is adjusted when administered to a subject known to be a “poor metabolizer” of the active agent in composition (e.g., having a genetic variation in CYP2D6, known to be the major metabolizer of the methylenedioxy moiety of MDMA, where the entactogen is MDMA).

In some embodiments, a genetic variation (or a genetic marker signifying a variation) is an exclusion criteria for the administration of a disclosed compound. In an embodiment, the genetic variation is a genetic variation in metabotropic glutamate receptor type 5 (mGluR5), which has been implicated in mood and anxiety symptoms in humans. In an embodiment, the genetic variation is one or more single nucleotide polymorphisms (SNPs) in the FKBP5 gene that are associated with elevated levels of FKBP51 protein relative to persons lacking such SNPs. The FKBP5 gene has been implicated in responses to stress and trauma, and such SNPs are correlated with susceptibility to certain depression and anxiety disorders and PTSD. In an embodiment, the genetic variation is a genetic variation such as a SNP in a membrane transporter, such as SERT, DAT, NET, or VMAT. In one embodiment, the mammal being treated has altered epigenetic regulation of a gene the expression of which is associated with a mental health condition or susceptibility to a mental health treatment, such as the SIGMAR1 gene for the non-opioid sigma-1 receptor.

In embodiments, modification to the disclosed compositions and methods may be determined by the age of the patient, the weight of the patient, the comorbidities if any of the patient, other medications the patient is taking (routinely or presently), any patient-specific aspects that could affect the way in which medications interact with the patient, such as variations in metabolism and variations in patient response, and any other such variations known to one of skill.

“Therapist” refers to a person who treats a patient using the disclosed compositions and methods, whether that person is a psychiatrist, clinical psychologist, clinical therapist, psychotherapist, or other trained counselor, facilitator, or guide, and who may or may not be a trained addiction specialist or counselor, substance abuse specialist or counselor, or alcohol abuse specialist or counselor. Generally, the therapist will be certified in the use of the treatment manual for the EAP, such MDMA-assisted therapy administered, and will have completed the appropriate training in delivering that form of EAP, such as MDMA-assisted therapy. This will enable a therapist to respond in a safe and supportive manner during the non-drug (e.g., non-MDMA) and drug-assisted (e.g., MDMA) sessions. Studies with MDMA-assisted therapy have shown that most psychiatric emergencies, e.g., acute anxiety associated with the emergence of challenging emotional material during the therapeutic session, can be dealt with through “talking down” an anxious patient, with no necessary requirement for psychiatric medication. However, within the definition of “therapist” are psychiatrists licensed to manage psychiatric emergencies by administering “rescue medications” such as short-acting benzodiazepines if clinically indicated.

“Treating” or “treatment” of a disorder includes: (i) inhibiting the disorder, i.e., arresting or reducing the development or progression of the disorder or its clinical symptoms; or (ii) relieving the disorder, i.e., causing regression of the disorder or its clinical symptoms. Inhibiting the disorder, for example, would include prophylaxis. Hence, one of skill will understand that a therapeutic amount necessary to effect treatment for purposes of this invention will, for example, be an amount that provides for objective indicia of improvement in patients having clinically-diagnosable symptoms. Generally, “objective indicia” will be those recognized by clinicians who diagnose and treat patients having the condition, and therefore readily appreciated in the art.

In some embodiments, “objective indicia” may include, but are not limited to, an improvement in weight, body temperature, heart rate (HR), respiratory rate, blood oxygenation, blood pressure (BP) and its variables, including, but not limited to: systolic (SBP), diastolic (DBP), mean arterial (MAP), and pulse (PP); continuous non-invasive beat-by-beat blood pressure (CNIBP); measurements from an electrocardiogram (ECG), including, but not limited to, RR interval or its variability, QT interval or its variability, heart rate variability (HRV) (or measured by devices other than an ECG); hemodynamic response (HR), and levels of glucose, cortisol, serotonin, dopamine, cholesterol; electroencephalography (EEG) measures such as quantitative EEG (qEEG); electrocochleogram (ECochG), electromyography (EMG), electrooculography (EOG), magnetoencephalography (MEG); electrocorticography (ECoG); magnetic resonance imaging (MRI); functional MRI (fMRI); computed tomography (CT); positron emission tomography (PET); nuclear magnetic resonance (NMR) spectroscopy or magnetic resonance spectroscopy (MSR); single-photon emission computed tomography (SPECT); near infrared spectroscopy (NIRS); event-related optical signal (EROS); computed axial tomography; diffuse optical imaging (DOI); cranial ultrasound; or functional ultrasound imaging (fUS) (together, “EEG measures”); brain derived neurotrophic factor (BDNF); genetic markers including relating to CYP enzymes or drug metabolism; genetic variation in mGluR5 or FKBP5; and numerous more, as will be appreciated by those of skill. In some embodiments, the objective indicia of improvement may be an improvement in one's psychological state, wherein “psychological state” may refer to the condition or state of one's mind including, but limited to, its physiological and subjective characteristics. In some embodiments, psychological state is synonymous with the terms or phrases “mental condition,” “mental state,” “psychological condition,” “neural status,” “cognitive state,” “state of mind,” “frame of mind,” “emotional state,” etc. Physiologically, in the context herein, psychological state broadly refers to the condition of the nervous system and its components, including, but not limited to, the brain and its subunits. Regardless, the effect, or improvement, may be prophylactic in terms of completely or partially preventing a disorder or symptom thereof and/or may be therapeutic in terms of a partial or complete cure for a disorder, an adverse effect attributable to the disorder, and/or a comorbidity simultaneously present with the disorder.

A “comorbidity” present in a patient diagnosed with a condition includes a substance use disorder (SUD), such as those described herein; a behavioral addiction (BA), such as those described herein; and a psychiatric disorder, such as antisocial personality disorder, borderline personality disorder, depression, anxiety, schizophrenia, attention deficit hyperactivity disorder, bipolar disorder, obsessive compulsive disorder, binge eating disorder, and PTSD, or any mental health disorder as disclosed herein. A “comorbidity” present with AUD includes, in some embodiments, psychiatric disorders such as antisocial personality disorder, borderline personality disorder, depression, anxiety, schizophrenia, attention deficit hyperactivity disorder, bipolar disorder, obsessive compulsive disorder, binge eating disorder, and PTSD.

Moreover, “treatment” as used herein covers any treatment of a disorder in a mammal, and preferably in a human, and includes: (a) preventing a disorder from occurring in a subject who may be predisposed to the disorder but has not yet been diagnosed with it; (b) inhibiting a disorder, i.e., arresting its development; (c) relieving a disorder, i.e., causing regression thereof; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder or comorbid with a disorder. Other such measurements, benefits, and surrogate or clinical endpoints, alone or in combination, would be understood to those of skill.

Any disclosed method can be used in combination with one or more therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it such participation can occur with or without the participation or guidance of a therapist or other professional.

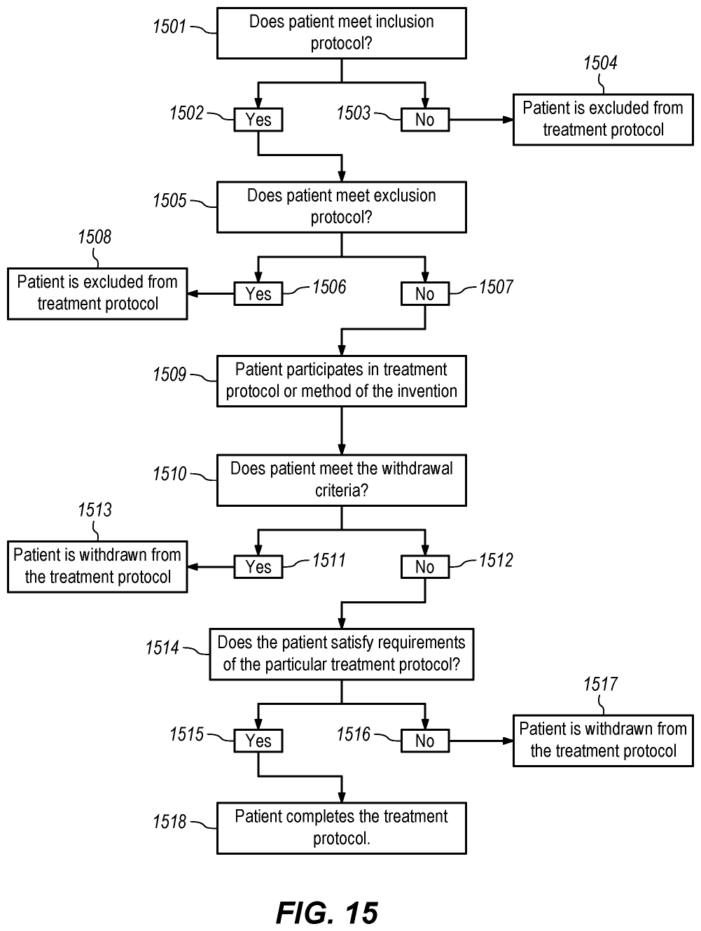

An exemplary treatment protocol is outlined in FIG. 15, and is further detailed as follows. In this exemplary embodiment, a patient will first be assessed to see if they meet the inclusion protocol, which contains criteria outlined in TABLE 1 (1501). If not (1503), the patient is excluded from the treatment protocol (1504). If all inclusion criteria are met (1502), however, the patient is further assessed to determine if any of the exclusion criteria (also listed in TABLE 1) are met (1505). Unlike inclusion criteria, in which all must be met, only one exclusion criteria need be present (1506) to exclude the patient from participating in the treatment protocol or method of the invention (1508). If no exclusion criteria are found to be met (1507), however, the patient may participate in the treatment protocol/method of the invention (1509). While participating in a treatment protocol or method of the invention, the patient will further be assessed for withdrawal criteria (1510), also listed in TABLE 1. As with exclusion criteria, if one withdrawal criteria is met (1511), the patient is withdrawn from the treatment protocol or method of the invention (1513). So long as no withdrawal criteria are met (1512), the patient may continue with the treatment protocol or method of the invention, and it thus be determined as to whether or not the patient satisfies the requirements of the particular treatment protocol or method of the invention (1514). If so (1515), the patient thereby completes the treatment protocol (1518). However, if the patient does not satisfy the requirements of the particular treatment protocol/method of the invention (1516), the patient is withdrawn from the treatment protocol, or method of the invention (1517).

For example, patients undergoing EAP, such as MDMA-assisted therapy, maybe withdrawn prior to or during a course of therapy if certain withdrawal criteria were to occur. However, after patients have completed a course of therapy (e.g., an exemplary 8-week course or 13-week course of EAP or MDMA-assisted therapy as in embodiments below, including those of Examples 4 and 8) they generally are not withdrawn based on these criteria. Patients are always free to withdraw at any time from treatment without giving reasons and without prejudicing further treatment.

Examples of “inclusion criteria,” “exclusion criteria,” and “withdrawal criteria” within the meaning of the invention include those in TABLE 1 below. It will be readily appreciated that such criteria are exemplary, optional, and non-limiting, unless stated or necessarily implied otherwise, or where needed in embodiments to provide technical effect.

TABLE 1
Exemplary Inclusion, Exclusion, and Withdrawal Criteria
Exemplary Inclusion Criteria

| | |
|---|---|
| 1 | Informed consent |
| 2 | Primary diagnosis (e.g., as defined by DSM-5) of alcohol use disorder (including, e.g., |
| | dependent use or harmful use AUD) |
| 3 | Successful alcohol detoxification (no longer consuming any alcoholic substances) (for |
| | “dependent use” patients); successful completion of a preparatory group therapy course (for |
| | “harmful use” patients; for some “dependent use” patients) |
| 4 | Between 18 and 65 years old |
| 5 | Be able to identify in advance a supportive friend, family member, or significant other: |
| | who could meet with the clinician and receive information about the treatment at one |
| | of the preparation sessions prior to the patient's first MDMA session |
| | who could accompany the patient to study visits if required |
| | who could act as a “locator” and be contacted by the therapy team in order to remind |
| | the patient about follow-up appointments or collect outcome data (such as drinking |
| | behavior) in the event that the patient themselves cannot be contacted |
| 6 | Proficient in speaking and reading the language in which the therapy is provided |
| 7 | Agree to comply with requirements of therapy protocol |

Exemplary Exclusion Criteria

| | |
|---|---|
| 1 | Lacking capacity |
| 2 | Personal history of, or a current, primary psychotic disorder, bipolar affective disorder type 1 |
| | or personality disorder |
| 3 | Present a serious suicide risk, e.g., as determined using the clinical judgment of one or more |
| | qualified mental health professionals within a therapy team. Risk may be determined using |
| | information from the Columbia-Suicide Severity Risk Scale (C-SSRS) which allows |
| | classification of severity of suicidal ideation and behavior. The C-SSRS scale classifies |
| | severe risk as (a) current suicidal ideation with intent and/or plan, and (b) suicidal behavior |
| | in the last 3 months. A clinical judgment regarding the level of risk and subsequent decisions |
| | regarding eligibility and care could use a combination of the information provided by the |
| | C-SSRS, the patient's history of previous risk behaviors, any presenting mental health |
| | difficulties, and environmental and clinical factors. A final decision may include a discussion |
| | with the qualified mental health professionals within the therapy team. |
| 4 | Relevant abnormal clinical findings at the screening visit. These include: |
| | History of cardiac disease or stroke |
| | History of severe liver disease, as evidenced by abnormal liver function test results, |
| | particularly reduction in albumin (normal > to 3.5 gm/dl) |
| | History of epilepsy |
| | History of Malignant Hyperthermia (Central Core Disease) |
| | Current untreated/unstable hypertension |
| 5 | Regular user of recreational MDMA or “ecstasy,” e.g., more than five times in the last five |
| | years or at least twice in the 6 months prior to the start of the study |
| 6 | Currently taking or unwilling/unable to stop any medications inhibiting CYP2D6, and the |
| | following medications: monoamine oxidase inhibitors, Ritonavir, paroxetine, fluoxetine, |
| | citalopram, regular benzodiazepines or any other medications likely to interact with MDMA; |
| | to be stopped during course of MDMA-assisted therapy only (e.g., during exemplary 8 or 13 |
| | week courses below) |
| 7 | Regular use of or particularly dependence on other drugs such as benzodiazepines, synthetic |
| | cannabinoids, cocaine, or heroin |
| 8 | For females of childbearing age and potential: must not be pregnant and/or breast-feeding, |
| | until the end of the treatment phase |
| 9 | Patients that might face additional risks from immunosuppression (for example patients with |
| | immunological diseases, patients with active infection or history of infections within four |
| | weeks of MDMA administration, etc.) |

Exemplary Withdrawal Criteria

| | |
|---|---|
| 1 | Development of concurrent physical illness or deterioration of mental health warranting |
| | additional interventions and support that necessitates removal from therapy |
| 2 | The patient becomes pregnant |
| 3 | Start of medications that may interact with MDMA (see exclusion criteria above) |
| 4 | Levels of drinking that interfere with the patient's ability to engage with the treatment. If a |
| | patient has an isolated lapse to drinking, participation may be continued once stable enough |
| | to engage in the treatment. |
| 5 | If a patient is using illicit substances that are considered to interact adversely with the |
| | MDMA sessions or other aspects of the psychotherapy course |

In some embodiments, patients will be required to taper and stop any medications with a known or suspected risk of drug interaction with the entactogen used in the EAP, for example MDMA. “Drug interaction” refers to a case wherein two or more drugs react with one another in the body and, as a result, either potentiate the effects of one or both of the drugs (i.e., increase the strength with which the drugs act in the body such that their effects are greater than would be expected on their own), or cause unexpected, and often adverse, side effects. In some embodiments, drug interactions also refer to drug and food, or drug and beverage interactions, wherein the drug administered to the patient interacts with a given food or beverage (namely, the compounds within the food or beverage), to cause an unexpected, often adverse reaction. In some embodiments, drug interactions also refer to drug and condition interactions, wherein administration of a drug may worsen a diagnosed, or undiagnosed medical condition.

Generally, medication will only be tapered and stopped if deemed appropriate based on clinical decision and discussion with the therapy team. Tapering may be completed through direct tapering, which involves slowly reducing the amount of the drug or substance taken over a given amount of time (which is dependent upon the drug or substance taken, the individual, the length of use and other such metrics that would be known to those of skill); substitution tapering, wherein the drug or substance is replaced with a drug or substance that, in some embodiments, causes a similar physiological response, but has a longer half-life, so tapering is more effective; and titration tapering, wherein the drug or substance is dissolved in a specific amount of a fluid, generally but not necessarily water (Coalition Recovery, 2019).

Regardless of the tapering method, the washout period of medications to be stopped will be dependent on the half-life of that particular medication. For example, if MDMA-assisted therapy is utilized, a sufficient washout period of 2 weeks (5 weeks for fluoxetine) is allowed prior to the first MDMA session and medications are not restarted until completion of the final MDMA session, unless clinically required. Exemplary psychotropic medication that may be excluded include: any drugs inhibiting the liver enzyme CYP2D6, antidepressants of the serotonin and/or noradrenaline reuptake inhibitor type (SSRI/SNRI), Mirtazapine, Ritonavir and regular use of benzodiazepines. These psychotropic medications may elevate the risk of serotonin syndrome when combined with an entactogen, such as MDMA, and may reduce its therapeutic effects (Hysek 2012b). Exemplary medications that may be permitted include: pregabalin, gabapentin, occasional benzodiazepines and Z-hypnotics (e.g., zolpidem, zopiclone, zaleplon, or eszopiclone). Commonly used medications during and after the detoxification processes that generally will be permitted include baclofen, acamprosate, naltrexone, and disulfiram. Evidence suggests a decrease in CYP3A4 activity after a single dose of MDMA of 1.5 mg/kg (Yubero-Lahoz 2011), and in some embodiments CYP3A4 medications may be contraindicated. Adjustments may be made for patients who are considered “slow” or “fast” metabolizers, e.g, for CYP2D6 and/or CYP3A4 (see, e.g., Zanger 2013).

“Assessment” refers to any means or method used with a patient, whether before, during, after, or unrelated in time to a specific treatment protocol, to measure, estimate, or evaluate a nature, ability, symptom, disorder, or other characteristic of the patient, whether qualitatively or quantitatively, and whether performed by the therapist or other clinician (e.g., an interview), by the patient his or herself (e.g., a self-reported questionnaire), by a third-party human or by a medical or other device (e.g., a medical sensor or biosensor, a watch or fitness tracker), and whether graded by a human decision-maker or an artificial intelligence (AI), using machine learning, or with a computer algorithm. Non-limiting examples of assessments include those in TABLE 2 below.

TABLE 2
Exemplary Patient Assessments

| | |
|---|---|
| 1 | The Severity of Alcohol Dependence Questionnaire (SADQ) (Stockwell et al. 1983). |
| | This is a 20 item clinical screening tool designed to measure the presence and level of |
| | alcohol dependence, taking approximately five minutes. This scale is designed to assess |
| | people that are currently drinking and therefore generally (“generally,” here and elsewhere, |
| | means “in a preferred embodiment,” but does not mean “necessarily”) will only be |
| | administered once, at screening before patients undergo alcohol detoxification. |
| 2 | The Mini International Neuropsychiatric Interview 5 (MINI 5) (Sheehan et al. 1998) to |
| | screen for comorbid psychiatric disorders. This generally will be completed in full at |
| | screening, once, taking approximately 20 minutes. |
| 3 | The Columbia Suicide Severity Rating Scale (C-SSRS) (Mundt, JC et al. 2013), to screen |
| | for acute and recent suicide and self-harm thoughts and behaviors, taking approximately |
| | five minutes to complete. Generally to be completed at screening, at baseline after detox, |
| | before each of the two MDMA-assisted sessions, the days after each MDMA session and in |
| | the exemplary embodiments, at 3, 6 and 9-month follow-up. |
| 4 | The Alcohol Dependence Syndrome section of the SCID-5-CT (Clinical Trials Version) |
| | (First et al. 2015), this semi-structured interview generally will be conducted by a trained |
| | member of the therapy team and completed once, at screening, taking approximately ten |
| | minutes. |
| 5 | The Patient Health Questionnaire (PHQ-9) (Kroenke et al. 2001). A brief |
| | self-administered screening questionnaire for depressive symptoms. It will be administered |
| | at screening, baseline, the final therapy session and at 3, 6, and 9 months' follow-up (i.e., |
| | here and elsewhere, if follow-up is performed), taking approximately five minutes. |
| 6 | Generalized Anxiety Disorder 7 (GAD-7) (Spitzer et al. 2006) is a |
| | self-reported questionnaire for screening and severity measuring of generalized anxiety |
| | disorder (GAD). It generally will be administered at screening, baseline, the final therapy |
| | session and at 3, 6, and 9 months' follow-up, taking approximately five minutes. |
| 7 | Short Inventory of Problems for Alcohol (SIP) (Kiluk et al. 2013) SIP for alcohol is a |
| | 15-item instrument that assesses the self-attributed consequences of drinking taking |
| | approximately 5 minutes. This generally will be completed at screening, baseline, the final |
| | therapy session and at 3, 6, and 9 months' follow-up. |
| 8 | The Alcohol Timeline Follow Back (TLFB) (Sobell 2001) is a method of assessing daily |
| | drinking and recreational drug use over time. Patients generally will be given the TLFB |
| | diary after screening, data will be collected at each therapy session. After completion of |
| | therapy, a researcher generally will call on a monthly basis to collect data throughout the |
| | study. Further discussion of the diary generally will take place formally at 3, 6, and 9 |
| | months follow up. |
| 9 | The Clinical Institute Withdrawal Assessment for Alcohol-Revised Version |
| | (CIWA-Ar) (Saitz et al. 1994). This is a ten-item scale used in the assessment and |
| | management of alcohol withdrawal. Each item on the scale correlates to the severity of |
| | alcohol withdrawal. This generally will be administered at baseline (after detox) and before |
| | each MDMA-assisted session, taking approximately 3 minutes. |
| 10 | The Penn Alcohol Craving Scale (PACS) (Flannery et al. 1999) is used to assess |
| | frequency, intensity and duration of thoughts about drinking, completed at the screening and |
| | baseline assessment and at 3, 6, and 9 months' follow-up. |
| 11 | Obsessive Compulsive Drinking Scale (OCDS) (Anton 1995) is used to measure |
| | obsessive and compulsive thoughts in relation to alcohol. Completed at the screening and |
| | baseline assessment and at 3, 6, and 9 months' follow-up. |
| 12 | Pittsburgh Sleep Quality Index (PSQI) (Buysse 1989) is used to assess the level of sleep |
| | disturbance, completed at screening, the baseline assessment and at 3, 6, and 9 months' |
| | follow-up. |
| 13 | Interpersonal reactivity Index (IRI) (Davis 1980) comprises 28 items answered on a 5 |
| | point scale. This scale measures different aspects of empathy and provides different |
| | subscales relating to these. This generally will be administered at baseline, the final therapy |
| | session, 3, 6, and 9 months' follow-up to explore if these core aspects of empathy are |
| | modified by MDMA Assisted therapy. |
| 14 | The Short Form (36) Health Survey (SF-36) is a gold standard patient-reported measure |
| | of quality of life that can be completed at baseline, 3, 6, and 9 months. |
| 15 | The Self-Compassion Scale (SCS) (Neff 2003) Comprises 26 items answered on a 5 point |
| | scale. This scale measures core aspects of self-compassion including components of |
| | mindfulness. This generally will be administered at baseline, the final therapy session, 3, 6, |
| | and 9 months' follow-up. |
| 16 | The Trauma History Questionnaire (THQ) (Green 1996) is a self-report measure that |
| | examines experiences with potentially traumatic events using a yes/no format. For each |
| | event endorsed, respondents are asked to provide the frequency of the event as well as their |
| | age at the time of the event. It generally will be administered once, at baseline. |

An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment may include use of an electronic psychiatric notes system, where relevant clinical information is recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like). “Computer-assisted” may refer to one or more aspects of a psychosocial, behavioral, or EAP, such as MDMA-assisted therapy.

“Therapeutic effect” or “therapeutic efficacy,” as used herein, means the response(s) in a mammal, and preferably a human, after treatment that are judged to be desirable and beneficial. Hence, depending on the disorder to be treated, or improvement in physiological or psychological functioning sought, and depending on the particular constituent(s) in the disclosed compositions under consideration, those responses shall differ, but would be readily understood by those of ordinary skill. For AUD, including for both “harmful use” AUD and “dependent use” AUD, and for comorbid SUDs and comorbid BAs, these responses will be understood based on the teachings herein and the general knowledge in the art.

Measures of therapeutic effect includes any outcome measure, endpoint, effect measure, or measure of effect within clinical or medical practice or research which is used to assess the effect, both positive and negative, of an intervention or treatment, whether patient-reported, gathered through laboratory tests such as blood work, urine samples etc., or through medical examination. In some embodiments, therapeutic effect may include the collection of objective measures, including weight, body temperature, heart rate, respiratory rate, blood oxygenation, BP and its variables, including SBP, SBP, MAP, and PP; CNIBP; ECG measurements, including RR interval or its variability, QT interval or its variability, HRV (including measured by devices other than an ECG); hemodynamic response, and levels of glucose, cortisol, serotonin, dopamine, cholesterol; EEG measures; BDNF; genetic markers including relating to CYP enzymes or drug metabolism; genetic variation in mGluR5 or FKBP5; and many others, as will be readily appreciated by those of skill.

Measures of therapeutic effect for purposes of the disclosure also will be understood to include, as non-limiting examples, in an embodiment pertaining to an SUD: (1) Weekly units of a substance such as alcohol consumed post-detox (i.e., counting from the last day of the detoxification course), or post “baseline” as defined herein, wherein a unit of alcohol, e.g., refers to a standard drink that contains about 14 g of pure alcohol (ethanol), or other measure of relapse status; (2) Quality of life, including subjective sleep; (3) Psychosocial functioning; (4) Prescribed medication use; (5) Number and frequency of recreational drugs (if any); (6) Cravings relating to MDMA and any subsequent use of illicit ecstasy; and (7) Symptoms of SUD, such as AUD, or any comorbid disorder(s). Those of skill in the art will appreciate that other equivalent or similar measures of therapeutic effect also may be used, and others are described herein and may be claimed. In some embodiments, a therapeutic effect may be shown by one or more of: (a) a reduction of substance use, (b) a reduction of substance cravings, (c) a promotion of substance use abstinence, (d) a prevention of relapse into substance use, or (e) an improvement of at least one symptom of substance use disorder. In some embodiments, a therapeutic effect may be shown by one or more of: (a) an increase in quality of life, (b) an increase in psychosocial functioning, (c) a decrease in use or frequency of a prescription medication, (d) a decrease in use or frequency of a recreational drug, (e) a decrease in obsessive compulsive thoughts, (f) a decrease in suicidality, (g) an increase in feelings of empathy, or (h) an increase in self-compassion.

In some embodiments, such as an embodiment wherein treatment is of a BA, a therapeutic effect may be shown by one or more of: a reduction in the frequency of a compulsive or problematic behavior, a reduction in drive or urge to engage in a compulsive or problematic behavior, prolonged abstinence from a compulsive or problematic behavior, a prevention of relapse into a compulsive or problematic behavior, a reduction in severity of symptoms of a compulsive or problematic behavior, a reduction in the time spent on a compulsive or problematic behavior; an increase in quality of life, including subjective sleep; and an increase in psychosocial functioning.

In some embodiments, a therapeutic effect may be shown by an improvement of at least one symptom of a comorbid psychiatric disorder, such as antisocial personality disorder, borderline personality disorder, depression, anxiety, schizophrenia, attention deficit hyperactivity disorder, bipolar disorder, obsessive compulsive disorder, binge eating disorder, or PTSD. In some embodiments, a measure of therapeutic effect may be “durable,” e.g., where shown after a specific period of time, for example at least 1 month, at least 3 months, at least 6 months, at least 9 months, at least 1 year, or greater than 1 year, where such time is from the end of detoxification, if detoxification is completed, from baseline, or from the first psychotherapy session. In some embodiments, a measure of therapeutic effect will be shown for a period of time measured from when the subject has completed the EAP regimen, such as the MDMA-assisted therapy regimen (i.e., measured from the last drug-assisted or non-drug assisted psychotherapy session).

An “effective amount,” a “therapeutically effective amount,” a “therapeutically effective dose,” or a “pharmacologically effective amount,” refers to an amount of an active agent that is non-toxic and sufficient to provide the desired therapeutic effect with performance at a reasonable benefit/risk ratio attending any medical treatment. The effective amount will vary depending upon the subject and the disease condition being treated or health benefit sought, the weight and age of the subject, the severity of the disease condition or degree of health benefit sought, the manner of administration, and the like, all of which can readily be determined by one of skill. As it relates to the invention, “therapeutically effective amount,” refers to the amount of a disclosed compound used in a disclosed composition or method (i.e., MDMA or another disclosed entactogen) sufficient to effect treatment when administered to a subject in need of such treatment. Administration of a composition in a “therapeutically effective amount” or an “effective amount” to a subject means administration of an amount of the composition of the invention in a manner as disclosed herein, sufficient to achieve the desired effect. In some embodiments, a “therapeutically effective amount,” or an “effective amount,” thus would be the amount sufficient to cause a measurable modulation which, in some embodiments, refers to the exertion of a modifying or controlling influence on something which, as it relates to the invention, may be a neurotransmitter system (such as, but not limited to, the serotonergic, dopaminergic, and/or noradrenergic system), where that measurable modulation is known to result in or is shown to result in treatment of a disorder. When an “effective amount” means an amount effective in treating the stated disorder itself, or in the symptoms thereof in a subject, “therapeutic effect” would be understood to mean the responses(s) in the subject after treatment judged to be desirable and beneficial.

“Pharmacovigilance” refers to any efforts to monitor the physiological, psychological, or other effects associated with an entactogen, e.g., MDMA or another active agent used in a disclosed composition or method, especially in order to identify and evaluate adverse events or adverse reactions. For example, at every visit during a treatment period a patient may be asked open-ended questions about how they are feeling and any side effects or adverse events documented. Therapists may have access to a check-list of common side effects to aid documentation.

Commonly reported effects of MDMA reported in Phase 1 studies in healthy volunteers include elevation in blood pressure and heart rate, increased anxiety or dysphoria, and dilated pupils. Some reports indicated decreased rather than increased alertness. Other common effects reported in controlled studies of MDMA include reduced appetite, dizziness, tight jaw or bruxism (tooth-grinding), difficulty concentrating, impaired gait or balance, dry mouth, and thirst. Patients in some studies also reported or exhibited changes in cognition, such as increases in speed of thought or thought blocking, facilitated imagination or facilitated recall, and unusual thoughts or ideas. Other less commonly reported effects include paraesthesias (unusual body sensations) such as tingling or feeling hot or cold. MDMA has been shown to produce anxiety in some healthy volunteers. These effects are transient and recede with the waning of drug effects. One study found that women were more likely than men to experience the most common effects of MDMA, though men were more likely than women to experience the specific effects of nausea and sweating. Effects in women undergoing a single session of MDMA-assisted therapy for PTSD were mild and appear to be similar to those in healthy controls. Clinical judgment generally will be used to define any effects as adverse where appropriate and these will be documented using standard procedures, where such documentation is required or desired.

“Adverse event” (AE) refers to any untoward medical occurrence in a patient administered a medicinal product and which does not necessarily have a causal relationship with this treatment. An AE can therefore be any unfavorable and unintended sign (including an abnormal laboratory finding), symptom, or disease temporally associated with the use of an investigational medicinal product (IMP), whether or not considered related to the IMP. A moderate rise in pulse and blood pressure is common when taking MDMA. For example, MDMA at doses of 100-125 mg has been associated with increases in heart rate of 26-30 bpm and blood pressure (diastolic 14.4-25 mmHg) (Harris 2002, Mas 1999, Mithoefer et al. 2011). These can be monitored following a regime such as that known in the art (see, e.g., Oehen et al. 2013, Chabrol & Oehen 2013). In some embodiments, blood pressure and pulse can be measured throughout a MDMA-assisted session, starting at baseline before the drug is given and hourly thereafter for six hours. In the event of a patient presenting with acutely elevated BP (e.g., systolic BP [SBP]>200 mm Hg or diastolic BP [DBP]>120 mm Hg) the patient is referred for emergency hospital care. The upper age range of patients may be limited, e.g., to 65 years, and patients may be screened for cardiac abnormalities.

“Adverse reaction” (AR) refers to all untoward and unintended responses to an IMP related to any dose administered. All ARs judged as having reasonable causal relationship to a medicinal product qualify as adverse reactions. The expression “reasonable causal relationship” means to convey in general that there is evidence or argument to suggest a causal relationship. As an example, there has been one serious adverse reaction associated with 125 mg of MDMA during assisted psychotherapy. This individual was observed to have an increased frequency of ventricular extrasystoles (PVCs) on the third of three MDMA-assisted sessions; this had not occurred on two previous sessions (after having been given 125 mg and then 62.5 mg MDMA). The decision was taken not to administer the booster 62.5 mg MDMA dose on this occasion and a single dose of a beta blocker was administered. Monitoring as normal was undertaken with full recovery the following day. Extensive cardiac investigations revealed no evidence of persisting pathology. This patient was noted to have a family history of cardiovascular disease (biological father coronary artery bypass). There have been no other unexpected drug-related serious adverse events in any of the human MDMA research studies to date (Doblin et al. 2014).

“Unexpected adverse reaction” (UAR) refers to an AR, the nature or severity of which is not consistent with the applicable product information (e.g., investigator's brochure for an unapproved investigational product, summary of product characteristics (SmPC) for an authorized product, or package insert or other product labeling for an approved drug). When the outcome of the adverse reaction is inconsistent with applicable product information this adverse reaction should be considered “unexpected.” Side effects documented in the SmPC which occur in a more severe form than anticipated are also considered to be unexpected.

“Serious adverse event” (SAE) or “serious adverse reaction” (SAR) refer to any untoward medical occurrence or effect that at any dose: results in death; is life-threatening (i.e., an event in which the subject was at risk of death at the time of the event, but not an event which hypothetically might have caused death if it were more severe); requires hospitalization, or prolongation of existing inpatients' hospitalization; results in persistent or significant disability or incapacity; or is a congenital anomaly or birth defect. However, this list is non-exclusive, and medical judgment will be exercised in deciding whether an AE or AR is serious in other situations. Important AEs or ARs that are not immediately life-threatening or do not result in death or hospitalization but may jeopardize the subject or may require intervention to prevent one of the other outcomes listed in the definition above, also should be considered serious.

“Suspected unexpected serious adverse reaction” (SUSAR) refers to any suspected adverse reaction related to an IMP that is both unexpected and serious (i.e., by reference to the definitions for UAR and SAR as disclosed herein).

In discussing AEs and ARs as above, it will be appreciated that there are distinct differences between the uses of recreational ecstasy compared to clinical MDMA, or likewise between recreational use of entactogens generally and the clinical use thereof. The former is a drug preparation of varying quality and dose that commonly comprises one or more potential toxic adulterants. Although a number of serious adverse events, including fatalities, have been reported after recreational ecstasy use in unsupervised and uncontrolled settings, even these events are relatively rare given the prevalence of ecstasy use. These include hyperthermia, including hyperthermia arising from “serotonin syndrome,” psychiatric problems, hepatotoxicity, including acute liver failure arising from hyperthermia and liver disease and hyponatremia. Accordingly, in the compositions and disclosed methods, “MDMA” (or an “entactogen”) unless context expressly dictates otherwise, refers to MDMA (or the entactogen) that is pure or substantially pure, or a composition containing such pure or substantially pure MDMA.

“Set and setting” may play a role in the development of some ecstasy-related adverse events, such as vigorous physical exertion, lack of attention to somatic cues, and too little or too much hydration resulting in hyperthermia or hyponatremia. The term “set and setting” is commonly attributed to Timothy Leary, a psychologist well known for promoting psychedelics and psychedelic research in the 1960s. While consideration of set and setting is traced further back to use of plant-based psychedelics in Indigenous cultures, it continues to be considered to be crucial in modern use of psychedelic-assisted therapies in clinical settings and research trials (Hartogsohn, 2017). The concept of “set” refers to the internal aspects of the person undergoing a psychedelic experience such as their personality, mood, intentions and expectations (Metzer and Leary, 1967). “Setting” encompasses the physical, social and cultural environment in which the psychedelic experience takes place (Hartogsohn, 2017).

These factors can be controlled during MDMA or other entactogen administration, whether by the supervising clinicians or a member of the therapy team, or by the design of the treatment protocol itself. Such factors are considered and controlled in the disclosed methods, as will become readily apparent. Both set and setting considerations are paramount to support an individual's therapeutic course. Careful preparation is crucial to allow a patient to feel ready to trust and let go in their drug-assisted session so that they can go towards and be with whatever internal experiences show up. In addition, supportive integration is fundamental to allow the individual to describe for themselves their internal experiences during the drug-assisted session and make meaning from these in ways that helps them take committed action towards their values. Integration goes beyond just the formal sessions that take place within the therapy setting, and a patient should be supported to consider how their ongoing processing can be facilitated via community networks and resources.

Gaining a strong therapeutic alliance within a relatively short period of time can help the patient to feel supported and safe; using core therapeutic skills such as active listening, reflections using the patient's own words where appropriate and giving time to discuss any questions or anxieties will facilitate the patient-therapist relationship. Wherever possible the therapist will attend the initial assessment to maximize the opportunity for rapport building, as well as beginning the early psychological formulation process. The assessment phase will help to determine whether a patient appears to be psychologically suited to the therapeutic intervention, notwithstanding other key eligibility criteria. Part of this consideration can take into account the person's support network within their community and their willingness and ability to utilize and/or build on this for support.

The physical environment will be purposefully designed with safety and support in mind, while being minimally distracting to allow the patient to focus on their internal experience during drug-assisted sessions. Such design will be as readily appreciated in the art, and as used in psychedelic-assisted therapy for many years. In line with both early and modern research with psychedelics, dating back at least decades, therapy rooms are warm and comfortable spaces with furniture (and especially, soft furniture) allowing the patient to recline with cushions and blankets if they choose. The room should be a quiet space with minimal distractions with the ability to play music both within the room and in the headphones simultaneously. According to Kaelen et al. (2018), this helps with: “(1) providing a sense of continuity in case the headphones are abruptly removed or muted, (2) enabling the therapists to empathize with the patient's current state, as well as observe how they were responding to a particular piece of music, and (3) allowing a deeper immersion in the music and depth of sound.” Music is a well-established feature of psychedelic assisted therapies and is described by Kaelen et al. (2018) as “the hidden therapist” due to its potential to impact upon the individual's internal experience in meaningful ways while under the influence of a mind-altering compound.

“Causality” and “causal relationship” for an AE also may be assessed, i.e., an AE may be assessed as to whether or not there is a reasonable possibility of a causal relationship to the composition and/or its administration or method of use (e.g., a protocol of MDMA-assisted therapy). In any large group of patients having AUD or comorbid SUDs or BAs, a large number of AEs are expected due to the nature of the patients' condition. As the intervention is composed of psychotherapy sessions as well as EAP sessions, those performing the methods herein will in some embodiments document causality according to intervention where possible. The assignment of the causality may be made by the physician, investigator, clinical psychologist, or other member of the medical or treatment team responsible for the care of the patient using the definitions in TABLE 3 below. On occasion, doubt about the causality may exist. In such circumstances, all members of the medical or treatment team can speak together or with other knowledgeable clinicians. In the case of continued discrepant views, additional outside clinicians can be asked to advise.

TABLE 3
Definitions Regarding the Assignment of Causality to an AE

| Relationship | Description |
|---|---|
| Unrelated | There is no evidence of any causal relationship |
| Unlikely | There is little evidence to suggest there is a causal relationship (e.g., the event |
| | did not occur within a reasonable time after administration of the medication). |
| | There is another reasonable explanation for the event (e.g., the patient's clinical |
| | condition, other concomitant treatment). |
| Possible | There is some evidence to suggest a causal relationship (e.g., because the event |
| | occurs within a reasonable time after administration of the medication). |
| | However, the influence of other factors may have contributed to the event (e.g., |
| | the patient's clinical condition, other concomitant treatments). |
| Probable | There is evidence to suggest a causal relationship and the influence of other |
| | factors is unlikely. |
| Definitely | There is clear evidence to suggest a causal relationship and other possible |
| | contributing factors can be ruled out. |
| Not | There is insufficient or incomplete evidence to make a clinical judgment of the |
| assessable | causal relationship. |

“Psychedelic-assisted psychotherapy” refers to a range of related approaches that involve at least one session where the patient ingests a “psychedelic” and is monitored, supported, and/or otherwise engaged by one or more trained mental health professionals while under the effects of the drug (see, e.g., Schenberg 2018). Protocols have been developed for the standardization of procedures which emphasize a high degree of care (see, e.g., Johnson 2008), such as the therapeutic approach used by MAPS to treat patients with PTSD using MDMA (e.g., described in Mithoefer 2017). A variety of methods of psychedelic-assisted psychotherapy will be known to those in the field and are disclosed, for example, in Grof 2008; Passie 2012; Johnson et al. 2008; Sessa & Fischer 2015; Schmid et al. 2020; Greer & Tolbert 1998; Mithoefer 2017; Mithoefer 2013; U.S. Pat. App. No. 2020/0360311A1, discussing drug-assisted psychotherapy practices. It will be appreciated that when the term “psychedelic-assisted psychotherapy” is used, the drug compound administered with the psychotherapy is often not a “psychedelic” as the class is sometimes understood (e.g., the class of “classic” psychedelics), but may be a drug having “entactogenic” or “empathogenic” effects like MDMA, or drugs having “dissociative” effects such as ketamine and esketamine; however, this single umbrella classification disguises the many significant differences in implementation and simplifies the broad diversity of patient experiences and patient outcomes.

In some embodiments, a disclosed composition consists essentially of a disclosed entactogen. In some embodiments, a disclosed composition consists essentially of MDMA. In some embodiments, a disclosed composition comprises an additional active agent. In some embodiments, the additional active agent provides an additional therapeutic effect. In some embodiments, the additional active agent provides more than one additional therapeutic effect. In some embodiments, the additional active agent provides a synergistic effect. In some embodiments, the additional active agent provides more than one synergistic effect. In some embodiments, the additional active agent increases an existing therapeutic effect. In some embodiments, the additional active agent increases more than one existing therapeutic effect.

“Additional therapeutic effects” that may be provided in some embodiments of the invention include, but are not limited to, antioxidant, anti-inflammatory, analgesic, antineuropathic, antinociceptive, antimigraine, anxiolytic, antidepressant, antipsychotic, anti-PTSD, immunostimulant, anti-cancer, antiemetic, orexigenic, antiulcer, antihistamine, antihypertensive, anticonvulsant, antiepileptic, bronchodilator, neuroprotective, nootropic, entactogenic, empathogenic, entheogenic, euphoric, psychedelic, sedative, and stimulant effects.

“Synergistic effects” will be understood as including increases in potency, bioactivity, bioaccessibility, bioavailability, or therapeutic effect (including one or more additional therapeutic effects), that are greater than the additive contributions of the components acting alone, and/or are greater than the contribution of the isolated compounds on their own. In some embodiments, “synergistic” refers to a pharmaceutical composition or combination therapy that is more effective than the additive effects of any two or more single agents. A synergistic effect, e.g., permits the effective treatment of a disease using lower amounts (doses) of individual therapy. This includes lower doses of the first pharmaceutical agent or the second pharmaceutical agent (“apparent one-way synergy”), or lower doses of both pharmaceutical agents (“two-way synergy”), than would normally be required when either drug is used alone. In effect, the lower doses result in lower toxicity without reduced efficacy. A synergistic effect may additionally result in improved efficacy, including an improved avoidance or reduction of disease as compared to any single therapy.

“Synergistic effects” also will be understood to include increases in potency, bioactivity, bioaccessibility, bioavailability, or therapeutic effect (including one or more additional therapeutic effects), that are greater than the additive contributions of the components acting alone, and/or are greater than the contribution of the isolated compounds on their own. Numerous methods known to those of skill exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang et al. 2019), or the equation of Loewe additivity (Loewe & Muischnek, 1926, Arch. Exp. Pathol Pharmacol. 114: 313-326). A synergistic effect also may be calculated using methods such as the Sigmoid-Emax equation (Holford & Scheiner, 1981, Clin. Pharmacokinet. 6: 429-453) and the median-effect equation (Chou & Talalay, 1984, Adv. Enzyme Regul. 22:27-55). The corresponding graphs associated with the equations referred to above are the concentration-effect curve and combination index curve, respectively. Each equation referred to above may be applied to experimental data to generate a corresponding graph to aid in assessing the effects of the drug combination.

In embodiments, increasing an existing therapeutic effect, providing an additional therapeutic effect, increasing a desired property such as stability or shelf-life, decreasing an unwanted effect or property, altering a property in a desirable way (e.g., pharmacokinetics or pharmacodynamics), modulating a desired system or pathway (e.g., a neurotransmitter system), or otherwise inducing synergy, is achieved by the inclusion of an additional active agent. Additional active agents may include any one or more of amino acids, antioxidants, anti-inflammatory agents, analgesics, antineuropathic and antinociceptive agents, antimigraine agents, anxiolytics, antidepressants, antipsychotics, anti-PTSD agents, immunostimulants, anti-cancer agents, antiemetics, orexigenics, antiulcer agents, antihistamines, antihypertensives, anticonvulsants, antiepileptics, bronchodilators, neuroprotectants, nootropics, other entactogens and empathogens, entheogens, psychedelics, monoamine oxidase inhibitors, sedatives, stimulants, and vitamins. These ingredients may be in ion, freebase, or salt form, include polymorphs, and may be isomers.

In embodiments, an additional active agent may be any one or more of an opioid antagonist (e.g., nalmefene, naltrexone), a CB₁ antagonist (e.g., rimonabant), a CRH₁ receptor antagonist (e.g., verucerfont, pexacerfont), a NK_{1R} antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an α7 nicotinic acetylcholine receptor agonist (e.g., varenicline), a 5-HT₃ antagonist (e.g., ondansetron), a 5-HT_{1A} receptor partial agonist (e.g., aripiprazole), a 5-HT_{2A} receptor antagonist (e.g., quetiapine, olanzapine, mirtazapine), a 5-HT reuptake inhibitor (e.g., trazodone), a SERT inhibitor (e.g., duloxetine), an α1 adrenoreceptor antagonist (e.g., doxazosin, prazosin), a glucocorticoid receptor antagonist (e.g., mifepristone), an α1 adrenoreceptor agonist (e.g., guanfacine), an AChE inhibitor (e.g., citicoline), a dopamine D₂ receptor antagonist (e.g., tiapride), an α2 adrenoreceptor agonist (e.g., clonidine), an NMDA receptor antagonist (e.g., acamprosate) and an aldehyde dehydrogenase inhibitor (e.g., disulfiram), or pharmaceutically acceptable salts thereof.

In embodiments, an additional active agent is a phenethylamine disclosed in Shulgin & Shulgin, PiHKAL: A Chemical Love Story, Transform Press (1994), or a tryptamine disclosed in Shulgin & Shulgin, TiHKAL: The Continuation, Transform Press (1997), both incorporated by reference in their entirety, or a pharmaceutically acceptable salt, hydrate, solvate, polymorph, co-crystal, metabolite, analog, prodrug, stereoisomer, or tautomer thereof, or combination thereof.

In some embodiments, the additional active agent is a serotonergic agent. In some embodiments, a “serotonergic agent” will be a compound that binds to, blocks, or otherwise influences (e.g., via an allosteric reaction) activity at one or more serotonin receptors, including any one or more serotonin receptor subtypes. In preferred embodiments, a serotonergic agent is not a psychedelic. In some embodiments, a serotonergic agent binds to a serotonin receptor. In some embodiments, a serotonergic agent indirectly affects a serotonin receptor, e.g., via interactions affecting the reactivity of other molecules at the serotonin receptor. In some embodiments, a serotonergic agent is an agonist, e.g., a compound activating a serotonin receptor. In some embodiments, a serotonergic agent is an antagonist, e.g., a compound binding but not activating a serotonin receptor, e.g., blocking a receptor. In some embodiments, a serotonergic agent is an effector molecule, e.g., a compound binding to an enzyme for allosteric regulation. In some embodiments, a serotonergic agent acts (either directly or indirectly) at more than one type of receptor, including receptors other than serotonergic or other monoaminergic receptors. In some embodiments, a serotonergic agent blocks the serotonin transporter (SERT) and results in an elevation of the synaptic concentration of serotonin, and an increase of neurotransmission. In some embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In some embodiments, a serotonergic agent inhibits the activity of one or both monoamine oxidase enzymes, resulting in an increase in concentrations of serotonin and an increase in neurotransmission. In some embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites. Additional serotonergic agents will be known to those in the art, and can be found, for example, on DrugBank (https://go.drugbank.com).

In some embodiments, the additional active compound is an NMDA modulator. In some embodiments, an “NMDA modulator” is a compound that binds to, blocks, or otherwise influences (e.g., via an allosteric reaction) activity at the N-methyl-D-aspartate (NMDA) glutamate receptor subtype. In some embodiments, the NMDA modulator is a partial NMDA receptor agonist, such as D-cycloserine (DCS). In some embodiments, the NMDA modulator is a partial agonist and is NRX-1074 or rapastinel (GLYX-13). In some embodiments, the NMDA modulator is any of plazinemdor, rapastinel, LY-2140023, NYX-458, NYX-783, NYX-2925, NRX-1074, SAGE-718, a substituted 1,2,3-triazole NMDA modulator such as disclosed in WO2017/139428A1, a spiro-lactam NMDA modulator such as disclosed in WO2018/026798A1, and an NMDA modulator disclosed in WO2017/066590A1 (see, e.g., id. at [0006]). In some embodiments, the NMDA modulator is an NMDA antagonist. In some embodiments, an “NMDA antagonist” is a compound that antagonizes, or inhibits the action of, the NMDA receptor. In some embodiments the NMDA antagonist is memantine, amantadine, rimantadine, nitromemantine (YQW-36), or acamprosate. In some embodiments the NMDA antagonist is pethidine, levorphanol, methadone, dextropropoxyphene, tramadol, or ketobemidone. In some embodiments the NMDA antagonist is dextromethorphan (DXM), dextrorphan, or dextrallorphan (DXA). In some embodiments, the NMDA antagonist is gacyclidine (GK-11), neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (MK-0657), atomoxetine, LY-235959, CGP 61594, CGP 37849, CGP 40116 or CGP 37849, LY-233536, PEAQX (NVP-AAM077), ibogaine or noribogaine (or an ibogaine metabolite, ibogaine analog, or ibogaine derivative), Ro 25-6981, GW468816, EVT-101, indantadol, perzinfotel (EAA-090), SSR240600, 2-MDP (U-23807A) or AP-7. In some embodiments, the NMDA antagonist is ketamine (and its analogs, e.g. tiletamine), phencyclidine (and its analogs, e.g. tenocyclidine, eticyclidine, rolicyclidine), or methoxetamine (and its analogs). In some embodiments the NMDA antagonist is ketamine, S-ketamine, R-ketamine, or a non-racemic mixture of ketamine enantiomers. In some embodiments, the NMDA antagonist is a ketamine metabolite, such as any of the 12 HNK metabolites formed from the metabolism of ketamine in vivo, including any of the stereoselective metabolites of S- or R-ketamine, such as (R,S)-norketamine (NK), (R,S)-dehydronorketamine, hydroxyketamines, and hydroxy-norketamines (HNKs), including (2S,6S;2R,6R)-HNK, (2R,4R;2S,4S-2S,6R;2R,6S)-HNK, and (2R,4S;2S,4R-2S,5S;2R,5R)-HNK (Farmer, 2020). In some embodiments, the NMDA antagonist is a ketamine analog such as methoxetamine (2-MeO-2-deschloroketamine, MXE), 3-MeO-PCE, KEA-1010, N-ethyldeschloroketamine (2′-Oxo-PCE, O-PCE), 2-fluoro-deschloroketamine [2-(2-fluorophenyl)-2-methylamino-cyclohexanone] (2-FDCK), deschloro-ketamine (2-phenyl-2-methylamino-cyclohexanone), DXE (2′-Oxo-PCM, aka DCK), alkyne-norketamine (A-NK), and the like. In some embodiments, the NMDA antagonist is an arylcyclohexylamine or an arylcyclohexylamine derivative, such as disclosed in, e.g., WO2022/047256A1, US2022/0041540A1, and WO2021/255737A1, incorporated by reference as if fully set forth.

Additional NMDA modulators and NMDA antagonists will be known to those in the art, and can be found, for example, on DrugBank (see, e.g., Accession Number DBCAT002723, https.//go.drugbank.com/categories/DBCAT002723); other serotonergic agents are known likewise.

Compounds disclosed herein may contain one or more asymmetric centers and give rise to enantiomers, diastereomers, and other stereoisomeric forms. Each chiral center may be defined, in terms of absolute stereochemistry, as (R)- or (S)-. The invention is meant to include all such possible isomers, as well as mixtures thereof, including racemic and optically pure forms. Thus, for example, chiral compounds will be understood to refer to racemic mixtures, as well as any of the individual enantiomers, and all enantiomerically enriched mixtures thereof, in any proportions. Optically active (R)- and (S)-, (−)- and (+)-, or (D)- and (L)-isomers may be prepared using chiral synthons or chiral reagents, or resolved using conventional techniques. Various methods are known in the art for preparing optically active forms and determining activity. Such methods include standard tests described herein and other similar tests which are well known in the art.

Compounds disclosed herein may be provided in a composition that is enantiomerically enriched, such as a mixture of enantiomers in which one enantiomer is present in excess, including an enantiomeric excess in any amount greater than 0%, and in embodiments in an excess of at least about 50%, at least about 55%, at least about 60%, at least about 65%, at least about 70%, at least about 75%, at least about 80%, at least about 85%, at least about 90%, at least about 95%, at least about 98%, at least about 99%, at least about 99.5%, at least about 99.9%, up to (and including) 100%. In some embodiments are enantiomerically enriched mixtures in an enantiomeric excess of less than about 50%, such as less than about 45%, less than about 40%, less than about 35%, less than about 30%, less than about 25%, less than about 20%, less than about 15%, less than about 10%, less than about 5%, including less than about 1%, less than about 0.5%, and up to 0%.

In some embodiments comprising a non-racemic mixture, the compound will be an entactogen. In some embodiments comprising a non-racemic mixture, the compound will be MDMA. In some embodiments comprising a mixture with an enantiomeric excess of at least about 50% or less than about 50%, the compound will be an entactogen. In some embodiments comprising a mixture with an enantiomeric excess of at least about 50% or less than about 50%, the compound will be MDMA. In embodiments are compounds, such as an entactogen, e.g., MDMA, which are present in a composition or used in a method, and which are a pure or substantially pure individual enantiomer, such as a mixture having an enantiomeric excess of greater than about 75%, about 76%, about 77%, about 78%, about 79%, about 80%, about 81%, about 82%, about 83%, about 84%, about 85%, about 86%, about 87%, about 88%, about 89%, or preferably above about 90%, such as about 91%, about 92%, about 93%, about 94%, about 95%, about 96%, about 97%, about 98%, about 99%, about 99.5%, about 99.9%, and up to about 100% and including 100%.

When any compounds described herein contain olefinic double bonds or other centers of geometric asymmetry, and unless specified otherwise, it is intended that the compounds include both E and Z geometric isomers. Likewise, tautomeric forms are included.

The individual compounds of the disclosed compositions will be understood to also encompass pharmaceutically acceptable salts of such compounds. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. Exemplary salts include, but are not limited to, 2-hydroxyethanesulfonate, 2-naphthalenesulfonate, 2-napsylate, 3-hydroxy-2-naphthoate, 3-phenylpropionate, 4-acetamido-benzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride/chloride, hydrofluoride/fluoride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like, as would be immediately evident to one of skill (Berge et al. 1977). Preferred pharmaceutically acceptable salts, where the compound is MDMA, include those employing a hydrochloride anion (e.g., an HCl salt).

Different embodiments of the invention include the following examples: Pharmaceutically acceptable complex derivatives of each drug in each group, including solvates, salts, esters, enantiomers, isomers (stereoisomers and/or constitutional, including, but not limited to, ones based on substituting deuterium or a halogen, such as bromine, chlorine, fluorine, and iodine; for any hydrogen), derivatives or prodrugs of the compounds of the invention. Prodrugs of the active agents also will be appreciated to be within the scope of the invention. The term “prodrug” refers to a precursor of a biologically active pharmaceutical agent. Prodrugs undergo a chemical or a metabolic conversion to become a biologically active pharmaceutical agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety, such as a glycoside or acetyl group, to form the biologically active pharmaceutical agent. Other examples include addition of hydroxy groups (Tsujikawa et al. 2011. Xenobiotica, 41(7), 578-584; Yamamoto et al. 1984. Xenobiotica, 14(11), 867-875), acyloxyalkoxycarbonyl derivatives, amino acids, or peptides (Vig et al. 2013. Advanced Drug Delivery Reviews, 65(10), 1370-1385), which are generally added to the amine, and can be removed within the body by chemical reactions or enzymes, but other prodrugs and precursors, at the amine and other sites, should be understood to be within the scope of the invention (Simplicio, Clancy, & Gilmer. 2008. Molecules, 13(3), 519-547; Shah, Chauhan, Chauhan, & Mishra (Eds.). 2020. Recent Advancement in Prodrugs. CRC Press). Types of prodrugs contemplated to be within the scope and spirit of the invention therefore include compounds that are transformed in various organs or locations in the body (e.g., liver, kidney, G.I., lung, tissue) to release the active agent. For example, liver prodrugs will include active agents conjugated with a polymer or chemical moiety that is not released until acted upon by liver cytochrome enzymes; CYP metabolism includes dealkylation, dehydrogenation, reduction, hydrolysis, oxidation, and the breakdown of aromatic rings. Kidney prodrugs will include active agents conjugated to L-gamma-glutamyl or N-acetyl-L-gamma glutamic moieties so that they are metabolized by gamma-glutamyl transpeptidase before they are bioactive; alternatively, they may be conjugated to alkylglucoside moieties to create glycosylation-based prodrugs. Digestive or G.I. prodrugs will include those where an active agent is, e.g., formulated into microspheres or nanospheres that do not degrade until the spheres are subjected to an acidic pH; formulated with an amide that will resist biochemical degradation until colonic pH is achieved; or conjugated with a linear polysaccharide such as pectin that will delay activation until the pharmaceutical composition reaches the bacteria in the colon. Besides such exemplary prodrug forms, many others will be known to one of skill. For instance, exemplary entactogen prodrugs which may be used in the disclosed compositions and methods include those disclosed in, e.g., WO2022/053696A1, WO2022/106947A1, WO2023/283373, WO2022/235587, WO2023/283364, and WO2023/023347, which are incorporated by reference as if fully set forth.

Among derivatives of a compound are included its “physiologically functional derivatives,” which refers to physiologically tolerated chemical derivatives of the compound having the same physiological function thereof, for example by being convertible in the body thereto, and which on administration to a mammal such as a human is able to form (directly or indirectly) the compound or an active metabolite thereof (acting therefore, like a prodrug), or by otherwise having the same physiological function, despite one or more structural differences. Meaning, a therapeutically effective dose of a prodrug would necessarily be a therapeutically effective dose of the corresponding biologically-active compound. Examples of physiologically functional derivatives may include esters (including diacid hemiesters), amides, carbamates, ureas, and heterocycles.

In some embodiments, the compounds of the invention are synthetic. “Synthetic” means a compound which is manufactured artificially in a laboratory, by means of chemical synthesis (e.g., by a series of chemical processes or reactions using chemical substrates, reagents, and optionally one or more catalysts) or biosynthesis (e.g., from a bioengineered organism, and thus including those compounds also referred to as “biosynthetic” or as involving “synthetic biology” or “synbio”). For example, bacteria, yeast, and other host cells can be bioengineered to produce synthetic compounds by inserting genes that produce appropriate enzymes and/or by altering the natural metabolic pathway to achieve the production of the desired compound(s), e.g., from sugars, the main carbon source available to yeast, or from other precursor molecules. Cell-free protein synthesis (CFPS) and in vitro protein synthesis are also contemplated. These compounds can then be obtained and purified.

Generally, the disclosed compounds are administered as part of a pharmaceutical composition or formulation, and will be prepared for inclusion in such composition or formulations as isolated or purified compounds. In certain preferred embodiments, a disclosed compound is in a substantially pure or isolated preparation. The terms “isolated,” “purified,” or “substantially pure” as used herein, refer to material that is substantially or essentially free from components that normally accompany the material when the material is synthesized, manufactured, or otherwise produced, according to a particular degree of purity, and when measured by a method described herein or of general knowledge in the art. Any compound of the disclosure can be purified. A compound (or a composition comprising a compound) as described herein will generally be at least about 90% pure, including at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, at least about 99.9% pure, or up to 100% pure. A “substantially pure” compound (or a composition comprising a compound) may be defined as a compound or composition having a chromatographic purity (of the desired compound) of greater than about 90%, more preferably greater than about 95%, more preferably greater than about 96%, more preferably greater than about 97%, more preferably greater than about 98%, more preferably greater than about 99%, more preferably greater than about 99.25%, more preferably greater than about 99.50%, more preferably greater than about 99.75%, and most preferably greater than about 99.90%, greater than about 99.95%, or greater than about 99.99%, as determined by area normalization of HPLC profile or other similar detection method.

A substantially pure compound of the invention is, preferably, substantially free of any other active agents which are not intended to be administered to a subject. In this context “substantially free” can be taken to mean that no impurities, adulterants, or active agent(s) other than the active agent intended to be administered to a subject are detectable by HPLC or other similar detection method, or are below a desired threshold of detection such as defined above. Other active agents that may be found in a compound that is not substantially pure include those known in the art to be present in recreational or non-clinical MDMA, such as by-products and side-products of non-GMP synthesis, or compounds sold as or with “MDMA” while not in fact MDMA (e.g., other phenethylamines, cathinones, etc.) (see, e.g., Saleemi 2017). In some embodiments, the composition of the invention therefore will comprise less than about 5% impurities or adulterants, including additional (undesired) active agents such as described above, and in some embodiments will comprise less than 4%, less than 3%, less than 2%, less than 1%, less than 0.5%, less than 0.4%, less than 0.3%, less than 0.2%, less than 0.1%, less than 0.05%, less than 0.025%, or less than 0.01% impurities or adulterants, including in some embodiments no measurable impurities or adulterants.

### B. COMPOSITIONS AND FORMULATIONS

“Pharmaceutical compositions” are compositions comprising disclosed compound(s) together in an amount (for example, in a unit dosage form) with a pharmaceutically acceptable carrier, diluent, or excipient. It should be understood that some embodiments do not have a single carrier, diluent, or excipient alone, but include multiple carriers, diluents, and/or excipients.

Compositions can be prepared by standard pharmaceutical formulation techniques such as disclosed in Remington: The Science and Practice of Pharmacy (2005) 21^{st} ed., Mack Publ. Co., Easton, Pa.; The Merck Index (1996) 12^{th} ed., Merck Publ. Group, Whitehouse, N.J.; Pharm. Principles of Solid Dosage Forms (1993), Technomic Publ. Co., Inc., Lancaster, Pa.; and Ansel and Stoklosa, Pharm. Calculations (2001) 11^{th} ed., Lippincott Williams & Wilkins, Baltimore, Md.; and Poznansky et al. Drug Delivery Systems (1980), R. L. Juliano, ed., Oxford, N. Y, pp. 253-315).

“Pharmaceutically acceptable” as used in connection with one or more ingredients means that the ingredients are generally safe and, within the scope of sound medical judgment, suitable for use in contact with the cells of humans and other animals without undue toxicity, irritation, allergic response, or complication, and commensurate with a reasonable risk/benefit ratio.

These compositions can be prepared as formulations suitable for administration by a variety of routes, non-limiting examples of which include enteral means, wherein “enteral means” or “enteral administration means” includes, but is not limited to oral solid and oral liquid dosage forms, sublingual, and buccal administration means; as well as parenteral means, wherein “parenteral means” or “parenteral administration means” includes but is not limited to, intravenous, intra-arterial, intraperitoneal, intraosseous, intramuscular, intrathecal, intracerebroventricular, rectal, vaginal, ocular, nasal, cutaneous, topical, otic, ocular, transdermal, and subcutaneous administration means. As appreciated by one of skill, the compounds employed in the disclosed methods are effectively administered as oral solid and oral liquid dosage forms; sublingually or buccally; as injections, including intravenous, intra-arterial, intraperitoneal, intraosseous, intramuscular, intrathecal, and intracerebroventricular; rectally, vaginally, ocularly, nasally, cutaneously, topically, oticly, transdermally, and subcutaneously. Additionally, the disclosed compounds are effectively administered via other means, and prepared as any acceptable compositions known to those of skill. Such compositions may be prepared in any manner known in the pharmaceutical arts that comprise at least one active agent (Sheth et al., 1980).

In making the compositions employed in the present invention the active ingredients are often mixed with an excipient, diluted by an excipient, or enclosed within such a carrier which can be in the form of a capsule, sachet, paper, or other container. When the excipient serves as a diluent, it can be a solid, semi-solid, or liquid material, which acts as a vehicle, carrier, or medium for the active ingredient. Thus, the compositions can be in the form of tablets, pills, lozenges, sachets, cachets, elixirs, suspensions, emulsions, solutions, syrups, aerosols (as a solid or in a liquid medium), ointments, soft or hard gelatin capsules, suppositories, sterile injectable solutions, and sterile packaged powders. Different embodiments include immediate, delayed, extended, and controlled release forms. Many other variations are possible and known to those skilled in the art.

In some embodiments of the invention are multiple routes of administration, which may differ between patients according to patient preferences, comorbidities, side effect profiles, pharmacokinetic and pharmacodynamic considerations, and other factors. In some embodiments are the presence of other substances with the active agents, known to those skilled in the art, such as fillers, carriers, gels, skin patches, lozenges, or other modifications in the preparation to facilitate absorption through various routes (e.g., gastrointestinal, transdermal, etc.), to extend the effect of the drugs, and/or attain higher or more stable serum levels or enhance the therapeutic effect of the active agents in the combination.

In preparing a formulation, it may be necessary to mill the active agent to provide the appropriate particle size prior to combining with the other ingredients. If an active agent is substantially insoluble, it ordinarily is milled to a particle size of less than about 200 mesh.

Excipients, as defined herein, may include, but are not limited to, fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

The pharmaceutical compositions described herein can be formulated into any suitable dosage form, including aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed-release formulations, immediate-release formulations, modified release formulations, extended-release formulations, pulsatile release formulations, multi particulate formulations, and mixed immediate release and controlled release formulations. Generally speaking, one will desire to administer an amount of the active agent of the invention that is effective to achieve a plasma level commensurate with the concentrations found to be effective in vivo for a period of time effective to elicit the desired therapeutic effect(s).

In some embodiments, the compositions are formulated in a unit dosage form. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a subject for an extended or brief period of time.

In some embodiments, the disclosed compositions are formulated in a pharmaceutically acceptable oral dosage form. Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations.

In some embodiments, the oral solid dosage forms of the invention may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations of the invention may be administered as a single capsule or in multiple capsule dosage form. In some embodiments, the pharmaceutical formulation is administered in two, three, four, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s). Supplementary active agents include preservatives, antioxidants, antimicrobial agents including biocides and biostats such as antibacterial, antiviral and antifungal agents.

In some embodiments, the pharmaceutical composition may be an “immediate release formulation,” wherein a therapeutically effective amount of the pharmaceutical composition is administered to the subject in a way that facilitates rapid release. Immediate-release formulations may be prepared by combining a superdisintegrant such as croscarmellose sodium and different grades of microcrystalline cellulose in different ratios. In some embodiments, to aid disintegration, sodium starch glycolate may be added.

Tablets of the invention can be prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein. Generally recognized compendia of methods include Remington 2020 and Sheth et al. 1980.

In some embodiments, solid dosage forms are prepared by mixing the active agents of the invention with one or more pharmaceutical excipients to form a “bulk blend” composition. The bulk blend composition is homogeneous, i.e., the active agents are dispersed evenly throughout so that the bulk blend may be readily subdivided into equally effective unit dosage forms, such as tablets, pills, and capsules. The individual unit dosages may also comprise film coatings, which disintegrate upon oral ingestion or upon contact with diluents. These formulations can be manufactured by conventional pharmaceutical techniques. Conventional pharmaceutical techniques for preparation of solid dosage forms include, but are not limited to, the following methods, which may be used alone or in combination: (1) dry mixing, (2) direct compression, (3) milling, (4) dry or non-aqueous granulation, (5) wet granulation, or (6) fusion (see, e.g., Lachman et al. 1986). Other methods include spray drying, pan coating, melt granulation, granulation, fluidized bed spray drying or coating (e.g., Wurster coating), tangential coating, top spraying, tableting, and extruding.

Depending on the desired release profile, oral solid dosage forms may be prepared as immediate release formulations, or as modified release formulations, such as controlled release, extended release, sustained release, or delayed release. In some embodiments, the release profile is determined by obtaining objective measurements from a patient, the objective measurements including but are not limited to, weight, body temperature, heart rate, respiratory rate, blood oxygenation, BP and its variables, including, but not limited to: SBP, SBP, MAP, and PP; CNIBP; ECG measurements, including RR interval or its variability, QT interval or its variability, HRV (including measured by devices other than an ECG); hemodynamic response, and levels of glucose, cortisol, serotonin, dopamine, cholesterol; EEG measures; BDNF; genetic markers including relating to CYP enzymes or drug metabolism; genetic variation in mGluR5 or FKBP5; and numerous more, as will be readily appreciated by those of skill.

In some embodiments of modified release formulations, the half-life compared to the half-life of an immediate release formulation is greater by at least 5%, at least 10%, at least 15%, at least 20%, at least 25%, at least 30%, at least 35%, at least 50%, at least 75%, at least 100%, or values in between. In some embodiments of modified release formulations, the formulations are designed to result in a comparable “area under the curve,” or AUC₀₋₂₄, and a similar safety and efficacy profile, but having a delayed time to maximum concentration (tₘₐₓ) of at least 5%, at least 10%, at least 15%, at least 20%, at least 25%, at least 30%, at least 35%, at least 50%, at least 75%, at least 100%, or values in between, as would be appreciated by one of skill. In some preferred embodiments, a formulation is designed to be a product with a specific time course based on an optimum “therapeutic window,” such as less than about 30 minutes (“mins”), about 30 mins, about 45 mins, about 60 mins, about 90 mins, about 2 hours (“hrs”), about 3 hrs, about 4 hrs, about 5 hrs, about 6 hrs, about 7 hrs, about 8 hrs, and greater than 8 hrs, including lengths of time in between.

In some embodiments, oral solid dosage forms are formulated as a delayed release dosage form by utilizing an enteric coating to affect release in the small intestine of the gastrointestinal tract. An enteric-coated oral dosage form may be a compressed or molded or extruded tablet/mold (coated or uncoated) containing granules, powder, pellets, beads or particles of the active ingredient and/or other composition components, which are themselves coated or uncoated. The enteric-coated oral dosage form may also be a capsule (coated or uncoated) containing pellets, beads or granules of the solid carrier or the composition, which are themselves coated or uncoated. In some embodiments, enteric coatings may be used to prepare other controlled release dosage forms, including but not limited to extended release and pulsatile release dosage forms. Pulsatile release dosage forms may be formulated using techniques known in the art, such as those described in U.S. Pat. Nos. 5,011,692, 5,017,381, 5,229,135, and 5,840,329. Other suitable dosage forms are described in U.S. Pat. Nos. 4,871,549, 5,260,068, 5,260,069, 5,508,040, 5,567,441 and 5,837,284.

In some embodiments, the controlled release dosage form is a pulsatile release solid oral dosage form comprising at least two groups of particles, each containing active agents of the invention. “Particles” refers to homogenous groupings of substantially the same active agent. The first group of particles, upon ingestion by a subject, provides a substantially immediate dose of the active agents of the invention, and may either be uncoated, or comprise a coating and/or sealant. Using such means, a single unit dosage form can provide both a first and a second dosage amount in the single form (i.e., a first dosage amount in an immediate release form, and a second dosage amount in a delayed release form). In some embodiments, gastroretentive sustained release tablets are formulated by using a combination of hydrophilic polymer (e.g., hydroxypropyl methylcellulose), together with at least one swelling agent (e.g., crospovidone, sodium starch glycolate, and croscarmellose sodium), and an effervescent substance (e.g., sodium bicarbonate). Using known methods, gastroretentive tablets can be formulated to prolong the gastric emptying time and extend the mean residence time (MRT) in the stomach for optimal drug release and absorption (see, e.g., Arza 2009). Coatings for providing a controlled, delayed, or extended release may be applied to the disclosed compositions or to a core containing the compositions, and may comprise a pharmaceutically acceptable ingredient in an amount sufficient to provide a delayed release from, for example, about 1 hour to about 7 hours following ingestion before release of the active agents. Suitable coatings include one or more differentially degradable coatings including pH-sensitive coatings (enteric coatings), or non-enteric coatings having variable thickness to provide differential release of the active agents. Many other types of modified release systems will be known to those of skill. Non-limiting examples of additional delivery systems include both polymer- and nonpolymer-based systems, silastic systems, peptide-based systems, wax coatings, bioerodible dosage forms, and compressed tablets using conventional binders (see, e.g., Liberman et al. 1990; Singh et al. 2002; U.S. Pat. Nos. 4,327,725; 4,624,848; 4,968,509; 5,461,140; 5,456,923; 5,516,527; 5,622,721; 5,686,105; 5,700,410; 5,977,175; 6,465,014; and 6,932,983).

In some embodiments, the disclosed compositions are formulated as a pharmaceutically acceptable oral liquid form. Oral liquid dosage forms include tinctures, drops, emulsions, syrups, elixirs, suspensions, and solutions, and the like. Oral liquid dosage forms may be formulated with any pharmaceutically acceptable excipient known to those of skill for the preparation of liquid dosage forms, and with solvents, diluents, carriers, excipients, and the like chosen as appropriate to the solubility and other properties of the active agents and other ingredients. Solvents may include water, glycerin, simple syrup, alcohol, medium chain triglycerides, and combinations thereof.

Oral liquid dosage forms are especially advantageous in treating geriatric and pediatric populations, as the liquid formulation is much easier to swallow. The liquid formulation is also absorbed more rapidly than a solid dosage form, as it need not be dissolved by the digestive system before it is available for absorption.

Oral liquid dosage forms may be monophasic or biphasic, the former being a substantially homogenous solution dissolved in water or non-aqueous solvent, while the latter refers to oral liquid dosage forms in which the active ingredients do not fully dissolve in common solvents. Thus, over time, the solid particles (i.e., the active agents) within the oral liquid dosage form may form a precipitate at the bottom of the container-requiring vigorous shaking to redisperse the active ingredients. Examples of monophasic liquid forms include syrups, linctuses, spirits/essences, elixirs, and fluid extracts. Examples of biphasic liquid forms include oral suspensions, oral emulsions, and mixtures.

Liquid dosage forms for oral administration may be prepared as liquid suspensions or solutions using a sterile liquid, such as but not limited to, an oil, water, an alcohol, combinations of pharmaceutically suitable surfactants, suspending agents, and emulsifying agents. Liquid formulations also may be prepared as single dose or multi-dose beverages. Suspensions may include oils. Such oils include, but are not limited to peanut oil, sesame oil, cottonseed oil, corn oil, and olive oil. Suitable oils also include carrier oils such as MCT and long chain triglyceride (LCT) oils. Suspension preparation may also contain esters of fatty acids such as ethyl oleate, isopropyl myristate, fatty acid glycerides, and acetylated fatty acid glycerides. Suspension formulations may include alcohols, such as ethanol, isopropyl alcohol, hexadecyl alcohol; glycerol, and propylene glycol. Ethers, such as polyethylene glycol; petroleum hydrocarbons, such as mineral oil and petrolatum; and water may also be used in suspension formulations. Suspension can thus include an aqueous liquid or a non-aqueous liquid, an oil-in-water liquid emulsion, or a water-in-oil emulsion.

Dosage forms for oral administration may be aqueous suspensions such as aqueous oral dispersions, emulsions, solutions, and syrups (see, e.g., Singh et al. 2002). In addition to the active agents, the liquid dosage forms may comprise additives, such as one or more (a) disintegrating agents, (b) dispersing agents, (c) wetting agents, (d) preservatives, (e) viscosity enhancing agents, (f) sweetening agents, and/or (g) flavoring agents. In addition to the additives listed above, the liquid formulations of the invention, in some embodiments, may also comprise inert diluents commonly used in the art such as water or other solvents, solubilizing agents, emulsifiers, flavoring agents, and/or sweeteners. Co-solvents and adjuvants also may be added to a formulation.

In some embodiments, effervescent powders containing the disclosed compositions may be prepared. Effervescent salts are used to disperse medicines in water for oral administration. Effervescent salts also may be packaged as single dose or multi-dose drink mixes, alone or in combination with other ingredients, such as vitamins or electrolytes. Effervescent salts are granules or coarse powders containing a medicinal agent in a dry mixture, usually composed of sodium bicarbonate and sodium carbonate, citric acid, and/or tartaric acid. When salts of the invention are added to water, the acids and the base react to liberate carbon dioxide gas, thereby causing “effervescence.” Any acid-base combination that results in the liberation of carbon dioxide may be used, if the ingredients are suitable for pharmaceutical use and result in a pH of about 6.0 or higher.

In some embodiments, the disclosed compositions are formulated in a pharmaceutically acceptable transdermal application, and delivered transdermally. Generally speaking, transdermal delivery involves contacting the formulations of the invention with a subject's skin under conditions effective for the active agent(s) to penetrate the skin and cause an effect. Formulations may be prepared as ointments, creams, suspensions, lotions, pastes, gels, sprays, foams, oils, and the like, and any combination thereof.

In some embodiments, the disclosed compositions are formulated for sublingual or buccal administration. In some embodiments, a sublingual or buccal formulation will be a fast-dissolving dosage form (FDDF). Exemplary FDDFs include loosely compressed tablets comprising a relatively large amount of wicking or disintegrating agents, tablets comprising a relatively large amount of effervescent agents, lyophilized tablets, and dosage forms using rapidly soluble gelatin-based matrices, or using gelatin or mannitol as carriers or matrix forming agents, and which are designed to release the active ingredient in the oral cavity (see, e.g., Seagar, H., “Drug-Delivery Products and Zydis Fast Dissolving Dosage Form,” J. Pharm. Pharmaco, vol. 50, p. 375-382 (1998)). In some embodiments of a FDDF, the FDDF is a freeze-dried oral solid dosage form, including an orally dissolving tablet (ODT), such as a ZYDIS® oral solid dosage form, including a ZYDIS ODT, such as a ZYDIS Ultra (Catalent, Swindon, U.K.). In some embodiments, a FDDF is an ODT. In some embodiments, the ODT is a ZYDIS ODT such as described in U.S. Pat. No. 9,192,580, which is incorporated by reference as if fully set forth herein. In embodiments, an ODT will comprise a disclosed entactogen, such as MDMA, a non-gelling matrix former, a filler, a binder, and a pH modifying agent (i.e., a buffer), and optionally any one or more of antioxidants, photostabilization agents, permeation enhancers, coloring agents, sweeteners, and flavorants or flavoring agents. In some embodiments, a solid oral dosage form is a solid oral immediate release formulation, such as a capsule, a tablet, or an ODT, wherein the solid oral immediate release formulation comprises a disclosed entactogen, such as MDMA, including as disclosed by either of WO2023/023192A1 or WO2023/023182A1, incorporated by reference as if fully set forth herein.

An exemplary transdermal delivery form is a transdermal “patch” which contains the pharmaceutical compositions. Transdermal patches may be used to provide continuous or discontinuous infusion of the compounds of the invention in controlled amounts. Such patches may be constructed for continuous, gradual, pulsatile, or on demand delivery of pharmaceutical agents. A “patch” within the meaning of the invention may be simply a medicated adhesive patch, i.e., a patch impregnated with a composition of the invention for application onto the skin. Thus, a patch may be a single-layer or multi-layer drug-in-adhesive patch, wherein the one or more adhesive layers also contain the active agents. A patch may also be a “matrix” (or “monolithic”) patch, wherein the adhesive layer surrounds and overlays the drug layer (wherein a solution or suspension of the active agents is in a semisolid matrix). A “reservoir” patch may also be used, comprising a drug layer, typically as a solution or suspension of the active agents in a liquid compartment (i.e., the reservoir), separate from an adhesive layer. A patch also may be part of a delivery system, for instance used with an electronic device communicatively coupled to the mobile device of a user, and coupled with a mobile application (e.g., to control the delivery rate from the reservoir, and optionally to provide information about delivery back to the application or user). Various transdermal patch technologies may be accordingly utilized.

In embodiments, the disclosed compositions may be prepared as formulations designed for subcutaneous, intravenous, intra-arterial, intraperitoneal, intraosseous, intramuscular, intrathecal, or intracerebroventricular injection. Injection formulations may be prepared by dissolving, suspending, or emulsifying the active agent(s) in an aqueous or nonaqueous solvent, non-limiting examples of which include oils, such as vegetable oil, synthetic aliphatic acid glycerides, and esters of higher aliphatic acids or propylene glycol; and may also contain additives such as solubilizers, stabilizers, and suspending, preserving, wetting, emulsifying, dispensing, and isotonic agents.

In some embodiments, the disclosed compositions are formulated in a pharmaceutically acceptable nanostructured formulation, such as a nanoemulsion, a nanocapsule, a nanoparticle conjugate, or a nano-encapsulated oral or nasal spray. Preparations of the disclosed compositions as certain nanostructured formulations may be done by reference to the general knowledge of the art (see, e.g., Jaiswal 2015).

The prefix “nano” as used in the terms describing various embodiments of a nanostructured formulation denotes a size range in the nanometer (“nm”) scale. Accordingly, sizes of such nanoparticle delivery vehicles include those in the range of about 1 to about 100 nm, about 100 to about 200 nm, about 200 to about 400 nm, about 400 to about 600 nm, about 600 to about 800 nm, and about 800 to about 1000 nm, as well as “microparticles” in the range of about 1000 to about 2000 nm (1-2 micrometer (“m”) scale). Particles of certain sizes may be particularly advantageous depending on the method of administration, as will be immediately appreciated by one of skill (e.g., for oral liquid emulsion versus for transdermal or topical application). In some embodiments, lipid-based nanoparticles (LBNPs) such as liposomes, solid lipid nanoparticles (SLN), or nanostructured lipid carriers (NLC) are used.

The disclosed compositions may be prepared as pharmaceutically acceptable formulations suitable as oral solid or oral liquid dosage forms; for administration sublingually, buccally, topically, rectally, vaginally, ocularly, oticly, nasally, cutaneously, topically, or transdermally; for intravenous, intra-arterial, intraperitoneal, intraosseous, intramuscular, intrathecal, intracerebroventricular, or subcutaneous injection, where such injections comprise physiologically acceptable sterile aqueous or non-aqueous solutions, dispersions, suspensions or emulsions, liposomes, and sterile powders for reconstitution into sterile injectable solutions or dispersions.

The disclosed pharmaceutical compositions may be administered via enteral or parenteral means, wherein enteral means includes, but is not limited to, oral solid and oral liquid dosage forms, sublingual, and buccal administration; and parenteral administration means includes, but is not limited to, intravenous, intra-arterial, intraperitoneal, intraosseous, intramuscular, intrathecal, intracerebroventricular, rectal, vaginal, ocular, nasal, cutaneous, topical, otic, transdermal, and subcutaneous administration; in addition to other equivalent means known to those of skill.

The term “administering” or “administration” broadly refers to providing a compound or pharmaceutical composition of the invention to a subject suffering from, or at risk of, the diseases or conditions to be treated or prevented. As used herein, the terms “subject,” “user,” “patient,” and “individual” are used interchangeably, and refer to any mammal although preferably a human. Such terms will be understood to include one who has an indication for which a compound, composition, or method described herein may be efficacious, or who otherwise may benefit by the invention. In general, all of the disclosed compounds, compositions, and methods will be appreciated to work for all individuals, although individual variation is to be expected, and will be understood.

Routes of administration for the disclosed compounds and compositions include enteral and parenteral. Enteral administration includes administration involving any part of the gastrointestinal tract. The examples may include those by mouth (orally), including oral solid and oral liquid dosage forms, and may be preferably formulated as tablets or capsules. Parenteral administration includes administration by any means not involving the gastrointestinal tract, including intravenously, intra-arterially, by intraosseous infusion, intramuscularly, intracerebrally, intracerebroventricularly, intrathecally, oticaly, ocularly, rectally, vaginally, and subcutaneously.

In some embodiments, parenteral administration may include sublingual and/or buccal administration. In some embodiments, a pharmaceutical composition may be administered to a subject via injection. In some embodiments, a pharmaceutical composition is administered to a subject via nasal systems or the mouth through, for example, an oral solid and/or oral liquid dosage forms; by inhalation, via a nasal spray or oral inhaler; by nebulization, via a nebulizer; or buccally or sublingually. In some embodiments, a pharmaceutical composition is administered to a subject via a combination of administration means. In some embodiments, the pharmaceutical composition is administered to a subject via one or more enteral administration means. In some embodiments, the pharmaceutical composition is administered to a subject via one or more parenteral administration means. In some embodiments, the pharmaceutical composition is administered to a subject by at least one enteral administration means, and at least one parenteral administration means. In embodiments, an equivalent route of administration known to one of skill is utilized.

By way of non-limiting and merely suggestive example, the following formulations and associated routes of administration may be used in methods of the invention:

### Example 1: Formulation of Capsules with 125 mg or 62.5 mg of an Entactogen

Gelatin capsules, containing the below milligram amounts of an entactogen are made as follows:

Exemplary 125 mg Entactogen Capsule

| Ingredient | Quantity (mg/capsule) |
|---|---|
| Entactogen, e.g., MDMA | 125.0 |
| Starch | 73.0 |
| Magnesium stearate | 2.0 |

Exemplary 62.5 mg Entactogen Capsule

| Ingredient | Quantity (mg/capsule) |
|---|---|
| Entactogen, e.g., MDMA | 62.5 |
| Starch | 36.5 |
| Magnesium stearate | 1.0 |

The entactogen, such as MDMA, is synthesized through known methods or commercially sourced. The entactogen, starch, and magnesium stearate are blended, passed through a No. 20 mesh U.S. sieve, and filled into size 4 hard or soft gelatin capsules in 200 mg or 100 mg quantities as above.

### Example 2: Formulation of Tablets with 125 mg or 62.5 mg MDMA

Tablets, containing the below milligram amounts of an entactogen, are made as follows:

Exemplary 125 mg Entactogen Tablet

| Ingredient | Quantity (mg/tablet) |
|---|---|
| Entactogen, e.g., MDMA | 125.0 |
| Cellulose, microcrystalline | 100.0 |
| Colloidal silicon dioxide | 65.0 |
| Stearic acid | 10.0 |

Exemplary 62.5 mg Entactogen Tablet

| Ingredient | Quantity (mg/tablet) |
|---|---|
| Entactogen, e.g., MDMA | 62.5 |
| Cellulose, microcrystalline | 50.0 |
| Colloidal silicon dioxide | 32.5 |
| Stearic acid | 5.0 |

The components are blended and compressed into tablets, weighing 300 mg or 150 mg total. In some embodiments, the entactogen is MDMA.

It will be readily appreciated that the above formulation examples are illustrative only. Accordingly, the entactogen may be substituted with the same entactogen in a different dosage amount (e.g., 125 mg, 120 mg, 115 mg, 110 mg, 105 mg, 100 mg, 95 mg, 90 mg, 85 mg, 80 mg, 75 mg, 70 mg, 65 mg, 62.5 mg, 60 mg, 55 mg, 50 mg, 45 mg, 40 mg, 35 mg, 30 mg, 25 mg, 20 mg, 15 mg, 10 mg, 5 mg, and values in between), or with another entactogen. It will be understood that reference to particular compounds is merely illustrative, and both active and inactive compounds in any Example may be substituted by other compounds of the invention. The entactogen may be racemic, an enantiomerically enriched composition in an enantiomeric excess in any amount greater than 0% (i.e., a non-racemic mixture), or a pure or substantially pure individual enantiomer.

In some embodiments, the entactogen is MDMA. In some embodiments, the MDMA is racemic MDMA. In some embodiments, the MDMA is an enantiomerically enriched composition of MDMA, in an enantiomeric excess in any amount greater than 0%, or a pure or substantially pure individual enantiomer, such as an enantiomer in an enantiomeric excess of greater than about 75%, about 76%, about 77%, about 78%, about 79%, about 80%, about 81%, about 82%, about 83%, about 84%, about 85%, about 86%, about 87%, about 88%, about 89%, or preferably above about 90%, such as about 91%, about 92%, about 93%, about 94%, about 95%, about 96%, about 97%, about 98%, about 99%, about 99.5%, about 99.9%, and up to about 100% and including 100%, as well as values in between.

While in some embodiments, the entactogen is MDMA, in other embodiments, a compound will be another entactogen. An “entactogen” (or an “empathogen”) refers to any member of the class of psychoactive compounds that produce distinctive “entactogenic” (or “empathogenic”) effects. In some embodiments, one or more of the entactogenic effects are the same as, comparable to, or similar to those of MDMA. In some embodiments, all of the entactogenic effects are the same as, comparable to, or similar to those of MDMA. In some embodiments, the entactogenic effects when considered as a whole are the same as, comparable to, or similar to those of MDMA.

Certain non-limiting features of entactogens are now described. Without being bound by theory, entactogens produce increases on all scales of the MEQ, and a standard response to an entactogen includes experiences of oneness and emotional openness. Additionally, entactogens significantly modulate social processing and have acute prosocial effects like increased empathy and communication (Preller et al., Front Psychiatry. 2019; 10:881). Another unique feature of entactogens is that a subject experiences reduced defense mechanisms and anxiety and increased self-acceptance, empathy, and peacefulness. Such effects are reflected in scores of greater magnitude on measurements of mystical type experiences, e.g., the Oceanic Boundlessness scale of the 5-Dimensional Altered States of Consciousness (5D-ASC) questionnaire and the MEQ. In some cases, these pleasant emotional experiences may be contrasted with a strong endocrine and autonomic effect, including significantly increased blood pressure and heart rate (Gouzoulis-Mayfrank, Heffter Rev Psychedelic Res 2001; 2: 64-72).

In some embodiments, an entactogen for use in EAP is any of a substituted amphetamine, a substituted benzofuran, a substituted phenethylamine, or a substituted tryptamine. For reference, and as non-limiting examples, MDMA is a substituted amphetamine, 6-APB is a substituted phenethylamine and a substituted benzofuran (demonstrating that a compound may fall into multiple classes, as will be appreciated by those of skill), and 5-MeO-MiPT is one example of a substituted tryptamine with entactogen properties. Further examples of entactogens will be known to those in the art, and in some embodiments will include substances within or derived from the 1,3-benzodioxole, cathinone, benzofuran, aminoindane, indole, and amphetamine classes (see, e.g., Oeri 2021). In certain preferred embodiments the entactogen is MDMA, such as for use in MDMA-assisted therapy, including single enantiomers and non-racemic mixtures thereof. In some embodiments, an entactogen is, as further non-limiting examples, 1-(1,3-benzodioxol-5-yl)-2-butanamine (BDB); 1-(1,3-benzodioxol-5-yl)-N-methyl-2-butanamine (MBDB); 3,4-methylene-dioxyamphetamine (MDA); 3,4-methylenedioxy-N-ethyl-amphetamine (MDEA), including single enantiomers and non-racemic mixtures thereof. In some embodiments, an entactogen is 3-methylmethcathinone (3-MMC), mephedrone (4-MMC), methylone, ethylone, butylone, α-ethyltryptamine (αET), α-methyltryptamine (WMT), 5-iodo-2-aminoindane (5-IAI), methylenedioxyaminoindane or 5,6-Dimethoxy-2-aminoindane (MDAI), 5-(2-Aminopropyl)-2,3-dihydro-1H-indene (5-APDI, IAP), 6-(2-Aminopropyl)benzofuran (6-APB) (benzofury), 5-(2-aminopropyl)benzofuran (5-APB), 1-(benzofuran-5-yl)-N-methylpropan-2-amine (5-MAPB), 3,4-methylenedioxy-N-hydroxyamphetamine (MDOH), and 4-fluoroamphetamine (4-FA). In some embodiments, an entactogen is any of 5-methoxy-2-aminoindane (MEAI), N-Allyl-3,4-methylenedioxy-amphetamine (MDAL), N-Butyl-3,4-methylenedioxyamphetamine (MDBU), N-benzyl-3,4-methylenedioxyamphetamine (MDBZ), N-cyclopropylmethyl-3,4-methylenedioxyamphetamine (MDCPM), N,N-dimethyl-3,4-methylene-dioxyamphetamine (MDDM), N-(2-Hydroxyethyl)-3,4-methylenedioxy amphetamine (MDHOET), N-isopropyl-3,4-methylenedioxyamphetamine (MDIP); N-methyl-3,4-ethylenedioxyamphetamine (MDMC), N-methoxy-3,4-methylenedioxyamphetamine (MDMEO), N-(2-methoxyethyl)-3,4-methylene-dioxyamphetamine (MDMEOET), α,α,N-trimethyl-3,4-methylenedioxyphenethylamine or 3,4-methylenedioxy-N-methylphentermine (MDMP), 3,4-methylenedioxyphenethylamine (MDPEA), α,α-dimethyl-3,4-methylenedioxyphenethylamine or 3,4-methylenedioxyphentermine (MDPH;), N-propargyl-3,4-methylenedioxyamphetamine (MDPL), gamma hydroxybutyrate (GHB), sodium oxybate (XYREM®), calcium, magnesium, potassium, and sodium oxybates (XYWAV®); N-propyl-3,4-methylenedioxyamphetamine (MDPR), including single enantiomers and non-racemic mixtures thereof.

In some embodiments the entactogen is a substituted amphetamine, substituted phenethylamine, or substituted benzofuran, such as any of 3-chloromethamphetamine (3-CMA), 4-fluoromethamphetamine (4-FMA), 4-methylamphetamine (4-MA), 4-methylmethamphetamine (4-MMA), 4-methylthioamphetamine (4-MTA), 5-(2-aminopropyl)-2,3-dihydrobenzofuran (5-APDB), 1-(benzofuran-5-yl)-N-ethylpropan-2-amine (5-EAPB), 5-(2-aminopropyl)indole (5-IT), 1-(2,3-dihydrobenzofuran-5-yl)-N-methylpropan-2-amine (5-MAPDB), 6-(2-aminopropyl)-2,3-dihydrobenzofuran (6-APDB), 1-(benzofuran-6-yl)-N-ethylpropan-2-amine (6-EAPB), 5-(2-aminopropyl)indole (6-IT), 1-(2,3-dihydrobenzofuran-6-yl)-N-methylpropan-2-amine (6-MAPDB), 3,4-ethylidenedioxyamphetamine (EDA), 2,3-methylenedioxyamphetamine (2,3-MDA), tenamfetamine (3,4-MDA), tetralinylaminopropane (TAP), or naphthylaminopropane (PAL-287).

In some embodiments, an entactogen is any substituted methylenedioxy-phenethylamine or “MDxx” compound, as will be understood by those in the art. In further embodiments, an entactogen is an analog or derivative of an MDxx compound, such as a compound where the methylenedioxyphenyl ring is retained but the phenethyl portion is modified, or a compound which retains the 3,4-cyclised amphetamine core common to the MDxx compounds, but has the 1,3-benzodioxole ring replaced by related heterocycles. In some embodiments, an entactogen is a “2C-x” compound with entactogenic or primarily entactogenic effects.

In some embodiments, an entactogen will be any other compound generally known to those of ordinary skill in the art as producing “entactogenic” or “empathogenic” effects, and which herein is used in EAP, and also may be used in “psychedelic”-assisted therapy, further including (and it shall be understood that all disclosed entactogens herein also shall further include) single enantiomers and enantiomeric mixtures; salts and solid forms such as polymorphs, hydrates, solvates, and co-crystals; amorphous forms; deuterated and halogenated versions; and prodrugs, metabolites, analogs, and derivatives of any of the above, including combinations thereof, and further including novel chemical compounds or NCEs having similar structures and/or effects, which may be modified to retain one or more entactogenic effects, but have one or more other novel effects (e.g., minimized side effects, reduced neurotoxicity, optimized onset or course of action, optimized pharmacokinetic or pharmacodynamic properties, and the like).

Moreover, for any of the compounds of the invention, substitution of the compound by its ion, free base, or salt (including in some preferred embodiments, a hydrochloride salt), a polymorph, or an isomer, shall be understood to provide merely an alternative embodiment still within the scope of the invention (with modifications to the formulation and dosage amounts made according to the teachings herein and ordinary skill). Further, compositions within the scope of the invention should be understood to be open-ended and may include additional active or inactive compounds and ingredients. In some embodiments, the composition will contain one or more additional compounds that provide a synergistic effect.

The type of formulation employed for the administration of the compounds employed in the disclosed methods generally are dictated by the compound(s) employed, the type of pharmacokinetic profile desired from the route of administration and the compound(s), and the state of the patient. It will be readily appreciated that any of the above embodiments and classes of embodiments can be combined to form additional embodiments and formulations.

### C. DOSAGE

In some aspects are disclosed methods for using therapeutically effective amounts of the disclosed compositions in a mammal, and preferably a human. Such methods include those for modulating neurotransmission and for treating a disorder such as a mental health or psychiatric disorder, and specifically AUD, or for a comorbid disorder, such as an addiction-related condition.

In some embodiments, where a disclosed composition includes a disclosed entactogen, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges. In some embodiments, where a disclosed composition includes a disclosed entactogen, including where such composition is used as a “booster” dose, it may be present in an amount so that a single dose is (in a milligram dosage amount calculated based on the kilogram weight of the patient) between about 0.01 mg/kg and 0.1 mg/kg, such as about 0.01 mg/kg, about 0.02 mg/kg, about 0.03 mg/kg, about 0.04 mg/kg, about 0.05 mg/kg, about 0.06 mg/kg, about 0.07 mg/kg about 0.08 mg/kg about 0.09 mg/kg, and about 0.1 mg/kg, as well as ranges between these values. In some embodiments, a single dose is between about 0.1 mg/kg and 1.0 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg about 0.8 mg/kg about 0.9 mg/kg, and about 1.0 mg/kg, as well as ranges between these values.

In some embodiments, where a disclosed composition includes a disclosed entactogen, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

The pharmaceutical compositions disclosed herein comprise therapeutic amounts of an entactogen, such as MDMA, and/or other active or inactive ingredients. In some embodiments, where a pharmaceutical composition includes an entactogen, such as MDMA, it may be present in an amount so that a single dose is (whether or not such dose is present in a unit dosage form), 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, and including values in between (each of the above dose amounts taken together, “a dose between 1 mg to 200 mg”).

In some embodiments, a patient may be administered an initial dose of between 1 mg to 200 mg of the entactogen, such as MDMA, where the initial dose is a dose between 1 mg to 200 mg”), and then subsequently administered a booster dose of between 0.5 mg to 100 mg of the entactogen, such as MDMA, including 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 87, 88 mg, 89 mg, 90 mg, 91 mg, 92 mg, 93 mg, 94 mg, 95 mg, 96 mg, 97 mg, 98 mg, 99 mg, 100 mg, and values in between. In some embodiments, the booster dose of the entactogen, such as MDMA, will be 12 of the initial dose of the entactogen, such as MDMA, such that, in an exemplary embodiment, if a subject were administered 100 mg of the entactogen, such as MDMA, in an initial dose, the second such dose administered to the subject would be 50 mg. In some embodiments, such as in certain aspects of Examples 4 or 8, wherein a subtherapeutic or “active” control of 20 or 10 mg is used, capsules may be prepared as in Example 1 (or tablets as in Example 2), but with amounts of the active and inactive ingredients adjusted accordingly. In some embodiments, the booster dose of the entactogen, such as MDMA, is greater than 100 mg, such as 125 mg, 150 mg, and 175 mg, and including up to 200 mg.

In embodiments, a subject is administered a single dose of the entactogen, such as MDMA, wherein the single dose of the entactogen, such as MDMA, is a dose between 1 mg to 200 mg.

In some embodiments, a single dose of the entactogen, such as MDMA, will be between 25 mg and 175 mg, preferably between 50 mg and 150 mg, and more preferably between 62.5 mg and 125 mg, inclusive. In one embodiment, an EAP regime of two doses of the entactogen, such as MDMA, is used, with an “initial dose” of 125 mg and a “booster dose” two hours later of 62.5 mg. In another embodiment, an EAP regime of two doses of the entactogen, such as MDMA, is used, with an initial dose of 100 mg and a “booster” dose two hours later of 50 mg. In another embodiment, an EAP regime of two doses of the entactogen, such as MDMA, is used, with an initial dose of 80 mg and a “booster” dose two hours later of 40 mg. In some embodiments where two doses are administered to a patient, the booster dose will be 0.5× (12) or about 0.5× (12) of the initial dose (see, e.g., Sessa et al. 2021). In embodiments, the booster dose will be chosen based on objective or subjective patient measures, based on patient selection or choice at or preceding the time of administration, or based on the exercise of clinical judgment at or preceding the time of administration. In embodiments, patients may receive a third or further dose (i.e., a second or further booster dose), which may be the same, a greater, or a lesser dose than the first booster dose.

Doses of an entactogen, where the entactogen is MDMA, ranging from 100-125 mg reach peak plasma concentration in humans after 2-3 hours. When patients with PTSD were administered this dosing regime, onset of action occurred 45-75 minutes after the initial dose and peak drug effects were reported at 2-5 hours (Mithoefer et al. 2013). For those receiving one dose, the effects lasted 4-5 hours, which was extended with the booster dose to 5-6 hours. In a prior study, subjective effects were reported after approximately 45 mins, peaking around 1.5-2 hours, and decreasing to baseline values 5-6 hours later (Harris 2002). The elimination half-life of MDMA is between 7-9 hours, with similar results for doses ranging from 50-125 mg (Mas et al. 1999), and for other entactogens, as will be understood to those in the art. In brain imaging studies, peak subjective effects have been reported at 100 mins and have lasted approximately 4.5 hours after 100 mg MDMA in healthy volunteers (Carhart-Harris 2013).

In embodiments, where a disclosed composition includes an additional active agent, such as disclosed herein, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

It will be understood that dosages may vary depending upon the treatment protocol itself, the onset, progression, severity, frequency, duration, probability of, or susceptibility of the symptom to which treatment is directed, clinical endpoint desired, previous, simultaneous or subsequent treatments, general health, age, gender, and race of the subject, bioavailability, potential adverse systemic, regional, or local side effects, the presence of other disorders or diseases in the subject, and other factors that will be appreciated by the skilled artisan (e.g., medical or familial history). For example, the upper age range of subjects may be limited, e.g., to 65 years, and patients may be screened for cardiac abnormalities, and for medications that may affect the metabolism of the entactogen, such as MDMA (e.g., through CYP2D6 or CYP3A4), or genetic variations that may affect metabolism of the entactogen, such as MDMA.

Dose amount, frequency or duration may be increased or reduced, as indicated by the clinical outcome desired, status of the pathology or symptom, any adverse side effects of the treatment or therapy, or concomitant medications. For example, as MDMA at doses of 100-125 mg has been associated with increases in heart rate (of 26-30 bpm) and blood pressure (of diastolic 14.4-25 mmHg) (Harris 2002, Mas 1999, Mithoefer et al. 2011) these may be carefully monitored following the regime disclosed elsewhere (Oehen et al. 2013, Chabrol & Oehen 2013), and patients experiencing particular symptoms may not receive a booster dose. The skilled artisan with the teaching of this disclosure in hand will appreciate the factors that may influence the dosage, frequency, and timing required to provide an amount sufficient or effective for providing a therapeutic effect or benefit, and to avoid or minimize adverse effects.

It will be understood that, in some embodiments, the dose actually administered will be determined by a physician, in light of the relevant circumstances, the method of delivery, (i.e., methods that are systemic and that are not subject to first pass effect will require less of a dose than those which are metabolized prior to entering the bloodstream; the age of the patient, the weight of the patient, whether the patient has any comorbidities (i.e., any other medical conditions simultaneously present within a patient), other medications the patient is taking (routinely or presently), and any patient-specific aspects that could affect the way in which the pharmaceutical composition interacts with the patient, such as variations in metabolism, variations in patient response, etc., and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, the compositions may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Dosage levels thus may differ from patient to patient, for individual patients across time, and for different pharmaceutical compositions and formulations, but shall be able to be determined with ordinary skill. Determination of appropriate dosing shall include not only the determination of single dosage amounts, but also the determination of the number and timing of doses, and the time(s) of day or time(s) during a psychotherapeutic session preferable for their administration.

In some embodiments, especially where a formulation is prepared in single unit dosage form, suggested dosage amounts shall be known by reference to the format of the preparation itself. In other embodiments, suggested dosage amounts may be known by reference to the means of administration or by reference to the packaging and labeling, package insert(s), marketing materials, training materials, or other information and knowledge available to those of skill or the public. Another aspect of this disclosure therefore provides pharmaceutical kits containing a pharmaceutical composition or formulation of the invention, suggested administration guidelines or prescribing information therefor, and a suitable container. Individual unit dosage forms can be included in multi-dose kits or containers. Pharmaceutical formulations also can be packaged in single or multiple unit dosage forms for uniformity of dosage and ease of administration.

### D. ENTACTOGEN-ASSISTED PSYCHOTHERAPY (EAP)

In some aspects, provided herein are methods of using entactogens in conjunction with psychotherapy. Use of an entactogen in a psychotherapy regimen is referred to herein as “entactogen-assisted psychotherapy” or “EAP,” or equivalently “entactogen-assisted therapy.” Herein, administering an entactogen to a subject in conjunction with psychotherapy (EAP) may be referred to as “administering EAP,” “administration of EAP,” “facilitating EAP,” “facilitation of EAP,” or “using EAP,” i.e., “administering,” “facilitating,” and “using” in this context may be used interchangeably. In some embodiments, an EAP regimen comprises one or more sessions where an entactogen is administered to a subject. In some embodiments, an entactogen-assisted psychotherapy regimen comprises one or more sessions where no drug is administered, herein referred to as a “non-drug session” or a “non-drug psychotherapy session.” In some embodiments, the psychotherapy component of a entactogen-assisted psychotherapy regimen comprises talk therapy, e.g., any one or more of cognitive behavioral therapy, Acceptance and Commitment Therapy (ACT), and motivational interviewing. In some embodiments, the psychotherapy component of a entactogen-assisted psychotherapy regimen comprises a modified form of cognitive behavioral therapy comprising elements of ACT. In some embodiments, the psychotherapy is administered by a therapist.

In some embodiments, an entactogen for administration to a patient receiving EAP is a substituted amphetamine, a substituted benzofuran, a substituted phenethylamine, or a substituted tryptamine. In some embodiments, an entactogen for administration to a patient receiving EAP is any of MDMA, BDB, MBDB, MDA, MDEA, 3-MMC, 4-MMC, methylone, ethylone, butylone, αET, αMT, 5-IAI, MDAI, 5-APDI, 6-APB, 5-APB, 5-MAPB, MDAI, MDOH, and 4-FA. In some embodiments, the entactogen is MDMA.

a. Methods of Use

In some aspects, provided herein are methods of using entactogen-assisted psychotherapy (EAP), e.g., to treat a condition in a patient. In some embodiments, EAP is used to treat a substance use disorder in a patient. In some embodiments, EAP is used to treat a behavioral addiction in a patient. In some embodiments, EAP is used to treat one or more comorbidities in a patient having a condition. In some embodiments, the condition is related to addiction. In some embodiments, EAP is used to treat one or more comorbidities in a patient diagnosed with any one or more of a substance use disorder, a behavioral addiction, a mental health disorder, or a neurodegenerative condition. In some embodiments, the one or more comorbidities treated by administration of EAP is any one or more of a substance use disorder, a behavioral addiction, and a mental health disorder.

In some embodiments, administering EAP to a patient (interchangeably herein, a subject) having a substance use disorder treats one or more of a comorbid substance use disorder, behavioral addiction, and mental health disorder. In some embodiments, the substance use disorder is selected from alcohol use disorder, opioid use disorder, nicotine dependence and tobacco use disorder, sedative, hypnotic, and anxiolytic use disorder, and stimulant use disorder.

In some embodiments, administering EAP to a patient having a behavioral addiction treats one or more of a comorbid substance use disorder, behavioral addiction, and mental health disorder. In some embodiments, the behavioral addiction is selected from gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction.

In some embodiments, administering EAP to a patient having a mental health disorder treats one or more of a comorbid substance use disorder, behavioral addiction, and mental health disorder. In embodiments, the mental health disorder is selected from depression, major depressive disorder (MDD), treatment-resistant depression (TRD), atypical depression, postpartum depression, catatonic depression, a depressive disorder due to a medical condition, premenstrual dysphoric disorder, seasonal affective disorder, dysthymia, anxiety and phobia disorders, generalized anxiety disorder (GAD), agoraphobia, panic disorder, separation anxiety disorder, social anxiety disorder, post-traumatic stress disorder, adjustment disorders, feeding and eating disorders, including binge eating, bulimia, and anorexia nervosa, other binge behaviors, body dysmorphic syndromes, drug abuse or dependence disorders, disruptive behavior disorders, impulse control disorders, gaming disorders, gambling disorders, memory loss, dementia of aging, attention deficit hyperactivity disorder, personality disorders, including antisocial, avoidant, borderline, histrionic, narcissistic, obsessive compulsive, paranoid, schizoid and schizotypal personality disorders, attachment disorders, autism, social anxiety in an autistic patient, and dissociative disorders.

In some embodiments, administering EAP to a patient having a neurodegenerative condition treats one or more of a comorbid substance use disorder, behavioral addiction, and mental health disorder. In some embodiments, the neurodegenerative disorder is selected from adrenoleukodystrophy, AIDS dementia complex, Alexander disease, Alper's disease, Alzheimer's disease, amyloid disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington's disease, Kennedy's disease, Krabbe disease, Lewy body dementia, Lyme disease, Machado-Joseph disease, mild cognitive impairment (MCI), motor neuron disease, movement disorders, multiple sclerosis, Multiple systems atrophy, neuroacanthocytosis, Niemann-Pick disease, Parkinson's disease, Parkinson's disease dementia, Pelizaeus-Merzbacher Disease, Pick's disease, primary lateral sclerosis including its juvenile form, prion disease, progranulinopathy, progressive bulbar palsy, progressive supranuclear palsy, protein aggregation disease, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, synucleinopathy, Tabes dorsalis, tauopathy, Tay-Sachs disease, toxic encephalopathy, transmissible spongiform encephalopathy, vascular dementia, and X-linked spinal muscular atrophy.

b. Treatment of Substance Use Disorders

In some embodiments, an entactogen is used to treat a substance use disorder (SUD). In some embodiments, the entactogen is used in conjunction with psychotherapy to treat a SUD.

In some embodiments, SUDs are characterized by excessive use of nicotine, alcohol, and narcotics including prescription drugs and drugs of abuse. Such use may lead to one or more of social, academic, and occupational impairment. Commonly abused substances include, e.g., alcohol, tobacco (nicotine), cannabis, sedatives, hypnotics, anxiolytics, inhalants, opiates, opioids, and stimulants (Jahan & Burgess, “SUDs,” Treasure Island (FL): StatPearls Publishing; 2022).

In embodiments, the SUD is selected from AUD, opioid use disorder, nicotine dependence and tobacco use disorder, sedative, hypnotic, and anxiolytic use disorder, stimulant use disorder.

In some embodiments, the SUD is a comorbidity in a patient having any one or more of a mental health disorder, neurodegenerative condition, an additional SUD, or a behavioral addiction.

In some embodiments, the SUD is not a comorbidity in a patient having another condition.

In some embodiments, a patient with an SUD is treated with a disclosed entactogen.

In some embodiments the entactogen is MDMA.

In some embodiments, administering EAP results in any one or more of a reduction of substance use, a reduction of substance cravings, a promotion of abstinence from substance use, (d) a prevention of relapse into substance use, and an improvement of at least one symptom of substance use disorder or a reduction in the severity of at least one diagnostic criterion for the substance use disorder. In some embodiments, the entactogen administered to a patient as part of facilitating EAP to treat a substance use disorder is selected from a substituted amphetamine, a substituted benzofuran, a substituted phenethylamine, or a substituted tryptamine. In some embodiments, the entactogen administered to a patient as part of facilitating EAP to treat a substance use disorder is any of MDMA, BDB, MBDB, MDA, MDEA, 3-MMC, 4-MMC, methylone, ethylone, butylone, αET, αMT, 5-IAI, MDAI, 5-APDI, 6-APB, 5-APB, 5-MAPB, MDAI, MDOH, and 4-FA. In some embodiments, the entactogen is MDMA.

In some embodiments, reducing substance use or a reduction of substance use refers to reducing the amount or frequency of substance use. In some embodiments, reducing substance use refers to a reduction in an amount of substance use per day or a reduction in the frequency of substance use, such as a reduction in the percentage of substance use days or a percentage of heavy substance use days. In some embodiments, reducing substance use refers to an increase in the time between consuming at least the substance, such that less total units of the substance are consumed within a given timeframe.

Abstinence from a substance refers to the cessation of substance consumption for an extended period of time, for example, for at least 1 day, such as 2 days or more, 3 days or more, 4 days or more, 5 days or more, 6 days or more, or 7 days or more. The term “promoting abstinence from a substance” or “promotion of abstinence from substance use” refers to helping to maintain abstinence from substance use, in particular after at least 1 day of not using the substance of abuse, for example maintaining abstinence from substance use for a period of days, including a period of a week or more, including more than one week, more than two weeks, more than three weeks, and a month or more, including more than one month, more than two months, more than three months, more than four months, more than five months, more than six months, more than seven months, more than eight months, more than nine months, more than 10 months, more than 11 months, and a year or more, including more than one year, more than two years, more than three years, more than four years, and more than five years, including values in between. In some embodiments, promoting substance use abstinence refers to maintaining abstinence from substance use, in particular, after at least 1 day of not using a substance of abuse, for example maintaining abstinence from substance use until at least the 3 month, 6 month, and/or 9 month follow-ups, as are disclosed within, or for longer than 9 months, or for longer than 1 year.

Relapse into substance use refers to substance intake (i.e., the consumption of a substance of abuse or the “taking” of said substance) following a period of abstinence from the substance of abuse, for example following a period of abstinence of at least 1 day or more, such as 3 days, 1 week, 2 weeks, 3 weeks, 1 month, 3 months, 6 months, 9 months, 1 year, or greater than 1 year, and values in between. In some embodiments, preventing relapse into substance use or refers to the prevention of substance intake by a patient after the patient has ceased the intake of the substance for at least 24 hours. In some embodiments, preventing relapse into substance use refers to the prevention of substance intake by a patient after the patient has ceased the intake of the substance of abuse for less than 24 hours. In some embodiments, preventing relapse into substance use refers to the permanent cessation of consuming that substance of abuse. In some embodiments, wherein a patient is administered a composition and/or a disclosed method, such as EAP, preventing relapse into substance use refers to an increase in time prior to the resumption of substance intake as compared to the time prior to the resumption of substance intake by a patient who is not administered a compound and/or a disclosed method, such as EAP. In some embodiments, the increase in time prior to the resumption can be, e.g., at least 1 day, such as 2 days or more, 3 days or more, 4 days or more, 5 days or more, 6 days or more, or a week or more, including more than one week, more than two weeks, more than three weeks, and a month or more, including more than one month, more than two months, more than three months, more than four months, more than five months, more than six months, more than seven months, more than eight months, more than nine months, more than 10 months, more than 11 months, and a year or more, including more than one year, more than two years, more than three years, more than four years, and more than five years, including values in between.

i. Alcohol Use Disorder (AUD)

In some embodiments, EAP is used to treat alcohol use disorder (AUD). In some embodiments, EAP is used to treat AUD that is comorbid with any of another substance use disorder, a behavioral addiction, a mental health disorder, or a neurodegenerative condition. In some embodiments, using EAP to treat AUD comprises administering a disclosed entactogen to a patient. As elsewhere, a “disclosed entactogen” may include, among others, a substituted amphetamine, a substituted benzofuran, a substituted phenethylamine, or a substituted tryptamine. In some embodiments, a disclosed entactogen is any of MDMA, BDB, MBDB, MDA, MDEA, 3-MMC, 4-MMC, methylone, ethylone, butylone, αET, αMT, 5-IAI, MDAI, 5-APDI, 6-APB, 5-APB, 5-MAPB, MDAI, MDOH, and 4-FA. In some embodiments, the entactogen is MDMA.

“Alcohol use disorder” or “AUD,” in some embodiments, refers to the disorder defined in the Diagnostic and Statistical Manual of Mental Disorders, 5th Edition (DSM-5). Under DSM-5, anyone meeting any 2 of 11 listed criteria during the same 12-month period receives a diagnosis of AUD, the criteria being whether in the past year, a patient has: (1) Had times when you [i.e., the patient] ended up drinking more, or longer, than you intended? (2) More than once wanted to cut down or stop drinking, or tried to, but couldn't? (3) Spent a lot of time drinking? Or being sick or getting over other aftereffects? (4) Wanted a drink so badly you couldn't think of anything else? (5) Found that drinking—or being sick from drinking—often interfered with taking care of your home or family? Or caused job troubles? Or school problems? (6) Continued to drink even though it was causing trouble with your family or friends? (7) Given up or cut back on activities that were important or interesting to you, or gave you pleasure, in order to drink? (8) More than once gotten into situations while or after drinking that increased your chances of getting hurt (such as driving, swimming, using machinery, walking in a dangerous area, or having unsafe sex)? (9) Continued to drink even though it was making you feel depressed or anxious or adding to another health problem? Or after having had a memory blackout? (10) Had to drink much more than you once did to get the effect you want? Or found that your usual number of drinks had much less effect than before? (11) Found that when the effects of alcohol were wearing off, you had withdrawal symptoms, such as trouble sleeping, shakiness, restlessness, nausea, sweating, a racing heart, or a seizure? Or sensed things that were not there? The severity of AUD (mild, moderate, or severe) is based on the number of criteria met. For instance, in the 2015 National Survey on Drug Use and Health survey, which polled 43,561 individuals, 11.8% met the criteria for AUD-67% of which were “mild” cases (2-3 symptoms), 18.8% had a “moderate” disorder (4-5 symptoms), and 13.8% possessed a “severe” disorder (6 or more symptoms) (Witkiewitz, Litten, and Leggio, 2019).

In some embodiments, AUD refers to a problematic pattern of alcohol use accompanied by clinically significant impairment or distress (Kranzler and Soyka, 2018). In some embodiments, AUD refers to a disease characterized by a loss of control over alcohol drinking accompanied by changes in brain regions related to the execution of motivated behaviors and to the control of stress and emotionality (e.g., the midbrain, the limbic system, the prefrontal cortex, and the amygdala) (Witkiewitz, Litten, and Leggio, 2019).

Although herein the terms “alcohol use disorder” and “AUD” generally shall refer to the criteria in the DSM-5, or a patient with a diagnosis based thereon, it will be appreciated that the compositions and disclosed methods are equally applicable to patients having the equivalent underlying disorder, whether that disorder is diagnosed based on the criteria in DSM-5 or in DSM-IV (which described two distinct disorders-“alcohol abuse” and “alcohol dependence”—with specific criteria for each), whether the diagnosis is based on other clinically acceptable criteria (e.g., as “Alcohol Dependence Syndrome,” see Edwards, G. (1986), The Alcohol Dependence Syndrome: a concept as stimulus to enquiry. Brit. J. Addiction, 81:171-183), or whether the patient has not yet had a formal clinical diagnosis.

“Alcohol dependence” refers to the more severe form of AUD involving physical alcohol dependence. Use of terms such as “physically dependent” alcoholism, “physically dependent” alcohol addiction, or “physically dependent” AUD, therefore will be understood to refer to the psychiatric disorder or condition in which an individual has become physically dependent upon, or physically addicted to, alcohol (e.g., Alcohol Dependence Syndrome). Typically alcohol dependence is characterized by (often serious) withdrawal symptoms on cessation of alcohol, or drinking to avoid withdrawal symptoms, tolerance and the persistent desire to drink, and continuing drinking despite negative consequences (NICE 2013).

“Harmful use of alcohol” or “harmful use” AUD refers to the definition in the WHO's International Classification of Diseases, 10th Revision (ICD-10 Classification of Mental and Behavioral Disorders) (ICD-10; WHO, 1992): “[A] pattern of psychoactive substance use that is causing damage to health. The damage may be physical (e.g., hepatitis) or mental (e.g., depressive episodes secondary to heavy alcohol intake). Harmful use commonly, but not invariably, has adverse social consequences; social consequences in themselves, however, are not sufficient to justify a diagnosis of harmful use.”

The distinction between the different severities of AUD can vary over time within a single patient's relationship with alcohol; however, one of ordinary skill will appreciate how to make an appropriate and reasonable clinical determination. Clinical signs and tools used to distinguish between those AUD patients with non-physical “harmful use” and those who are physically dependent on alcohol include whether or not there is evidence of physical dependence (including evidence of severe sweating, tremor, vomiting, nausea or confusion on cessation of drinking for >48 hours); whether or not there is evidence of a strong craving to drink alcohol in order to combat the symptoms of alcohol withdrawal; and whether one scores 15 or less (non-dependent, harmful use) or greater than 15 (dependent) on the Severity of Alcohol Dependence Questionnaire (SADQ). As the NICE Guidelines explain: “Although alcohol dependence is defined in ICD-10 and DSM-IV in categorical terms for diagnostic and statistical purposes as being either present or absent, in reality dependence exists on a continuum of severity. Therefore, it is helpful from a clinical perspective to subdivide dependence into categories of mild, moderate, and severe. People with mild dependence (e.g., those scoring 15 or less on the Severity of Alcohol Dependence Questionnaire; SADQ) usually do not need assisted alcohol withdrawal. People with moderate dependence (with a SADQ score of between 15 and 30) usually need assisted alcohol withdrawal, which can typically be managed in a community setting unless there are other risks. People who are severely alcohol dependent (with a SADQ score of 30 or more) will need assisted alcohol withdrawal, typically in an inpatient or residential setting. In this guideline these definitions of severity are used to guide selection of appropriate interventions.” In other embodiments, the categories of mild, moderate, and severe may be diagnosed based on the symptoms as defined by the DSM-5, wherein “mild” refers to two or three symptoms, “moderate” refers to four or five symptoms, and “severe” refers to six or more symptoms.

AUD patients characterized as “harmful use,” refer to those engaging in “harmful alcohol use,” which is characterized by a pattern of use that causes damage to health, which may be physical or mental; and has adverse social consequences, including those affecting personal relationships, professional relationships, familial relationships, etc. (note, however, that adverse social consequences themselves do not justify a diagnosis of “harmful use”). Thus, in some embodiments, “harmful use” or “harmful alcohol use” refer to a condition where a patient scores between 0 and 15 on the SADQ. In some embodiments, “harmful use” or “harmful alcohol use” refer to a condition where a patient scores between 0 and 15 on the SADQ, and where the patient exhibits at least one of damage to health (e.g., physical, mental, or physical and mental health), and strained/damaged social relationships.

The negative physiological effects of alcohol use are well-known and include damage to the gastrointestinal tract (Bishehsari, 2017; Egerer, 2005) and the liver (Ding, 2019). These effects may result in altered absorption and drug metabolism, depending on several factors, including whether alcohol consumption has resulted in changes to the structure and function of the gut and/or the liver. Alcohol use has also been shown to cause hypertension (Husain, 2014) and to compromise the cardiovascular system (Piano, 2017). Considering the hepatic metabolism of MDMA, including its conversion into hepatotoxic metabolites (de la Torre 2004; Carvalho, 2010), the safety, such as the type and severity of adverse events, and the utility of MDMA-assisted psychotherapy in patients who consume alcohol, such as those diagnosed with AUD, may be unpredictable.

“Alcohol use disorder patient” or “AUD patient” refers to a patient diagnosed with AUD (such as dependent use AUD or harmful use AUD), as defined herein. In some embodiments, the term “AUD patient” refers to a patient diagnosed with AUD who is in abstinence from alcohol, for example, for at least 1 day, such as 2 days or more, 3 days or more, 4 days or more, 5 days or more, 6 days or more, or 7 days or more. The phrase “AUD patient in abstinence from alcohol” refers to a patient diagnosed with AUD in abstinence from alcohol, i.e., having not consumed alcohol, for a given period of time, for example, for at least 1 day, such as 2 days or more, 3 days or more, 4 days or more, 5 days or more, 6 days or more, or 7 days or more. The term “AUD patient post-detox” refers to a patient diagnosed with AUD after having undergone a detoxification course, wherein a “detoxification course” or simply “detoxification” refers, in some embodiments, to the abrupt cessation of alcohol intake in individuals that suffer from alcohol dependence.

In some embodiments, detoxification includes the administration of drugs to prevent symptoms of “withdrawal,” which refers to a physiological response experienced by those having a physical dependence on alcohol, the symptoms of which include, but are not limited to, anxiety, shakiness, sweating, vomiting, fever, increased heart rate, seizures, hallucinations, and delirium tremens (National Clinical Guideline Centre, 2010). In some embodiments, a detoxification course may last for a given duration of time, for example, for at least 1 day, such as 2 days or more, 3 days or more, 4 days or more, 5 days or more, 6 days or more, or 7 days or more. In some embodiments detoxification or a detoxification course will be as specifically disclosed herein.

“Binge drinking” refers to a pattern of drinking that brings a person's blood alcohol concentration (BAC) to at least 0.08 g/dl. A “binge drinker,” i.e., one who binge drinks, is referred to as an “alcohol abuser” or “abuser of alcohol,” wherein the act of binge drinking itself may be referred to as “alcohol abuse” or “abuse of alcohol.” Abusers of alcohol may not drink on a consistent basis; for example, they may only drink once a week, but, when drinking, they may drink heavily, which may cause adverse health effects, such as, but not limited to, suffering from alcohol intoxication, unintentional injuries, such as car accidents, falls, burns, and alcohol poisoning; violence, including homicide, suicide, domestic violence, and sexual assault; sexually transmitted diseases; unintended pregnancy and poor pregnancy outcomes, including miscarriage and stillbirth; fetal alcohol spectrum disorders, sudden infant death syndrome, chronic diseases such as, but not limited to, high blood pressure, stroke heart disease, and liver disease; cancer, including but not limited to breast cancer, mouth cancer, throat cancer, esophageal cancer, liver cancer, and colon cancer; memory and learning problems, and other behavioral addictions and substance use disorders (CDC, 2019).

The frequency at which one binge drinks may determine whether or not the individual is considered a “heavy drinker” or “heavy alcohol user.” “Heavy drinker” refers to someone with a heavy alcohol use pattern. The National Institute on Alcohol Abuse and Alcoholism (NIAAA) and the Substance Abuse and Mental Health Services Administration (SAMHSA) define “heavy alcohol use” as binge drinking on 5 or more days in the past month. NIAAA defines binge drinking as a pattern of drinking that brings BAC levels to at least 0.08 g/dL. This typically occurs after 4 alcoholic drinks for women and 5 alcoholic drinks for men within a period of about 2 hours. SAMHSA defines “binge drinking” as 5 or more alcoholic drinks for males or 4 or more alcoholic drinks for females on the same occasion (i.e., at the same time or within a couple of hours of each other) on at least 1 day in the past month.

“Alcohol,” as used herein, for example in relation to “drinks,” “alcoholic drinks,” or “drinking,” refers to ethyl alcohol (i.e., ethanol). “Alcohol craving” refers to a conscious desire or urge to consume alcohol. “Alcohol use” refers to alcohol consumption, and “alcohol consumption” refers to the standards known in the art, such as those of NIAAA or WHO.

“Reducing alcohol use” or “reduction of alcohol use” refers to reducing the amount or frequency of alcohol use, for example as assessed by urinalysis (e.g., by measuring metabolites of alcohol in urine, such as Ethyl Glucuronide (EtG)) or as assessed by using self-reported alcohol use with standardized tools like the Timeline Follow-Back self-report (TLFB) (Sobell & Sobell, 1996). In some embodiments, “reducing alcohol use” or “reduction of alcohol use” refers to a reduction in drinks per day, a reduction in drinks per drinking day, or a reduction in the frequency of drinking, such as a reduction in the percentage of drinking days or percentage of heavy drinking days. In some embodiments “reducing alcohol use” or the “reduction of alcohol use” refers to an increase in the time between consuming at least one drink of alcohol, such that less total units of alcohol are consumed within a given timeframe. A “unit of alcohol” refers to a standard drink, which contains about 14 g of pure alcohol (ethanol). For typical alcoholic beverages, this refers to 12 ounces of 5% alcohol beer, 5 ounces of 12% wine, and 1.5 ounces of 40% distilled spirits (NIH). “Drinking,” “drinks,” or “alcoholic drinks,” is understood in the context of “standard drinks,” such as spirits or blends that are intended for human consumption, wherein a “standard drink” equals 12 g (15.2 mL) ethanol. “Heavy drinking day” refers to a day with a total alcohol consumption ≥60 g (76.05 mL) of ethanol for men and ≥40 g (50.7 mL) for women. In some embodiments, “reducing alcohol use” or the “reduction of alcohol use” refers to increasing the time to a “heavy drinking day,” wherein “heavy drinking day” refers to a given 24-hour period in which the pattern of drinking meets that of “binge drinking,” as defined herein.

“Alcohol abstinence” or “in abstinence from alcohol” refers to the cessation of alcohol consumption for an extended period of time, for example, for at least 1 day, such as 2 days or more, 3 days or more, 4 days or more, 5 days or more, 6 days or more, or 7 days or more. The term “promoting alcohol abstinence” or “promotion of alcohol abstinence” refers to helping to maintain abstinence from alcohol use, in particular after at least 1 day of not drinking alcohol, for example maintaining abstinence from alcohol use for a period of days, including a period of a week or more, including more than one week, more than two weeks, more than three weeks, and a month or more, including more than one month, more than two months, more than three months, more than four months, more than five months, more than six months, more than seven months, more than eight months, more than nine months, more than 10 months, more than 11 months, and a year or more, including more than one year, more than two years, more than three years, more than four years, and more than five years, including values in between. In some embodiments, “promoting alcohol abstinence” or “promotion of alcohol abstinence” refers to helping to maintain abstinence from alcohol use, in particular after at least 1 day of not drinking alcohol, for example maintaining abstinence from alcohol use until at least the 3 month, 6 month, and/or 9 month follow-ups, as are disclosed within, or for longer than 9 months, or for longer than 1 year.

“Relapse into alcohol use” or “relapse into alcohol consumption” refers to alcohol intake (i.e., the consumption of alcohol or the “taking” of alcohol) following a period of alcohol abstinence, for example following a period of alcohol abstinence of at least 1 day or more, such as 3 days, 1 week, 2 weeks, 3 weeks, 1 month, 3 months, 6 months, 9 months, 1 year, or greater than 1 year, and values in between. In some embodiments, “preventing relapse into alcohol use” or “preventing relapse into alcohol consumption” refers to the prevention of alcohol intake by an AUD patient after the patient has ceased the intake of alcohol for at least 24 hours. In some embodiments, “preventing relapse into alcohol use” or “preventing relapse into alcohol consumption” refers to the prevention of alcohol intake by an AUD patient after the patient has ceased the intake of alcohol for less than 24 hours. In some embodiments, “preventing relapse into alcohol use” or “preventing relapse into alcohol consumption” refers to the permanent cessation of alcohol intake. In some embodiments, wherein a patient is administered a disclosed composition, “preventing relapse into alcohol use” or “preventing relapse into alcohol consumption” refers to an increase in time prior to the resumption of alcohol intake as compared to the time prior to the resumption of alcohol intake by a patient who is not administered a compound of the invention. In some embodiments, the increase in time prior to the resumption can be, e.g., at least 1 day, such as 2 days or more, 3 days or more, 4 days or more, 5 days or more, 6 days or more, or a week or more, including more than one week, more than two weeks, more than three weeks, and a month or more, including more than one month, more than two months, more than three months, more than four months, more than five months, more than six months, more than seven months, more than eight months, more than nine months, more than 10 months, more than 11 months, and a year or more, including more than one year, more than two years, more than three years, more than four years, and more than five years, including values in between.

“AUD associated with high risk drinking for acute problems” refers to AUD associated with daily alcohol consumption ≥60 g/day (76.05 mL/day) of ethanol for men and ≥40 g/day (50.7 mL/day) for women (International Guide for Monitoring Alcohol Consumption and Related Harm, WHO/MSD/MSB/00.4; WHO 2000, p. 51).

ii. Opioid Use Disorder (OUD)

In some embodiments, EAP is used to treat opioid use disorder (OUD). In some embodiments, EAP is used to treat OUD that is comorbid with any of another substance use disorder, a behavioral addiction, a mental health disorder, or a neurodegenerative condition. In some embodiments, using EAP to treat OUD comprises administering a disclosed entactogen to a patient. In some embodiments, the entactogen is MDMA.

Receptors of the opioid system, mu, kappa, delta, and opioid receptor like-1 (ORL1) are G-protein coupled receptors that activate inhibitory G-proteins (Al-Hasani & Bruchas, Anesthesiology. 2011; 115(6):1363-1381). Opioids activate the mesolimbic reward system, which promotes signaling in the ventral tegmental area and results in dopamine release (Kosted & George, Sci Pract Perspect., 2002; 1(1):13-20). Representative examples of opioids include codeine, heroin, hydrocodone, hydromorphone, methadone, meperidine, morphine, and oxycodone. It will be appreciated that “opiates” are included among “opioids” for purposes of the disclosure. In embodiments, an entactogen is used to treat OUD (which will include opiate use disorder).

OUD is characterized by an overwhelming desire to use opioids, the development of tolerance to opioids, and withdrawal syndrome once opioids are discontinued. Criteria for opioid use disorder are available to one of skill. In one example, the DSM-5 describes criteria, for example: Opioids are often taken in larger amounts or over a longer period of time than intended; Tolerance, as defined by either of the following: (a) a need for markedly increased amounts of opioids to achieve intoxication or desired effect or (b) markedly diminished effect with continued use of the same amount of an opioid; Withdrawal, as manifested by either of the following: (a) the characteristic opioid withdrawal syndrome or (b) the same (or a closely related) substance is taken to relieve or avoid withdrawal symptoms. In some embodiments, a disclosed entactogen alleviates or reduces the signs or symptoms of opioid use disorder. In some embodiments, a disclosed entactogen may be used as an adjunct to clinical opioid therapy.

Treatments for OUD include opioid receptor agonists, e.g., methadone and buprenorphine, and opioid receptor antagonists, e.g., naltrexone (Kampman et al., J. Addict. Med., 2015; 9(5):358-367). However, such treatments are associated with limited success in preventing relapse and, in some cases, carry abuse liability themselves. The dopamine system has been implicated in opioid reward (Fields & Margolis, Trends Neurosci., 2015; 38(4):217-225; Steidl et al., Neurosci. Biobehav. Rev., 2017; 83:72-82). D3 antagonists may be useful to treat opioid addiction and to potentiate the effects of prescribed opiates (Galaj et al., Neurosci. Biobehav. Rev., 2020; 114: 38-52). A compound's effects on opioid-seeking behavior may be evaluated according to methods available to one of skill, including self-administration models, e.g., the IV self-administration reinstatement rodent model described by Fattore et al., Methods Mol Biol. 2021; 2201:231-245.

In some embodiments, treating OUD comprises reducing opiate use or results in a reduction of opiate use. In some embodiments, treating OUD comprises reducing opiate cravings or results in a reduction of opiate cravings. In some embodiments, treating OUD comprises promoting abstinence from opiate use or results in abstinence from opiates. In embodiments, treating OUD comprises preventing relapse into opiate use. In embodiments, treating OUD comprises improving at least one symptom of OUD or reducing the severity of at least one diagnostic criterion of OUD.

In some embodiments, a reduction in opiate use refers to reducing the amount or frequency of opiate use, for example as assessed by urinalysis or saliva testing of opiate use biomarkers. Biomarkers of opiate use are described in, e.g., Stefanidou et al., Int J Clin Pract. 2010; 64(12):1712-8; Kennedy & Dani, J Anal Toxicol. 2020; 44(4):410-413; Valadbeigi & Ilbeigi, Rapid Commun Mass Spectrom. 2021 Mar. 30; 35(6):e9044

iii. Nicotine Dependence and Tobacco Use Disorder (NDTUD)

In some embodiments, EAP is used to treat nicotine dependence and tobacco use disorder (NDTUD). In some embodiments, EAP is used to treat NDTUD that is comorbid with any of another substance use disorder, a behavioral addiction, a mental health disorder, or a neurodegenerative condition. In some embodiments, using EAP to treat NDTUD comprises administering a disclosed entactogen to a patient. In some embodiments, the entactogen is MDMA. Nicotine addiction is mediated by activation of neuronal nicotinic acetylcholine receptors in the dopamine pathway, and nicotine exposure results in release of dopamine and an increase in extracellular dopamine levels (Laviolette & van der Kooy, Nat. Rev. Neurosci., 2004; 5(1):55-65; Nisell et al., Synapse, 1994; 16(1):36-44; Pierce & Kumaseran, Neurosci. Biobehav. Rev., 2006; 30(2):215-38). Tobacco is a common vehicle for nicotine and so nicotine dependence may be referred to as tobacco use disorder. In some embodiments, a disclosed entactogen is used to treat nicotine dependence. In some embodiments, a disclosed entactogen is used to treat tobacco use disorder. Criteria for nicotine dependence or tobacco use disorder are available to one of skill. See, e.g., Baker et al., Addiction, 2012; 107(2):263-275. In one example, the DSM-5 describes tobacco use disorder and criteria for the same, for example: Unsuccessful efforts to quit or reduce intake of tobacco; Inordinate amount of time acquiring or using tobacco products; Cravings for tobacco.

In some embodiments, a disclosed entactogen alleviates or reduces the signs or symptoms of nicotine dependence or tobacco use disorder. In some embodiments, treating nicotine dependence or tobacco use disorder comprises reducing use of nicotine or tobacco or results in a reduction of nicotine or tobacco use. In some embodiments, treating nicotine dependence or tobacco use disorder comprises reducing nicotine or tobacco cravings or results in a reduction of nicotine or tobacco cravings. In some embodiments, treating nicotine dependence or tobacco use disorder comprises promoting abstinence from nicotine or tobacco use or results in abstinence from nicotine or tobacco. In some embodiments, treating nicotine dependence or tobacco use disorder comprises preventing relapse into nicotine or tobacco use. In some embodiments, treating nicotine dependence or tobacco use disorder comprises improving at least one symptom of nicotine dependence or tobacco use disorder or reducing the severity of at least one diagnostic criterion of nicotine dependence or tobacco use disorder.

In embodiments, nicotine cravings are assessed by evaluating nicotine seeking behavior. A compound's effects on nicotine seeking behavior may be evaluated according to methods available to one of skill, including self-administration, place conditioning, and intracranial self-stimulation paradigms in rodents (O'Dell & Khroyan, Pharmacol. Biochem. Behav., 2009; 91(4): 481-488).

iv. Sedative, Hypnotic, and Anxiolytic Use Disorder (SHA)

In some embodiments, EAP is used to treat sedative, hypnotic, and anxiolytic use disorder (SHA). In some embodiments, EAP is used to treat SHA that is comorbid with any of another substance use disorder, a behavioral addiction, a mental health disorder, or a neurodegenerative condition. In embodiments, using EAP to treat SHA comprises administering a disclosed entactogen to a patient. In some embodiments, the entactogen is MDMA.

Sedatives, hypnotics, and anxiolytics can cause CNS depression, which may be fatal. Exemplary CNS depressants include benzodiazepines, such as alprazolam, clonazepam, lorazepam, diazepam, chlordiazepoxide, and barbiturates, such as phenobarbital, pentobarbital, butabarbital. Other classes of drugs have properties that share a similar mechanism of action with benzodiazepine and barbiturates, including alcohol. These agents mediate gamma-aminobutyric acid (GABA) effects, producing inhibitory effects within the central nervous system. In some embodiments, a disclosed entactogen is used to treat sedative, hypnotic, and anxiolytic use disorder.

Criteria for sedative, hypnotic, and anxiolytic use disorder are available to one of skill. In one example, the DSM-5 describes sedative, hypnotic, and anxiolytic use disorder and criteria for the same, for example: Sedatives, hypnotics, or anxiolytics are often taken in larger amounts or over a longer period than was intended; There is a persistent desire or unsuccessful efforts to cut down or control sedative, hypnotic, or anxiolytic use; A great deal of time is spent in activities necessary to obtain the sedative, hypnotic, or anxiolytic; use the sedative, hypnotic, or anxiolytic; or recover from its effects.

In some embodiments, a disclosed entactogen alleviates or reduces the signs or symptoms of sedative, hypnotic, and anxiolytic use disorder. In some embodiments, treating sedative, hypnotic, and anxiolytic use disorder comprises reducing use of one or more of a sedative, hypnotic, and anxiolytic or results in a reduction of use of the same. In some embodiments, treating sedative, hypnotic, and anxiolytic use disorder comprises reducing cravings for one or more of a sedative, hypnotic, and anxiolytic or results in a reduction of cravings for the same. In some embodiments, treating sedative, hypnotic, and anxiolytic use disorder comprises promoting abstinence from one or more of a sedative, hypnotic, and anxiolytic or results in abstinence from the same. In some embodiments, treating sedative, hypnotic, and anxiolytic use disorder comprises preventing relapse into use of one or more of a sedative, hypnotic, and anxiolytic. In some embodiments, treating sedative, hypnotic, and anxiolytic use disorder comprises improving at least one symptom of sedative, hypnotic, and anxiolytic use disorder or reducing the severity of at least one diagnostic criterion of sedative, hypnotic, and anxiolytic use disorder.

v. Stimulant Use Disorder

In some embodiments, EAP is used to treat stimulant use disorder. In embodiments, EAP is used to treat stimulant use disorder that is comorbid with any of another substance use disorder, a behavioral addiction, a mental health disorder, or a neurodegenerative condition. In embodiments, using EAP to treat stimulant use disorder comprises administering a disclosed entactogen to a patient. In some embodiments, the entactogen is MDMA.

Stimulants increase synaptic levels of the monoamines dopamine, serotonin, and norepinephrine. Both the dopaminergic and the noradrenergic systems play critical roles in the effects of stimulants, including reward (Sofuoglu & Sewell, Addict Biol., 2009; 14(2): 119-129; Wise, Brain Res., 1978; 152(2):215-47). Non-limiting examples of stimulants include amphetamines, methamphetamine, and cocaine. Nicotine/tobacco may also be considered a stimulant. In some embodiments, a disclosed entactogen is used to treat stimulant use disorder. In some embodiments, a disclosed entactogen is used to treat cocaine use disorder. In some embodiments, a disclosed entactogen is used to treat methamphetamine use disorder.

Criteria for stimulant use disorder are available to one of skill. For example, exemplary DSM-5 criteria include: The stimulant is often taken in larger amounts or over a longer period than was intended; There is a persistent desire or unsuccessful efforts to cut down or control stimulant use; A great deal of time is spent in activities necessary to obtain the stimulant, use the stimulant, or recover from its effects. In some embodiments, a disclosed entactogen alleviates or reduces the signs or symptoms of stimulant use disorder.

In some embodiments, a disclosed entactogen alleviates or reduces the signs or symptoms of stimulant use disorder. In some embodiments, treating stimulant use disorder comprises reducing use of stimulants or results in a reduction of stimulant use. In some embodiments, treating stimulant use disorder comprises reducing stimulant cravings or results in a reduction of stimulant cravings. In some embodiments, treating stimulant use disorder comprises promoting abstinence from stimulants or results in abstinence from stimulants. In some embodiments, treating stimulant use disorder comprises preventing relapse into stimulant use. In some embodiments, treating stimulant use disorder comprises improving at least one symptom of stimulant use disorder or reducing the severity of at least one diagnostic criterion of stimulant use disorder. In some embodiments, the stimulant is cocaine. In embodiments, the stimulant is an amphetamine, such as methamphetamine.

In some embodiments, a reduction of stimulant use or abstinence therefrom may be determined using biological samples. In one example, a patient's hair or urine may be assessed to evaluate the presence of cocaine and benzoylecgonine, a metabolite thereof. See, e.g., Bravo et al., Toxins (Basel). 2022 Apr. 13; 14(4):278 and Pan et al., Fa Yi Xue Za Zhi. 2018; 34(4):375-378. In some embodiments, stimulant cravings may be assessed using methods available to one of skill, including self-administration models, e.g., the hold down procedures described by Zimmer & Roberts, Psychiatric Disorders, 2011; 279-290.

Current treatment options for stimulant use disorder include contingency management, CBT, acupuncture, antidepressants, dopamine agonists, antipsychotics, anticonvulsants, disulfiram, opioid agonists, N-Acetylcysteine, and psychostimulants (Ronsley et al., PLoS One, 2020; 15(6): e0234809). Psychostimulants, for example, bupropion and dexamphetamine, are treatment options. In contrast, dopamine modulators, including agonists and antagonists appear to lack efficacy in stimulant use disorders, such as cocaine use disorder (Cochrane Database Syst Rev. 2015 May; 2015(5): CD003352; Cochrane Database Syst Rev. 2016 March; 2016(3): CD006306).

c. Treatment of Behavioral Addictions

In some embodiments, an entactogen is used to treat a behavioral addiction. In some embodiments, the entactogen is used in conjunction with psychotherapy to treat a behavioral addiction. In some embodiments, the behavioral addiction is selected from gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction, as well as such other behavioral addictions and disorders as will be known in the art. In some embodiments, the behavioral addiction is a comorbidity in a patient having any one or more of a mental health disorder, neurodegenerative condition, substance use disorder, or an additional behavioral addiction. In some embodiments, the behavioral addiction is not a comorbidity in a patient having another condition. In embodiments, a disclosed entactogen is administered to a patient as part of facilitating EAP to treat a behavioral addiction. In some embodiments, the entactogen is MDMA.

i. Gambling Disorder

In some aspects, the methods are useful in treating a patient with gambling disorder. In some embodiments, EAP is used to treat gambling disorder. In some embodiments, EAP is used to treat gambling disorder that is comorbid with any of a substance use disorder, another behavioral addiction, a mental health disorder, or a neurodegenerative condition. Whether a patient has gambling disorder can be determined according to ordinary skill. For example, in some embodiments, a patient may be determined to have gambling disorder based on the definition in the 11th Revision of the International Classification of Diseases (ICD-11). The ICD-11 defines Gambling Disorder as being characterized by a pattern of persistent or recurrent gambling behavior, which may be online (i.e., over the internet) or offline, manifested by: (1) impaired control over gambling (e.g., onset, frequency, intensity, duration, termination, context); (2) increasing priority given to gambling to the extent that gambling takes precedence over other life interests and daily activities; and (3) continuation or escalation of gambling despite the occurrence of negative consequences. The pattern of gambling behavior may be continuous or episodic and recurrent. The pattern of gambling behavior results in significant distress or in significant impairment in personal, family, social, educational, occupational or other important areas of functioning. The gambling behavior and other features are normally evident over a period of at least 12 months in order for a diagnosis to be assigned, although the required duration may be shortened if all diagnostic requirements are met and symptoms are severe.

In other embodiments, a patient may be determined to have gambling disorder based on the definition in the Diagnostic and Statistical Manual of Mental Disorders, 5th Edition (DSM-5). The DSM-5 defines gambling disorder as an addictive disorder in which individuals present with a cluster of symptoms of varying severity that cause psychological and psychical harms and social dysfunction due to their gambling. For diagnosis, four or more of the following symptoms must be present in a 12-month period: (a) needs to gamble with increasing amounts of money in order to achieve the desired excitement, (b) is restless or irritable when attempting to cut down or stop gambling, (c) has made repeated unsuccessful efforts to control, cut back, or stop gambling; (d) is often preoccupied with gambling (e.g., having persistent thoughts of reliving past gambling experiences, handicapping or planning the next venture, thinking of ways to get money with which to gamble), (e) often gambles when feeling distressed (e.g., helpless, guilty, anxious, depressed), (f) after losing money gambling, often returns another day to get even (“chasing” one's losses), (g) lies to conceal the extent of involvement with gambling, (h) has jeopardised or lost a significant relationship, job, or educational or career opportunity because of gambling, and (i) relies on others to provide money to relieve desperate financial situations caused by gambling. Last, the gambling behavior must not be better explained by a manic episode.

In other embodiments, a gambling disorder patient may have the equivalent underlying disorder, whether that disorder is diagnosed based on the criteria in the ICD-11 or DSM-5, whether the disorder is diagnosed based on a past edition of any system such as the ICD-10 or the DSM-IV (which described pathological gambling as an impulse control disorder with its own specific criteria), whether the disorder is diagnosed based on a future edition of any system, still forthcoming, whether the diagnosis is based on other clinically acceptable criteria, or whether the patient has not yet had a formal clinical diagnosis. It will be readily appreciated that this will be true for all behavioral addictions disclosed herein.

Gambling disorder may be characterized as being mild, moderate, and severe. The severity of gambling disorder may be determined according to the classification scale of the DSM-5, or otherwise using ordinary skill. For example, under the DSM-5, a patient with the presence of four to five symptoms has a mild case, a patient with the presence of six or seven symptoms has a moderate case, and a patient with eight or nine symptoms has a severe case. In embodiments, a patient may have gambling disorder characterized as being mild. In other embodiments, a patient may have a gambling disorder characterized as being moderate. In other embodiments, a patient may have gambling disorder characterized as being severe.

In embodiments, the term “gambling disorder patient” (or “patient with gambling disorder”) refers to a patient diagnosed with gambling disorder, such as defined herein.

Herein, “reducing gambling” or “reduction in gambling” refers to reducing the amount or frequency of gambling, such as assessed by standardised screening tools like the Gambling Timeline Follow-Back (G-TLFB) self-report (Sobell 2001, 1996). In embodiments, “reducing gambling” or “reduction in gambling” refers to a reduction in daily gambling, a reduction in the time spent gambling per day, or a reduction in the frequency of gambling, such as a reduction in the percentage of gambling days in general, or a reduction in the percentage of heavy gambling days. In embodiments, “reducing gambling” or “reduction in gambling” refers to an increase in the time to any gambling or time to the first heavy gambling day.

Herein, “gambling abstinence” or “in abstinence from gambling” refers to not engaging in gambling. In embodiments, “promoting gambling abstinence” or “promotion of gambling abstinence” refers to help maintaining abstinence from gambling, in particular after at least 1 day of not gambling, for example, maintaining abstinence from gambling for a period of at least 1 day, at least 1 week, at least 1 month, or at least 1 year.

Herein, “gambling disorder patient in abstinence from gambling” refers to a patient with gambling disorder in abstinence from gambling for a period, for example, for at least 1 day, at least 1 week, at least 1 month, or at least 1 year.

Herein, “relapse into gambling” refers to engaging in gambling following a period of abstinence from gambling, for example following a period of gambling abstinence of at least 1 day, at least 1 week, at least 1 month, or at least 1 year.

Herein, “preventing relapse into gambling” refers to the prevention of engaging in gambling by a gambling disorder patient after the patient has stopped gambling, in particular after 1 day or more of not gambling. In embodiments, the term encompasses a delay in the resumption of gambling as compared to the time to resumption by a patient that is not administered a compound of the invention using the disclosed methods. The delay in resumption can be for at least 1 day, at least 1 week, at least 1 month, or at least 1 year.

In embodiments, the patient with gambling disorder is administered an entactogen. In embodiments, the patient with gambling disorder receives entactogen assisted psychotherapy (EAP). In embodiments, the patient with gambling disorder undergoes an EAP treatment regimen. In embodiments, the administration of the entactogen is effective to treat gambling disorder. In embodiments, the EAP is effective to treat gambling disorder. In embodiments, the EAP treatment regimen is effective to treat gambling disorder. In embodiments, MDMA is administered. In embodiments, MDMA is effective to treat gambling disorder.

In embodiments, the administration of an entactogen according to the disclosed methods will be effective to treat gambling disorder as shown by a reduction in the severity of gambling disorder. In embodiments, a patient who has gambling disorder characterized as being mild when measured at baseline will no longer have a mild gambling disorder. In embodiments, a patient who has gambling disorder characterized as being moderate when measured at baseline will no longer have a moderate gambling disorder. In embodiments, a patient who has gambling disorder characterized as being severe when measured at baseline will no longer have a severe gambling disorder.

A reduction in the severity of gambling disorder can be determined based on an assessment for Gambling Disorder. In some embodiments, the assessment for Gambling Disorder is the Problem Gambling Severity Index (PGSI). In embodiments, a patient who scores 1-2 on the PGSI when measured at baseline will have a reduction in PGSI score of at least 1 point after treatment with the disclosed methods. In embodiments, a patient who scores 3-7 on the PGSI when measured at baseline will have a reduction in PGSI score of at least 3 points after treatment with the disclosed methods. In embodiments, a patient who scores 8 or more on the PGSI when measured at baseline will have a reduction in PGSI score of at least 5 points after treatment with the disclosed methods.

In some embodiments, the assessment for Gambling Disorder is any of the Lie-Bet Tool to Rule Out Problem Gambling, the National Opinion Research Center Diagnostic Screen for Gambling Disorders, Loss of Control, Lying, and Preoccupation screen (NODS-Clip), the National Opinion Research Center Diagnostic Screen for Gambling Disorders preoccupation, Escape, Risked Relationships And Chasing Screen (NODS-PERC), the Brief Bio-Social gambling Screen (BBGS), the DSM-V Screen for Pathological Gambling, the NORC Diagnostic Screen, the South Oaks Gambling Screen, and the South Oaks Gambling Screen revised for adolescents.

In embodiments, the administration of an entactogen according to the disclosed methods will be effective to treat gambling disorder as shown by one or more of: (a) a reduction in gambling, (b) a reduction in drive or urge to engage in gambling, (c) a promotion of abstinence from gambling, (d) a prevention of relapse into gambling, (e) a reduction in at least one symptom of gambling disorder.

In embodiments, administration of an entactogen according to the disclosed methods will result in a measurable reduction in gambling or in the drive or urge to engage in gambling, for example as compared to a baseline determination made before such administration. In embodiments, the reduction in gambling is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from gambling or prevention of relapse into gambling, when measured from the start of treatment, such as the first administration. In embodiments, the promotion of abstinence from gambling results in abstinence for at least 1 day, at least 1 week, at least 1 month, or at least 1 year, from the start of treatment. In embodiments, the reduction in gambling, reduction in drive or urge to engage in gambling, promotion of abstinence from gambling, prevention of relapse into gambling, and reduction in at least one symptom of gambling disorder lasts for at least 1 day, at least 1 week, at least 1 month, or at least 1 year.

In embodiments, administration of an entactogen according to the disclosed methods will result in a reduction in at least one symptom of gambling disorder, for example as compared to a baseline determination made before such administration. In embodiments, the reduction in at least one symptom of gambling disorder may be determined by changes in patient completed self-assessments, and clinical outcome assessment or index scores, wherein a decrease in score of one or more points indicates a decrease in the severity of symptoms. In embodiments, the clinical outcome assessment or index includes the PGSI. In embodiments, administration of an entactogen according to the disclosed methods will result in an elimination of at least one symptom of gambling disorder, for example as compared to a baseline determination made before such administration, and where elimination means that the patient no longer has the symptom or meets the criteria therefor, and which may be for a period of time of, for example, at least 1 day, at least 1 week, at least 1 month, or at least 1 year.

In embodiments, administration of an entactogen according to the disclosed methods will result in the patient having a reduction in at least one symptom of gambling disorder, including impaired control over gambling, increasing priority given to gambling to the extent that gambling takes precedence over other life interests and daily activities, continuation or escalation of gambling despite the occurrence of negative consequences, needs to gamble with increasing amounts of money in order to achieve the desired excitement, is restless or irritable when attempting to cut down or stop gambling, has made repeated unsuccessful efforts to control, cut back, or stop gambling; is often preoccupied with gambling, often gambles when feeling distressed, after losing money gambling, often returns another day to get even; lies to conceal the extent of involvement with gambling, has jeopardised or lost a significant relationship, job, or educational or career opportunity because of gambling; and relies on others to provide money to relieve desperate financial situations caused by gambling.

In embodiments, administration of an entactogen according to the disclosed methods will result in the patient having an elimination of at least one symptom of a gambling disorder, including impaired control over gambling, increasing priority given to gambling to the extent that gambling takes precedence over other life interests and daily activities, continuation or escalation of gambling despite the occurrence of negative consequences, needs to gamble with increasing amounts of money in order to achieve the desired excitement, is restless or irritable when attempting to cut down or stop gambling, has made repeated unsuccessful efforts to control, cut back, or stop gambling; is often preoccupied with gambling, often gambles when feeling distressed, after losing money gambling, often returns another day to get even; lies to conceal the extent of involvement with gambling, has jeopardised or lost a significant relationship, job, or educational or career opportunity because of gambling; and relies on others to provide money to relieve desperate financial situations caused by gambling.

In embodiments, the patient with gambling disorder has a comorbid psychiatric condition. In embodiments, treating the patient with gambling disorder is also effective to treat one or more comorbid psychiatric conditions in the patient.

ii. Gaming Disorder

In some aspects, the methods are useful in treating a patient with gaming disorder. In some embodiments, EAP is used to treat gaming disorder. In some embodiments, EAP is used to treat gaming disorder that is comorbid with any of a substance use disorder, another behavioral addiction, a mental health disorder, or a neurodegenerative condition. Whether a patient has gaming disorder can be determined according to ordinary skill. For example, in some embodiments, a patient may be determined to have gaming disorder based on the definition in the ICD-11. The ICD-11 defines Gaming Disorder (GD) as a pattern of gaming behavior, for example, “digital-gaming” or “video-gaming.”

Exemplary criteria include (1) a persistent pattern of gaming behavior (‘digital gaming’ or ‘video-gaming’), which may be predominantly online (i.e., over the internet or similar electronic networks) or offline, manifested by all of the following: (a) impaired control over gaming behavior (e.g., onset, frequency, intensity, duration, termination, context); (b) increasing priority given to gaming behavior to the extent that gaming takes precedence over other life interests and daily activities, (c) and continuation or escalation of gaming behavior despite negative consequences (e.g., family conflict due to gaming behavior, poor scholastic performance, negative impact on health); (2) the pattern of gaming behavior may be continuous or episodic and recurrent but is manifested over an extended period of time (e.g., 12 months); (3) the gaming behavior is not better accounted for by another mental disorder (e.g., Manic Episode) and is not due to the effects of a substance or medication; (4) the pattern of gaming behavior results in significant distress or impairment in personal, family, social, educational, occupational, or other important areas of functioning (ICD-11). For a diagnosis, the behavior must be of sufficient severity to result in significant impairment in personal, family, social, educational, occupational or other important areas of functioning and would normally have been evident for at least 12 months (WHO, “Addictive behaviors: Gaming disorder,” 2020). In other embodiments, a patient may be determined to have a gaming disorder based on questionnaires known to those of skill, such as the Gaming Disorder Scale for Adolescents (GADIS-A) (Paschke et al., J. Clin. Med., 2020; 9(4), 993).

In embodiments, the Internet Gaming Disorder Scale (IGD-20) and Internet Gaming Disorder Scale-Short Form (IGDS9-SF) may be utilised to distinguish disordered and non-disordered gaming, wherein scores of 71 or above on the IGD-20 and 32 points or above on the IGDS9-SF may be considered disordered, and scores below those values are considered non-disordered.

In some embodiments, a patient may be determined to have gaming disorder based on the DSM-5, which includes Internet gaming disorder (IGD) as a non-substance addiction. Nine criteria for IGD were recommended in the DSM-5, where such gaming also causes “significant impairment or distress” in several aspects of the patient's life: (a) preoccupation with gaming, (b), withdrawal symptoms when gaming is taken away or not possible (sadness, anxiety, irritability), (c) tolerance, the need to spend more time gaming to satisfy the urge, (d) inability to reduce playing, unsuccessful attempts to quit gaming, (e) giving up other activities, loss of interest in previously enjoyed activities due to gaming, (f) continuing to game despite problems, (g) deceiving family members or others about the amount of time spent on gaming, (h) the use of gaming to relieve negative moods, such as guilt or hopelessness, (i) risk, having jeopardised or lost a job or relationship due to gaming. Under the proposed criteria, a diagnosis of internet gaming disorder would require experiencing five or more of these symptoms within a year. The condition can include gaming on the internet, or on any electronic device, although most people who develop clinically significant gaming problems play primarily on the internet.

In embodiments, the term “gaming disorder patient” (or “patient with gaming disorder”) refers to a patient diagnosed with a gaming disorder, such as defined herein.

Herein, “reducing gaming” or “reduction in gaming” refers to reducing the amount or frequency of gaming, such as assessed by standardised screening tools like the IGD-20 and IGDS9-SF. In embodiments, “reducing gaming” or “reduction in gaming” refers to a reduction in daily gaming, a reduction in the time spent gaming per day, or a reduction in the frequency of gaming, such as a reduction in the percentage of gaming days in general, or a reduction in the percentage of heavy gaming days. In embodiments, “reducing gaming” or “reduction in gaming” refers to an increase in the time to any gaming or time to the first heavy gaming day.

Herein, “gaming abstinence” or “in abstinence from gaming” refers to not engaging in gaming. In embodiments, “promoting gaming abstinence” or “promotion of gaming abstinence” refers to help maintaining abstinence from gaming, in particular after at least 1 day of not gaming, for example, maintaining abstinence from gaming for a period of at least 1 day, at least 1 week, at least 1 month, or at least 1 year.

Herein, “gaming disorder patient in abstinence from gaming” refers to a patient with gaming disorder in abstinence from gaming for a period, for example, for at least 1 day, at least 1 week, at least 1 month, or at least 1 year.

Herein, “relapse into gaming” refers to engaging in gaming following a period of abstinence from gaming, for example following a period of gaming abstinence of at least 1 day, at least 1 week, at least 1 month, or at least 1 year.

Herein, “preventing relapse into gaming” refers to the prevention of engaging in gaming by a gaming disorder patient after the patient has stopped gaming, in particular after 1 day or more of not gaming. In embodiments, the term encompasses a delay in the resumption of gaming as compared to the time to resumption by a patient that is not administered a compound of the invention using the disclosed methods. The delay in resumption can be for at least 1 day, at least 1 week, at least 1 month, or at least 1 year.

In embodiments, the patient with gaming disorder is administered an entactogen. In embodiments, the patient with gaming disorder receives EAP. In embodiments, the patient with gaming disorder undergoes an EAP treatment regimen. In embodiments, the administration of the entactogen is effective to treat gaming disorder. In embodiments, the EAP is effective to treat gaming disorder. In embodiments, the EAP treatment regimen is effective to treat gaming disorder. In embodiments, MDMA is administered. In embodiments, MDMA is effective to treat gaming disorder.

In embodiments, the administration of an entactogen according to the disclosed methods will be effective to treat gaming disorder as shown by a reduction in the severity of gaming disorder. In embodiments, a patient who has gaming disorder characterized as being mild when measured at baseline will no longer have a mild gaming disorder. In embodiments, a patient who has gaming disorder characterized as being moderate when measured at baseline will no longer have a moderate gaming disorder. In embodiments, a patient who has gaming disorder characterized as being severe when measured at baseline will no longer have a severe gaming disorder. A reduction in the severity of gaming disorder can be determined based on ordinary skill, such as using an assessment.

In embodiments, the administration of an entactogen according to the disclosed methods will be effective to treat gaming disorder as shown by reducing a patient classified as a disordered gamer pre-treatment to a classification as a non-disordered gamer post-treatment. In embodiments, a patient who scores 71 points or higher on the IGD-20 when measured at baseline will have a reduction in the IGD-20 score of at least 10 points. In embodiments, a patient who scores 71 points or higher on the IGD-20 when measured at baseline will have a post-treatment IGD-20 score of less than 71 points. In embodiments, a patient who scores 32 points or higher on the IGDS9-SF when measured at baseline will have a reduction in the IGDS9-SF score of at least 5 points. In embodiments, a patient who scores 32 points or higher on the IGD-20 when measured at baseline will have a post-treatment IGD-20 score of less than 32 points.

In embodiments, the administration of an entactogen according to the disclosed methods will be effective to treat gaming disorder as shown by one or more of: (a) a reduction in gaming, (b) a reduction in drive or urge to engage in gaming, (c) a promotion of abstinence from gaming, (d) a prevention of relapse into gaming, (e) a reduction in at least one symptom of gaming disorder.

In embodiments, administration of an entactogen according to the disclosed methods will result in a measurable reduction in gaming or in the drive or urge to engage in gaming, for example as compared to a baseline determination made before such administration. In embodiments, the reduction in gaming is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from gaming or prevention of relapse into gaming, when measured from the start of treatment, such as the first administration. In embodiments, the promotion of abstinence from gaming results in abstinence for at least 1 day, at least 1 week, at least 1 month, or at least 1 year, from the start of treatment. In embodiments, the reduction in gaming, reduction in drive or urge to engage in gaming, promotion of abstinence from gaming, prevention of relapse into gaming, and reduction in at least one symptom of gaming disorder lasts for at least 1 day, at least 1 week, at least 1 month, or at least 1 year.

In embodiments, administration of an entactogen according to the disclosed methods will result in a reduction in at least one symptom of gaming disorder, for example as compared to a baseline determination made before such administration. In embodiments, the reduction in at least one symptom of gaming disorder may be a decrease of one or more points of a patient completed self-assessment, as described herein, and/or a decrease of one or more points in a clinical outcome assessment or index score, such as the IGD-20 and the IGDS9-SF. In embodiments, administration of an entactogen according to the disclosed methods will result in an elimination of at least one symptom of gaming disorder, for example as compared to a baseline determination made before such administration. In embodiments, administration of an entactogen according to the disclosed methods will result in an elimination of at least one symptom of gaming disorder, for example as compared to a baseline determination made before such administration.

In embodiments, administration of an entactogen according to the disclosed methods will result in the patient having a reduction in at least one symptom of a gaming disorder, including impaired control over gaming behavior, increasing priority given to gaming behavior to the extent that gaming takes precedence over other life interests and daily activities, continuation or escalation of gaming behavior despite negative consequences, preoccupation with gaming, withdrawal symptoms when gaming is taken away or not possible, tolerance, the need to spend more time gaming to satisfy the urge; inability to reduce playing, unsuccessful attempts to quit gaming; giving up other activities, loss of interest in previously enjoyed activities due to gaming; continuing to game despite problems, deceiving family members or others about the amount of time spent on gaming, the use of gaming to relieve negative moods, such as guilt or hopelessness; risk having jeopardized or lost a job or relationship due to gaming.

In embodiments, administration of an entactogen according to the disclosed methods will result in the patient having an elimination of at least one symptom of a gaming disorder, including impaired control over gaming behavior, increasing priority given to gaming behavior to the extent that gaming takes precedence over other life interests and daily activities, continuation or escalation of gaming behavior despite negative consequences, preoccupation with gaming, withdrawal symptoms when gaming is taken away or not possible, tolerance, the need to spend more time gaming to satisfy the urge; inability to reduce playing, unsuccessful attempts to quit gaming; giving up other activities, loss of interest in previously enjoyed activities due to gaming; continuing to game despite problems, deceiving family members or others about the amount of time spent on gaming, the use of gaming to relieve negative moods, such as guilt or hopelessness; risk having jeopardized or lost a job or relationship due to gaming.

In embodiments, the patient with gaming disorder has a comorbid psychiatric condition. In embodiments, treating the patient with gaming disorder according to the methods is also effective to treat one or more comorbid psychiatric conditions in the patient.

iii. Compulsive Sexual Behavior Disorder (CSBD)

In some aspects, the provided methods are useful in treating a patient with compulsive sexual behavior disorder (CSBD). In some embodiments, EAP is used to treat CSBD. In some embodiments, EAP is used to treat CSBD that is comorbid with any of a substance use disorder, another behavioral addiction, a mental health disorder, or a neurodegenerative condition. Whether a patient has CSBD can be determined according to ordinary skill.

For example, a patient may be determined to have CSBD based on the definition in the ICD-11, which defines compulsive sexual behavioral disorder as a persistent pattern of failure to control intense, repetitive sexual impulses or urges resulting in repetitive sexual behavior. Exemplary criteria according to the ICD-11 for compulsive sexual behavior disorder include: (1) a persistent pattern of failure to control intense, repetitive sexual impulses or urges resulting in repetitive sexual behavior, manifested in one or more of the following: (a) engaging in repetitive sexual behavior has become a central focus of the individual's life to the point of neglecting health and personal care or other interests, activities and responsibilities; (b) the individual has made numerous unsuccessful efforts to control or significantly reduce repetitive sexual behavior, (c) the individual continues to engage in repetitive sexual behavior despite adverse consequences (e.g., marital conflict due to sexual behavior, financial or legal consequences, negative impact on health); and (d) the person continues to engage in repetitive sexual behavior even when the individual derives little or no satisfaction from it; (2) the pattern of failure to control intense, repetitive sexual impulses or urges and resulting repetitive sexual behavior is manifested over an extended period of time (e.g., 6 months or more); (3) the pattern of failure to control intense, repetitive sexual impulses or urges and resulting repetitive sexual behavior is not better accounted for by another mental disorder (e.g., Manic Episode) or other medical condition and is not due to the effects of a substance or medication; (4) the pattern of repetitive sexual behavior results in marked distress or significant impairment in personal, family, social, educational, occupational, or other important areas of functioning. Distress that is entirely related to moral judgments and disapproval about sexual impulses, urges, or behaviors is not sufficient to meet this requirement (ICD-11). Sexual addiction may also be referred to as compulsive sexual behavior, hypersexuality, or hypersexuality disorder.

The presence of CSBD may be determined by the classification scale of the ICD-11, or based on ordinary skill. In embodiments, the Compulsive Sexual behavior Inventory (CSBI-13), a 13-item, five point Likert scale, may be utilized to determine if a patient has CSBD. In embodiments, a score above a 35 indicates the presence of CSBD (Coleman, n.d.).

In embodiments, the term “compulsive sexual behavior disorder patient” or “CSBD patient” (or “patient with compulsive sexual behavior disorder” or “patient with CSBD”) refers to a patient diagnosed with CSBD, such as defined herein.

Herein, “reducing compulsive sexual behavior” or “reduction in compulsive sexual behavior” refers to reducing the amount or frequency of compulsive sexual behavior, such as assessed by standardized screening tools like the CSBI-13. In embodiments, “reducing compulsive sexual behavior” or “reduction in compulsive sexual behavior” refers to a reduction in daily compulsive sexual behavior, a reduction in the time spent engaging in compulsive sexual behavior per day, or a reduction in the frequency of compulsive sexual behavior, such as a reduction in the percentage of compulsive sexual behavior days in general, or a reduction in the percentage of heavy compulsive sexual behavior days. In embodiments, “reducing compulsive sexual behavior” or “reduction in compulsive sexual behavior” refers to an increase in the time to any compulsive sexual behavior or time to the first heavy compulsive sexual behavior day.

Herein, “compulsive sexual behavior abstinence” or “in abstinence from compulsive sexual behavior” refers to not engaging in compulsive sexual behavior. In embodiments, “promoting compulsive sexual behavior abstinence” or “promotion of compulsive sexual behavior abstinence” refers to help maintaining abstinence from compulsive sexual behavior, in particular after at least 1 day of not exhibiting compulsive sexual behavior, for example, maintaining abstinence from compulsive sexual behavior for a period of at least 1 day, at least 1 week, at least 1 month, or at least 1 year.

Herein, “compulsive sexual behavior disorder patient in abstinence from compulsive sexual behavior” refers to a patient with compulsive sexual behavior disorder in abstinence from compulsive sexual behavior for a period, for example, for at least 1 day, at least 1 week, at least 1 month, or at least 1 year.

Herein, “relapse into compulsive sexual behavior” refers to engaging in compulsive sexual behavior following a period of abstinence from compulsive sexual behavior, for example following a period of compulsive sexual behavior abstinence of at least 1 day, at least 1 week, at least 1 month, or at least 1 year.

Herein, “preventing relapse into compulsive sexual behavior” refers to the prevention of engaging in compulsive sexual behavior by a compulsive sexual behavior disorder patient after the patient has stopped exhibiting compulsive sexual behavior, in particular after 1 day or more of not exhibiting compulsive sexual behavior. In embodiments, the term encompasses a delay in the resumption of compulsive sexual behavior as compared to the time to resumption by a patient that is not administered a compound of the invention using the disclosed methods. The delay in resumption can be for at least 1 day, at least 1 week, at least 1 month, or at least 1 year.

In embodiments, the patient with CSBD is administered an entactogen. In embodiments, the patient with CSBD receives EAP. In embodiments, the patient with CSBD undergoes an EAP treatment regimen. In embodiments, the administration of the entactogen is effective to treat CSBD. In embodiments, the EAP is effective to treat CSBD. In embodiments, the EAP treatment regimen is effective to treat CSBD. In embodiments, MDMA is administered. In embodiments, MDMA is effective to treat CSBD.

In embodiments, the administration of an entactogen according to the disclosed methods will be effective to treat CSBD as shown by a reduction in the severity of CSBD. In embodiments, a patient who has CSBD characterized as being mild when measured at baseline will no longer have a mild CSBD. In embodiments, a patient who has CSBD characterized as being moderate when measured at baseline will no longer have a moderate CSBD. In embodiments, a patient who has CSBD characterized as being severe when measured at baseline will no longer have a severe CSBD A reduction in the severity of CSBD can be determined based on ordinary skill, such as using an assessment.

In embodiments, the administration of an entactogen according to the disclosed methods will be effective to treat CSBD as shown by a reduction in CSBI score. In embodiments, a patient who scores 35 or higher on the Compulsive Sexual behavior Inventory (CSBI-13) when measured at baseline will have a reduction in the CSBI-13 of at least 5 points.

In embodiments, the administration of an entactogen according to the disclosed methods will be effective to treat CSBD as shown by one or more of: (a) a reduction in compulsive sexual behavior, (b) a reduction in drive or urge to engage in compulsive sexual behavior, (c) a promotion of abstinence from compulsive sexual behavior, (d) a prevention of relapse into compulsive sexual behavior, (e) a reduction in at least one symptom of CSBD.

In embodiments, administration of an entactogen according to the disclosed methods will result in a measurable reduction in compulsive sexual behavior or in the drive or urge to engage in compulsive sexual behavior, for example as compared to a baseline determination made before such administration. In embodiments, the reduction in compulsive sexual behavior is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from compulsive sexual behavior or prevention of relapse into compulsive sexual behavior, when measured from the start of treatment, such as the first administration. In embodiments, the promotion of abstinence from compulsive sexual behavior results in abstinence for at least 1 day, at least 1 week, at least 1 month, or at least 1 year, from the start of treatment. In embodiments, the reduction in compulsive sexual behavior, reduction in drive or urge to engage in compulsive sexual behavior, promotion of abstinence from compulsive sexual behavior, prevention of relapse into compulsive sexual behavior, and reduction in at least one symptom of compulsive sexual behavior disorder lasts for at least 1 day, at least 1 week, at least 1 month, or at least 1 year.

In embodiments, administration of an entactogen according to the disclosed methods will result in a measurable improvement in at least one symptom of CSBD, for example as compared to a baseline determination made before such administration. In embodiments, the reduction in at least one symptom of CSBD may be a decrease of one or more points of a patient completed self-assessment, as described herein, and/or a decrease of one or more points in a clinical outcome assessment or index score, such as the CSBI-13. In embodiments, administration of an entactogen according to the disclosed methods will result in an elimination of at least one symptom of compulsive sexual behavior disorder, for example as compared to a baseline determination made before such administration.

In embodiments, administration of an entactogen according to the disclosed methods will result in the patient having a reduction in at least one symptom of compulsive sexual behavior disorder, including engaging in repetitive sexual behavior has become a central focus of the individual's life to the point of neglecting health and personal care or other interests, activities and responsibilities; the individual has made numerous unsuccessful efforts to control or significantly reduce repetitive sexual behavior, the individual continues to engage in repetitive sexual behavior despite adverse consequences; and the person continues to engage in repetitive sexual behavior even when the individual derives little or no satisfaction from it.

In embodiments, administration of an entactogen according to the disclosed methods will result in the patient having an elimination of at least one symptom of compulsive sexual behavior disorder, including engaging in repetitive sexual behavior has become a central focus of the individual's life to the point of neglecting health and personal care or other interests, activities and responsibilities; the individual has made numerous unsuccessful efforts to control or significantly reduce repetitive sexual behavior, the individual continues to engage in repetitive sexual behavior despite adverse consequences; and the person continues to engage in repetitive sexual behavior even when the individual derives little or no satisfaction from it.

In embodiments, the patient with CSBD has a comorbid psychiatric condition. In embodiments, treating the patient with CSBD according to the disclosed methods is also effective to treat one or more comorbid psychiatric conditions in the patient.

iv. Compulsive Buying-Shopping Disorder

In some aspects, the provided methods are useful in treating a patient with compulsive buying-shopping disorder. In some embodiments, EAP is used to treat compulsive buying-shopping disorder. In some embodiments, EAP is used to treat compulsive buying-shopping disorder that is comorbid with any of a substance use disorder, another behavioral addiction, a mental health disorder, or a neurodegenerative condition. In embodiments, “compulsive buying-shopping disorder” includes related compulsive buying or compulsive shopping disorders, such as compulsive buying behavior disorder, compulsive buying disorder (CBD), compulsive buying behavior (CBB), compulsive online shopping disorder, compulsive spending disorder, spending addiction, and “oniomania.” Whether a patient has compulsive buying-shopping disorder can be determined according to ordinary skill.

For example, a patient may be determined to have compulsive buying-shopping disorder based on the definition provided by Black, 2001. Black, 2001 defines “compulsive buying disorder” as excessive or poorly controlled preoccupations, urges or behaviors regarding shopping and spending, which lead to adverse consequences (Black, CNS Drugs, 2001; 15(1):17-27; Granero, Front Psychol., 2016; 7:914). Exemplary diagnostic criteria include (a) feeling overly preoccupied with shopping and spending, (b) feeling that one's shopping behavior is excessive, inappropriate or uncontrolled, and (c) one's shopping desires, urges, fantasies or behaviors being overly time consuming, causing them to feel upset or guilty, or leading to serious problems in their life including financial/legal problems and relationship loss (Black, 2001).

The ICD-11 categorizes compulsive buying-shopping disorder as an unspecified mental, behavioral, or neurodevelopmental disorder. Diagnostic criteria include (a) a repeated failure to resist a strong impulse, drive, or urge to perform an act that is rewarding to the person, at least in the short-term, despite longer-term harm either to the individual or to others; (b) marked distress about the behavior pattern; and/or (c) significant impairment in personal, family, social, educational, occupational, or other important areas of functioning.

Compulsive buying-shopping disorder may be characterized as being mild, moderate, and severe. The severity of compulsive buying-shopping disorder may be determined via the modified Yale Brown Obsessive-Compulsive Scale, the YBOCS-Shopping Scale (YBOCS-SV), or based on ordinary skill. In embodiments, a patient may have compulsive buying-shopping disorder characterized as being mild. In other embodiments, a patient may have compulsive buying-shopping disorder characterized as being moderate. In other embodiments, a patient may have compulsive buying-shopping disorder characterized as being severe.

In embodiments, the term “compulsive buying-shopping disorder patient” (or “patient with compulsive buying-shopping disorder”) refers to a patient diagnosed with compulsive buying-shopping disorder, such as defined herein.

Herein, “reducing compulsive buying” or “reduction in compulsive buying” refers to reducing the amount or frequency of compulsive buying, such as assessed by standardised screening tools like the YBOCS-SV. In embodiments, “reducing compulsive buying” or “reduction in compulsive buying” refers to a reduction in daily compulsive buying, a reduction in the time spent compulsive buying per day, or a reduction in the frequency of compulsive buying, such as a reduction in the percentage of compulsive buying days in general, or a reduction in the percentage of heavy compulsive buying days. In embodiments, “reducing compulsive buying” or “reduction in compulsive buying” refers to an increase in the time to any compulsive buying or time to the first heavy compulsive buying day.

Herein, “compulsive buying abstinence” or “in abstinence from compulsive buying” refers to not engaging in compulsive buying. In embodiments, “promoting compulsive buying abstinence” or “promotion of compulsive buying abstinence” refers to help maintaining abstinence from compulsive buying, in particular after at least 1 day of not engaging in compulsive buying, for example, maintaining abstinence from compulsive buying for a period of at least 1 day, at least 1 week, at least 1 month, or at least 1 year.

Herein, “compulsive buying-shopping disorder patient in abstinence from compulsive buying” refers to a patient with compulsive buying-shopping disorder in abstinence from compulsive buying for a period, for example, for at least 1 day, at least 1 week, at least 1 month, or at least 1 year.

Herein, “relapse into compulsive buying” refers to compulsive buying following a period of abstinence from compulsive buying, for example following a period of compulsive buying abstinence of at least 1 day, at least 1 week, at least 1 month, or at least 1 year.

Herein, “preventing relapse into compulsive buying” refers to the prevention of compulsive buying by a compulsive buying-shopping disorder patient after the patient has stopped compulsive buying, in particular after 1 day or more of not engaging in compulsive buying. In embodiments, the term encompasses a delay in the resumption of compulsive buying as compared to the time to resumption by a patient that is not administered a compound of the invention using the disclosed methods. The delay in resumption can be for at least 1 day, at least 1 week, at least 1 month, or at least 1 year.

In embodiments, the patient with compulsive buying-shopping disorder is administered an entactogen. In embodiments, the patient with compulsive buying-shopping disorder receives EAP. In embodiments, the patient with compulsive buying-shopping disorder undergoes an EAP treatment regimen. In embodiments, the administration of the entactogen is effective to treat compulsive buying-shopping disorder. In embodiments, the EAP is effective to treat compulsive buying-shopping disorder. In embodiments, the EAP treatment regimen is effective to treat compulsive buying-shopping disorder. In embodiments, MDMA is administered. In embodiments, MDMA is effective to treat compulsive buying-shopping disorder.

In embodiments, the administration of an entactogen according to the disclosed methods will be effective to treat compulsive buying-shopping disorder as shown by a reduction in the severity of compulsive buying-shopping disorder. In embodiments, a patient who has compulsive buying-shopping disorder characterized as being mild when measured at baseline will no longer have a mild compulsive buying-shopping disorder. In embodiments, a patient who has compulsive buying-shopping disorder characterized as being moderate when measured at baseline will no longer have a moderate compulsive buying-shopping disorder. A reduction in the severity of compulsive buying-shopping disorder can be determined based on ordinary skill, such as using an assessment. For example, a patient who has compulsive buying-shopping disorder characterized as being severe when measured at baseline will no longer have a severe compulsive buying-shopping disorder. In embodiments, a patient who scores 20 or higher on the YBOCS-SV when measured at baseline will have a reduction in the YBOCS-SV of at least 3. In embodiments, a patient who scores 30 or higher on the YBOCS-SV when measured at baseline will have a reduction in the YBOCS-SV of at least 5.

In embodiments, the administration of an entactogen according to the disclosed methods will be effective to treat compulsive buying-shopping disorder as shown by one or more of: (a) a reduction in compulsive buying, (b) a reduction in drive or urge to engage in compulsive buying, (c) a promotion of abstinence from compulsive buying, (d) a prevention of relapse into compulsive buying, (e) a reduction in at least one symptom of compulsive buying-shopping disorder.

In embodiments, administration of an entactogen according to the disclosed methods will result in a measurable reduction in compulsive buying or in the drive or urge to engage in compulsive buying, for example as compared to a baseline determination made before such administration. In embodiments, the reduction in compulsive buying is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from compulsive buying or prevention of relapse into compulsive buying, when measured from the start of treatment, such as the first administration. In embodiments, the promotion of abstinence from compulsive buying results in abstinence for at least 1 day, at least 1 week, at least 1 month, or at least 1 year, from the start of treatment. In embodiments, the reduction in compulsive buying, reduction in drive or urge to engage in compulsive buying, promotion of abstinence from compulsive buying, prevention of relapse into compulsive buying, and reduction in at least one symptom of compulsive buying-shopping disorder lasts for at least 1 day, at least 1 week, at least 1 month, or at least 1 year.

In embodiments, administration of an entactogen according to the disclosed methods will result in a measurable improvement in at least one symptom of compulsive buying-shopping disorder, for example as compared to a baseline determination made before such administration. In embodiments, the reduction in at least one symptom of compulsive buying-shopping disorder may be a decrease of one or more points of a patient completed self-assessment, as described herein, and/or a decrease of one or more points in a clinical outcome assessment or index score, such as the YBOCS-SV In embodiments, administration of an entactogen according to the disclosed methods will result in an elimination of at least one symptom of compulsive buying-shopping disorder, for example as compared to a baseline determination made before such administration.

In embodiments, administration of an entactogen according to the disclosed methods will result in the patient having a reduction in at least one symptom of compulsive buying-shopping disorder, including a repeated failure to resist a strong impulse, drive, or urge to perform an act that is rewarding to the person, at least in the short-term, despite longer-term harm either to the individual or to others; marked distress about the behavior pattern, significant impairment in personal, family, social, educational, occupational, or other important areas of functioning; feeling overly preoccupied with shopping and spending, feeling that one's shopping behavior is excessive, inappropriate or uncontrolled; one's shopping desires, urges, fantasies or behaviors being overly time consuming, causing them to feel upset or guilty, or leading to serious problems in their life including financial/legal problems and relationship loss.

In embodiments, administration of an entactogen according to the disclosed methods will result in the patient having an elimination of at least one symptom of compulsive buying-shopping disorder, including a repeated failure to resist a strong impulse, drive, or urge to perform an act that is rewarding to the person, at least in the short-term, despite longer-term harm either to the individual or to others; marked distress about the behavior pattern, significant impairment in personal, family, social, educational, occupational, or other important areas of functioning; feeling overly preoccupied with shopping and spending, feeling that one's shopping behavior is excessive, inappropriate or uncontrolled; and one's shopping desires, urges, fantasies or behaviors being overly time consuming, causing them to feel upset or guilty, or leading to serious problems in their life including financial/legal problems and relationship loss.

In embodiments, the patient with compulsive buying-shopping disorder has a comorbid psychiatric condition. In embodiments, treating the patient with compulsive buying-shopping disorder is also effective to treat one or more comorbid psychiatric conditions in the patient.

v. Technology Addiction

In some aspects, the provided methods are useful in treating a patient with technology addiction. In some embodiments, EAP is used to treat technology addiction. In some embodiments, EAP is used to treat technology addiction that is comorbid with any of a substance use disorder, another behavioral addiction, a mental health disorder, or a neurodegenerative condition. Technology addiction includes compulsive technology use (CTU), as well as specific types including internet addiction, smartphone addiction, compulsive mobile app use, mobile email addiction, and information addiction.

Whether a patient has technology addiction can be determined according to ordinary skill. Exemplary criteria include those disclosed by Sternlicht and Sternlicht, 2022, which include: (a) inability to moderate or abstain from technology or a specific digital medium, (b) preoccupation with thinking about using technological devices, (c) compulsive technological use or experiencing cravings and urges to use digital devices, (d) neglecting important life areas such as work, school or relationships at the expense of technology, (e) continuing to use digital devices despite it contributing to consequences in your life, (f) losing interest in social and leisure activities that you once enjoyed at the expense of technology, (g) using digital devices in dangerous situations such as while driving a car or walking across a city street, (h) experiencing unwanted mental health symptoms such as depression, anxiety, stress or irritability at the expense of technological usage, (i) using digital devices to induce pleasure or experience gratification, (j) lying or hiding digital usage from family, friends or colleagues as a result of guilt or shame; and (k) using digital devices for longer durations than intended or finding yourself using digital devices with increased frequency over time.

Evidence shows that plasma dopamine levels are positively correlated with aspects of internet addiction (Liu & Luo, Int J Clin Exp Med. 2015; 8(6): 9943-9948). Internet activities, for example, winning a level of a video game or getting “likes” on a picture may enhance dopamine release.

Technology addiction may be characterized as being mild, moderate, and severe. The severity of technology addiction may be determined by the Digital Addiction Scale (DAS), wherein addiction is rated on a scale of 1-5, 5 being the highest addiction level (Kesici & Tunc, 2018), or based on ordinary skill. In embodiments, a patient may have technology addiction characterized as being mild. In embodiments, a patient may have technology addiction characterized as being moderate. In embodiments, a patient may have technology addiction characterized as being severe.

In embodiments, the term “technology addiction patient” refers to a patient diagnosed with technology addiction, such as defined herein.

Herein, “reducing compulsive technology use” or “reduction in compulsive technology use” refers to reducing the amount or frequency of compulsive technology use, such as assessed by standardised screening tools like the DAS. In embodiments, “reducing compulsive technology use” or “reduction in compulsive technology use” refers to a reduction in daily compulsive technology use, a reduction in the time spent compulsively using technology per day, or a reduction in the frequency of compulsive technology use, such as a reduction in the percentage of compulsive technology use days in general, or a reduction in the percentage of heavy compulsive technology use days. In embodiments, “reducing compulsive technology use” or “reduction in compulsive technology use” refers to an increase in the time to any compulsive technology use or time to the first heavy compulsive technology use day.

Herein, “technology addiction abstinence” or “in abstinence from technology” refers to not engaging in compulsive technology use. In embodiments, “promoting compulsive technology use abstinence” or “promotion of compulsive technology use abstinence” refers to help maintaining abstinence from compulsive technology use, in particular after at least 1 day of not engaging in compulsive technology use, for example, maintaining abstinence from compulsive technology use for a period of at least 1 day, at least 1 week, at least 1 month, or at least 1 year.

Herein, “technology addiction patient in abstinence from compulsive technology use” refers to a patient with technology addiction in abstinence from compulsive technology use for a period, e.g., for at least 1 day, at least 1 week, at least 1 month, or at least 1 year.

Herein, “relapse into compulsive technology use” refers to engaging in compulsive technology use following a period of abstinence from compulsive technology use, for example following a period of compulsive technology use abstinence of at least 1 day, at least 1 week, at least 1 month, or at least 1 year.

Herein, “preventing relapse into compulsive technology use” refers to the prevention of engaging in compulsive technology use by a technology addiction patient after the patient has stopped exhibiting compulsive technology use, in particular after 1 day or more of not exhibiting compulsive technology use. In embodiments, the term encompasses a delay in the resumption of compulsive technology use as compared to the time to resumption by a patient that is not administered a compound of the invention using the disclosed methods. The delay in resumption can be for at least 1 day, at least 1 week, at least 1 month, or at least 1 year.

In embodiments, the patient with technology addiction is administered an entactogen. In embodiments, the patient with technology addiction receives EAP. In embodiments, the patient with technology addiction undergoes an EAP treatment regimen. In embodiments, the administration of the entactogen is effective to treat technology addiction. In embodiments, the EAP is effective to treat technology addiction. In embodiments, the EAP treatment regimen is effective to treat technology addiction. In embodiments, MDMA. In embodiments MDMA is effective to treat technology addiction.

In embodiments, the administration of an entactogen according to the disclosed methods will be effective to treat technology addiction as shown by a reduction in the severity of technology addiction. In embodiments, a patient who has technology addiction characterized as being mild when measured at baseline will no longer have a mild technology addiction. In embodiments, a patient who has technology addiction characterized as being moderate when measured at baseline will no longer have a moderate technology addiction. In embodiments, a patient who has technology addiction characterized as being severe when measured at baseline will no longer have a severe technology addiction. A reduction in the severity of technology addiction can be determined based on ordinary skill, such as using an assessment. For example, in embodiments, a patient who has a DAS score of 2 or higher when measured at baseline will have a reduction in DAS score of at least 1 point. In embodiments, a patient who has a DAS score of 3 or higher when measured at baseline will have a reduction in DAS score of at least 2 points. In embodiments, a patient who has a DAS score of 5 when measured at baseline will have a reduction in DAS score of at least 3 points.

In embodiments, the administration of an entactogen according to the disclosed methods will be effective to treat technology addiction as shown by one or more of: (a) a reduction in compulsive technology use, (b) a reduction in drive or urge to engage in compulsive technology use, (c) a promotion of abstinence from compulsive technology use, (d) a prevention of relapse into compulsive technology use, (e) a reduction in at least one symptom of technology addiction.

In embodiments, administration of an entactogen according to the disclosed methods will result in a measurable reduction in compulsive technology use or in the drive or urge to engage in compulsive technology use, for example as compared to a baseline determination made before such administration. In embodiments, the reduction in compulsive technology use is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from compulsive technology use or prevention of relapse into compulsive technology use, when measured from the start of treatment, such as the first administration. In embodiments, the promotion of abstinence from compulsive technology use results in abstinence for at least 1 day, at least 1 week, at least 1 month, or at least 1 year, from the start of treatment. In embodiments, the reduction in compulsive technology use, reduction in drive or urge to engage in compulsive technology use, promotion of abstinence from compulsive technology use, prevention of relapse into compulsive technology use, and reduction in at least one symptom of technology addiction lasts for at least 1 day, at least 1 week, at least 1 month, or at least 1 year.

In embodiments, administration of an entactogen according to the disclosed methods will result in a measurable improvement in at least one symptom of technology addiction, for example as compared to a baseline determination made before such administration. In embodiments, the reduction in at least one symptom of technology addiction may be a decrease of one or more points of a patient completed self-assessment, as described herein, and/or a decrease of one or more points in a clinical outcome assessment or index score, such as the DAS. In embodiments, administration of an entactogen according to the disclosed methods will result in an elimination of at least one symptom of technology addiction, for example as compared to a baseline determination made before such administration.

In embodiments, administration of an entactogen according to the disclosed methods will result in the patient having a reduction in at least one symptom of technology addiction, including inability to moderate or abstain from technology or a specific digital medium, preoccupation with thinking about using technological devices, compulsive technological use or experiencing cravings and urges to use digital devices, neglecting important life areas such as work, school or relationships at the expense of technology, continuing to use digital devices despite it contributing to consequences in your life, losing interest in social and leisure activities that you once enjoyed at the expense of technology, using digital devices in dangerous situations such as while driving a car or walking across a city street, experiencing unwanted mental health symptoms such as depression, anxiety, stress or irritability at the expense of technological usage, using digital devices to induce pleasure or experience gratification, lying or hiding digital usage from family, friends or colleagues as a result of guilt or shame; and using digital devices for longer durations than intended or finding yourself using digital devices with increased frequency over time.

In embodiments, administration of an entactogen according to the disclosed methods will result in the patient having an elimination of at least one symptom of technology addiction, including inability to moderate or abstain from technology or a specific digital medium, preoccupation with thinking about using technological devices, compulsive technological use or experiencing cravings and urges to use digital devices, neglecting important life areas such as work, school or relationships at the expense of technology, continuing to use digital devices despite it contributing to consequences in your life, losing interest in social and leisure activities that you once enjoyed at the expense of technology, using digital devices in dangerous situations such as while driving a car or walking across a city street, experiencing unwanted mental health symptoms such as depression, anxiety, stress or irritability at the expense of technological usage, using digital devices to induce pleasure or experience gratification, lying or hiding digital usage from family, friends or colleagues as a result of guilt or shame; and using digital devices for longer durations than intended or finding yourself using digital devices with increased frequency over time.

In embodiments, the patient with technology addiction has a comorbid psychiatric condition. In embodiments, treating the patient with technology addiction is also effective to treat one or more comorbid psychiatric conditions in the patient.

vi. Kleptomania

In some aspects, the provided methods are useful in treating a patient with kleptomania. In some embodiments, EAP is used to treat kleptomania. In some embodiments, EAP is used to treat kleptomania that is comorbid with any of a substance use disorder, another behavioral addiction, a mental health disorder, or a neurodegenerative condition.

Whether a patient has kleptomania can be determined according to ordinary skill. For example, a patient may be determined to have kleptomania based on the definition in the DSM-V. The DSM-V defines kleptomania as a disorder characterized by the recurrent failure to resist impulses to steal items even though the items are not required for personal use or are of little value to the individual. The prevalence of kleptomania in the general population is rare, roughly between 0.3% to about 0.6%, and is higher in females than in males by about 3:1 (APA, 2013). Kleptomania is estimated to occur in about 4% to about 24% of individuals arrested for shoplifting (APA, 2013).

DSM-V diagnostic criteria for kleptomania include (a) recurrent failure to resist impulses to steal objects that are not needed for personal use or for their monetary value, (b) increasing sense of tension immediately before committing the theft, and (c) pleasure, gratification, or relief at the time of committing the theft. Additionally, the stealing not being committed to express anger or vengeance and not in response to a delusion or a hallucination, and the stealing not being better explained by conduct disorder, a manic episode, or antisocial personality disorder (APA, 2013).

The ICD-11 defines kleptomania as a recurrent failure to control strong impulses to steal objects in the absence of an apparent motive (e.g., objects are not acquired for personal use or monetary gain). ICD-11 diagnostic criteria for kleptomania include (a) a recurrent failure to control strong impulses to steal objects, (b) lack of an apparent motive for stealing objects (e.g., objects are not acquired for personal use or monetary gain), (c) the individual experiences increased tension or affective arousal prior to instances of theft or attempted theft, (d) the individual experiences pleasure, excitement, relief, or gratification during and immediately following the act of stealing, and (e) the acts of theft or attempted theft are not better accounted for by a disorder of intellectual development, another mental disorder (e.g., a manic episode), or substance intoxication (WHO, 2022).

Kleptomania may be characterized as being mild, moderate, and severe. The severity of kleptomania may be determined by the Kleptomania Symptom Assessment Scale (K-SAS), wherein scores ranging from 31 to 44 reflect severe kleptomania symptoms, scores ranging from 21 to 30 indicate moderate symptoms, scores ranging from 8-20 indicate mild symptoms, and scores below 8 indicate remission (Hollander and Berlin, 2008); or based on ordinary skill. In embodiments, a patient may have kleptomania characterized as being mild. In other embodiments, a patient may have kleptomania characterized as being moderate. In other embodiments, a patient may have kleptomania characterized as being severe. In embodiments, the Clinical Global Impressions scale (both severity and improvement measures), Sheehan Disability Scale (SDS), and Global Assessment of Functioning (GAF) may additionally be utilised.

In embodiments, “kleptomania disorder patient” (or “patient with kleptomania disorder”) refers to a patient diagnosed with kleptomania, such as defined herein.

Herein, “reducing stealing” or “reduction in stealing” refers to reducing the amount or frequency of stealing, such as assessed by standardised screening tools like K-SAS. In embodiments, “reducing stealing” or “reduction in stealing” refers to a reduction in daily stealing, a reduction in time spent stealing per day, or a reduction in the frequency of stealing, such as a reduction in the percentage of stealing days in general, or a reduction in the percentage of heavy stealing days. In embodiments, “reducing stealing” or “reduction in stealing” refers to an increase in time to any stealing or time to the first heavy stealing day.

Herein, “kleptomania abstinence” or “in abstinence from stealing” refers to not stealing. In embodiments, “promoting kleptomania abstinence” or “promotion of kleptomania abstinence” refers to help maintaining abstinence from stealing, in particular after at least 1 day of not stealing, for example, maintaining abstinence from stealing for a period of at least 1 day, at least 1 week, at least 1 month, or at least 1 year.

Herein, “kleptomania patient in abstinence from stealing” refers to a patient with kleptomania in abstinence from stealing for a period, for example, for at least 1 day, at least 1 week, at least 1 month, or at least 1 year.

Herein, “relapse into stealing” refers to stealing following a period of abstinence from stealing, for example following a period of stealing abstinence of at least 1 day, at least 1 week, at least 1 month, or at least 1 year.

Herein, “preventing relapse into stealing” refers to the prevention of stealing by a kleptomania patient after the patient has stopped stealing, in particular after 1 day or more of not stealing. In embodiments, the term encompasses a delay in the resumption of stealing as compared to the time to resumption by a patient that is not administered a compound of the invention using the disclosed methods. The delay in resumption can be for at least 1 day, at least 1 week, at least 1 month, or at least 1 year.

In embodiments, the patient with kleptomania is administered an entactogen. In embodiments, the patient with kleptomania receives EAP. In embodiments, the patient with kleptomania undergoes an EAP treatment regimen. In embodiments, the administration of the entactogen is effective to treat kleptomania. In embodiments, the EAP is effective to treat kleptomania. In embodiments, the EAP treatment regimen is effective to treat kleptomania. In embodiments, MDMA is administered. In embodiments, MDMA is effective to treat kleptomania.

In embodiments, the administration of an entactogen according to the disclosed methods will be effective to treat kleptomania as shown by a reduction in the severity of kleptomania. In embodiments, a patient who has kleptomania characterized as being mild when measured at baseline will no longer have mild kleptomania. In embodiments, a patient who has kleptomania characterized as being moderate when measured at baseline will no longer have moderate kleptomania. In embodiments, a patient who has kleptomania characterized as being severe when measured at baseline will no longer have severe kleptomania. A reduction in the severity of kleptomania can be determined based on ordinary skill, such as using an assessment. For example, in embodiments, a patient who scores at least 21 on the K-SAS when measured at baseline will have a reduction in the K-SAS score of at least 4 points. In embodiments, a patient who scores 31 or higher on the K-SAS when measured at baseline will have a reduction in the K-SAS score of at least 6 points.

In embodiments, the administration of an entactogen according to the disclosed methods will be effective to treat kleptomania as shown by one or more of: (a) a reduction in stealing, (b) a reduction in drive or urge to engage in stealing, (c) a promotion of abstinence from stealing, (d) a prevention of relapse into stealing, (e) a reduction in at least one symptom of kleptomania.

In embodiments, administration of an entactogen according to the disclosed methods will result in a measurable reduction in stealing or in the drive or urge to engage in stealing, for example as compared to a baseline determination made before such administration. In embodiments, the reduction in stealing is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from stealing or prevention of relapse into stealing, when measured from the start of treatment, such as the first administration. In embodiments, the promotion of abstinence from stealing results in abstinence for at least 1 day, at least 1 week, at least 1 month, or at least 1 year, from the start of treatment. In embodiments, the reduction in stealing, reduction in drive or urge to engage in stealing, promotion of abstinence from stealing, prevention of relapse into stealing, and reduction in at least one symptom of kleptomania lasts for at least 1 day, at least 1 week, at least 1 month, or at least 1 year.

In embodiments, administration of an entactogen according to the disclosed methods will result in a measurable improvement in at least one symptom of kleptomania, for example as compared to a baseline determination made before such administration. In embodiments, the reduction in at least one symptom of kleptomania may be a decrease of one or more points of a patient completed self-assessment, as described herein, and/or a decrease of one or more points in a clinical outcome assessment or index score, such as the K-SAS. In embodiments, administration of an entactogen according to the disclosed methods will result in an elimination of at least one symptom of kleptomania, for example as compared to a baseline determination made before such administration.

In embodiments, administration of an entactogen according to the disclosed methods will result in the patient having a reduction in at least one symptom of kleptomania, including recurrent failure to resist impulses to steal objects that are not needed for personal use or for their monetary value, increasing sense of tension immediately before committing the theft; pleasure, gratification, or relief at the time of committing the theft; a recurrent failure to control strong impulses to steal objects, lack of an apparent motive for stealing objects, the individual experiences increased tension or affective arousal prior to instances of theft or attempted theft, and the individual experiences pleasure, excitement, relief, or gratification during and immediately following the act of stealing.

In embodiments, administration of an entactogen according to the disclosed methods will result in the patient having an elimination of at least one symptom of kleptomania, including recurrent failure to resist impulses to steal objects that are not needed for personal use or for their monetary value, increasing sense of tension immediately before committing the theft; pleasure, gratification, or relief at the time of committing the theft; a recurrent failure to control strong impulses to steal objects, lack of an apparent motive for stealing objects, the individual experiences increased tension or affective arousal prior to instances of theft or attempted theft, and the individual experiences pleasure, excitement, relief, or gratification during and immediately following the act of stealing.

In embodiments, the patient with kleptomania has a comorbid psychiatric condition. In embodiments, treating the patient with kleptomania is also effective to treat one or more comorbid psychiatric conditions in the patient.

vii. Pyromania

In some aspects, the methods are useful in treating a patient with pyromania. In some embodiments, EAP is used to treat pyromania. In some embodiments, EAP is used to treat pyromania that is comorbid with any of a substance use disorder, another behavioral addiction, a mental health disorder, or a neurodegenerative condition.

Whether a patient has pyromania can be determined according to ordinary skill. For example, a patient may be determined to have pyromania based on the definition in the DSM-V. The DSM-V defines pyromania as a disorder characterized by multiple episodes of deliberate and purposeful fire setting associated with tension or affective arousal.

DSM-V diagnostic criteria for pyromania include (a) deliberate and purposeful fire setting on more than 1 occasion, (b) tension or affective arousal before the act, (c) fascination with, interest in, curiosity about, or attraction to fire and its situational contexts (e.g., paraphernalia, uses, consequences), and (d) pleasure, gratification, or relief when setting fires or when witnessing or participating in their aftermath. Additionally, the fire setting is not done for monetary gain, as an expression of sociopolitical ideology, to conceal criminal activity, to express anger or vengeance, to improve one's living circumstances, in response to a delusion or hallucination, or as a result of impaired judgment (e.g., major neurocognitive disorder, intellectual disability, substance intoxication); and the fire setting is not better explained by conduct disorder, a manic episode, or antisocial personality disorder.

The ICD-11 defines pyromania as a recurrent failure to control strong impulses to set fires, resulting in multiple acts of, or attempts at, setting fire to property or other objects, in the absence of an apparent motive (e.g., monetary gain, revenge, sabotage, political statement, attracting attention or recognition) (WHO, 2022).

ICD-11 diagnostic criteria for pyromania include (a) a recurrent failure to control strong impulses to set fires, resulting in multiple acts of, or attempts at, setting fire to property or other objects, (b) lack of an apparent motive for the acts of, or attempts at, fire setting (e.g., monetary gain, revenge, sabotage, political statement, attracting recognition), (c) persistent fascination or preoccupation with fire and related stimuli (e.g., watching fires, building fires, fascination with firefighting equipment), (d) the individual experiences increased tension or affective arousal prior to instances of, or attempts at, fire setting, (e) the individual experiences pleasure, excitement, relief or gratification during, and immediately following the act of setting the fire, witnessing its effects, or participating in its aftermath, (f) acts of, or attempts at, fire setting are not better accounted for by a disorder of intellectual development, another mental disorder (e.g., a manic episode), or substance intoxication.

In embodiments, the fire setting scale, fire proclivity scale, and St. Andrews Fire and Risk Instrument (SAFARI) may be utilised to distinguish firesetters with non-firesetters. The fire setting scale and fire proclivity scale are each discussed in Gannon and Barrowcliffe, 2010, which is herein incorporated by reference, while SAFARI is discussed in Long et al., 2013, which is also herein incorporated by reference.

In embodiments, the term “pyromania disorder patient” (or “patient with pyromania disorder”) refers to a patient diagnosed with pyromania, such as defined herein.

Herein, “reducing starting fires” or “reduction in fire starting” refers to reducing the amount or frequency of fire starting, such as assessed by standardised screening tools like the fire proclivity scale, the fire setting scale, and SAFARI. In embodiments, “reducing fire starting” or “reduction in fire starting” refers to a reduction in daily fire starting, a reduction in the time spent starting fires per day, or a reduction in the frequency of fire starting, such as a reduction in the percentage of fire starting days in general, or a reduction in the percentage of heavy fire starting days. In embodiments, “reducing fire starting” or “reduction in fire starting” refers to an increase in the time to any fire starting or time to the first heavy fire starting day.

Herein, “pyromania abstinence” or “in abstinence from fire starting” refers to not engaging in fire starting. In embodiments, “promoting pyromania abstinence” or “promotion of pyromania abstinence” refers to help maintaining abstinence from fire starting, in particular after at least 1 day of not starting fires, for example, maintaining abstinence from fire starting for a period of at least 1 day, at least 1 week, at least 1 month, or at least 1 year.

Herein, “pyromania patient in abstinence from fire starting” refers to a patient with pyromania in abstinence from fire starting for a period, for example, for at least 1 day, at least 1 week, at least 1 month, or at least 1 year.

Herein, “relapse into fire starting” refers to starting fires following a period of abstinence from starting fires, for example following a period of fire starting abstinence of at least 1 day, at least 1 week, at least 1 month, or at least 1 year.

Herein, “preventing relapse into fire starting” refers to the prevention of fire starting by a pyromania patient after the patient has stopped starting fires, in particular after 1 day or more of not starting fires. In embodiments, the term encompasses the permanent stoppage of fire starting. In embodiments, the term encompasses a delay in the resumption of fire starting as compared to the time to resumption by a patient that is not administered a compound of the invention using the disclosed methods. The delay in resumption can be for at least 1 day, at least 1 week, at least 1 month, or at least 1 year.

In embodiments, the patient with pyromania is administered an entactogen. In embodiments, the patient with pyromania receives EAP. In embodiments, the patient with pyromania undergoes an EAP treatment regimen. In embodiments, the administration of the entactogen is effective to treat pyromania. In embodiments, the EAP is effective to treat pyromania. In embodiments, the EAP treatment regimen is effective to treat pyromania. In embodiments, MDMA is administered. In embodiments, MDMA is effective to treat pyromania.

In embodiments, the administration of an entactogen according to the disclosed methods will be effective to treat pyromania as shown by a reduction in the severity of pyromania. In embodiments, a patient who has pyromania characterized as being mild when measured at baseline will no longer have a mild pyromania. In embodiments, a patient who has pyromania characterized as being moderate when measured at baseline will no longer have a moderate pyromania. In embodiments, a patient who has pyromania characterized as being severe when measured at baseline will no longer have a severe pyromania A reduction in the severity of pyromania can be determined based on ordinary skill, such as using an assessment.

In embodiments, a patient who was initially classed as a firesetter may, after treatment, be classed as a non-firesetter.

In embodiments, the administration of an entactogen according to the disclosed methods will be effective to treat pyromania as shown by one or more of: (a) a reduction in fire starting, (b) a reduction in drive or urge to engage in fire starting, (c) a promotion of abstinence from fire starting, (d) a prevention of relapse into fire starting, (e) a reduction in at least one symptom of pyromania.

In embodiments, administration of an entactogen according to the disclosed methods will result in a measurable reduction in starting fires or in the drive or urge to engage in fire starting, for example as compared to a baseline determination made before such administration. In embodiments, the reduction in starting fires is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from starting fires or prevention of relapse into starting fires, when measured from the start of treatment, such as the first administration. In embodiments, the promotion of abstinence from starting fires results in abstinence for at least 1 day, at least 1 week, at least 1 month, or at least 1 year, from the start of treatment. In embodiments, the reduction in fire starting, reduction in drive or urge to engage in fire starting, promotion of abstinence from fire starting, prevention of relapse into fire starting, and reduction in at least one symptom of pyromania lasts for at least 1 day, at least 1 week, at least 1 month, or at least 1 year.

In embodiments, administration of an entactogen according to the disclosed methods will result in a measurable improvement in at least one symptom of pyromania, for example as compared to a baseline determination made before such administration. In embodiments, the reduction in at least one symptom of pyromania may be a decrease of one or more points of a patient completed self-assessment, as described herein, and/or a decrease of one or more points in a clinical outcome assessment or index score, such as any of the fire setting scale, fire proclivity scale, and St. Andrews Fire and Risk Instrument (SAFARI). In embodiments, administration of an entactogen according to the disclosed methods will result in an elimination of at least one symptom of pyromania, for example as compared to a baseline determination made before such administration.

In embodiments, administration of an entactogen according to the disclosed methods will result in the patient having a reduction in at least one symptom of pyromania, including deliberate and purposeful fire setting on more than 1 occasion, tension or affective arousal before the act, fascination with, interest in, curiosity about, or attraction to fire and its situational contexts; pleasure, gratification, or relief when setting fires or when witnessing or participating in their aftermath; a recurrent failure to control strong impulses to set fires, resulting in multiple acts of, or attempts at, setting fire to property or other objects; lack of an apparent motive for the acts of, or attempts at, fire setting; persistent fascination or preoccupation with fire and related stimuli, the individual experiences increased tension or affective arousal prior to instances of, or attempts at, fire setting; the individual experiences pleasure, excitement, relief or gratification during, and immediately following the act of setting the fire, witnessing its effects, or participating in its aftermath.

In embodiments, administration of an entactogen according to the disclosed methods will result in the patient having an elimination of at least one symptom of pyromania, including deliberate and purposeful fire setting on more than 1 occasion, tension or affective arousal before the act, fascination with, interest in, curiosity about, or attraction to fire and its situational contexts; pleasure, gratification, or relief when setting fires or when witnessing or participating in their aftermath; a recurrent failure to control strong impulses to set fires, resulting in multiple acts of, or attempts at, setting fire to property or other objects; lack of an apparent motive for the acts of, or attempts at, fire setting; persistent fascination or preoccupation with fire and related stimuli, the individual experiences increased tension or affective arousal prior to instances of, or attempts at, fire setting; the individual experiences pleasure, excitement, relief or gratification during, and immediately following the act of setting the fire, witnessing its effects, or participating in its aftermath.

In embodiments, the patient with pyromania has a comorbid psychiatric condition. In embodiments, treating the patient with pyromania is also effective to treat one or more comorbid psychiatric conditions in the patient.

viii. Internet Addiction

In some aspects, the methods are useful in treating a patient with internet addiction. Internet addiction also may include related conditions such as the compulsive use of electronic devices, compulsive texting, and compulsively checking social media. In some embodiments, EAP is used to treat internet addiction. In some embodiments, EAP is used to treat internet addiction that is comorbid with any of a substance use disorder, another behavioral addiction, a mental health disorder, or a neurodegenerative condition.

Whether a patient has internet addiction can be determined according to ordinary skill. For example, a patient may be determined to have internet addiction based on the definition provided by Cash et al., 2012. Per Cash et al., internet addiction is accompanied by changes in mood, preoccupation with the Internet and digital media, the inability to control the amount of time spent interfacing with digital technology, the need for more time to achieve a desired mood, withdrawal symptoms when not engaged, and a continuation of the behavior despite family conflict, a diminishing social life and adverse work or academic consequences (Cash et al., 2012).

Exemplary diagnostic criteria for internet addiction proposed by Young, 1998 include presence of the following: (a) a preoccupation with the Internet (thinks about previous online activity or anticipates next online session), (b) need to use the Internet with increased amounts of time in order to achieve satisfaction, (c) repeatedly making unsuccessful efforts to control, cut back, or stop Internet use; (d) is restless, moody, depressed, or irritable when attempting to cut down or stop Internet use; (e) staying online longer than originally intended; (f) having jeopardised or risked the loss of a significant relationship, job, educational or career opportunity because of the Internet; (g) having lied to family members, therapist, or others to conceal the extent of involvement with the Internet; (h) and using the Internet as a way of escaping from problems or of relieving a dysphoric mood (e.g., feelings of helplessness, guilt, anxiety, depression) (Young, 1998). Per Young, patients meeting five or more of the criteria are classified as dependent internet users, while those answering yes to less than five are classified as nondependent internet users (1998).

Internet addiction may be characterized as being mild, moderate, and severe. The severity of internet addiction may be determined by the internet addiction test (IAT), wherein a score between below 30 indicates a normal level of internet consumption, scores between 31 and 49 indicate mild level addiction, scores between 50 and 79 indicate moderate addiction, and scores of 80 or above indicate severe internet dependency (Samaha et al., 2018); or based on ordinary skill. In embodiments, a patient may have internet addiction characterized as being mild. In other embodiments, a patient may have internet addiction characterized as being moderate. In other embodiments, a patient may have internet addiction characterized as being severe. In embodiments, the Internet addiction scale (IAS), the Young of the Internet Addiction Questionnaire (YDQI), and the Chen's Internet addiction scale (CIAS) may also be utilised to assess the presence and/or severity of internet addiction.

In embodiments, the term “internet addiction patient” refers to a patient diagnosed with internet addiction, such as defined herein.

Herein, “reducing compulsive internet use” or “reduction in compulsive internet use” refers to reducing the amount or frequency of compulsive internet use, such as assessed by standardised screening tools like the IAS, YDQI, CIAS, and IAT. In embodiments, “reducing compulsive internet use” or “reduction in compulsive internet use” refers to a reduction in daily compulsive internet use, a reduction in the time spent compulsively using the internet per day, or a reduction in the frequency of compulsive internet use, such as a reduction in the percentage of compulsive internet use days in general, or a reduction in the percentage of heavy compulsive internet use days. In embodiments, “reducing compulsive internet use” or “reduction in compulsive internet use” refers to an increase in the time to any compulsive internet use or time to the first heavy compulsive internet use day.

Herein, “internet addiction abstinence” or “in abstinence from the internet” refers to not engaging in compulsive internet use. In embodiments, “promoting compulsive internet use abstinence” or “promotion of compulsive internet use abstinence” refers to help maintaining abstinence from compulsive internet use, in particular after at least 1 day of not engaging in compulsive internet use, for example, maintaining abstinence from compulsive internet use for a period of at least 1 day, at least 1 week, at least 1 month, or at least 1 year.

Herein, “internet addiction patient in abstinence from compulsive internet use” refers to a patient with internet addiction in abstinence from compulsive internet use for a period, for example, for at least 1 day, at least 1 week, at least 1 month, or at least 1 year. Herein, “relapse into compulsive internet use” refers to engaging in compulsive internet use following a period of abstinence from compulsive internet use, for example following a period of compulsive internet use abstinence of at least 1 day, at least 1 week, at least 1 month, or at least 1 year.

Herein, “preventing relapse into compulsive internet use” refers to the prevention of engaging in compulsive internet use by an internet addiction patient after the patient has stopped exhibiting compulsive internet use, in particular after 1 day or more of not exhibiting compulsive internet use. In embodiments, the term encompasses a delay in the resumption of compulsive internet use as compared to the time to resumption by a patient that is not administered a compound of the invention using the disclosed methods. The delay in resumption can be for at least 1 day, at least 1 week, at least 1 month, or at least 1 year.

In embodiments, the patient with internet addiction is administered an entactogen. In embodiments, the patient with internet addiction receives EAP. In embodiments, the patient with internet addiction undergoes an EAP treatment regimen. In embodiments, the administration of the entactogen is effective to treat internet addiction. In embodiments, the EAP is effective to treat internet addiction. In embodiments, the EAP treatment regimen is effective to treat internet addiction. In embodiments, MDMA is administered. In embodiments, MDMA is effective to treat internet addiction.

In embodiments, the administration of an entactogen according to the disclosed methods will be effective to treat internet addiction as shown by a reduction in the severity of internet addiction. In embodiments, a patient who has internet addiction characterized as being mild when measured at baseline will no longer have a mild internet addiction. In embodiments, a patient who has internet addiction characterized as being moderate when measured at baseline will no longer have a moderate internet addiction. In embodiments, a patient who has internet addiction characterized as being severe when measured at baseline will no longer have a severe internet addiction. A reduction in the severity of internet addiction can be determined based on ordinary skill, such as using an assessment. For example, in embodiments, a patient who scores 30 or higher on the IAT when measured at baseline will have a reduction in IAT score of at least 4 points. In embodiments, a patient who scores 50 or higher on the IAT when measured at baseline will have a reduction in IAT score of at least 7 points. In embodiments, a patient who scores 80 points or higher on the IAT when measured at baseline will have a reduction in IAT score of at least 12 points.

In embodiments, the administration of an entactogen according to the disclosed methods will be effective to treat internet addiction as shown by one or more of: (a) a reduction in compulsive internet use, (b) a reduction in drive or urge to engage in compulsive internet use, (c) a promotion of abstinence from compulsive internet use, (d) a prevention of relapse into compulsive internet use, (e) a reduction in at least one symptom of internet addiction.

In embodiments, administration of an entactogen according to the disclosed methods will result in a measurable reduction in compulsive internet use or in the drive or urge to engage in compulsive internet use, for example as compared to a baseline determination made before such administration. In embodiments, the reduction in compulsive internet use is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from compulsive internet use or prevention of relapse into compulsive internet use, when measured from the start of treatment, such as the first administration. In embodiments, the promotion of abstinence from compulsive internet use results in abstinence for at least 1 day, at least 1 week, at least 1 month, or at least 1 year, from the start of treatment. In embodiments, the reduction in compulsive internet use, reduction in drive or urge to engage in compulsive internet use, promotion of abstinence from compulsive internet use, prevention of relapse into compulsive internet use, and reduction in at least one symptom of internet addiction lasts for at least 1 day, at least 1 week, at least 1 month, or at least 1 year.

In embodiments, administration of an entactogen according to the disclosed methods will result in a measurable improvement in at least one symptom of internet addiction, for example as compared to a baseline determination made before such administration. In embodiments, the reduction in at least one symptom of internet addiction may be a decrease of one or more points of a patient completed self-assessment, as described herein, and/or a decrease of one or more points in a clinical outcome assessment or index score, such as the IAS. In embodiments, administration of an entactogen according to the disclosed methods will result in an elimination of at least one symptom of internet addiction, for example as compared to a baseline determination made before such administration.

In embodiments, administration of an entactogen according to the disclosed methods will result in the patient having a reduction in at least one symptom of internet addiction, including a preoccupation with the Internet, need to use the Internet with increased amounts of time in order to achieve satisfaction, repeatedly making unsuccessful efforts to control, cut back, or stop Internet use; is restless, moody, depressed, or irritable when attempting to cut down or stop Internet use; staying online longer than originally intended; having jeopardised or risked the loss of a significant relationship, job, educational or career opportunity because of the Internet; having lied to family members, therapist, or others to conceal the extent of involvement with the Internet; and using the Internet as a way of escaping from problems or of relieving a dysphoric mood.

In embodiments, administration of an entactogen according to the disclosed methods will result in the patient having an elimination of at least one symptom of internet addiction, including a preoccupation with the Internet, need to use the Internet with increased amounts of time in order to achieve satisfaction, repeatedly making unsuccessful efforts to control, cut back, or stop Internet use; is restless, moody, depressed, or irritable when attempting to cut down or stop Internet use; staying online longer than originally intended; having jeopardised or risked the loss of a significant relationship, job, educational or career opportunity because of the Internet; having lied to family members, therapist, or others to conceal the extent of involvement with the Internet; and using the Internet as a way of escaping from problems or of relieving a dysphoric mood.

In embodiments, the patient with internet addiction has a comorbid psychiatric condition. In embodiments, treating the patient with internet addiction is also effective to treat one or more comorbid psychiatric conditions in the patient.

ix. Pornography Addiction

In some aspects, the methods are useful in treating a patient with pornography addiction. In some embodiments, EAP is used to treat pornography addiction. In some embodiments, EAP is used to treat pornography addiction that is comorbid with any of a substance use disorder, another behavioral addiction, a mental health disorder, or a neurodegenerative condition.

Whether a patient has pornography addiction can be determined according to ordinary skill. For example, a patient may be determined to have pornography addiction based on the definition provided by Parker, 2021. Per Parker, pornography addiction is a compulsive need to view pornography despite negative consequences (2021). Exemplary diagnostic criteria for pornography addiction include: (a) you are consumed with thoughts of porn even when you are not actively watching it, (b) you view porn on your cell phone at work or in social situations where you might be seen, (c) you feel a compulsion to watch more pornography, (d) you feel ashamed, guilty, or depressed about your porn viewing, (e) you continue to watch porn despite the harm it has had, is having, or may have on your relationships, work, or home life, (f) you experience reduced sexual satisfaction with partners when pornography is not involved, (g) you hide your porn and porn viewing from your partner and family members, (h) you get upset when asked to cut back on or stop looking at porn, (i) you lose track of time when viewing porn, and (j) you have tried to quit watching porn but have not been successful (Parker, 2021).

The Brief Pornography Screener (BPS) and/or the Problematic Porn Consumption Scale (PPCS) may be utilized to distinguish between problematic pornography use, and non-problematic pornography consumption. A score on the BPS above 4 is considered problematic, while a score of above 76 on the PPCS is considered problematic.

In embodiments, the term “pornography addiction patient” refers to a patient diagnosed with pornography addiction, such as defined herein.

Herein, “reducing problematic pornography consumption” or “reduction in problematic pornography consumption” refers to reducing the amount or frequency of problematically consuming pornography, such as assessed by standardized screening tools like the BPS and PPCS. In embodiments, “reducing problematic pornography consumption” or “reduction in problematic pornography consumption” refers to a reduction in daily problematic pornography consumption, a reduction in the time spent problematically consuming pornography per day, or a reduction in the frequency of problematic pornography consumption, such as a reduction in the percentage of problematic pornography consumption days in general, or a reduction in the percentage of heavy problematic pornography consumption days. In embodiments, “reducing problematic pornography consumption” or “reduction in problematic pornography consumption” refers to an increase in the time to any problematic pornography consumption or time to the first heavy problematic pornography consumption day.

Herein, “pornography addiction abstinence” or “in abstinence from problematic pornography consumption” refers to not engaging in problematic pornography consumption. In embodiments, “promoting problematic pornography consumption abstinence” or “promotion of problematic pornography consumption abstinence” refers to help maintaining abstinence from problematic pornography consumption, in particular after at least 1 day of non-problematic pornography consumption, for example, maintaining abstinence from problematic pornography consumption for a period of at least 1 day, at least 1 week, at least 1 month, or at least 1 year.

Herein, “pornography addiction patient in abstinence from problematic pornography consumption” refers to a patient with pornography addiction in abstinence from problematic pornography consumption for a period, for example, for at least 1 day, at least 1 week, at least 1 month, or at least 1 year.

Herein, “relapse into problematic pornography consumption” refers to engaging in problematic pornography consumption following a period of abstinence from problematic pornography consumption, for example following a period of problematic pornography consumption abstinence of at least 1 day, at least 1 week, at least 1 month, or at least 1 year.

Herein, “preventing relapse into problematic pornography consumption” refers to the prevention of engaging in problematic pornography consumption by a pornography addiction patient after the patient has stopped problematic pornography consumption, in particular after 1 day or more of non-problematic pornography consumption. In embodiments, the term encompasses a delay in the resumption of problematic pornography consumption as compared to the time to resumption by a patient that is not administered a compound of the invention using the disclosed methods. The delay in resumption can be for at least 1 day, at least 1 week, at least 1 month, or at least 1 year.

In embodiments, the patient with pornography addiction is administered an entactogen. In embodiments, the patient with pornography addiction receives EAP. In embodiments, the patient with pornography addiction undergoes an EAP treatment regimen. In embodiments, the administration of the entactogen is effective to treat pornography addiction. In embodiments, the EAP is effective to treat pornography addiction. In embodiments, the EAP treatment regimen is effective to treat pornography addiction. In embodiments, MDMA is administered. In embodiments, MDMA is effective to treat pornography addiction.

In embodiments, the administration of an entactogen according to the disclosed methods will be effective to treat pornography addiction as shown by a reduction in the severity of pornography addiction. In embodiments, a patient who has pornography addiction characterized as being mild when measured at baseline will no longer have a mild pornography addiction. In embodiments, a patient who has pornography addiction characterized as being moderate when measured at baseline will no longer have a moderate pornography addiction. In embodiments, a patient who has pornography addiction characterized as being severe when measured at baseline will no longer have a severe pornography addiction A reduction in the severity of pornography addiction can be determined based on ordinary skill, such as using an assessment.

In embodiments, the administration of an entactogen according to the disclosed methods will be effective to reduce the patient's pornography consumption. In embodiments, a patient who has pornography addiction characterized as being problematic by one or more of the BPS and/or the PPCS when measured at baseline will no longer have pornography addiction characterized as being problematic. In embodiments, a patient who scores 4 or higher on the BPS when measured at baseline will have a reduction in the BPS of at least 1 point. In embodiments, a patient who scores 76 or higher on the PPCS when measured at baseline will have a reduction in the PPCS of at least 10 points.

In embodiments, the administration of an entactogen according to the disclosed methods will be effective to treat pornography addiction as shown by one or more of: (a) a reduction in pornography addiction, (b) a reduction in drive or urge to engage in problematic pornography consumption, (c) a promotion of abstinence from problematic pornography consumption, (d) a prevention of relapse into pornography addiction, (e) a reduction in at least one symptom of pornography addiction.

In embodiments, administration of an entactogen according to the disclosed methods will result in a measurable reduction in problematic pornography consumption or in the drive or urge to engage in problematic pornography consumption, for example as compared to a baseline determination made before such administration. In embodiments, the reduction in problematic pornography consumption is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from problematic pornography consumption or prevention of relapse into problematic pornography consumption, when measured from the start of treatment, such as the first administration. In embodiments, the promotion of abstinence from problematic pornography consumption results in abstinence for at least 1 day, at least 1 week, at least 1 month, or at least 1 year, from the start of treatment. In embodiments, the reduction in problematic pornography consumption, reduction in drive or urge to engage in problematic pornography consumption, promotion of abstinence from problematic pornography consumption, prevention of relapse into problematic pornography consumption, and reduction in at least one symptom of pornography addiction lasts for at least 1 day, at least 1 week, at least 1 month, or at least 1 year.

In embodiments, administration of an entactogen according to the disclosed methods will result in a measurable improvement in at least one symptom of pornography addiction, for example as compared to a baseline determination made before such administration. In embodiments, the reduction in at least one symptom of pornography addiction may be a decrease of one or more points of a patient completed self-assessment, as described herein, and/or a decrease of one or more points in a clinical outcome assessment or index score, such as the BPS, PPUS, and/or PPCS. In embodiments, administration of an entactogen according to the disclosed methods will result in an elimination of at least one symptom of internet addiction, for example as compared to a baseline determination made before such administration.

In embodiments, administration of an entactogen according to the disclosed methods will result in the patient having a reduction in at least one symptom of pornography addiction, including being consumed with thoughts of porn even when not actively watching it, viewing porn on their cell phone at work or in social situations where they might be seen, a compulsion to watch more pornography, feeling ashamed, guilty, or depressed about their porn viewing; continuing to watch porn despite the harm it has had, is having, or may have on their relationships, work, or home life, experiencing reduced sexual satisfaction with partners when pornography is not involved, hiding their porn and porn viewing from their partner and family members, getting upset when asked to cut back on or stop looking at porn, losing track of time when viewing porn, and trying to quit watching porn but not being successful.

In embodiments, administration of an entactogen according to the disclosed methods will result in the patient having an elimination of at least one symptom of pornography addiction, including being consumed with thoughts of porn even when not actively watching it, viewing porn on their cell phone at work or in social situations where they might be seen, a compulsion to watch more pornography, feeling ashamed, guilty, or depressed about their porn viewing; continuing to watch porn despite the harm it has had, is having, or may have on their relationships, work, or home life, experiencing reduced sexual satisfaction with partners when pornography is not involved, hiding their porn and porn viewing from their partner and family members, getting upset when asked to cut back on or stop looking at porn, losing track of time when viewing porn, and trying to quit watching porn but not being successful.

In embodiments, the patient with pornography addiction has a comorbid psychiatric condition. In embodiments, treating the patient with pornography addiction is also effective to treat one or more comorbid psychiatric conditions in the patient.

x. Binge Eating Disorder

In some aspects, the methods are useful in treating a patient with binge eating disorder (BED). In some embodiments, EAP is used to treat BED. In some embodiments, EAP is used to treat BED that is comorbid with any of a substance use disorder, another behavioral addiction, a mental health disorder, or a neurodegenerative condition.

Whether a patient has binge eating disorder can be determined according to ordinary skill. For example, a patient may be determined to have binge eating disorder based on the definition in the ICD-11. The ICD-11 defines binge eating disorder as a discrete period of time (e.g., 2 hours) during which the individual experiences a loss of control over their eating behavior and eats notably more or differently than usual. Loss of control over eating may be described by the individual as feeling like they cannot stop or limit the amount or type of food eaten; having difficulty stopping eating once they have started; or giving up even trying to control their eating because they know they will end up overeating (WHO, 2022).

ICD-11 diagnostic criteria for binge eating disorder include (a) frequent, recurrent episodes of binge eating (e.g., once a week or more over a period of 3 months). Binge eating is defined as a discrete period of time (e.g., 2 hours) during which the individual experiences a loss of control over their eating behavior and eats notably more or differently than usual. Loss of control over eating may be described by the individual as feeling like they cannot stop or limit the amount or type of food eaten; having difficulty stopping eating once they have started; or giving up even trying to control their eating because they know they will end up overeating. (b) The binge eating episodes not regularly being accompanied by inappropriate compensatory behaviors aimed at preventing weight gain, (c) the symptoms and behaviors not being better accounted for by another medical condition (e.g., Prader-Willi Syndrome) or mental disorder (e.g., a Depressive Disorder) and are not due to the effects of a substance or medication on the CNS, including withdrawal effects, and (d) there is marked distress about the pattern of binge eating or significant impairment in personal, family, social, educational, occupational or other important areas of functioning (WHO, 2022).

The DSM-V defines binge eating disorder as recurrent episodes of binge eating that is defined as an amount of food that is definitely larger than most people would eat in a similar period of time under similar circumstances accompanied by a sense of lack of control over eating during the episode (APA, 2013).

Per the DSM-V, an episode of binge eating is characterized by both of the following: (1) eating, in a discrete period of time (e.g.—within any 2-hour period), an amount of food that is definitely larger than what most people would eat in a similar period of time under similar circumstances, (2) a sense of lack of control over eating during the episode (e.g.—a feeling that one cannot stop eating or control what or how much one is eating). The binge eating episodes are associated with at least 3 of the following: (a) eating much more rapidly than normal, (b) eating until feeling uncomfortably full, (c) eating large amounts of food when not feeling physically hungry, (d) eating alone because of being embarrassed by how much one is eating, (e) feeling disgusted with oneself, depressed, or very guilty after overeating. In addition, marked distress regarding binge eating is present, the binge eating occurs, on average, at least once a week for three months; and the binge eating is not associated with the recurrent use of inappropriate compensatory behaviors (such as purging) and does not occur exclusively during the course Bulimia Nervosa or Anorexia Nervosa.

Binge eating disorder may be characterized as being mild, moderate, and severe. The severity of binge eating disorder may be determined by the Binge Eating Scale (BES), wherein a score below 17 indicates no binge eating disorder, a score of between 18-20 indicates mild binge eating disorder, a score of between 21-27 indicates moderate binge eating disorder, and a score above 27 indicates severe binge eating disorder (Grupski et al., 2013), or based on ordinary skill. In embodiments, a patient may have binge eating disorder characterized as being mild. In other embodiments, a patient may have binge eating disorder characterized as being moderate. In other embodiments, a patient may have binge eating disorder characterized as being severe.

In embodiments, “binge eating disorder patient” (or “patient with binge eating disorder”) refers to a patient diagnosed with binge eating disorder, such as defined herein.

Herein, “reducing binge eating” or “reduction in binge eating” refers to reducing the amount or frequency of binge eating, such as assessed by standardised screening tools like the BES. In embodiments, “reducing binge eating” or “reduction in binge eating” refers to a reduction in daily binge eating, a reduction in the time spent binge eating per day, or a reduction in the frequency of binge eating, such as a reduction in the percentage of binge eating days in general, or a reduction in the percentage of heavy binge eating days. In embodiments, “reducing binge eating” or “reduction in binge eating” refers to an increase in the time to any binge eating or time to the first heavy binge eating day.

Herein, “binge eating abstinence” or “in abstinence from binge eating” refers to not engaging in binge eating. In embodiments, “promoting binge eating abstinence” or “promotion of binge eating abstinence” refers to help maintaining abstinence from binge eating, in particular after at least 1 day of not binge eating, for example, maintaining abstinence from binge eating for a period of at least 1 day, at least 1 week, at least 1 month, or at least 1 year.

Herein, “binge eating disorder patient in abstinence from binge eating” refers to a patient with binge eating disorder in abstinence from binge eating for a period, for example, for at least 1 day, at least 1 week, at least 1 month, or at least 1 year.

Herein, “relapse into binge eating” refers to engaging in binge eating following a period of abstinence from binge eating, for example following a period of binge eating abstinence of at least 1 day, at least 1 week, at least 1 month, or at least 1 year.

Herein, “preventing relapse into binge eating” refers to the prevention of engaging in binge eating by a binge eating disorder patient after the patient has stopped binge eating, in particular after 1 day or more of not binge eating. In embodiments, the term encompasses a delay in the resumption of binge eating as compared to the time to resumption by a patient that is not administered a compound of the invention using the disclosed methods. The delay in resumption can be for at least 1 day, at least 1 week, at least 1 month, or at least 1 year.

In embodiments, the patient with binge eating disorder is administered an entactogen. In embodiments, the patient with binge eating disorder receives EAP. In embodiments, the patient with binge eating disorder undergoes an EAP treatment regimen. In embodiments, the administration of the entactogen is effective to treat binge eating disorder. In embodiments, the EAP is effective to treat binge eating disorder. In embodiments, the EAP treatment regimen is effective to treat binge eating disorder. In embodiments, MDMA is administered. In embodiments, MDMA is effective to treat binge eating disorder.

In embodiments, the administration of an entactogen according to the disclosed methods will be effective to treat BED as shown by a reduction in the severity of binge eating disorder. In embodiments, a patient who has binge eating disorder characterized as being mild when measured at baseline will no longer have a mild binge eating disorder. In embodiments, a patient who has binge eating disorder characterized as being moderate when measured at baseline will no longer have a moderate binge eating disorder. In embodiments, a patient who has binge eating disorder characterized as being severe when measured at baseline will no longer have a severe binge eating disorder. A reduction in the severity of binge eating disorder can be determined based on ordinary skill, such as using an assessment. For example, in embodiments, a patient who scores 18 or higher on the BES when measured at baseline will have a reduction in BES score of at least 2 points. In embodiments, a patient who scores 21 or higher on the BES when measured at baseline will have a reduction in BES score of at least 3 points. In embodiments, a patient who scores 27 or higher on the BES when measured at baseline will have a reduction in BES score of at least 4 points.

In embodiments, the administration of an entactogen according to the disclosed methods will be effective to treat binge eating disorder as shown by one or more of: (a) a reduction in binge eating, (b) a reduction in drive or urge to engage in binge eating, (c) a promotion of abstinence from binge eating, (d) a prevention of relapse into binge eating, (e) a reduction in at least one symptom of binge eating disorder.

In embodiments, administration of an entactogen according to the disclosed methods will result in a measurable reduction in binge eating or in the drive or urge to engage in binge eating, e.g., as compared to a baseline determination made before such administration. In embodiments, the reduction in binge eating is at least 15%, including 20%, 25%, 30%, 40%, 50%, 60%, 70%, 80%, 90%, up to and including 100%, when measured from baseline.

In embodiments, administration of an entactogen according to the disclosed methods will result in a durable promotion of abstinence from binge eating or prevention of relapse into binge eating, when measured from the start of treatment, such as the first administration. In embodiments, the promotion of abstinence from binge eating results in abstinence for at least 1 day, at least 1 week, at least 1 month, or at least 1 year, from the start of treatment. In embodiments, the reduction in binge eating, reduction in drive or urge to engage in binge eating, promotion of abstinence from binge eating, prevention of relapse into binge eating, and reduction in at least one symptom of binge eating disorder lasts for at least 1 day, at least 1 week, at least 1 month, or at least 1 year.

In embodiments, administration of an entactogen according to the disclosed methods will result in the patient having a reduction in at least one symptom of binge eating disorder, including frequent, recurrent episodes of binge eating, feeling like they cannot stop or limit the amount or type of food eaten; having difficulty stopping eating once they have started; giving up even trying to control their eating because they know they will end up overeating; marked distress about the pattern of binge eating or significant impairment in personal, family, social, educational, occupational or other important areas of functioning; eating, in a discrete period of time an amount of food that is definitely larger than what most people would eat in a similar period of time under similar circumstances, a sense of lack of control over eating during the episode; eating much more rapidly than normal, eating until feeling uncomfortably full, eating large amounts of food when not feeling physically hungry, eating alone because of being embarrassed by how much one is eating, and feeling disgusted with oneself, depressed, or very guilty after overeating.

In embodiments, administration of an entactogen according to the disclosed methods will result in the patient having an elimination of at least one symptom of binge eating disorder, including frequent, recurrent episodes of binge eating, feeling like they cannot stop or limit the amount or type of food eaten; having difficulty stopping eating once they have started; giving up even trying to control their eating because they know they will end up overeating; marked distress about the pattern of binge eating or significant impairment in personal, family, social, educational, occupational or other important areas of functioning; eating, in a discrete period of time an amount of food that is definitely larger than what most people would eat in a similar period of time under similar circumstances, a sense of lack of control over eating during the episode; eating much more rapidly than normal, eating until feeling uncomfortably full, eating large amounts of food when not feeling physically hungry, eating alone because of being embarrassed by how much one is eating, and feeling disgusted with oneself, depressed, or very guilty after overeating.

In embodiments, administration of an entactogen according to the disclosed methods will result in the patient no longer having episodes of binge eating, having distress about the pattern of binge eating or significant impairment in personal, family, social, educational, occupational or other important areas of functioning; eating rapidly, feeling uncomfortably full, eating large amounts of food when not feeling physically hungry, eating alone because of being embarrassed by how much they are eating, feeling disgusted with oneself, depressed, or very guilty after overeating; having distress regarding binge eating, and having episodes of binge eating at least once per week.

In embodiments, the patient with binge eating disorder has a comorbid psychiatric condition. In embodiments, treating the patient with binge eating disorder is also effective to treat one or more comorbid psychiatric conditions in the patient.

d. Methods of Administering EAP

The disclosed compositions and methods, such as EAP, are useful to treat AUD, SUDs, and BAs. In some embodiments, the provided compositions and methods, such as EAP, treat comorbid SUDs and BAs. In some embodiments, the treated comorbidities coexist with any one or more of an SUD, BA, mental health disorder, and neurodegenerative condition. In some embodiments, the provided compositions and methods, such as EAP, treat psychiatric conditions comorbid with BAs.

In some embodiments, a patient who may be administered EAP is assessed for the presence of comorbid conditions or comorbidities prior to administration of EAP. In some embodiments, the patient having comorbidities is diagnosed with a coexisting substance use disorder, behavioral addiction, mental health disorder, neurodegenerative condition. In some embodiments, the comorbidity is a substance use disorder. In some embodiments, the comorbidity is a behavioral addiction. In some embodiments, the comorbidity is a mental health disorder. In some embodiments, where one or more comorbidities is observed, the symptoms of such comorbidities are assessed at one or more points following administration of EAP. In some embodiments, comorbidities are assessed at about or at least 1 week, 2 weeks, 3 weeks, 4 weeks, 1 month, 2 months, 3 months, 4 months, 5 months, 6 months, 7 months, 8 months, 9 months, 10 months, 11 months, or at least 1 year following administration of the final dose of the entactogen as part of an EAP regimen. In some embodiments, where one or more comorbidities is observed, the symptoms of such comorbidities are reduced, e.g., in severity or frequency, following administration of EAP.

In some embodiments, prior to beginning an EAP therapy protocol, such as an MDMA-assisted therapy protocol, a patient will be enrolled in a preparation group. In such embodiments, a person is first assessed to determine whether they are eligible for treatment at screening (i.e., whether they meet all inclusion criteria, as defined herein, and do not meet any exclusion criteria, as defined herein). If so, the person may participate in the preparation group.

In some embodiments, the preparation group will span four weeks and include content such as “building confidence” which includes discussing problems substance use (such as drinking or alcohol consumption) and/or problematic and/or compulsive behaviors have caused in the patient's family, job, social life, health, etc.; why change is necessary (completing a cost benefit analysis worksheet), discussing when something went well for the patient, completing a drinking diary, where substance use and/or problematic and/or compulsive behaviors are monitored daily for one week; and completing “homework” tasks, such as a diary and a worksheet detailing the patient's strengths and weaknesses; “resources, barriers, and goal setting” which includes discussing goal setting as it relates to substance use and/or problematic and/or compulsive behaviors, reviewing potential barriers and resources that may aid in the reduction of substance use, and/or engaging in problematic and/or compulsive behaviors, such as discussing the patient's strengths and qualities, and identifying social and community support outlets; completing homework tasks, such as setting a goal related to substance use and/or problematic and/or compulsive behaviors, completing a daily diary, and completing a worksheet that details social and community support outlets; “increasing rewarding activities and building support” which includes identifying challenges that may hinder the accomplishment of goals, and discussing how those challenges were/would be managed; reviewing the social and community support worksheet, and working as a group to build upon the resources listed within; setting rewarding goals, including planning pleasant activities, etc.; completing homework tasks, which may include setting a goal related to substance use and/or a problematic and/or compulsive behavior, and beginning a pleasant activities planner; and “building the foundations for recovery” which includes discussing how the patient will manage moving forward, including identifying anticipated challenges and brainstorming ways to manage those, identifying any changes the patient would like to make in their recovery program, and identifying such changes if necessary; completing a recovery support map, and sharing/discussing it with someone who supports the patient. In other embodiments, a preparation group may contain more or less content such as the above, and/or be for a shorter or longer period of time.

In some embodiments, if a patient successfully completes the preparation group course (wherein “completes” in this context may refer to, e.g., the patient successfully reducing their substance use (such as alcohol intake) and/or frequency of engaging in a problematic and/or compulsive behavior as compared to levels (e.g., alcohol intake levels) prior to the start of the preparation group course and/or the patient actively participating in the preparation group, such that the patient readily collaborates with the other members of the preparation group and fully or substantially completes each task required in the preparation group), and the patient remains eligible for treatment (e.g., meets all required “inclusion criteria” and does not meet at least one “exclusion criteria,”) the patient may begin a treatment protocol.

In some embodiments, the disclosed methods may reduce relapse rates (wherein “relapse” refers to a resumption of substance use at a level that is above the threshold of a disorder due to substance use, as quantified by the ICD-11 or ICD-10, such as a resumption of drinking at a level that is above the threshold of “harmful use” as quantified by the ICD-10). In some embodiments, the disclosed methods may reduce relapse rates by increasing the participant's “psychological flexibility,” wherein psychological flexibility refers to a person's ability to be responsive and adaptive to situations as they arise, to shift perspectives (as opposed to being rigid or ‘stuck’) and to act mindfully in accordance with core values (rather than reacting impulsively or on ‘auto-pilot’).

In embodiments, the disclosed methods will include therapy sessions employing acceptance and commitment therapy (ACT), an enhanced form of cognitive behavioral therapy (CBT), which focuses upon an individual's relationship with their internal experiences (e.g., thoughts, emotions, physical sensations, memories) rather than the content of these experiences, and uses mindfulness as one of the cornerstones from which to explore this. The main purpose of ACT is to increase psychological flexibility via six core processes of being mindfully present, stepping back and being willing to accept all internal experiences, defusing thoughts, taking an observer's perspective, awareness of values (e.g., what's important), and acting in accordance with those values.

In some embodiments, the disclosed methods are useful in helping an individual achieve a more fulfilling and meaningful life (such as via increased psychological flexibility). A reduction in the levels of distress are typically a welcome by-product of ACT interventions (Harris, 2009). ACT can be seen as supporting and building upon the core mechanism of psychedelic-assisted therapies which enhance psychological flexibility, alongside enhancing the relapse prevention objective in supporting people to achieve a more fulfilling life without alcohol.

Relapse prevention can be an integral part of treating conditions and/or comorbidities, such as SUDs (for example, alcohol treatment) and BAs, and comorbid SUDs and BAs. This intervention typically involves supporting individuals to develop greater insight regarding the potential triggers and high risk situations for using a substance (e.g., alcohol) and/or engaging in a problematic and/or compulsive behavior, identifying early warning signs and, crucially, developing effective coping skills and enhanced self-efficacy to manage these situations (Hendershot et al., 2011). Mindfulness-based relapse prevention (Marlatt et al., 2011) incorporates core elements of mindfulness skills into this intervention. Mindfulness can be defined as “paying attention in a particular way: on purpose, in the present moment, and non-judgmentally” (Kabat-Zinn, 1994) and has been embedded across a range of therapeutic interventions including CBT for depression (Teasdale et al., 2000) and stress-reduction (Kabat-Zinn et al., 1992). In relation to relapse prevention, mindfulness-based principles can be incorporated via helping individuals to pay attention to their internal experiences (e.g., thoughts and emotions) and their potential role in relapse and practice in incorporating mindfulness practices into daily life (Bowen et al., 2009).

In some embodiments, the disclosed methods employ one or more elements of mindfulness skills, for example in mindfulness-based relapse prevention, or otherwise employ mindfulness-based principles or mindfulness practices, including for purposes of relapse prevention, or to provide or support one or more other therapeutic effects, including: (a) a reduction of substance use, (b) a reduction of substance cravings, (c) a promotion of substance use abstinence, (d) a prevention of relapse into substance use, or (e) an improvement of at least one symptom of substance use disorder. In some embodiments, a therapeutic effect may be shown by one or more of: (a) an increase in quality of life, (b) an increase in psychosocial functioning, (c) a decrease in use or frequency of a prescription medication, (d) a decrease in use or frequency of a recreational drug, (e) a decrease in obsessive compulsive thoughts, (f) a decrease in suicidality, (g) an increase in feelings of empathy, or (h) an increase in self-compassion. In some embodiments, a therapeutic effect may be shown by one or more of: a reduction in the frequency of a compulsive or problematic behavior, a reduction in drive or urge to engage in a compulsive or problematic behavior, prolonged abstinence from a compulsive or problematic behavior, a prevention of relapse into a compulsive or problematic behavior, a reduction in severity of symptoms of a compulsive or problematic behavior, a reduction in the time spent on a compulsive or problematic behavior; an increase in quality of life, including subjective sleep; and an increase in psychosocial functioning.

In some embodiments, for example where the SUD is AUD, the disclosed methods employ one or more elements of mindfulness skills, for example in mindfulness-based relapse prevention, or employ mindfulness-based principles or mindfulness practices, including for purposes of relapse prevention, or to provide or support one or more other therapeutic effects, including: (a) reducing alcohol use, (b) reducing alcohol cravings, (c) promoting alcohol abstinence, (d) preventing relapse into alcohol use, or (e) improving at least one symptom of alcohol use disorder. In some embodiments, the one or more elements of mindfulness skills, or employment of mindfulness-based principles or mindfulness practices will provide or support one or more of: (a) increasing quality of life, (b) increasing psychosocial functioning, (c) decreasing use or frequency of a prescription medication, (d) decreasing use or frequency of a recreational drug, (e) decreasing obsessive compulsive thoughts, (f) decreasing suicidality, (g) increasing feelings of empathy, (h) increasing self-compassion, or (i) improving at least one symptom of a comorbid psychiatric disorder.

In some embodiments, the disclosed methods employ motivational interviewing (MI), a technique used to enhance motivation to change behavior that is widely used in addiction services. Miller & Rollnick (1991) describe a key element of this process as coming alongside the individual to explore ambivalence and elicit the person's own ideas about change. MI is characterized by therapists' unconditional acceptance of where the individual finds themselves and their perspective. In MI, a therapist's role is to work in partnership and collaboration (not to act as the expert, to lecture or argue), to be genuinely curious and ask questions that provide opportunities for the individual to hear themselves talk about change and to have compassion for the individual (to prioritize their needs and an authentic regard for their well-being).

### Example 3: Exemplary Treatment Protocols-“Harmful Use” AUD

Purpose: To assess the effects of EAP in treating a patient having an SUD, or an SUD and one or more comorbidities. For exemplary purposes, the SUD of this Example is harmful use AUD, and the exemplary entactogen is MDMA, neither of which should be construed as limiting, and an Example directed to dependent use AUD is described below; further examples directed to BAs and other SUDs will be understood in view of the teachings herein. Comorbidities, where present, may be any one or more of an additional SUD, a BA, or a mental health disorder. Additionally, whether a patient's comorbidities have improved as a result of entactogen-assisted therapy is addressed.

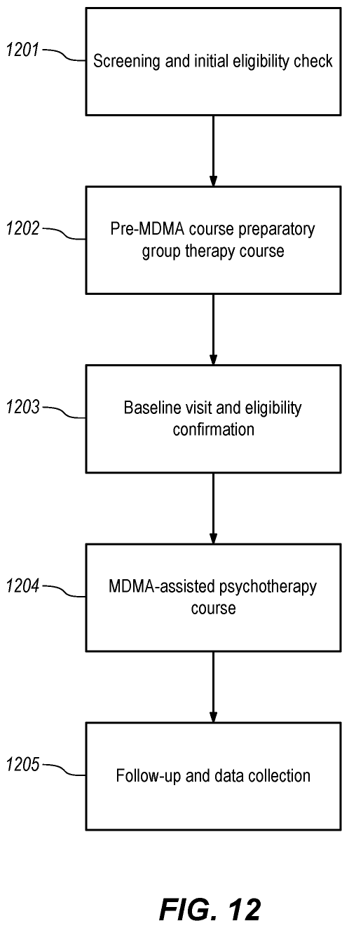

Methods: In certain exemplary treatment methods wherein the disclosed compositions are shown to be useful in methods for treating harmful use (non-physically dependent) AUD, the treatment flow can be conceived in phases as follows, and are specifically outlined in FIG. 12: screening and initial eligibility check (1201); pre-MDMA course preparatory group therapy course, e.g., a four-week course of weekly sessions (1202); Baseline visit and eligibility confirmation (1203); an MDMA-assisted psychotherapy course, e.g., over eight or 13 weeks (1204); follow-up and data collection, e.g., at 3 months, 6 months, and 9 months (1205).

It will be readily appreciated, however, that this exemplary treatment flow is merely illustrative of one use of the pharmaceutical disclosed compositions in a method of treating AUD, that a use need not include every illustrated aspect (e.g., the follow-up or data collection of step six), or be in the exact order below, and moreover, that the steps, stages, or phases below (and their delineation as such) are simply for purposes of assisting the reader with the conceptualization of a use of the invention, whereas the full breadth and scope of the invention shall be known by reference to the appended claims.

TABLE 4
Exemplary Treatment Flow-“Harmful Use” AUD

1. Screening and Eligibility Check
2. Pre-MDMA Course Preparatory Group Therapy Course
3. Baseline Assessment Visit and Eligibility Confirmation
4. MDMA-assisted therapy Course
5. Follow-Up and Data Collection

Screening and Initial Eligibility Check (101)

Patients who meet the diagnostic criteria for AUD, but are not physically dependent, meaning detoxification is not required, will be screened prior to entering the pre-MDMA course group therapy alcohol reduction program. Eligible patients will attend a baseline visit after the pre-course group program to ensure eligibility.

Potential patients will be invited to a screening visit where their eligibility for MDMA-assisted therapy using the disclosed methods will be determined. The screening visit will include informed consent, medical, psychiatric and substance use history, prescribed medication use, basic physiological observations and alcohol breath test, urine drugs, and pregnancy screen as clinically indicated. In order to monitor the risk of patients using MDMA outside of the therapeutic context, they are asked about their use of, or desire to use, illicit ecstasy, e.g., with standard questions such as those used by MAPS in MDMA-assisted therapy studies.

During screening the therapeutic process will be discussed and explained. The patients' understanding of the procedure, requirements, and commitments will be confirmed and any questions answered before informed consent is obtained. If video and/or audio recordings will be made during therapy sessions, patients will be asked to consent. If patients do not wish to have sessions recorded (or at any point decide to withdraw consent for the whole or part of the session) recordings will be stopped and deleted if requested. Potential patients also may be contacted and partake in a short telephone screen before invitation to a full screening visit, to minimize inconvenience to “obviously” ineligible patients.

In some embodiments, a psychiatric assessment is sought. In such embodiments, it will be performed by a trained psychiatrist. Assessments may include, e.g.: (1) the Mini International Neuropsychiatric Interview 5 (MINI 5) (Sheehan et al. 1998) to screen for comorbid psychiatric disorders; (2) diagnostic evaluation of the presence of Alcohol Use Disorder according to DSM-5 (assessed using SCID-5-CT (Clinical Trials Version) (First et al. 2015)); (3) diagnostic evaluation of the presence of AUD in the absence of physical dependence, as guided by the SADQ; and (4) clinical risk assessment for suicidality using the Columbia Suicide Severity Rating Scale (C-SSRS) (Mundt 2013). Clinical data collection may include years of alcohol use and any dependence, complications/consequences, and previous therapeutic inputs provided including any previous in-patient or community alcohol detox programs. Other questionnaires also may be completed.

In some embodiments, information about alcohol consumption for the three months previous to the screening visit is assessed using the Alcohol Timeline Follow Back (TLFB) method, the gold-standard in self-report substance use, which utilizes a calendar method and memory aids to collect retrospective estimates of daily drinking to obtain accurate daily estimates of the quantity of alcohol consumption (Sobell 2001). Patients may receive the same calendar to take away to track their drinking behavior for the duration of the therapy.

In some embodiments, another SUD besides AUD is treated in the EAP regime.

In some embodiments, a behavioral addiction is treated in the EAP regime.

In some embodiments, another entactogen besides MDMA is used in the EAP regime.

Pre-MDMA Course Preparatory Group Therapy Course (102)

In some embodiments, after screening, participants will attend weekly sessions of group therapy, which will be run by a trained professional with expertise in addiction treatment. In some embodiments, participants will attend weekly sessions of group therapy for four weeks, although shorter or longer courses may also be used, and in some embodiments the preparatory group therapy course may be skipped entirely. The purpose of the group will be to provide psychosocial support to assist individuals to gradually reduce their alcohol intake prior to starting the course of MDMA-assisted therapy.

A reduction in daily or weekly alcohol consumption should form part of a structured, individualized care plan involving careful preparation (NICE Guidelines). Adequate preparation also benefits from consideration of a person's support network, motivation and after-care planning to improve post-treatment outcomes (Kouimtsidis, 2017). Prior to being considered ready for the MDMA-assisted course, patients thus may attend weekly sessions of group therapy. In some exemplary embodiments, groups of between 5 and 10 participants can be facilitated by one or two trained staff with experience in delivering motivational interview style alcohol treatment group counseling. However, as would be apparent to one of skill, the number of participants and instructors may vary and, in some embodiments, the number of participants may be greater than 10, including at least 12, at least 14, at least 16, at least 18, and at least 20. Likewise, the number of participants may be less than 5, including 4, 3, 2, or 1. The number of trained staff with experience in delivering motivational interview-style alcohol treatment group counseling may, in some embodiments, be greater than 2, including 3, 4, 5, 6, 7, 8, 9, 10, or more than 10.

In some embodiments, rather than participating in an in-person group therapy course, participants attend a synchronous virtual group therapy course (i.e., participants attend all sessions “live,” but remote, e.g., via computer). In some embodiments, participants attend an asynchronous virtual group therapy course (i.e., attend at selected times of their choosing, where less than all participants may attend at the same time), and wherein one or more portions of the course will be pre-recorded. Participants attending a detoxification course (as discussed further below) may also attend a synchronous or asynchronous virtual course.

In some embodiments, each weekly group therapy session will last for between about 30 minutes to about 120 minutes. In some embodiments, weekly group therapy sessions will last for about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, and values in between. In some embodiments, weekly group therapy sessions may last for greater than 120 minutes, or may take place in a residential (or “rehab”) facility. In some embodiments, groups are closed and confidential. In some embodiments, patients are enrolled into the groups on a rolling basis and will leave after a minimum (e.g., of four) weeks attendance to be enrolled into the MDMA-assisted therapy course when deemed appropriate by the facilitators.

In some embodiments, if a patient is not deemed sufficiently engaged in the preparation phase (e.g., as deemed by the group facilitators) and/or has not managed to reduce their use of alcohol to zero, their enrollment into the MDMA-assisted therapy course can be delayed until they are considered to be adequately prepared or otherwise ready to progress. In some embodiments, wherein further preparation is required, it may include attending further group sessions which, in some embodiments, requires repeating the entire course while, in other embodiments, merely repeating one or more weeks of the course.

Baseline Assessment Visit and Eligibility Confirmation (103)

After a participant has successfully completed the pre-treatment preparation group therapy course, the participant will be assessed for eligibility to start the DMA-assisted therapy course. This is the “baseline” point. During this visit, continued consent is confirmed and vital signs reassessed (pulse, blood pressure, etc.). A brief physical examination and ECG is performed, and blood samples for routine laboratory tests (urea and electrolytes, full blood count, liver function tests and Gamma-GT) are also completed. A blood test is not required if the patient has had one in, e.g., the last three months.

MDMA-Assisted Therapy Course—Two MDMA Sessions

In an exemplary embodiment of a MDMA-assisted therapy treatment regimen (an “MDMA-assisted therapy course” or “MDMA-assisted therapy regimen”), a patient will receive ten total therapy sessions during the treatment phase: eight 60-minute psychotherapy sessions, and two 6-8 hour MDMA-assisted therapy sessions. Sessions will take place approximately 1-2 weeks apart and start as soon as possible after baseline.

In some embodiments, another SUD besides AUD is treated in the EAP regime.

In some embodiments, a behavioral addiction is treated in the EAP regime.

In some embodiments, another entactogen besides MDMA is used in the EAP regime.

TABLE 5
Exemplary Eight-Week MDMA-assisted therapy Course

| Week | Session | Activity |
|---|---|---|
| 1 | 1 | Non-Drug Prep Session (60-minute therapy session) |
| 2 | 2 | Non-Drug Prep Session (60-minute therapy session) |
| 3 | 3 | MDMA-Assisted Therapy Session 1 (~6-8 hours) |
| | 4 | Next-Day Non-Drug Integration/Follow-up Session (60 mins) |
| 4 | 5 | Non-Drug Integration Session (60-minute therapy session) |
| 5 | 6 | Non-Drug Integration Session (60-minute therapy session) |
| 6 | 7 | MDMA-Assisted Therapy Session 2 (~6-8 hours) |
| | 8 | Next-Day Non-Drug Integration/Follow-up Session (60 mins) |
| 7 | 9 | Non-Drug Integration Session (60-minute therapy session) |
| 8 | 10 | Non-Drug Integration & Final Evaluation Session (~90 mins) |

MDMA-assisted therapy sessions may be run by a therapist pair, e.g., one male and one female, although they also may be run by two male or two female therapists, one male and one non-binary therapist, one female and one non-binary therapist, two nonbinary therapists, a single therapist, or more than two therapists. In some embodiments, psychological support during each MDMA session preferably will be provided by two members comprising a “therapy team.” Where “therapy team” is used herein, it will be appreciated that modifications can be made to accommodate more than two therapists, such as 3, 4, or 5 therapists; or use a single therapist, and in such embodiments “team” will refer to that therapist alone (or where appropriate in context, to that therapist and another therapist or clinician not necessarily present with the patient during an MDMA-assisted or other therapy session, but nevertheless a member of the patient's overall care team). A therapy team may comprise a psychiatrist and a co-therapist who is a clinical psychologist or psychotherapist, but either member may be a “therapist” as the term is defined above.

A dyadic male-female pair is used in some embodiments herein. A dyadic male-female pair is a feature of certain MDMA-assisted therapy (Greer and Tolbert 1998); however, the co-therapist pair herein need not necessarily be male-female; same-sex pairs, and pairs comprising at least one member not identifying as a male or a female are also appropriate. Where a dyadic therapy pair is used, in some embodiments, a patient will get to know both therapists in the preparatory sessions before their first MDMA session. In embodiments, a facilitative relationship is thereby developed, which will aim to reduce discomfort or difficulties that might arise during the course of therapy.

Guidelines for each therapeutic session in exemplary embodiments follow. In some embodiments, the patient will drive the psychotherapy sessions according to the patient's individual needs. In some embodiments, the therapists guide the psychotherapy sessions. In some embodiments, the therapeutic approach may be guided in part with reference to the Manual for MDMA-assisted therapy in the Treatment of PTSD (Mithoefer, 2017).

In some embodiments, at the start of all psychotherapy sessions, patients are asked about alcohol use and complete an alcohol breath test to confirm sobriety during therapy. On all MDMA-assisted therapy sessions, an alcohol breath test and urinalysis (dipstick) for recreational drugs and pregnancy (if applicable) may be performed. A positive result for recreational drugs other than cannabis (THC) on a test would terminate the session.

What now follows is an exemplary session flow, as may be used in some embodiments of the invention; changes, modifications, and additions may be made according to the teachings herein and the general knowledge in the art, and will be appreciated.

Sessions 1 and 2: Therapists will discuss MDMA, how it works, the expected effects, risks and review of the historical evidence of MDMA as a tool for psychotherapy. In some embodiments, the patient will be invited to share aspects of their life history that they feel important for the therapists to know, given that, without being bound by theory, thoughts, feelings, images and memories about past experiences may come to the patient's mind during the MDMA session. Discussion, in some embodiments, will also include questions about the patient's internal experiences (e.g., thoughts, emotions, imagery) and their understanding of this. In such embodiments, the patient will be invited to discuss their current coping responses and any goals/hopes for participating in the study, alongside any intentions for what they would like to explore during the MDMA session. That said, such embodiments are merely exemplary, and should not be construed as limiting the invention as disclosed herein. Necessarily, in some embodiments, other such questions may be asked by one or more therapists to assess, among other things, the background, needs, and goals of the patient and, likewise, the structure of the psychotherapy session may be modified in response to the way in which the patient is responding.

Session 3 (MDMA-assisted session): Patients will arrive at an agreed time which, in some embodiments, is in the morning—for instance between approximately 8-9 am. However, the scheduled time need not be in the morning, and may instead be in the afternoon, the evening, or late at night. Regardless, continued consent will be registered and eligibility may be checked, including assessment of physical health, alcohol breath test and urine drugs screen for recreational drugs and pregnancy (if applicable). Vital sign measurements such as heart rate, blood pressure, and temperature may be taken at baseline before the MDMA is administered and before discharge (or more frequently if clinically indicated, including by use of medical sensors or biosensors that provide continuous, near-continuous, or periodic measurements). Consent to video and audio record the session may be obtained or confirmed; if it is withdrawn, recording should not take place (or only audio recording should take place, if such specific and limited consent is obtained).

In some embodiments, patients will receive an initial dose of about 125 mg MDMA (e.g., two capsules or tablets of 62.5 mg each), as in the formulations of Examples 1 and 2 or the equivalent. In other embodiments, patients may receive an initial dose of another amount, e.g., 100 mg or 80 mg. Patients will be invited to relax for about 90 minutes, lying down with eyeshades on if happy to do so. The patient may have the option to listen to music, either through headphones or into the room, and may continue to do so throughout the therapy session. During this initial phase of the effects of MDMA most patients prefer to have little communication but rather to acclimatize to the psychological and physical effects of the drug.

After 90 minutes the patient may be invited to sit and the facilitators may gently guide the patient into discussion of their particular individual aspects of their history.

At an appropriate time after receiving the initial dose of MDMA the patient may be given the option to receive a further supplemental dose of, in some embodiments, half the original dose (i.e., 62.5 mg MDMA, in this example; or, e.g., 50 mg or 40 mg). In an embodiment, the time of administration of the supplemental dose will be two hours following the administration of the initial dose. In embodiments, the time of administration of the booster dose will be determined so as to extend peak subjective effects. In embodiments, the time of administration of the booster dose will be determined so as to optimize the plasma concentration-time profile, e.g., by prolonging the time at higher plasma concentrations. The purpose of this is to prolong the psychological and direct pharmacological effects of the drug to allow for further time for psychotherapy, and thus such prolongation can be optimized or determined either by reference to the subjective (psychological) effects, or the pharmacokinetic (pharmacological) effects, or with other known methods in the art.

Generally, patients will not be encouraged to get up and move around during the session, but rather to remain sitting or lying throughout. However, if a patient wishes to get up, this can be facilitated if the therapy team judges it safe to do so. In some embodiments, physical touch from the therapists toward the patient is employed as a therapeutic tool. In such embodiments, this technique can be used as a supportive grounding technique and to allow some patients to externalize their psychological pain. This technique should be carefully managed with professional boundaries by the therapists and prior informed consent.

The patient may be required to remain in the facility until they are deemed fit to return home. They may, e.g., be assessed by the attendant doctor and signed off as medically fit for discharge. If not deemed ready to leave they may be required to remain in the therapy facility until deemed medically fit for discharge. The patient may then be met at the facility by their pre-arranged significant other, who can accompany them home. Generally, for safety reasons, a patient should not be permitted to leave on their own.

Session 4: The day following session 3, the patient may undertake a follow-up session with the therapy team. This generally will involve a 60-minute face-to-face follow-up session discussing aspects of MDMA-assisted therapy undertaken the day before. Patients will be given the contact details of a clinical member of the therapy team whom they can contact if required for further telephone support during the week after each MDMA session. In some embodiments, patients will receive one or more remote or virtual follow-ups after session 4, for example daily phone calls or using another follow-up means as described further below.

Sessions 5 and 6: The patient may have the opportunity to reflect upon the MDMA session and to explore the material that emerged. There are opportunities to reflect on new insights or perspectives gained and how this may impact on the issues unpinning their addictive behaviors and/or other areas of difficulty. Intentions for the next MDMA session may be discussed.

Session 7 (MDMA-assisted session): Same as session 3 above; outlined in TABLE 5.

Session 8: Same as session 4 above; outlined in TABLE 5.

Session 9 and 10: Same as sessions 5 and 6 above, as outlined in TABLE 5, wherein the patient has the opportunity to reflect upon the MDMA session and to explore the material that emerged, and to reflect on new insights or perspectives gained/how this may impact on the issues unpinning their addictive behaviors and/or other areas of difficulty, and discuss any intentions for the next MDMA session. In addition, patients may be invited to consider any meaningful goals and/or values that they would like to focus on and move toward beyond the treatment phase. In session 10, the final session in this exemplary embodiment, the patient may complete outcome measures after the therapy session has ended. For example, this may include the Trauma History Questionnaire (THQ) (Green 1996) with the therapist. The THQ will give the therapy team information about the patient's past history of trauma. The visit including the therapy session will take approx. 1.5 hours. In this embodiment, after a patient finishes the tenth visit, treatment is either considered complete, or the patient progresses to the follow-up phase, further described below.

MDMA-Assisted Therapy Course-Three MDMA Sessions

In another exemplary embodiment of a MDMA-assisted therapy treatment regime (also, an “MDMA-assisted therapy course” or an “MDMA-assisted therapy regimen”), a patient receives 16 total therapy sessions during the treatment phase: 13 60-minute psychotherapy sessions, and three 6-8 hour MDMA-assisted therapy sessions. Sessions will take place approximately 1-2 weeks apart and start as soon as possible after baseline (generally, within approximately one to three weeks).

In some embodiments, another SUD besides AUD is treated in the EAP regime.

In some embodiments, a behavioral addiction is treated in the EAP regime.

In some embodiments, another entactogen besides MDMA is used in the LAP regime.

TABLE 6
Exemplary Thirteen-Week MDMA-assisted therapy Course

| Week | Session | Activity |
|---|---|---|
| 1 | 1 | Non-Drug Prep Session (60-minute therapy session) |
| 2 | 2 | Non-Drug Prep Session (60-minute therapy session) |
| 3 | 3 | MDMA-Assisted Therapy Session 1 (~6-8 hours) |
| | 4 | Next-Day Non-Drug Integration/Follow-up Session (60 mins) |
| 4 | 5 | Non-Drug Integration Session (60-minute therapy session) |
| 5 | 6 | Non-Drug Integration Session (60-minute therapy session) |
| 6 | 7 | MDMA-Assisted Therapy Session 2 (~6-8 hours) |
| | 8 | Next-Day Non-Drug Integration/Follow-up Session (60 mins) |
| 7 | 9 | Non-Drug Integration Session (60-minute therapy session) |
| 8 | 10 | Non-Drug Integration Session (60-minute therapy session) |
| 9 | 11 | Non-Drug Integration Session (60-minute therapy session) |
| 10 | 12 | Non-Drug Integration Session (60-minute therapy session) |
| 11 | 13 | MDMA-Assisted Therapy Session 3 (~6-8 hours) |
| | 14 | Next-Day Non-Drug Integration/Follow-up Session (60 mins) |
| 12 | 15 | Non-Drug Integration Session (60-minute therapy session) |
| 13 | 16 | Non-Drug Integration & Final Evaluation Session (~90 mins) |

Phases one through four preceding this 13-week MDMA-assisted therapy course generally are the same as those outlined in the 8-week course of TABLE 5. Also as above, the MDMA-assisted therapy sessions may be run by a therapy team, wherein the team comprises a male and a female pair, or in alternative embodiments, two males, two females, or one or two individuals not identifying with either male or female. In addition, the protocols for each therapeutic session are as generally described above, with modifications such as those in response to, or accommodation of, the overall longer course of treatment and the additional MDMA session, as appreciated by those of skill from the teachings herein and the general knowledge in the art.

Sessions 1-9: Generally, as described above for the eight-week course of TABLE 5.

Sessions 10-12: Generally, as sessions 5 and 6 of the eight-week course of TABLE 5.

Sessions 13-14: Generally, as sessions 3 and 4, and 7 and 8 of the eight-week course outlined in TABLE 5.

Sessions 15 and 16: Generally, the same as sessions 9 and 10 in the eight-week course outlined in TABLE 5. Patients may continue to consider any meaningful goals and/or values that they would like to focus on and move toward beyond the treatment phase. In session 16, the final session in this exemplary embodiment, the patient may complete outcome measures after the therapy session has ended, such as the Trauma History Questionnaire (THQ). The visit including the therapy session will take approximately 1.5 hours. In this embodiment, after patients finish the sixteenth visit, treatment is either considered complete, or the patient progresses to the follow-up phase below. In alternative embodiments, one or more of sessions 10-12, for example, are eliminated or made optional, as may be one or more other sessions, or one or more sessions in the eight week treatment protocol above (e.g., session 6 or 9).

Follow-Up and Data Collection

“Follow-up” phase refers to the period following the last session of the MDMA-assisted therapy course (e.g., session 10 or session 16 above) or, if a patient discontinued treatment, from two weeks after the final psychotherapy session to the final follow-up visit. In an exemplary embodiment of the treatment protocol, outcomes will be assessed at 3 months, 6 months, and 9 months after the end of baseline or the initial detoxification. In such embodiments, the final follow-up visit is thus at 9 months post baseline or detoxification completion. In some embodiments, a final follow-up visit may be 12 months or longer post baseline or detoxification. In addition, outcomes may be assessed at other incremental timeframes, the frequency and amount of which depend on the specific needs of each participant, as would be apparent to those of skill.

Means of performing follow-up visits and gathering follow-up data (e.g., to determine therapeutic efficacy and measure therapeutic effect) include in-person visits, telemedicine and other virtual visits, phone calls, automated inquiries and check-ins (e.g., email questionnaires, mobile apps, etc.), and the like. Accordingly, it will be appreciated that a “visit” need not be an in-person or face-to-face visit with a member of the therapy team, or another clinician.

Audit data from clinical notes indicates that formal assessment data for patients seeking treatment for AUD (including for those who undergo community detoxification) is typically sparse and difficult to collect. Accordingly, in preferred embodiments herein, means are provided to not only collect high quality data about patient outcomes, but to do so in a way that increases compliance and successful data collection. Such means will ease the burdens of collecting follow-up data (to patients, caregivers, and others), minimize attrition rates (previously shown to be in the range of 10-35% by Hallgren K A, Witkiewitz 2013), and avoid the pitfalls of using statistical analysis to predict outcomes for those lost to follow-up by collecting good quality long term outcome data in these studies from the outset, e.g., by advantageously enhancing contact with patients or by using optimized means.

For example, when in person visits are used, patients will be invited to attend visits (estimated duration, 1.5 hours) at 3, 6, and 9 months post baseline or detox. All patients will receive a reminder before the visit. At such follow-up visits, patients will be asked to complete a urine test for recreational drugs, breathalyzer test for alcohol and complete questionnaires, or other appropriate follow-up assessment. Such visits may also be conducted at other incremental timeframes, the frequency and amount of which depend on the specific needs of each participant, as would be apparent to those of skill.

Follow-up telephone calls (estimated duration: 5-10 mins) also can be utilized to gather follow-up data to determine therapeutic efficacy and measure therapeutic effect. Systematic efforts will be made to contact patients to collect follow-up data; however, because some patients may be difficult to contact, patients can be asked (as an eligibility criterion) to identify a significant other who can be contacted in the event that the therapy team is unable to contact the patient to collect outcome data. When a patient is unreachable, and if the patient has consented, a significant other also may be asked to give information about outcome (drinking behavior etc.).

### Example 4: Open Label within-Subject Safety and Tolerability Feasibility Study of MDMA-Assisted Therapy in Patients with “Harmful Use” AUD

Purpose: A study to investigate the use of MDMA to treat AUD in patients without physical dependence on alcohol (“harmful use”) is described in this exemplary treatment protocol. Patients will not require a community alcohol detoxification or similar course before their MDMA-assisted psychotherapy course. Similar open label studies will be appreciated for other SUDs, and for behavioral addictions, based on the teachings herein and the knowledge in the art.

Methods: In this study, patients receive two sessions with MDMA (e.g., 187.5 mg each session, as two separate doses totaling 125 mg and then one of 62.5 mg). However, it should be noted, the dosage amount is purely exemplary in nature, and may be modified according to the specific outcome desired, and would be within the knowledge of one of skill. Psychological support will be provided before, during, and after each session. Safety and tolerability will be assessed alongside psychological and physiological outcome measures. Alcohol use behavior, mental well-being, and functioning data will be collected for nine months after baseline.

This study will demonstrate that MDMA treatment is well-tolerated by all participants. No unexpected adverse events will be observed. Psychosocial functioning will improve across the cohort. Regarding alcohol use, at nine months post baseline the average units of alcohol consumption by participants will compare favorably to other observational studies with similar populations of people with AUD. Such results, in view of the further description and disclosures provided herein, additionally demonstrate the therapeutic potential of MDMA-assisted psychotherapy for a population of people with non-physically dependent (“harmful use”) AUD (i.e., not requiring or undergoing detox).

In other embodiments, an entactogen other than MDMA will be used.

More specifically, the study of this Example is an open label within-subject safety and tolerability feasibility study, in patients with AUD. All patients will receive MDMA-assisted therapy. The main outcome measures will be the number of patients completing the eight week psychotherapy course, the number accepting the second booster dose of MDMA on drug assisted days and adverse events. Secondary outcome measures will include changes in drinking behavior (measured by “units per week consumed,” wherein a standard drink contains about 14 g of pure alcohol (ethanol), at 3 months, 6 months, and 9 months since baseline), measures of mental well-being, psycho-social functioning, quality of life and concomitant drug use.

Patients with a diagnosis of alcohol use disorder, who do not have physical dependence on alcohol, will be recruited. These patients may be recruited from both NHS and partner organizations, from primary care, by word of mouth or by answering adverts placed in appropriate locations. The sample size will be based on a power calculation of required group size to adequately test the hypothesis.

After completing a group therapy preparation course, patients receive an 8 week course of abstinence-based therapy comprising 10 psychotherapy sessions (such as described in further detail above). On two of these sessions (session 3 and 7) patients will be dosed with open-label MDMA during a 6-8 hour assisted therapy session. The eight week therapeutic course will start as soon as possible after a patient has completed the preparation course (with a delay up to two weeks).

In an alternative aspect, reflecting an alternative embodiment such as described above, patients will receive 16 therapy sessions, comprising 13 60-minute non-drug psychotherapy sessions and three, 6-8 hour MDMA-assisted therapy sessions. MDMA-assisted sessions will take place between three and five weeks apart (the interval between drug sessions 1 and 2 is three weeks, and the interval between drug sessions 2 and 3 is five weeks). This 13 week therapeutic course also will start as soon as possible after a patient has completed the four week group preparation course (with a delay up to two weeks).

Good Manufacturing Practice (GMP) MDMA (i.e., substantially pure racemic MDMA HCl) will be obtained from Sterling Pharmaceuticals (Newcastle) and formulated into the investigational medicinal product (62.5 mg MDMA in gelatin capsules) by Guy's and St Thomas' Hospital's Pharmacy Manufacturing Unit (London, UK). At the time of this filing, MDMA remains a scheduled drug substance; however, the clinic holds a schedule one license for Home Office approved storage, prescribing, and dispensing of MDMA. It will be within the knowledge of one of skill as to how to obtain such a license, if it is still required.

On each dosing session, patients will receive an initial oral dose of 125 mg MDMA (i.e., two capsules), followed two-hours later by an optional booster dose of 62.5 mg (i.e., one capsule). In some embodiments, the booster dose will serve to prolong the experience, allowing for greater time for psychotherapy under the influence of MDMA. In some embodiments, a patient may only receive a single initial dose of MDMA; in other embodiments, a patient may receive two doses of MDMA, the first being an initial dose, and the second being a booster dose.

Other sessions (i.e., sessions 1, 2, 4, 5, 6, and 8-10) will comprise one hour psychotherapy sessions, and employ aspects of Motivational Interviewing and “third wave” cognitive-behavioral approaches, for example, modified cognitive-behavioral approaches, such as Acceptance and Commitment Therapy. Patients will remain in the study for a duration of approximately 10 months.

Trial Procedures: AUD will be identified using the ICD-10 SCID questionnaire. Screening will comprise of written informed consent, an evaluation of the patient's physical and mental health background, a psychiatric interview (MINI), assessments of depression and anxiety severity will be assessed using the PHQ-9 and GAD-7 questionnaires. Severity of AUD will be established using the SADQ and SIP questionnaires. Patients will receive a thorough physical health check comprising an electrocardiogram (ECG), routine blood tests, blood pressure, heart rate, and physical examination. Following screening, eligible patients will first receive a four-week course of supportive group therapy (motivational group meetings) in order to gradually reduce their intake of alcohol. In order to confirm the patient has no symptoms of physical alcohol withdrawal, the Clinical Institute Withdrawal Assessment for Alcohol—Revised Version (CIWA-Ar) (Saitz et al 1994) will be carried out, as well as an alcohol breath test. Dip-stick urinalysis for recreational drugs can be completed at the research team's discretion. If female, dip-stick urinalysis for pregnancy will be completed. Patients are asked to complete post-preparation questionnaires.

Subsequently, eligible patients enter the eight week (or 13 week) course of psychotherapy. This will entail weekly 60-minute outpatient non-drug psychotherapy sessions, delivered by two clinicians trained in delivering MDMA-assisted therapy.

Dosing with MDMA will occur twice during the eight week course, during weeks 3 and 6. Physiological changes, observer and subject ratings of distress (SUDS) and the intensity of the acute psychoactive effects of MDMA will be measured throughout the MDMA-assisted session. Acute anxiety will be managed primarily psychologically, but sedative medication (oral lorazepam) will be available. Patients will optionally remain overnight in the treatment center after each MDMA-assisted session, overseen by medically-trained “night sitters,” on hand to support patients as required but instructed to avoid delivering any psychotherapeutic interventions. Patients will be seen the morning after each MDMA-assisted session for an integration psychotherapy session, and then telephoned daily for six days to assess changes to mood, suicidal risk factors (using C-SSRS) and quality of sleep (using the Leeds Sleep questionnaire). Following the end of the eight week (or alternatively, 13 week) therapeutic course participants will carry out additional follow-up questionnaires. They will then be seen again at 3, 6, and 9 months (since baseline) for longer-term follow-up data collection.

Data Analysis: All data will be recorded on paper case report forms (CRFs) and then digitized into MS Excel spreadsheets. Analysis and graphing will be performed using GraphPad (Prism) or MS Excel. When calculating Timeline Follow Back results, alcohol consumption levels at last observation will be used in the case of drop-outs or when participants undergo the group therapy preparation course more than once (Hamer and Simpson, 2009).

Results: Severity of AUD criteria at screening and baseline: as per the inclusion criteria, all eligible patients will score above the diagnostic threshold on the DSM-5 SCID questionnaire for AUD. AUD severity will also be measured using the SIP questionnaire and the SADQ questionnaire, and no patients found to be physically dependent on alcohol will be included.

Physiological and tolerability effects during MDMA sessions: Drug-assisted psychotherapy sessions with MDMA will be administered during the trial to the patients. Temperature, BP, and HR are measured at t=0, before taking MDMA, then half-hourly up to t=2 hours, then hourly thereafter for a minimum of six hours from the time of dosing.

All of these physiological parameters are expected to remain within normal limits for all these sessions. A mild, transient rise in BP, temperature and HR over the course of the MDMA session may be seen. No patients will experience sustained abnormal physiological disturbance, symptomatic experiences of raised blood pressure, heart rate or temperature or any other adverse events during MDMA sessions. No medical interventions will be required in respect of these or any other physiological events during MDMA sessions.

Subjective Units of Distress (SUDS) and Participant report of “Drug Effects” (as used in, e.g., Sessa et al. 2021, and known in the art) are measured hourly throughout the MDMA sessions. Most subjects are predicted to report mildly raised SUDS scores at the beginning of the sessions before taking MDMA-consistent with expected anxiety ahead of dosing-which subsequently is predicted to be reduced during the course of the session as the positive effects of MDMA emerge. Participants will give their own subjective score (0-10) of whether they felt Drug Effects, and the therapists also will record their own objective score of how “altered” the participant appeared. There will be no significant difference between Observers' and Participants' Drug Effects scores. Drug Effects will rise expectedly over the first two hours, with a further increase after the booster dose is given at t=2 hours, and a subsequent plateau then decline over the following six hours. By the end of the MDMA session day all drug effects will have returned to baseline.

Changes in drinking behavior: Data will be collected in respect of Units of Alcohol Consumed per Week (wherein a standard drink contains about 14 g of pure alcohol, i.e., ethanol) in the month before participants' baseline, immediately after baseline, throughout the eight week MDMA therapy course and for 3 months, 6 months, 9 months, one year, and over one year after baseline (including over 15 months, over 18 months, and over two years), and patients are expected to reduce their drinking behavior in significant amounts, and in amounts that significantly or markedly greater than any known or prior art treatment.

Comparisons will be made for the prevalence of average number of units consumed per week between each respective time point of 3, 6, and 9 months following the final therapy session. Appropriate statistical analyses will be executed on the resulting figures via Pearsons correlation coefficients to identify the associated change between each point of TLFB data collection and baseline. The differences will then be assessed via Fisher r-z transformation (Lowry, 2001-2003) to test the hypothesis that MDMA-assisted therapy compared to placebo-assisted psychotherapy is effective in this population of participants with AUD.

Seven-day follow-up after MDMA sessions: Considerable medical and popular press literature reports anecdotal observation of ecstasy users experiencing an acute “come down” effect and a drop in mood in the days after using the drug recreationally. In order to measure this prospectively with clinical MDMA, participants' mood states are measured by daily Profile Of Mood States (POMS) measurements for seven days after each MDMA session. Positive scores represent depressed affect, zero represents no change in mood or affect, and results below zero represent a positively felt mood. Average scores across both MDMA sessions for all participants will reveal no evidence of any mood disturbance during the week after each session of taking clinical MDMA. Participants instead will sustain a positive mood for seven days, contrasting with anecdotal reports from recreational ecstasy users.

Assessing Comorbid SUDs: Disclosed treatment methods for exemplary SUD harmful use AUD, in some embodiments, will additionally be effective in treating one or more comorbid substance use disorders (SUDs). In such embodiments, the exemplary treatment methods will result in one or more of a reduction of substance use, a reduction of substance cravings, the promotion of abstinence from a substance, and improvement or a reduction in at least one symptom of an SUD. The substance use disorder may be any one or more of opioid use disorder, nicotine dependence and tobacco use disorder, sedative, hypnotic, and anxiolytic use disorder; and stimulant use disorder, including others known to one of skill in the art.

A reduction in substance use or abstinence therefrom may be determined according to observer- or subject-reported methods. In some examples, a reduction in substance use may be determined by evaluating biological specimens, e.g., saliva, blood, and urine, to determine the level of a marker of substance use. In other examples, a subject may self-report the frequency of substance use, including a reduction thereof, using a questionnaire or survey, e.g., the Addiction Severity Index (ASI). In some cases, these approaches may be combined to determine a reduction in substance use of abstinence from the same. Improvement of at least one symptom of an SUD, or a reduction in the severity of at least one diagnostic criterion may be determined by consulting available diagnostic criteria, e.g., the DSM-5 and other applicable measures.

Assessing Comorbid BAs: The provided methods, in some embodiments, will additionally be effective in treating one or more comorbid behavioral addictions (BAs). In such embodiments, the exemplary treatment methods will result in any of a reduction of the behavior, abstinence from the behavior, and prevention of relapse into the behavior. Methods of determining a reduction in behavior, relapse, and abstinence are known to one of skill in the art, and exemplary methods are described herein. A behavioral addiction may be any of gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction, as well as others known to those of skill.

Assessing Comorbid Mental Health Conditions: The provided exemplary treatment methods will additionally be effective to treat one or more comorbid mental health disorders (used alternatively with “mental health conditions”). In embodiments, the exemplary treatment methods will result in an improvement, e.g., ameliorating or preventing at least one symptom of the mental health disorder and/or a reducing the severity of at least one diagnostic criterion for the mental health disorder. As non-limiting examples, the Montgomery-Asberg Depression Rating Scale (MADRS) may be used to evaluate depression, the General Anxiety Disorder-7 (GAD-7) may be used to assess anxiety, and the CAPS-5 may be used to evaluate PTSD.

In some embodiments, the mental health disorder is any of depression, major depressive disorder (MDD), treatment-resistant depression (TRD), atypical depression, postpartum depression, catatonic depression, a depressive disorder due to a medical condition, premenstrual dysphoric disorder, seasonal affective disorder, dysthymia, anxiety and phobia disorders, generalized anxiety disorder (GAD), agoraphobia, panic disorder, separation anxiety disorder, social anxiety disorder, post-traumatic stress disorder, adjustment disorders, feeding and eating disorders, including binge eating, bulimia, and anorexia nervosa, other binge behaviors, body dysmorphic syndromes, drug abuse or dependence disorders, disruptive behavior disorders, impulse control disorders, gaming disorders, gambling disorders, memory loss, dementia of aging, attention deficit hyperactivity disorder, personality disorders, including antisocial, avoidant, borderline, histrionic, narcissistic, obsessive compulsive, paranoid, schizoid and schizotypal personality disorders, attachment disorders, autism, social anxiety in an autistic subject, and dissociative disorders.

Data Collection for Comorbidities: Assessments of comorbidities will be made at screening, baseline, after the eight week MDMA therapy course and at 3, 6, and 9-month follow-ups. Scores are expected to demonstrate improvements, e.g., reductions in symptom severity of substance use disorders, behavioral addictions, or mental health disorders, at follow-up time points, including 3 months, 6 months, and 9 months post-baseline.

A variety of additional data relating to subjective treatment efficacy and quality of life will also be collected, including, e.g., Clinical Global Impressions scales (CGI-I, CGI-S, CGI-C and CGI-E), QOL10 (see, e.g., Muller et al., BMC Med Res Methodol. 2016; 16: 60), changes to the quality of sleep and changes to compassion and empathy scales. Effects of the treatment are expected to improve overall quality of life.

Other mental health measures and quality of life measures: Brief assessments of mood and anxiety will be made at screening, baseline, after the eight week MDMA therapy course and at 3, 6, and 9-month follow-ups, using the PHQ-9 and GAD-7 rating scales respectively. Scores are expected to demonstrate a reduction in both anxiety and depression after screening and baseline timepoints, followed by a transient rise in anxiety and depression scores 3 months after baseline and a further reduction at 6 months and a moderate rise again at 9 months post baseline. A variety of further data will be collected, including changes to the quality of sleep, quality of life measures and changes to compassion and empathy scales.

Suicidality: Participants will undergo the C-SSRS rating scale at screening, baseline, throughout the eight week therapy course, in the week after each MDMA session and at 3, 6, and 9 month follow-up visits. No participants will report current suicidal ideation, intent or plans or self-harm behavior during the course of the study.

Adverse Events: The acute effects of MDMA-assisted therapy will be well-tolerated by participants. No unexpected adverse events will occur. No participants will report any desire to use illicit ecstasy/illicit MDMA following receiving clinical MDMA as part of this trial. No psychotic symptoms will be observed in any patients.

Analysis: Through the Example, MDMA-assisted therapy is demonstrated to be useful in treating AUD, and in AUD in patients who are not physically dependent on alcohol. That is, EAP for an exemplary SUD, harmful use AUD, will be shown to achieve the described outcomes, and also in embodiments will be shown to treat comorbid SUDs, BAs, and mental health disorders.

In some embodiments, another entactogen besides MDMA is used in an EAP regime.

### Example 5: Randomized Double-Blind Between-Subject Controlled Study of MDMA-Assisted Therapy in Patients with “Harmful Use” AUD

In this Example is described a randomized, double-blind controlled study of efficacy of MDMA-assisted therapy for the treatment of patients with non-physically dependent AUD (“Harmful Use”). This is a randomized between-subject controlled study, in patients with non-physically dependent AUD. Half of the patients will receive MDMA-assisted therapy; the other half will receive placebo-assisted psychotherapy.

In this study it will be shown that MDMA-assisted therapy is an effective treatment for patients with AUD, who do not have physical dependence. In other words, the study of this Example will show that MDMA-assisted therapy is superior to placebo dose MDMA-assisted therapy and can reduce use of alcohol in patients without physical alcohol dependence and improve quality of life in patients with AUD. In other examples of this study, an EAP other than MDMA-assisted therapy is used, with a disclosed entactogen other than MDMA.

Outcomes regarding abstinence from alcohol, concomitant drug use (the number and frequency of both prescribed medication use and recreational drug use, including any craving related to MDMA and subsequent use of illicit ecstasy), quality of life, and psychosocial functioning will be evaluated. The main outcome measure will be efficacy, which will be measured in weekly units of alcohol consumed, (wherein a standard drink contains about 14 g of pure alcohol, i.e., ethanol) at 3, 6, and 9 months post baseline.

Here, as also in Examples 4 and 8, the study will adhere to the principles outlined in the Medicines for Human Use (Clinical Trials) Regulations 2004 (SI 2004/1031), amended regulations (SI 2006/1928) and the International Conference on Harmonisation Good Clinical Practice (ICH GCP) guidelines, the Data Protection Act, and such other regulatory requirements as appropriate and as would be understood to those of ordinary skill in the art.

A population of adult patients with a primary diagnosis of AUD, aged between 18 and 65, who do not have physical dependence (and therefore do not require medical detoxification) and who meet the inclusion and exclusion criteria, will be recruited (in a number sufficient to generate statistically significant results). Patients remain in the study for a duration of approx. 14 months.

As depicted in the table below, and as also described in the other Examples herein, patients will first receive a four-week course of supportive group therapy (motivational group meetings) in order to gradually reduce their intake of alcohol. Once abstinent, they will enter a 13-week course of MDMA-assisted therapy, comprising 15 psychotherapy sessions (or, in an alternate aspect, an eight-week course as in the embodiment above). Patients are randomized to either subtherapeutic/active control (placebo) or therapeutic dose. On three psychotherapy sessions (sessions 3, 7 and 13) patients will be given either active control (20 mg+10 mg subtherapeutic) dose of MDMA or active therapeutic (125 mg+62.5 mg) dose MDMA during the 6-8 hour assisted therapy sessions. Patients may optionally stay overnight, or go home after their MDMA session. Other sessions (1, 2, 4-6, 8-12, 14 and 15) will comprise non-drug-assisted 1-hour psychotherapy sessions.

The sessions in general will be as described above, and reference is thus made to the description in Examples 3 and 4. From Session 15 or if discontinued treatment, from 2 weeks post the final psychotherapy session to the final follow-up visit (9 months post baseline). Patients will be followed up at 3, 6, and 9 months from the date of baseline.

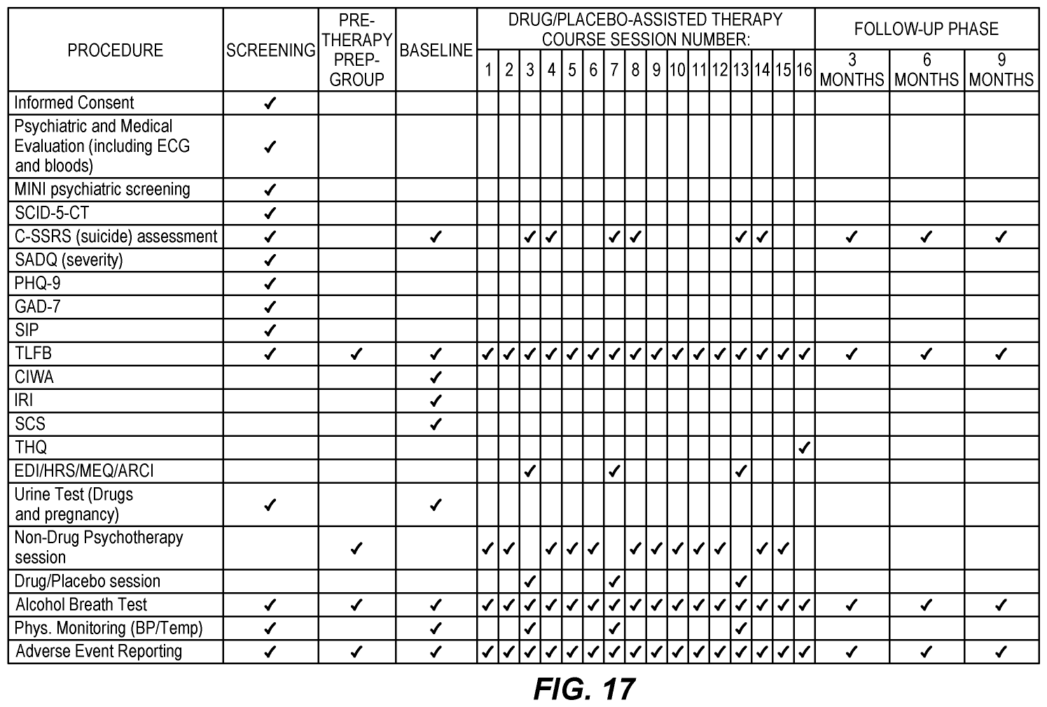

A further understanding of the study procedure, including the timing and schedule of the assessments, may be obtained by reference to FIG. 17.

Results are obtained as above, and by this Example, MDMA-assisted therapy is further demonstrated to be useful in treating AUD, and in particular AUD in patients who are not physically dependent on alcohol, and to have such other benefits as claimed.

Accordingly, and in view of all of the Examples herein, in some embodiments, the pharmaceutical compositions of the invention (and their use in the disclosed methods) are used to improve the symptoms of a mental health disorder, wherein the mental health disorder is a substance abuse disorder. In some embodiments, that substance use disorder is AUD, and specifically non-physically dependent “harmful use” AUD. The symptoms of the mental health disorder to be treated (e.g., the symptoms of non-physically dependent AUD) shall be able to be determined by one of skill, by reference to the general understanding of the art regarding that disorder, for example by reference to the DSM-5). Accordingly, the disclosed methods, and the disclosed compositions used in those methods, are understood as improving any such symptoms.

In some embodiments, the pharmaceutical compositions of the invention (and their use in the disclosed methods) will produce a therapeutic effect, and such effect may be (1) an improvement in a symptom of a comorbid psychiatric disorder; (2) an increase in quality of life; (3) an increase in psychosocial functioning; (4) a decrease in use or frequency of prescription medication; (5) a decrease in use or frequency of recreational drugs; (6) a decrease in units of alcohol consumed; (7) a reduction of cravings relating to alcohol; (8) a prevention of relapse.

### Example 6: Exemplary Treatment Protocols-“Dependent Use” AUD

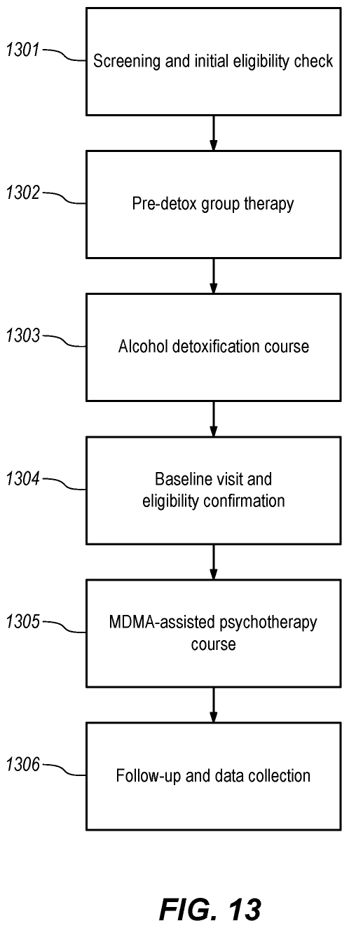

In certain exemplary treatment methods wherein the pharmaceutical compositions of the invention are shown to be useful in methods for treating AUD, the treatment flow can be conceived in phases as follows, and is outlined in FIG. 13: screening and initial eligibility check (1301); pre-detox group therapy, e.g., a four-week course of weekly sessions (1302); an alcohol detoxification course, e.g., over two weeks (1303); baseline visit and eligibility confirmation (1304); an MDMA-assisted therapy course, e.g., over eight or 13 weeks (1305); follow-up and data collection, e.g., at 3 months, 6 months, and 9 months (1306).

It will be readily appreciated, however, that this exemplary treatment flow is merely illustrative of one use of the pharmaceutical compositions in a method of treating AUD, that a use need not include every illustrated aspect (e.g., the follow-up or data collection of step six), and moreover, that the steps, stages, or phases below (and their delineation as such) are simply for purposes of assisting a reader with the conceptualization of a use of the invention, whereas the full breadth and scope thereof will be known by reference to the claims. Moreover, in other embodiments, treatment is of an SUD other than AUD, including an SUD for which a patient first undergoes detoxification from the substance of abuse, which may be any substance of a SUD.

TABLE 8
Exemplary Treatment Flow-“Dependent Use” AUD

1. Screening and Eligibility Check
2. Pre-detox Group Therapy
3. Alcohol Detoxification Course
4. Baseline Visit and Eligibility Confirmation
5. MDMA-assisted therapy Course
6. Follow-Up and Data Collection

Screening and Initial Eligibility Check (1301)

Patients with AUD wishing to undergo detoxification and subsequent MDMA-assisted therapy are screened prior to entering a pre-detox and detoxification program. Eligible patients will attend a baseline visit after detoxification to ensure eligibility.

Potential patients are invited to a screening visit where their eligibility for MDMA-assisted therapy using the disclosed methods will be determined. The screening visit will include informed consent, medical, psychiatric and substance misuse history, prescribed medication use, basic physiological observations and alcohol breath test and urine drugs screen as clinically indicated. In order to monitor the risk of patients using MDMA outside of the therapeutic context, they can be asked about their use of, or desire to use, illicit ecstasy for example using standard questions such as used by MAPS in MDMA-assisted therapy studies.

During screening the therapeutic process may be discussed and explained. The patients' understanding of the procedure, requirements, and commitments may be confirmed and any questions answered before informed consent is obtained. If video and/or audio recordings will be made during therapy sessions, patients should be asked to consent. If patients do not wish to have sessions recorded (or at any point decide to withdraw consent for the whole or part of the session) recordings should be stopped and deleted if requested. Potential patients may be contacted and partake in a short telephone screen before invitation to a full screening visit, to minimize inconvenience to “obviously” ineligible patients.

If psychiatric assessment is sought, it may be performed by a psychiatrist. Assessments may include the Mini International Neuropsychiatric Interview 5 (MINI 5) (Sheehan et al. 1998) to screen for comorbid psychiatric disorders, diagnostic evaluation of the presence of AUD according to DSM-5 (assessed using SCID-5-CT (Clinical Trials Version) (First et al. 2015)) and clinical risk assessment for suicidality using the Columbia Suicide Severity Rating Scale (C-SSRS) (Mundt 2013). Clinical data collection may include years of alcohol dependence, complications/consequences, and previous therapeutic inputs provided including any previous in-patient or community alcohol detox programs.

Pre-detoxification baseline questionnaires also may be completed. Information about alcohol consumption for the three months previous to the screening visit may be assessed using the Alcohol Timeline Follow Back (TLFB) method (Sobell 2001). This is a validated assessment using a calendar method and memory aids to collect retrospective estimates of daily drinking. Patients also may be given the same calendar to take away to help track their drinking behavior for the duration of the therapy.

In some embodiments, another SUD besides AUD is treated in the EAP regime.

In some embodiments, another entactogen besides MDMA is used in the EAP regime.

Pre-Detox Motivational Group Therapy Phase (1302)

After screening, patients attend four weekly sessions of motivational or supportive group therapy pre-detox, e.g., run by a trained counselor with expertise in addictions. The purpose of the group will be to provide psycho-social support to allow adequate preparation and consideration of appropriate aftercare for individuals with AUD wishing to have a detox. Pre-detox psycho-social work benefits from consideration of a person's support network, motivation and after-care planning to improve detoxification outcomes (Kouimtsidis, 2017).

Groups will be, e.g., between five and 10 patients and may be facilitated by one or two trained staff with experience in delivering pre-detox group counseling. Each group session may be for, e.g., 60 minutes and should take place at a predetermined location. In some embodiments, pre-detox group sessions will last for about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, about 120 minutes, and values in between. In some embodiments, pre-detox group sessions may last for greater than 120 minutes, or may take place in a residential (or “rehab”) facility. In some embodiments, groups are closed and confidential. Patients may be enrolled into the groups on a rolling basis, and will leave after a minimum (e.g., of four) weeks attendance to be enrolled into a detox course when deemed appropriate by the facilitators. If a patient fails to attend at least (in the example embodiment, four weeks) of pre-detox group counselling, their detox may be delayed until deemed ready to progress. Similarly, if a patient is not deemed sufficiently engaged in the preparation phase-generally, as deemed by the group facilitators-their detox may be delayed until they are considered to be adequately prepared (e.g., by attending further group sessions).

Alcohol Detoxification Phase (1303)

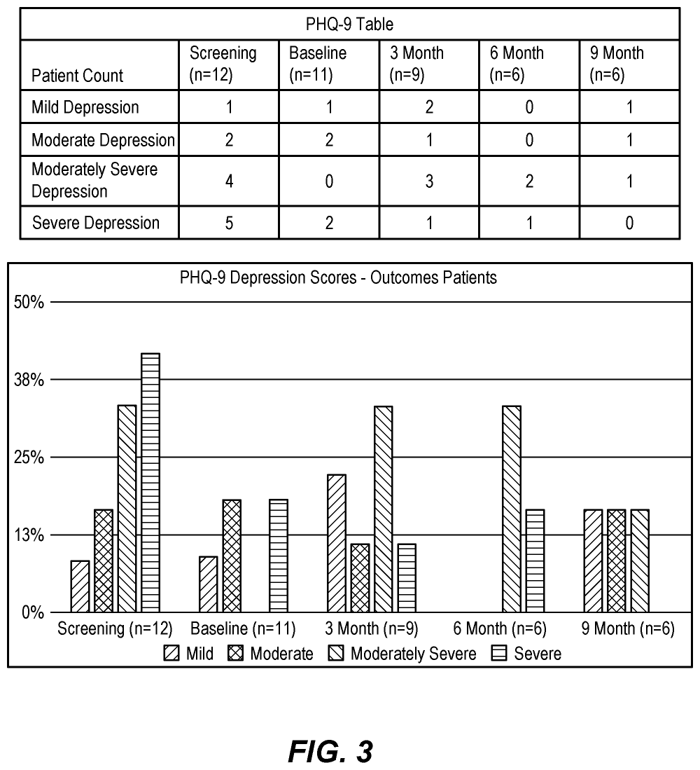

Alcohol (or other substance of abuse) detoxification may be achieved either by gradual reductions in daily alcohol use (which may begin from the start of the pre-detox group phase, with support from the group facilitators) and/or through a medical detox. The medical detox procedure, outlined in FIG. 3., is disclosed in further detail as follows:

First, a patient will undergo a medical assessment (1401). Physical examination and baseline physiological measures, e.g., blood pressure, will be recorded.

Second, and importantly, the patient will stop daily drinking (1402).

Third, the patient may attend a clinic daily (1403) to be prescribed a sliding scale of benzodiazepine treatment (such as chlordiazepoxide) over the next 10-14 days. Chlordiazepoxide or other pharmacotherapeutic treatment (e.g., another benzodiazepine) is prescribed according to good medical practice and dosing regimens known to those in the art.

Fourth, the patient may be assessed by a specialist addictions nurse at each daily visit throughout the detoxification (1404). Daily measurements of blood pressure may be performed. Daily measurements of the Clinical Institute Withdrawal Assessment for Alcohol-Revised Version (CIWA-Ar) (Saitz et al. 1994) (detox assessment measure) may be used to assess response to benzodiazepine administration and progress of the detox.

Fifth, after 10-14 days (depending on the results of the CIWA-Ar and starting dose of chlordiazepoxide or other pharmacotherapy used), the patient is assessed for success of the detox (1405). A medical opinion is sought to assess suitability to progress into treatment.

If the detox is found to be successful (1406)(1407), the detox is complete (1408) and the patient may proceed to treatment. If the detox is found to be incomplete, and thus not successful (1406)(1409) the clinical team may decide whether further interventions are required (1410) before progressing to MDMA-assisted therapy, or whether the patient must return to a prior step (1411).

Baseline Visit and Eligibility Confirmation (1304)

After detoxification is completed, patients may attend a baseline visit to confirm eligibility for continuation into MDMA-assisted therapy. During this visit, continued consent should be confirmed and vital signs can be reassessed. A brief physical examination and ECG may be performed, and blood samples for routine laboratory tests (urea and electrolytes, full blood count, liver function tests and Gamma-GT) also may be completed. A blood test may not be required if the patient has had one in, e.g., the last three months.

In order to confirm the patient has adequately overcome the symptoms of physical alcohol withdrawal, the Clinical Institute Withdrawal Assessment for Alcohol-Revised Version (CIWA-Ar) (Saitz et al. 1994) may be carried out, as well as an alcohol breath test. Dip-stick urinalysis for drugs of abuse can be completed. If female, dip-stick urinalysis for pregnancy may be completed. Patients may be asked to complete post-detoxification questionnaires. Patients may generally start MDMA-assisted therapy as soon as possible after completion of detoxification (generally, within approximately 1 to 3 weeks).

As above, the MDMA-assisted therapy course may be an eight-week MDMA-assisted therapy course with two MDMA sessions (as in TABLE 5) or a 13-week MDMA-assisted therapy course with three MDMA sessions (as in TABLE 6), or a modification of either, based on the teachings herein and the general knowledge in the art.

### Example 7. Exemplary Protocol for EAP for SUDs, Such as MDMA for AUD

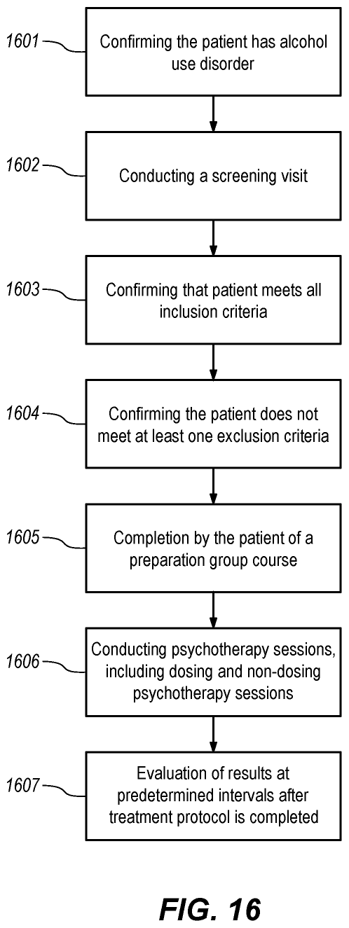

Attention should now be paid to FIG. 16, which outlines one embodiment of the protocol of this Example that is useful in treating an SUD, such as AUD or another described herein, in a patient in need thereof, and is further discussed below. It will be appreciated that modifications may be made as would be understood by those in the art (e.g., substituting Pre-Detox Group Therapy and Pre-Entactogen or Pre-MDMA Course Preparatory Group Therapy Course with an Alcohol Detoxification Course or another Substance Detoxification Course).

In some embodiments, the protocol of this Example will be used with one or more individuals (the term used interchangeably with patients) who have harmful use AUD; in other embodiments, the protocol of this Example will be used with one or more individuals who have dependent use AUD; in other embodiments, the protocol of this Example will be used with multiple individuals, some having harmful use AUD, and some having dependent use AUD; in all such embodiments, modifications are made as will be readily appreciated by those in the art.

The patient first may be confirmed to have AUD or another SUD (1601). This may be accomplished by assessing if the patient meets the diagnostic criteria for the SUD, e.g., at least 2 of the 11 DSM-V criteria for AUD during the same 12-month period, the criteria being whether the individual has: “(1) Had times when you [i.e., the patient] ended up drinking more, or longer, than you intended? (2) More than once wanted to cut down or stop drinking, or tried to, but couldn't? (3) Spent a lot of time drinking? Or being sick or getting over other aftereffects? (4) Wanted a drink so badly you couldn't think of anything else? (5) Found that drinking—or being sick from drinking—often interfered with taking care of your home or family? Or caused job troubles? Or school problems? (6) Continued to drink even though it was causing trouble with your family or friends? (7) Given up or cut back on activities that were important or interesting to you, or gave you pleasure, in order to drink? (8) More than once gotten into situations while or after drinking that increased your chances of getting hurt (such as driving, swimming, using machinery, walking in a dangerous area, or having unsafe sex)? (9) Continued to drink even though it was making you feel depressed or anxious or adding to another health problem? Or after having had a memory blackout? (10) Had to drink much more than you once did to get the effect you want? Or found that your usual number of drinks had much less effect than before? (11) Found that when the effects of alcohol were wearing off, you had withdrawal symptoms, such as trouble sleeping, shakiness, restlessness, nausea, sweating, a racing heart, or a seizure? Or sensed things that were not there?”

A screening visit is conducted (1602), wherein a determination will be made as to whether or not the individual is fit to undergo treatment. Whether or not the individual is fit to undergo treatment will be determined by obtaining objective measurements, such as using inclusion and exclusion criteria as discussed below and set forth herein, or additionally and optionally including any regarding the individual's weight, body temperature, HR, respiratory rate, blood oxygenation, BP and its variables, including, but not limited to: SBP, SBP, MAP, and PP; CNIBP; ECG measurements, including RR interval or its variability, QT interval or its variability, HRV (including measured by devices other than an ECG); hemodynamic response, and levels of glucose, cortisol, serotonin, dopamine, cholesterol; EEG measures; BDNF; genetic markers including relating to CYP enzymes or drug metabolism; genetic variation in mGluR5 or FKBP5; and numerous more, as known to those in the art.

The screening visit will also include assessing whether or not the individual meets all inclusion criteria (1603). As disclosed herein, the inclusion criteria include informed consent, a primary diagnosis of AUD or another SUD, being between the ages of 18 and 65 years old, identifying an individual able to accompany the patient to at least one study visit if required and be contacted by the study team in the event the patient could not be contacted, being proficient in speaking and in reading English, and agreeing to comply with the requirements of the protocol. Such inclusion criteria may be assessed by evaluating an individual's medical, psychiatric, and substance misuse history; prescribed medication use, basic physiological observations and alcohol breath test, and urine drugs screen, or others as clinically indicated.

In addition to meeting all inclusion criteria (1603) an individual in some embodiments will also not have not met at least one exclusion criteria, wherein the exclusion criteria include any one or more of lacking capacity, having a history of a primary psychotic disorder, bipolar affective disorder type 1, or a personality disorder; posing a serious suicide risk, having at least one abnormal clinical finding during the screening visit, being a regular user of ecstasy, currently taking medication likely to interact with MDMA or the entactogen, being a regular use of other drugs or having a dependence thereon (besides also alcohol), being pregnant, breastfeeding, having taken part in a study involving an investigational product in the last 3 months, having an immunological disease, having unstable hypertension, having severe liver disease, having cardiac disease, having an active infection, and having had an infection within four weeks of the entactogen administration.

Where all inclusion criteria are met (1603) and no exclusion criteria are met (1604), an individual may proceed as a patient and complete the four-week preparation group course (1605). As discussed below, the four-week preparation group course may employ group-therapy tactics to enable an individual to lessen their reliance on alcohol or another substance; “reliance” may refer to using alcohol or the other substance as a means to cope with negative experiences in daily life. The exemplary course described here will broadly cover four separate topics, including confidence building; resources, barriers, and goal setting; increasing rewarding activities and building support, and building the foundations for recovery.

So long as the patient completes the preparation group course (i.e., actively participates and does not resume alcohol consumption or substance use), the patient then completes an 11-week therapy protocol comprising 14 psychotherapy sessions (under the supervision of a therapist pair or “therapist team”), three of which are MDMA-assisted therapy (or other EAP) sessions (lasting from between about 360 minutes to about 480 minutes), wherein the patient is administered an initial dose of between about 100 mg to about 150 mg of MDMA (or other entactogen), and optionally a booster dose of about half of the initial dose (thus, if the initial dose was about 100 mg, the booster dose is about 50 mg); and 11 of which are about 60 minute non-drug psychotherapy sessions (1606). In some embodiments, the booster dose is administered to prolong the MDMA (or other entactogen) experience. In some embodiments, the booster dose is administered to increase the time with which the patient and the therapist pair have to interact while the patient is under the effects of MDMA (or the other entactogen).

Once all 14 psychotherapy sessions have been completed, the patient is further assessed at a visit three months, six months, and nine months after the conclusion of the therapy protocol (1607), or otherwise following treatment as defined below. Patients may also be assessed at other times following treatment, such as at one month, at one year, at greater than one year, at times in between, and at points periodically, semi-continuously, or continuously (e.g., by mobile apps, fitness trackers, regular email correspondence, and the like). In some embodiments, the amount of alcohol consumed (or substance used) per patient at the three, six, and nine month visits will be less than the alcohol (or substance) the patient consumed/used prior to beginning the protocol of this Example. In some such embodiments, the amount of alcohol consumed (or substance used) per patient is reduced (i.e., measured at any one or more of the three, six, and/or nine month visits) will be less than the alcohol (or substance) the patient consumed/used prior to the start of the preparation group or prior to the start of detox, or otherwise prior to undergoing the method of the invention (e.g., when measured at or before intake). In some preferred embodiments, the amount of alcohol consumed (or substance used) per patient at the three, six, and/or nine month visits will be 50% or less of the alcohol (or substance) the patient consumed/used prior to the start of the preparation group or prior to the start of detox, or otherwise prior to undergoing the method of the invention. In some preferred embodiments, the amount of alcohol consumed (or substance used) per patient at the three, six, and nine month visits will be a reduction of at least 50%, at least 55%, at least 60%, at least 65%, at least 70%, at least 75%, at least 80%, at least 85%, at least 90%, at least 95%, up to and including a reduction of 100%, i.e., a patient is no longer consuming/using. Other therapeutic effects or outcomes which a patient may experience will be as discussed below.

Regarding the preparation group course in more depth, if eligible at screening (which includes assessing whether the individual meets all inclusion criteria, as discussed herein, and does not meet at least one exclusion criteria, as discussed herein; as well as ensuring the individual is able to undergo treatment, which is assessed by collecting objective measurements including but not limited to weight, body temperature, heart rate, respiratory rate, blood oxygenation, BP and its variables, including SBP, SBP, MAP, and PP; CNIBP; ECG measurements, including RR interval or its variability, QT interval or its variability, HRV (including measured by devices other than an ECG); hemodynamic response, and levels of glucose, cortisol, serotonin, dopamine, cholesterol; EEG measures; BDNF; genetic markers including relating to CYP enzymes or drug metabolism; genetic variation in mGluR5 or FKBP5; and numerous more, as will be appreciated by those of skill); the participant will be enrolled in a four-week preparation group course to support them with making changes to their problematic alcohol or substance use. Each session lasts for 60 mins. and draws on motivational interviewing approaches, as detailed herein. It is anticipated that participants will further benefit from the peer support elements offered by the group format. As mentioned, this will be a rolling group, meaning participants can join at any week and attend for at least 4 weeks.

The content of the preparation group course will broadly cover four topics (below, described for AUD, but for another substance use, “drinking,” “drinks,” “alcohol,” “alcohol consumption,” and such terms can be replaced by like terms relating to the SUD being treated, as readily known):

1. Building Confidence
a. Discussing problems drinking has caused, with family, job, social life, health, etc.
b. Why change is necessary (completing a cost benefit analysis worksheet)
c. Building confidence, discussing when something went well for the patient
d. Completing a drinking diary, where drinking is monitored daily for one week
e. Completing “homework” tasks, such as the drinking diary, and a worksheet detailing strengths and weaknesses of the patient, completed by the patient

2. Resources, Barriers, and Goal Setting
a. Discussing goal setting, specifically having participants elicit goal setting ideas. Exemplary goals may include setting non-drinking days, changing to lower alcohol drinks, increasing the time between drinks, drinking water in between drinks, etc.
b. Reviewing potential barriers and resources that may aid in the reduction of alcohol consumption, such as discussing the patient's strengths and qualities, and identifying social and community support outlets
c. Completing homework tasks, such as setting a goal related to alcohol consumption, completing a daily diary, and completing a worksheet that details social and community support outlets
3. Increasing Rewarding Activities and Building Support
   a. Identifying challenges in achieving goals, and discussing how those challenges were/are managed
   b. Reviewing the social and community support worksheet, and working as a group to build upon the resources listed within
   c. Setting rewarding goals, including planning pleasant activities, etc.
   d. Completing homework tasks, which may include setting a goal related to drinking, and beginning a pleasant activities planner
4. Building the foundations for recovery
   a. Discussing how the patient will manage moving forward, including identifying anticipated challenges and brainstorming ways to manage those, and completing a “planning for the future” worksheet
   b. Identifying any changes the patient would like to make in their recovery program, and completing a worksheet identifying such changes, if so
   c. The patient then completes a recovery support map, and shares/discusses the map with someone who supports the patient

As mentioned, so long as the patient adequately engages in the preparation course, remains eligible (i.e., satisfies the aforementioned inclusion criteria), and illustrates a motivation to make changes to alcohol or other substance use during the preparatory course, the participant will commence a 14 session course of MDMA-assisted therapy (or other EAP) as soon as possible. The investigators will aim to start the course within one week if possible.

Regarding the 14 session, 11 week therapy protocol, all sessions may be conducted by the same two therapists per participant. In other embodiments, different therapists may provide the drug-assisted sessions. There will be a total of three ‘drug’ sessions (6-8 hour MDMA or other entactogen facilitated) wherein an initial dose of about 120 mg and, optionally, a booster dose of about 60 mg are administered, and 11 ‘non-drug’ (60 min. talking therapy) sessions (with dosages for other entactogens known to those of ordinary skill). Non-drug sessions may be conducted by one therapist on rare occasions if this is unavoidable. All drug sessions are facilitated by two therapists and the entactogen compositions are administered by a physician. One therapist at a time may leave the therapy room for brief periods (e.g., for comfort breaks). The participant should be accompanied for the duration of the session and for 6 hours after initial dosing.

All participants starting the main therapy course will have worked on reducing their alcohol intake or other substance use over the 4-week preparatory group program. Therefore, the main aims of the subsequent 14 session therapy course are to support participants to maintain/build upon these changes, prevent relapse into heavy drinking (or problematic substance use) and develop skills to lead a fulfilling life without problematic drinking.

Broadly, the three MDMA-assisted therapy (or EAP) sessions are conducted at weeks 3, 6, and 9. These sessions will last for approximately 6 to 8 hours (360 to 480 minutes), while the non-dosing physiotherapy sessions (which occur all 11 weeks) will last for approximately 1 hour (60 minutes). The therapy appointments prior to the participant's drug assisted sessions will give the therapist team and participant the opportunity to develop a good therapeutic alliance, which is also benefited by the participant having the opportunity to tell their story. This helps to create a collaborative understanding of problem development and maintenance, alongside identification of resources and protective factors. These early sessions give the participant an opportunity to ask any questions about the drug experience, to set an intention for what they would like to get from this experience, and for therapists to describe what they can expect in terms of practicalities and support on the day. The drug-assisted session content is led by the participant, with the therapists in a supporting role and occasionally making gentle suggestions as appropriate. Subsequent integration sessions (i.e., non-drug psychotherapy sessions) are an opportunity to reflect on the MDMA (or other entactogen) experience and to consider any insights and new perspectives that may be emerging and how these may be meaningful to the participant's day to day life.

The 14-week, 11-session therapy protocol (also an “MDMA-assisted therapy course or “MDMA-assisted therapy regimen”) is disclosed in the table below. It will be readily appreciated that this exemplary “MDMA for AUD” program can be modified according to the teachings herein for other entactogens, and/or for other SUDs, as well as for BAs.

TABLE 9
Description of Session Content for Exemplary MDMA for AUD Program

| Week | Visit | Session | |
|---|---|---|---|
| No. | No. | No. | Description of Session Content |
| 1 | 3 | 1 | Introduction, preparation, start of participant's story, therapy goals |
| | | 60 | and introduction to intention setting. |
| | | mins | Introduction to the therapy program (relapse prevention embedded in a |
| | | | framework of enhancing psychological flexibility, using MDMA-assisted |
| | | | therapy to support access to and processing of the factors that have |
| | | | contributed to the development and maintenance of problematic alcohol |
| | | | use). |
| | | | Discuss the role of MDMA as an adjunct to psychotherapy. Opportunity |
| | | | to discuss any questions or concerns that arise. |
| | | | Participant's personal story-contributing and maintaining factors |
| | | | relevant to alcohol use, strengths and resources (strengths and resources |
| | | | to be discussed in more detail in subsequent sessions). |
| | | | Discuss participant's overall goals for therapy and introduce idea of |
| | | | intention setting for their first MDMA assisted session. |
| 2 | 4 | 2 | Continue preparation, intention setting, teach and practice |
| | | 60 | relaxation technique, review of current recovery plan. |
| | | mins | Continue to discuss and develop a shared understanding of the |
| | | | participant's personal story. |
| | | | Discuss and plan intention for MDMA-assisted session. |
| | | | Practice at least one relaxation/anxiety management technique that can |
| | | | be used in the MDMA-assisted session. Discuss use of interpersonal |
| | | | touch for support during sessions and whether the participant is |
| | | | comfortable with this. |
| | | | Review of recovery support map developed during the preparation |
| | | | group. |
| 3 | 5 | 3 | Drug-assisted session 1. Therapists follow the participant's lead. No |
| | | 6-8 | new material introduced by therapists. Journaling encouraged after |
| | | hours | the session. |
| | | | 120 mg MDMA + 60 mg MDMA booster 1.5 to 2 hours later. |
| | | | The participant will always be accompanied by at least one therapist for |
| | | | the duration of the drug-assisted session. Each therapist may leave the |
| | | | room for short periods for comfort breaks but the majority of the session |
| | | | will be conducted with both therapists present. |
| | | | After taking the initial dose (overseen by a medical doctor), the |
| | | | participant will be invited to relax on the bed and listen to music through |
| | | | headphones and with eyeshades on. Therapists will typically make |
| | | | occasional check ins with the participant, usually at the same time that |
| | | | vital signs are taken. |
| | | | Between 90 minutes and two hours after receiving the initial dose, the |
| | | | participant will be given the option to receive a further supplemental |
| | | | dose of half the original dose (60 mg of MDMA). The purpose of this is |
| | | | to prolong the psychological and direct pharmacological effects of the |
| | | | drug to allow for further processing to occur. This dosing and pattern of |
| | | | dosing regime is standard for MDMA-assisted therapy, and has been |
| | | | employed in previous MDMA-assisted therapy pilot studies (Mitheofer |
| | | | 2011, Oehen 2012). |
| | | | Participants are encouraged to have an internal experience to a large |
| | | | extent during this session and spend reasonable amounts of time ‘inside’ |
| | | | with music playing through headphones and eyeshades on. If the |
| | | | participant wishes to talk during this session, this is led by them, with the |
| | | | therapists following their lead and responding with open, interested |
| | | | questions. At certain times, the therapists may encourage the participant |
| | | | to return to having an internal experience, to see what comes up for |
| | | | processing. The participant is not encouraged to get up and move around |
| | | | during the session, but rather to remain sitting or lying throughout. |
| | | | However, if a patient is keen to get up (e.g., they need to go to the |
| | | | bathroom), this can be facilitated if the study team judge this as safe to |
| | | | do so. |
| | | | Towards the end of the session, the participant may wish to talk more as |
| | | | they become more orientated towards their external environment. Once |
| | | | the participant feels ready, the therapy session can be drawn to a close |
| | | | and the participant can have some quiet time to reflect, rest and take light |
| | | | refreshments. Their significant other will be contacted to escort them |
| | | | home no less than 6 hours after the initial MDMA dose. |
| | | | Journaling to facilitate on-going processing is given as a home task. |
| | 6 | 4 | Integration of MDMA experience. Introduction to mindfulness and |
| | | 60 | mindfulness practice. |
| | | mins | The participant may choose to provide a narrative of this experience and |
| | | | the therapists will ask open-ended questions to support the participant to |
| | | | draw personal meanings and insights and to consider how this may |
| | | | impact on their behavior going forward. |
| | | | Mindfulness exercise to finish (mindful body scan). |
| 4 | 7 | 5 | Ongoing integration from medication-assisted session, High Risk |
| | | 60 | Situations for relapse and responses. |
| | | mins | Continue discussion re processing of material from the MDMA session. |
| | | | Discuss the workability of alcohol as a control strategy. |
| | | | Discuss potential High Risk Situations (HRS) for relapse and workable |
| | | | responses (including individual strengths and network and community |
| | | | resources). |
| 5 | 8 | 6 | Understanding how our thoughts hook us in. |
| | | 60 | Identify fusion with thoughts (getting ‘hooked’): rules, reasons, |
| | | mins | judgements, past, future, self. |
| | | | Introduce defusion techniques (getting ‘unhooked’) and practice |
| | | | defusion exercises. |
| | | | Noticing hooks and practising defusion from thoughts set as home task. |
| 6 | 9 | 7 | Drug-assisted session 2. Therapists follow the participant's lead. No |
| | | 6-8 | new material introduced by therapists. Journaling encouraged after |
| | | hours | the session. |
| | | | 120 mg MDMA + 60 mg MDMA booster 1.5 to 2 hours later. |
| | | | refer to session 3 for details |
| | 10 | 8 | Integration of MDMA experience. Mindfulness practice. |
| | | 60 | The participant may choose to provide a narrative of this experience and |
| | | mins | the therapists will ask open-ended questions to support the participant to |
| | | | draw personal meanings and insights and to consider how this may |
| | | | impact on their behavior going forward. |
| | | | Mindfulness exercise to finish (mindful breathing). |
| 7 | 11 | 9 | Opening up by letting go of the struggle |
| | | 60 | Identify problematic experiential avoidance of internal experiences. |
| | | mins | Introduce and practice acceptance techniques. |
| | | | Discuss intention for second MDMA-assisted session. |
| | | | Noticing avoidance and practicing acceptance of emotions as home task. |
| 8 | 12 | 10 | Values and committed action |
| | | 60 | Introduce values (as different to goals) and elicit values from the |
| | | mins | participant. |
| | | | What's important in their life? What they like to do more of/less of? |
| | | | What strengths do they already have to achieve this and which ones |
| | | | would they like to develop? |
| | | | Consider immediate, short, medium and long term goal setting in |
| | | | accordance with values. |
| | | | Identify barriers (FEAR; Fusion, Excessive goals, Avoidance of |
| | | | discomfort, Remoteness from values) and potential responses to barriers |
| | | | (DARE; Defusion, Acceptance of discomfort, Realistic goals, Embracing |
| | | | values). |
| | | | Complete a short-term SMART goal for home task. |
| 9 | 13 | 11 | Drug-assisted session 3. Therapists follow the participant's lead. No new |
| | | 6-8 | material introduced by therapists. Journaling encouraged after the |
| | | hours | session. |
| | | | 120 mg MDMA + 60 mg MDMA booster 1.5 to 2 hours later. |
| | | | refer to session 3 for details. |
| | 14 | 12 | Integration of MDMA experience. Mindfulness practice. |
| | | 60 | The participant may choose to provide a narrative of this experience and |
| | | mins | the therapists will ask open-ended questions to support the participant to |
| | | | draw personal meanings and insights and to consider how this may |
| | | | impact on their behavior going forward. |
| | | | Mindfulness exercise to finish (compassionate body scan). |
| 10 | 15 | 13 | Stepping out of auto-pilot, developing self-compassion and |
| | | 60 | responding to lapses. |
| | | mins | Noticing urges to drink alcohol by stepping out of autopilot. Relate to |
| | | | Seemingly Irrelevant Decisions (SIDs). |
| | | | Discuss everyday mindfulness practice to increase purposeful |
| | | | awareness. |
| | | | Introduce and practice SOBER breathing space. |
| | | | Discussion of inner critic and self-compassionate response. |
| | | | Build on self-compassion work to start lapse response plan. |
| | | | Everyday mindfulness and completion of lapse response plan as home |
| | | | task. |
| 11 | 16 | 14 | Review of the therapy course and ongoing recovery planning. |
| | | 60 | Plans for on-going recovery and integration work within the |
| | | mins | participant's community. |
| | | | Review of recovery plan. |

As it relates to the therapy sessions themselves, as mentioned, there are a total of 14 sessions spanning 11 weeks, wherein 3 of the 14 sessions are MDMA-dosing sessions wherein a patient is administered an initial dose, and optionally a booster dose, the initial dose comprising between about 100 mg to about 150 mg, and the booster dose comprising about half of the initial dose, such that if the initial dose is about 100 mg of MDMA, the booster dose would be about 50 mg of MDMA. In other words, the proportion of the initial dose as compared to the booster dose is 2:1, wherein the initial dose is twice as much as the booster dose. As stated herein, the booster dose is an “optional” dose, wherein the booster dose is not required and, in some embodiments of the invention, the therapy protocol includes three MDMA-assisted therapy sessions, wherein the optional booster dose is not administered to the patient. Likewise, in some embodiments, the optional booster dose is administered during one of the MDMA-assisted therapy sessions; in some embodiments, the optional booster dose is administered during two of the MDMA-assisted therapy sessions; and, finally, in some embodiments, the optional booster dose is administered during all of the MDMA-assisted therapy sessions.

In some embodiments, the optional booster dose is administered to prolong the subjective experience felt by the patient when administered an effective dose of MDMA. In embodiments, the optional booster dose is administered to allow for additional time for the patient and therapist team to discuss and work through any internalized emotions or feelings that surface for the patient.

In embodiments, the 3 MDMA-assisted therapy sessions last for between about 360 minutes to about 480 minutes. In embodiments, the non-dosing therapy sessions last for about 60 minutes.

In other embodiments, another entactogen is used and/or another SUD or a BA is treated.

Further information about individual sessions, in an exemplary embodiment, follows. In other embodiments of the below Table 10, other entactogens and/or other SUDs or BAs are treated.

TABLE 10
Summary of Exemplary MDMA for AUD Program

| | |
|---|---|
| Week 1 | Introduction: Overall MDMA for AUD Program and goals (or overall entactogen |
| Session 1 | for another SUD or for a BA program) |
| | Start to develop a shared understanding of your story in the context of alcohol use |
| | Start preparation for your first medication-assisted session |
| Week 2 | Continue to develop a shared understanding of your story in the context of |
| Session 2 | alcohol use |
| | Continue preparation for your first medication-assisted session |
| | Review of recovery plan developed in preparation group |
| Week 3 | Ready for your first experiential session |
| Session 3 | Journaling |
| Session 4 | Introducing mindfulness and practice |
| Week 4 | Opportunity to continue discussion regarding processing of material that emerged |
| Session 5 | during the experiential session. |
| | High Risk Situations (HRS) and coping responses |
| Week 5 | Unhooking from your thoughts |
| Session 6 | Preparation for your second experiential session |
| Week 6 | Ready for your second experiential session Journaling |
| Session 7 | Mindfulness practice |
| Session 8 | |
| Week 7 | Opportunity to continue discussion regarding processing of material that emerged |
| Session 9 | during the experiential session. |
| | Opening up by letting go of the struggle |
| Week 8 | What matters: identifying personal values |
| Session 10 | Goal setting in accordance with values Identifying barriers and responses |
| Week 9 | Ready for your third experiential session |
| Session 11 | Journaling |
| Session 12 | Mindfulness practice |
| Week 10 | Ready for your third experiential session |
| Session 13 | Journaling |
| | Mindfulness practice |
| Week 11 | Review of the therapy course: Putting it all together |
| Session 14 | |

Week 1, Session 1

The first weekly session will serve as an introduction to the MDMA for AUD therapy (or other entactogen for another SUD or for a BA) protocol, and will cover the purpose of the study (e.g., whether MDMA-assisted therapy is more effective for preventing relapse to harmful drinking compared to psychotherapy without MDMA, or whether EAP generally is more effective for treating an SUB or a BA), and will provide a general overview of the protocol, including that the sessions will focus on reduction strategies and promoting psychological flexibility.

The participant will then be invited to ask any questions. If the participant asks about safety, the administration of MDMA in thousands of modern studies across the world will be discussed, along with the fact that the drug is considered very safe when delivered in combination with therapy and in a highly controlled setting where appropriate monitoring is in place.

The therapy team then discusses with the patient how MDMA works as an adjunct to psychotherapy, which aids in developing a collaborative “formulation,” which is a provisional explanation or hypothesis of how an individual comes to present with a certain difficulty or circumstance at a particular point in time (Weerasekera, 1996). As it relates to the invention, a formulation should be a collaborative process between the patient and therapist team that will be added to, and altered, as the therapeutic process unfolds. It can be considered as a “road map” that will inform the direction of the therapy.

When meeting with a patient for assessment, a psychiatrist and a psychologically informed practitioner will jointly assess their suitability, and in doing so seek to understand their situation with the use of a formulation based around the 5 Ps (Macneil, et al, 2012): (1) What is the Problem that brought you here? (2) What are the Predisposing factors? E.g., life events, family history, traumas, etc. (3) What Precipitated you seeking help now? e.g., recent triggers. (4) What Perpetuates the problem? E.g. thoughts, feelings and behaviors that keep the problem going. (5) What Protective factors are there? E.g. what positives, strengths or assets the person has got going for them. Another formulation can focus on (1) Current difficulties; (2) Past difficulties; (3) Triggers; (4) Maintenance factors (physical, social or psychological factors); and (5) Protective factors (strengths, resources, people or services that are supportive, things learned from experiences, hobbies, etc.).

During the assessment session, the clinicians will listen to the patient's history through the lens of ACT (Acceptance and Commitment Therapy), noticing examples of cognitive fusion, experiential avoidance, loss of values or other examples of psychological inflexibility. This will help identify areas on the ACT hexaflex as having potential for change for the individual. If it is determined that the patient is suitable for the treatment, the case conceptualisation will be further developed with the use of an ACT-based formulation that will guide the therapist and patient in achieving greater psychological flexibility (see below). The therapy team and patient will then work together to form a shared understanding of the formulation, which includes the identification of difficulties, and areas of strength and resource. The therapy team and patient will also work to develop therapy goals for the patient, and introduce “intention setting,” where the patient sets an intention for the drug-assisted session. As described by Mithoefer (2017), it is useful to invite a patient to have a focus for the session but to hold this lightly so that they do not grip onto the intention so tightly it becomes restrictive and potentially disruptive to the natural process that unfolds. Rather, they have a focus in mind but are prepared to go with whatever comes up for them.

Subsequent integration sessions are a valuable opportunity to build upon the insights gained during the drug experience, and consider how these may translate into meaningful and lasting changes in accordance with what the individual feels really matters in their life. The therapeutic model for these integration sessions will be drawn from principles of ACT in order to promote and enhance ongoing psychological flexibility that has been initiated by the drug-assisted sessions. The therapy team will then discuss with the patient the use of ACT in the integration process. Broadly, the goal of combining drug-assisted therapy with ACT is to provide a therapeutic framework that will enable patients to alter patterns of cognition, feelings and behavior that create unworkable actions, and at the same time increasing engagement in line with the values that are important to them. The interaction between these two mechanisms of change (drug-assisted therapy and ACT) provide the environment to increase psychological flexibility and help improve the patient's life.

One premise is that drug-assisted therapy requires the active engagement of patients in their own healing, assisted by a therapeutic framework. This is in contrast to the idea of psychedelic therapy as a “magic bullet” intervention, whereby the administration of the drug causes the appearance of an “inner healer” to do the necessary repair work unassisted. ACT provides a platform by which a patient can actively engage in the process of recovery through specific practices informed by the hexaflex, which leads to increased psychological flexibility, accentuated by the effects of MDMA which allows for enhancement of neuronal plasticity. It is anticipated that the combination of drug effects and psychotherapy provide the optimal conditions for the patient to work on their recovery by enhancing the patient's own innate ability to heal themselves.

ACT encourages acceptance of internal and external discomfort and an ability to tolerate and fully experience them with openness, thereby decreasing the need to use coping strategies of experiential avoidance and unworkable actions. The ultimate aim is to live a full, meaningful life, which will indirectly lead to a reduction in the mental health symptoms that they present with, as well as reducing other associated problematic behaviors. Following the initial preparation stage the therapy protocol alternates between drug-assisted sessions and ACT-informed integration sessions based on the hexaflex; specifically to: (a) be in the present moment; (b) develop a more flexible experience of the self (self-as-context) rather than be fused to a particular personal narrative (self-as-content); (c) disengage from attempts to control thoughts and emotions and instead, observe and accept them as they are (acceptance and defusion), (d) clarify values in the areas of their life they would like to prioritize; and (e) engage in committed action in line with their values.

The ACT-based interventions involve a combination of techniques that enhance the effects of the drug-assisted dosing sessions in order to achieve greater psychological flexibility. These techniques and processes involve a synthesis of several factors in line with ACT: theoretical explanations about ACT principles; metaphors to illustrate key concepts; the use of worksheets; experiential exercises within session; revisiting the ACT specific formulation; and homework tasks between sessions. A guide with suggested exercises and the order in which these processes may be delivered can be provided. A participant will be asked to bring along their recovery support map developed during the preparation group to session 2.

Week 2, Session 2

The second session will generally continue from the first, building on the topics introduced in week one. If the development of the collaborative formulation is not completed during session 1, it is done so in session two. The therapist team will also practice at least one support technique and discuss/practice the use of “interpersonal touch” with the patient during this session.

A key skill of psychedelic-assisted psychotherapy work is to establish excellent patient-therapist rapport as quickly as possible. This will help the patient to feel comfortable enough in their drug-assisted session to be present and interested in whatever comes up for them, rather than trying to avoid or turn away from it. The therapist should spend a few minutes discussing supportive/grounding techniques during preparation; exploring whether the patient has any current ways of managing emotional discomfort that could be useful for the session. Make it clear to the patient that the aim of using a supportive technique is to help the patient to persevere through, rather than avoid, any challenging experiences as much as possible. Discuss the use of supportive touch and whether the patient would feel comfortable with this. Clarify boundaries, all touch is non-sexual and will be on the shoulder, arm or hand. Practice in preparation how the patient may reach out for supportive touch and disengage from the same. It is useful to have an agreement and practice beforehand in the event of challenging experiences arising that the patient would appreciate support with in order to remain in, rather than move away from, the experience.

The therapist team will also discuss supportive/grounding techniques with the patient, non-limiting examples of which include diaphragmatic breathing, wherein the patient is instructed to “take a long, full breath from your abdomen, rather than from your chest. In through your nose and out through your mouth. As you breathe, notice any sensations, thoughts, and images as they come and go as you gently breathe in and out from your tummy;” as well as imagery, wherein the patient is instructed to “close or lower your eyes. Invite a calm and relaxing scene to come to mind and try to make it as vivid as possible. Allow yourself to wander and fill in the details of the image.” The therapist may also utilize the following follow-up prompts: “What can you see/smell/feel? How does it feel to you? Where do you notice it in your body? What's it like?” If the patient appears to be distressed, the practitioner may also instruct the patient to “breathe with me—in through the nose, out through your mouth. Think of a place where you feel safe (can make suggestions if needed), imagine how safe and relaxed you feel, think of it in as much detail as you can. Describe it to me if you like.” Regardless of the method applied, the most important aspect is to ensure the patient is grounded such that their immediate environment can be used if the patient's distress appears to be overwhelming. In extreme situations, at least one of the therapist pair may use reassuring phrases, such as “you're here with us and you're safe, we're right here with you,” and may distract the patient, such that they are focused on external surroundings rather than internal feelings, including exemplary phrases such as “how does the couch feel as you lay on it?” “what can you see around the room?” “are there any noises you can hear?” Note, this technique should be used after other options such as supportive touch (e.g., hand holding), use of the breath, etc.

If the non-drug sessions have taken place in a different room to the one that will be used on dosing day, ensure that the patient is able to see the room before session 3.

The therapist pair will then briefly introduce to the patient what is meant by “psychological flexibility” in terms of three key concepts: “open up” (defusion & acceptance), “be present” (contact with present moment & self as context), and “do what matters” (values & committed action). The therapist pair is not required to spend extensive time explaining these concepts in detail, but can rather introduce them to the patient and explain how they are associated with reduced suffering and enhanced quality of life (rather than symptom reduction necessarily).

The therapist pair will then review the recovery plan developed in the preparation group with the patient, discussing key elements and requesting the patient discuss with them any misapprehended points. The therapist pair will then inquire about what seems to be working well, anything that may be particularly challenging, anything the patient feels could be added, and/or anything the patient would like to develop/change at this point. The therapist pair will ensure they have the opportunity to review and add to the recovery plan with the patient during the final session, when the patient will have learned and practiced skills that may be new to them, and that they may wish to continue developing as part of their ongoing recovery.

At this point, patients may be introduced to the concept of maintaining an experiential therapy journal where they are encouraged to write down anything they find significant during their experiential day. For example, any impactful emotions, perspectives, images, questions, or thoughts that arose. These can be discussed in integration sessions with the therapist pair.

Week 3, Session 3

For the first dosing session, the therapist pair arrives in advance of the patient to ensure the setting is prepared. In total, the therapist pair will ensure the room is clean, airy and comfortable; that blankets, pillows, and sheets are on the bed; that an eye mask and headphones are available; that lighting is dim and comfortable; that all audio equipment is tested and is working (including headphones); that all medical equipment is available and working (BP machine, thermometer); that medication is collected from a pharmacy/stored on-site, and appropriate access has been organized; that rescue medication is accessible; and that therapists' needs are taken care of.

The therapist pair will then conduct a pre-dosing checklist upon patient arrival, which includes reconfirming consent; obtaining blood pressure and pulse measurements; assessing suicide risk via C-SSRS; conducting a urine screen (for substances and pregnancy as appropriate); conducting an alcohol breath test; checking participant medications, (including any changes and what, if any, medication the patient has taken that day to ensure that, e.g., if hypertensive medication was taken that such is routine, etc.).

The patient will then be reminded of key information, including that they are expected to remain in the facility (and in the room) for at least 6 hours after the initial dose, that vital signs will be monitored at dosing, 1 hour after the initial dose, 2 hours after the initial dose (before offering the booster dose), 3 hours after the initial dose, and 6 hours after the initial dose (at the end of the session). That the patient can be supported to get to the bathroom as needed, and the patient is subsequently orientated to the default position of laying on the bed, while wearing eye shades and headphones. That the patient should embrace the experience as much as possible, to be open and curious to all experiences that arise. That the patient will never be on their own, and that at least one therapist will be with them at all times. The patient is reminded about how to reach out and/or ask for support, and the intention set for the session is reviewed. Finally, the therapist pair asks for any questions, reflections, comments, or concerns the patient may wish to talk about at this point.

During the drug-assisted session, the therapeutic interaction will benefit from being more patient-led and open, with the therapist pair maintaining a present and interested stance; holding the PFM in mind but refraining from delving deeply into these processes during the drug session (this can occur during subsequent integration).

When assessing vitals and recording measurements, the therapist pair may gently inquire about how the participant is doing, and if they have been largely ‘inside’ during the session. A gentle touch on the arm (if they have consented to touch) and a question such as, ‘just checking in how you're doing’ will usually be sufficient. Another point at which the participant may naturally wish to engage may come when the booster dose is offered. Here, the therapist pair may wish to inquire more about how the participant is progressing and where they currently find themselves. All communication is offered as a suggestion or invitation rather than an instruction. For example, ‘would you like to . . . ’, ‘might this be a good time to . . . ’ etc. Occasional interactions will help the therapist team judge whether some additional interaction may assist the therapeutic process, whilst always being respectful of the participant's own healing intelligence. This acknowledges and trusts that the participant has an innate internal ability to heal themselves, with the therapy used as a method to facilitate this process and support its natural unfolding. The therapists can use the therapeutic formulation to guide them as to how much support may be required to facilitate the participant's processing. For example, if the participant has identified a tendency to cope via shutting down or isolating themselves, a reminder of the therapist pair's presence and availability may be useful. However, if a participant may intellectualize as an avoidance strategy, they may be encouraged to spend more time ‘inside.’

The therapist pair may also inquire about any sensations in the body associated with what the patient is describing, which can also be helpful to ensure the patient stays focused on their internal experience. Additionally, asking about imagery can support an internal focus. For example, ‘when you describe feeling afraid, do you notice any sensations in your body?’ and ‘are there any images in your mind when you talk about that?’

If the participant describes intense challenging emotions, the therapist pair may offer verbal support and/or supportive touch. They may say, ‘see if you can stay with that feeling that's coming up for healing as much as you can, we're here with you’.

As the majority of actions undertaken by the therapist pair during a dosing session are to support the participant's internal processing and to stay with internal experiences that arise, the therapist pair will be acutely aware of any signs that the patient is unable to maintain an internal focus. When a need arises, the therapists may intervene with supportive actions, such as reminding the patient of their presence e.g., ‘we are here with you’, you are safe,’ using supportive touch (if pre-agreed), encouraging the use of breath, ‘breathe into it,’ ‘remember to use your breathing,’ etc. This instruction can become more directive as warranted (e.g., in accordance with levels of distress). For example, ‘breathe with me’ or ‘breathe in and out on my count’ may be used.

If the patient's levels of distress do not appear to be manageable over a prolonged period, the therapist team can call the study medic who will then make an assessment and consider whether the option of rescue medication is appropriate. In such cases, up to 4 mg of Lorazepam may be administered over a single visit (i.e., one session).

Towards the end of the dosing session, the therapist may make general inquiries about how the patient coped during the session, and how they are presently feeling. However, it is best to not press the patient for a full narrative of the experience at this time, as there may be on-going processing and/or the patient may wish to be quiet and have a period of reflection and introspection. If the patient does wish to talk about their experience at this point, the therapist may listen as a supportive presence but limit any interpretation at this stage while the patient may be in a phase of increased psychological flexibility, and processing may still be unfolding. Rather, it is recommended the therapist pair encourage the patient to have a quiet rest of the day, and to journal/make notes of the experience. Note, the patient may also wish to express their feelings in other ways, such as but not limited to art or music.

Week 3, Session 4

The first session after a dosing session will be geared towards integrating the MDMA experience with the prior therapy sessions. The therapist pair will discuss the purpose of the MDMA dosing session, and will have prompt questions for the patient. At this stage, the therapist pair will also introduce mindfulness techniques and practice the same.

Broadly, mindfulness will be introduced as a key element of ‘being present,’ and is a technique useful in paying attention to the present moment non-judgmentally. It is often a skill taught as part of relapse prevention, and allows patients to notice their “present moment.” It will be useful to ask if the participant has any previous experience practicing mindfulness, and how such experiences went. This session will end with a mindful “body scan” as readily known in the art.

Week 4, Session 5

During the fifth therapy session, the therapist team will continue previous discussions regarding processing the material that emerged during the MDMA dosing session. In addition, High Risk Situations (HRS) and coping responses for the same, will be introduced and discussed.

Week 5, Session 6

During the sixth therapy session, the therapist team will discuss the practice of “unhooking from one's thoughts,” and will prepare the patient for the second MDMA dosing session.

Week 6, Sessions 7 & 8

During the these therapy sessions, the therapist team will ensure the patient is ready for their second experiential MDMA dosing session, and will lead journaling and mindfulness practice.

Week 7, Session 9

During the ninth therapy session, the therapist team will continue to discuss material that emerged during the MDMA dosing session. The therapist team will also discuss the concept of “opening up by letting go of the struggle.”

Week 8, Session 10

During the tenth therapy session, the therapist team will discuss what matters to the patient, including but not limited to key personal values, and will assist the patient in goal setting in accordance with said values. Finally, the therapist team—Identifying barriers and responses

Week 9, Sessions 11 & 12

During the 11th and 12th therapy sessions, the therapist team will ensure the patient is prepared for their third MDMA dosing session, and will lead journaling and mindfulness practice.

Week 10, Session 13

During the thirteenth therapy session, the therapist team will instruct the patient how to respond to urges to drink, will aid the patient in “stepping out of automatic pilot,” (i.e., being more present), and will discuss “Seemingly Irrelevant Decisions,” (SIDS), which are a series of decisions made that, while appearing to be innocuous, lead a patient to a situation wherein alcohol may be freely consumed; “responding to lapses and relapses,” “developing self-compassion,” and how to develop a “SOBER breathing space” with the patient.

Broadly, a “SOBER breathing space” refers to an acronym useful in diagnosing, and responding to dangerous/tempting situations, wherein “S” refers to “stop,” so the patient is instructed to stop at a given “challenging point,” and make the choice to step out of automatic pilot. “O,” stands for “observe,” wherein the patient must become aware of what is happening within themselves. This includes appropriately diagnosing physical sensations, emotions, and, generally, what is happening within the body. This will enable the patient to notice any urges that may force them to act in a certain way. The patient must not force anything away or keep anything out, but instead acknowledge what is happening. “B,” stands for “breath,” wherein the patient is instructed to gather their intention and focus on their breathing. The anchors of the breath are used to come into the body and to stay present. “E,” stands for “expand,” wherein the patient expands the awareness they now feel to include a sense of the entire body, heart, and mind-including any tightness or tension, present emotions, etc. Finally, “R” stands for “respond,” wherein the patient applies the awareness they now feel to their current situation, to determine if the emotion, setting, or person the patient is with is dangerous for them and their recovery. The patient examines the choices they have, and is aware that they are in control of what choice they ultimately make. They then choose the option that best fits what the patient wants in life, including who they wish to be, and what is truly important to them. The patient then visualizes making that choice, and follows through with the decision.

Week 11, Session 14

During the fourteenth therapy session, the therapist team will review the therapy course with the patient. It is at this time the therapist pair is reminded to engage in self-care. This work can be extremely rewarding and also intense and tiring. It is essential that therapists look after themselves. This will help them to be as available and supportive to patients as possible. The therapist pair is encouraged to take some time to consider what the potential challenges of this work may be for them. There are likely to be some practical and personal considerations that can be helpful to discuss with their supervisor early on.

Broadly, when working with a co-therapist, it is important to have met or spoken with them before the patient session (this could be in person, over the telephone or via video call). This will give the opportunity to establish rapport together and also to consider practical considerations such as how to divide the work. It is important to feel comfortable to proceed with the patient and to discuss with the allocated supervisor if there are any doubts.

In some embodiments, the SUD is comorbid to another condition. In other embodiments, the SUD is not comorbid to another condition.

In other embodiments a behavioral addiction, instead of an SUD is treated. In some embodiments, the behavioral addiction is comorbid to another condition. In other embodiments, the behavioral addiction is not comorbid to another condition.

### Example 8: Open-Label, within-Subjects, Safety and Tolerability Feasibility Study of MDMA-Assisted Therapy in Patients with AUD

In this Example is described the first study to investigate the use of MDMA to treat AUD: an open-label safety and tolerability proof-of-concept study investigating the potential role for MDMA therapy in treating patients with AUD. The study was designed to assess if MDMA-assisted therapy can be delivered safely and can be tolerated by patients with AUD post-detoxification. Outcomes regarding abstinence from alcohol, quality of life and psychosocial functioning also were evaluated. Before this study, the qualities of MDMA therapy that could make it potentially well-suited for patients with addictions had been raised in theory by inventor hereof (Sessa 2017), and queried (“Will MDMA work for addictions?”), but they had never before been explored in a research study. Similar open label studies will be appreciated for other SUDs, and for behavioral addictions, based on the teachings herein and the knowledge in the art.

In the study of this Example, 14 patients with AUD completed a community alcohol detoxification course and received an eight week course of abstinence-based therapy, such as described above. Participants received two sessions with MDMA (187.5 mg each session, as two separate doses of 125 mg and then 62.5 mg). Psychological support was provided before, during, and after each session. Safety and tolerability were assessed alongside psychological and physiological outcome measures. Alcohol use behavior, mental well-being and functioning data were collected for nine months after alcohol detoxification.

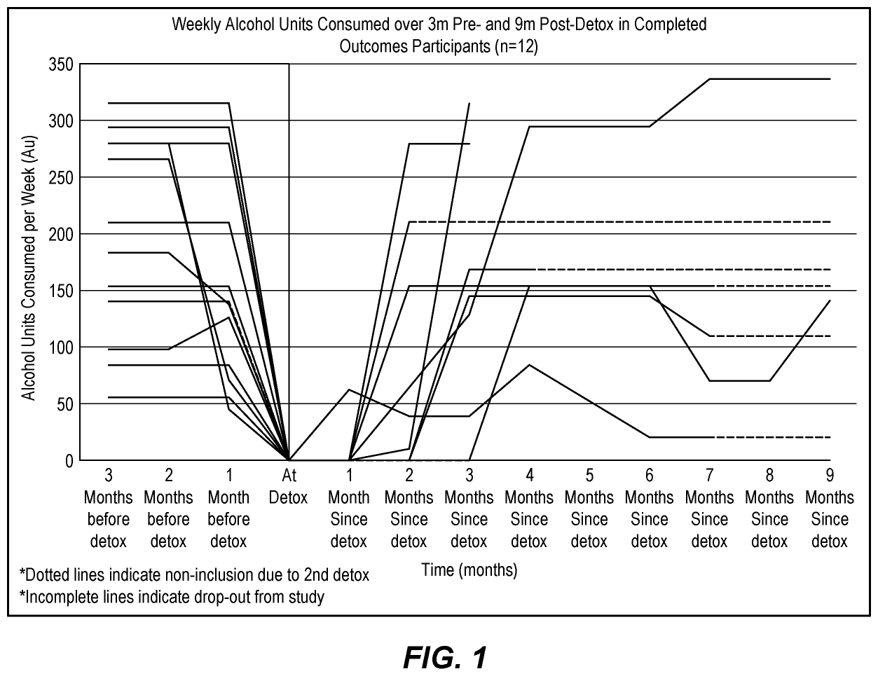

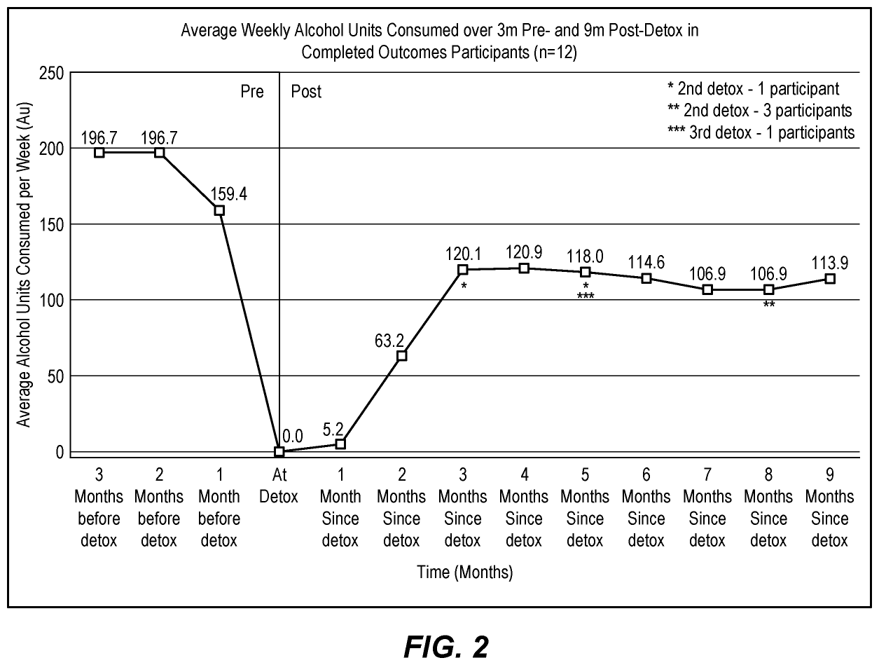

The study of this Example demonstrated that MDMA treatment was well-tolerated by all participants. No unexpected adverse events were observed. Psychosocial functioning improved across the cohort. Regarding alcohol use, at nine months post-detox the average units of alcohol consumption by participants was 18.7 units/week, compared with 130.6 unit/week pre-detox. This compares favorably to a previous observational study by the same team with a similar population of people with AUD (Sessa et al. 2020, and reproduced as FIGS. 1 and 2). For the first time, and in numerous unexpected and surprising ways, support for the safety and tolerability of this novel intervention for AUD post-detox was shown. Specifically, as see in FIG. 1, all but one participant was successful in remaining abstinent from alcohol for at least one month after detox (after averaging 196.7 units of alcohol per week pre-detox) and, while the consumption of alcohol increased from one month to 3 months (from an average of 5.2 units of alcohol consumed per week to an average of 120.1 units of alcohol consumed per week), the figure gradually decreased from 3 months to 7 months, reaching a low of 106.9 units of alcohol consumed per week, and ending at 113.9 units of alcohol consumed per week (as noted in FIG. 2).

Such results, in view of the further description and disclosures provided herein, demonstrate for the first time the therapeutic potential of MDMA-assisted therapy for this population of people with physically dependent AUD, who required and underwent detoxification.

This Example details an open-label, within-subjects, safety and tolerability feasibility study that was conducted in 14 patients aged 18-65 years old diagnosed with AUD who had recently undergone detoxification, and received MDMA-assisted therapy. The main outcome measures of this study were the number of patients completing the eight-week course, the number accepting the second booster dose of MDMA on drug-assisted days, and adverse events. Secondary outcome measures included changes in drinking behavior (measured by units per week consumed at 3, 6, and 9 months since completion of detoxification), measures of mental well-being, psychosocial functioning, quality of life, and concomitant drug use.

Study Design: Patients with a primary diagnosis of AUD who were seeking detoxification—with or without medical assistance-were recruited from the North Somerset Substance Misuse Service (Addaction). Patients received an eight-week course of recovery-based therapy comprising 10 psychotherapy sessions, two of which included dosing patients with open-label MDMA during a 6 to 8-hour assisted therapy session. For each dosing session, participants received an initial oral dose of about 125 mg MDMA, followed about 120 minutes later by a booster dose of about 62.5 mg MDMA, in which the booster dose served to prolong the experience, which allowed for greater time for psychotherapy under the influence of the drug.

Other sessions, including sessions 1, 2, 4, 5, 6, 8, 9, and 10, comprised one-hour psychotherapy sessions, wherein aspects of motivational interviewing and “third-wave” cognitive-behavioral approaches were employed. In all, patients remained in the study for approximately 10 months.

Inclusion and Exclusion Criteria: Inclusion criteria and exclusion criteria included those as set forth in TABLE 1. If one of the exclusion criteria were met, an individual was not selected to take part in the study. To be considered as a patient, all individuals had to meet all inclusion criteria.

Methods: AUD was identified using the DSM-IV SCID interview. Screening comprised of written informed consent, an evaluation of the patient's physical and mental health background, a psychiatric interview (MINI), and assessments of depression and anxiety severity using the Patient Health Questionnaire-9 (PHQ-9) and Generalized Anxiety Disorder Assessment (GAD-7) questionnaires. Severity of AUD was established using the Severity of Alcohol Questionnaire (SADQ) and the Short Inventory of Problems for Alcohol (SIP) questionnaire. However, it should be noted, additional questionnaires/assessments equivalent to those mentioned (as would be apparent to one of skill) may also be used. Patients received a thorough physical health check comprising an electrocardiogram, routine blood tests, blood pressure, heart rate and physical examination. Following screening, eligible patients underwent the process of detoxification either by gradually cutting down alcohol consumption over many weeks or with a medically assisted detoxification regime. The majority of participants were also taking medications for anxiety and/or depressive symptoms (e.g. selective serotonin reuptake inhibitors). According to the inclusion/exclusion criteria, associated medications known to attenuate the effects of MDMA were subsequently gradually reduced and stopped under medical supervision ahead of the first MDMA session. A further ‘baseline’ visit clarified successful detoxification using the Clinical Institute Withdrawal Assessment for Alcohol (CIWA) questionnaire before eligible participants entered the eight-week course of psychotherapy. This entailed weekly 60-minute outpatient non-drug psychotherapy sessions delivered by two clinicians (the named inventors Ben Sessa and Laurie Higbed) trained in delivering MDMA-assisted therapy by MAPS.

Dosing with MDMA occurred twice during the eight-week course on weeks 3 and 6. Physiological changes, observer and subject ratings of distress (Subjective Units of Distress (SUDS)) and the intensity of MDMA's acute psychoactive effects were measured throughout the drug-assisted session. Acute anxiety was managed primarily psychologically, but sedative medication (oral lorazepam) was available. Participants remained overnight in the treatment center after each drug-assisted session, overseen by medically trained “night sitters,” on hand to support participants as required, but instructed to avoid delivering any psychotherapeutic interventions.

Participants were seen the morning after each drug-assisted session for an integration psychotherapy session, and then telephoned daily for six days to assess changes to mood, suicidal risk factors (using the C-SSRS) and quality of sleep (using the Leeds Sleep Evaluation Questionnaire). Following the end of the eight-week therapeutic course, participants carried out additional follow-up questionnaires. They were then seen again at three, six, and nine months (since baseline) for longer-term follow-up data collection.

Data Analysis: All data were recorded on paper case report forms and then digitized into MS Excel spreadsheets. Analysis and graphing were performed with GraphPad Prism v.8.4.3 (GraphPad Software LLC, La Jolla, CA) or MS Excel. When calculating timeline follow-back results, alcohol consumption levels at last observation were used in the case of drop-outs or when participants had undertaken a second detoxification (Hamer and Simpson, 2009).

Results: Demographics: Thirty-six participants attended face-to-face screening visits, and 14 were enrolled (8 males and 6 females; the mean age being 48 years). All were white British. Four were employed, nine were unemployed and one was retired. The average age of first alcohol use was 13 years old. The average age when alcohol became problematic was 34 years old. Nearly two-thirds (64%) of participants reported a history of alcohol-related blackouts, 14% had experienced alcohol withdrawal-induced seizures, 86% of participants reported having experienced risky or vulnerable incidences due to alcohol and 75% of participants had had forensic/offending behavior secondary to their alcohol use.

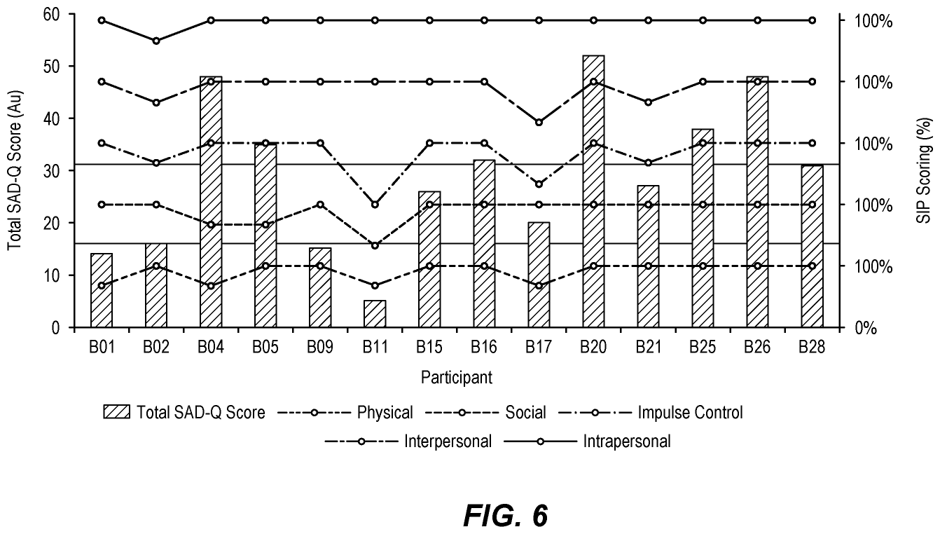

Severity of AUD criteria at screening and baseline: As per the inclusion criteria, all eligible patients scored above the diagnostic threshold on the DSM-5 SCID questionnaire for AUD. AUD severity was also measured using the SIP questionnaire and the SADQ questionnaire (see FIG. 6), with most eligible participants in the moderate to severe range. At the point of Baseline visit, (within one week of detox completion), 100% of eligible participants had successfully completed detoxification, assessed using the CIWA scale.

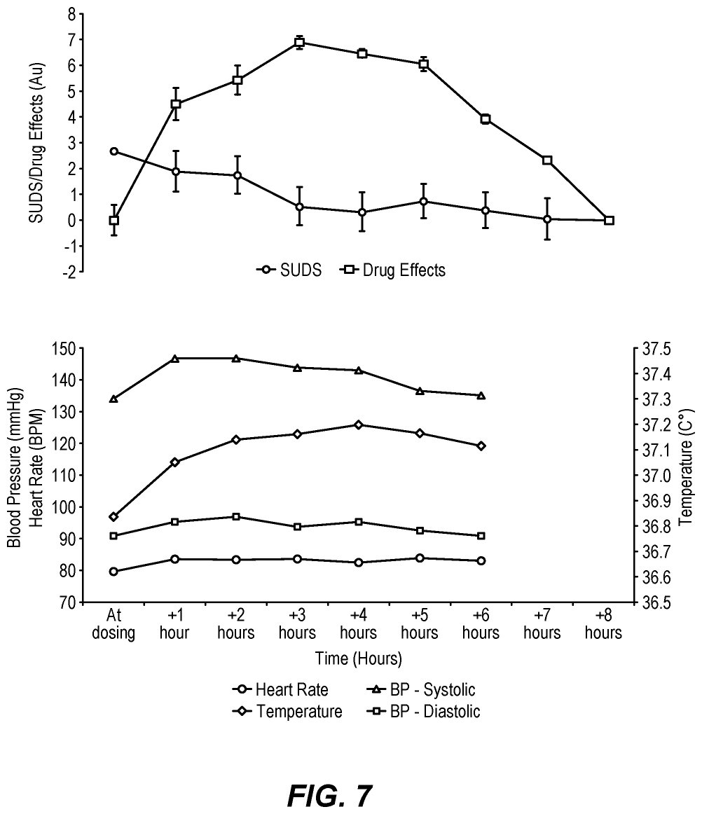

Physiological and tolerability effects during MDMA sessions: In total, 26 drug-assisted psychotherapy sessions with MDMA were administered during the trial. Temperature, blood pressure and heart rate were measured at t=0, before taking MDMA, then half-hourly up to t=2 hours, then hourly thereafter for a minimum of 6-hours from the time of dosing (see FIG. 7).

Except for one participant, all of these physiological parameters remained within normal limits for all these sessions. As expected, a mild, transient rise in blood pressure, temperature and heart rate over the course of the MDMA session was seen. No patients experienced sustained abnormal physiological disturbance, symptomatic experiences of raised blood pressure, heart rate or temperature or any other adverse events during MDMA sessions. No medical interventions were required in respect of these or any other physiological events during MDMA sessions.

One participant experienced a transient abnormal rise in blood pressure after taking the initial dose of 125 mg MDMA reaching a BP 183/118 at two hours after dosing, attributed to the participant forgetting to take her regular antihypertensive medication on the morning of dosing. Although she was asymptomatic and no medical intervention was required it was decided to withhold the two hour supplemental dose. Her blood pressure subsequently spontaneously returned to normal in the following two hours.

Subjective Units of Distress (SUDS) and Participant report of Drug Effects were also measured, hourly, throughout the MDMA sessions (see FIG. 7). Most subjects predictably reported mildly raised SUDS scores at the beginning of the sessions before taking MDMA-consistent with expected anxiety ahead of dosing-which subsequently reduced during the course of the session as the positive effects of MDMA emerged. Participants gave their own subjective score (0-10) of whether they felt Drug Effects, and the therapists also recorded their own objective score of how “altered” the participant appeared. There was no significant difference between Observers' and Participants' Drug Effects scores. Drug Effects rose expectedly over the first two hours, with a notable further increase after the booster dose was given at t=2 hours, and a subsequent plateau then decline over the following six hours. By the end of the MDMA session day all drug effects had returned to baseline. No participants reported any significant neurocognitive impairments associated with receiving MDMA in the weeks and months following participation in the study.

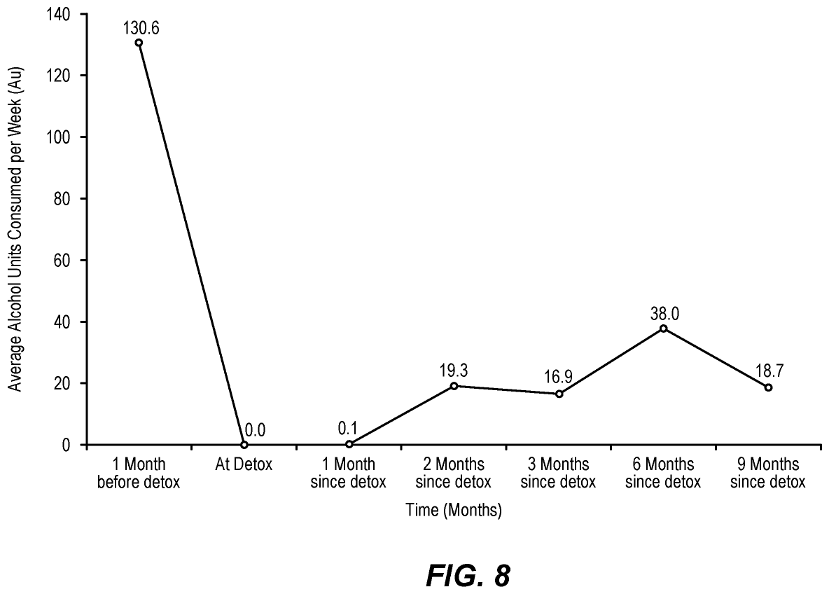

Changes in drinking behavior: While changes in drinking behavior were not a primary outcome measure, data was nevertheless collected in respect of units of alcohol consumed per week (wherein a standard drink contains about 14 g of pure alcohol, i.e., ethanol) in the month before participants' detoxification, immediately after detox (‘baseline’), throughout the eight-week MDMA therapy course and for up to nine months after detox. Of the 14 eligible participants who underwent the course of MDMA-assisted therapy, at the nine-month follow-up end point, 11 participants were drinking fewer than 14 units of alcohol per week (including nine who were totally abstinent from alcohol), and three participants had relapsed to drinking more than 14 units of alcohol per week. On average, participants were drinking 130.6 units of alcohol per week in the month before detoxification, and no units at the point of detox. After nine months, the average amount of consumed alcohol had risen back to 18.7 units per week (see FIG. 8).

Specifically, participants consumed a cumulative 16.9 units of alcohol per week at 3 months post-detox, 38.0 units of alcohol per week at 6 months post-detox, and 18.7 units of alcohol at 9 months post-detox. This yields decreases of 87.1% at 3 months post-detox, 71% at 6 months post-detox, and 85.7% at 9 months post-detox.

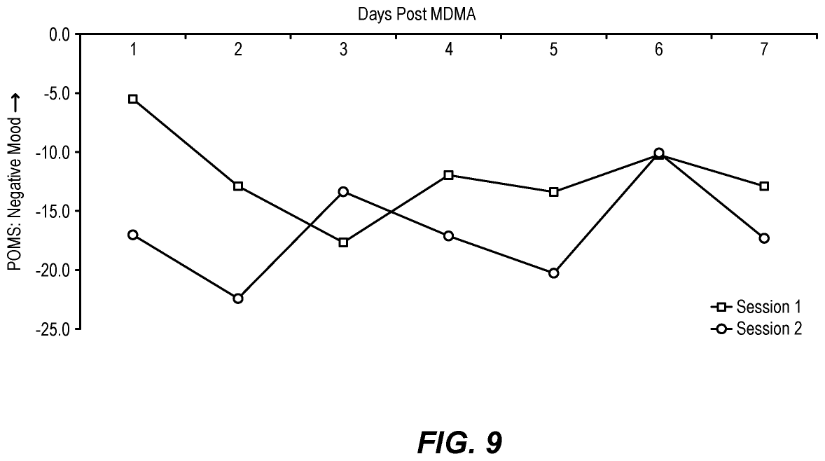

Seven-day follow-up after MDMA sessions: Considerable medical literature and the popular press report the anecdotal observation of ecstasy users experiencing an acute ‘come-down’ effect and a drop in mood in the days after using the drug recreationally. In order to measure this prospectively with clinical MDMA, we measured participants' mood states by daily Profile Of Mood States (POMS) measurements for seven days after each MDMA session (see FIG. 9). Positive scores represent depressed affect, zero represents no change in mood/affect and results below zero represent a positively felt mood. Average scores across both MDMA sessions for all 14 participants (26 MDMA sessions) revealed no evidence of any mood disturbance during the week after taking each session of clinical MDMA. Indeed, participants sustained a positive mood for seven days. This result contrasts with anecdotal reports from recreational ecstasy users.

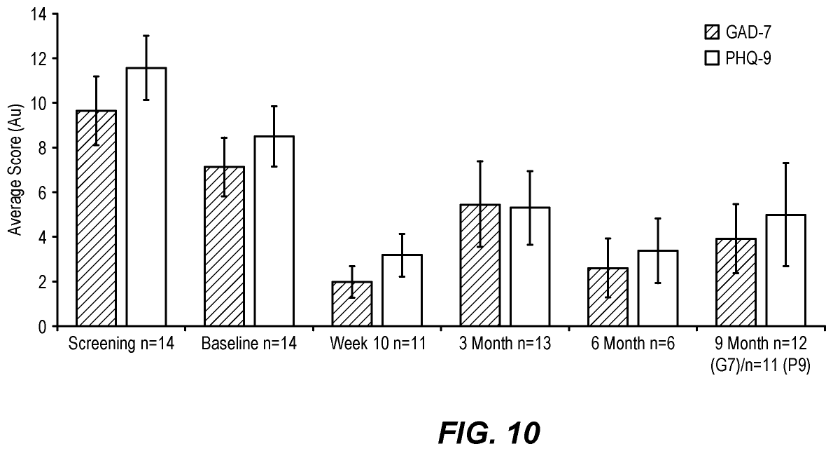

Other mental health measures and quality of life measures: Brief assessments of mood and anxiety were made at screening, baseline, after the eight-week MDMA therapy course and at the three-, six-, and nine-month follow-ups using the PHQ-9 and GAD-7 rating scales, respectively (see FIG. 10). Scores demonstrate a reduction in both anxiety and depression after screening and baseline time points, followed by a transient rise in anxiety and depression scores three months after baseline, a further reduction at six months and a moderate rise again at nine months post detox. A variety of further data were collected, including changes to the quality of sleep, quality of life measures and changes to compassion and empathy scales. These data will show improvements in all such secondary measures.

Suicidality: Participants underwent the C-SSRS at screening, baseline, throughout the eight-week therapy course, in the week after each MDMA session and at the three-, six-, and nine-month follow-up visits. No participants reported current suicidal ideation, intent or plans or self-harm behavior during the course of the study.

Adverse events: The acute effects of MDMA-assisted therapy were well tolerated by participants. No unexpected adverse events occurred. No participants reported any desire to use illicit ecstasy/illicit MDMA following receiving clinical MDMA as part of this trial. No psychotic symptoms were observed in any of the patients.

A variety of further data were collected, including changes to quality of sleep, quality of life measures, and compassion and empathy scales, demonstrating benefits and improvements.

Discussion: In this first safety and tolerability study, MDMA-assisted therapy was demonstrated for the first time to be useful in treating AUD, for example in patients needing detox and physically dependent on alcohol.

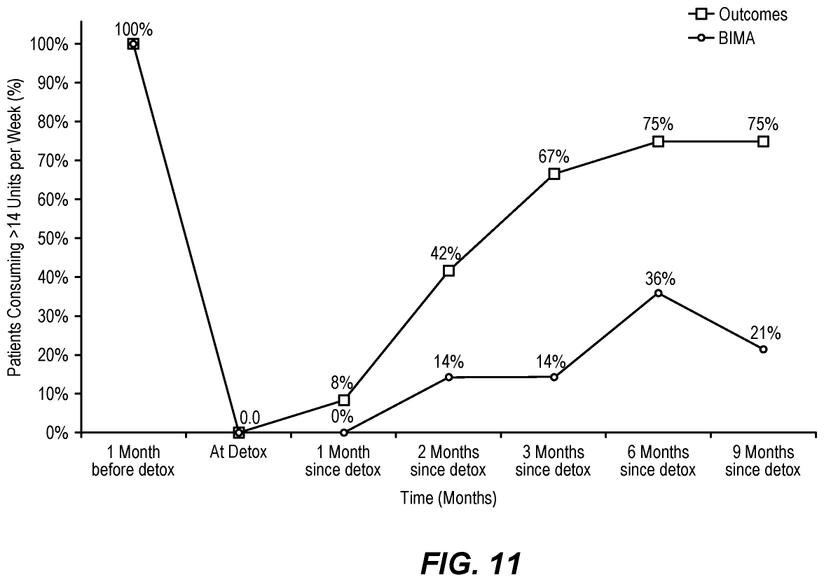

Prior to carrying out the study of this Example, the same study team carried out a non-interventional observational study, following 14 participants through their treatment-as-usual post-alcohol detox (the “Outcomes Study,” published as Sessa et al., 2020). The eligibility criteria and questionnaires used in the Outcomes Study had similarities to the study of this Example, but it was without the additional eight-week therapeutic course with MDMA-assisted therapy, which occurred post detox. Although patients were not randomized into the studies, FIG. 11 demonstrates the success of the participants of the study of this Example in terms of alcohol consumption over 9 months, compared with current best treatments available locally. Only 21% of participants who had undergone MDMA-assisted therapy were drinking in excess of 14 units of alcohol a week in comparison with the 75% observed in the Outcomes Study (see FIG. 11).

Through the Example, MDMA-assisted therapy is demonstrated to be useful in treating AUD, and in AUD in patients who are physically dependent on alcohol. Along the course of treatment, it was recognized that MDMA-assisted therapy focused on treating AUD unexpectedly improved outcomes of other comorbid addictions, including additional substance use disorders (SUDs) and behavioral addictions (BAs). Positive outcomes of comorbidities are a surprising additional indicator of the efficacy of EAP.

### Example 9: Randomized Double-Blind Between-Subject Controlled Study of MDMA-Assisted Therapy in Patients with “Dependent Use” AUD

In this Example is described a randomized, double-blind controlled study of efficacy of MDMA-assisted therapy for the treatment of patients with physically dependent AUD (“Dependent Use”). This is a randomized between-subject controlled study, in patients with physically dependent AUD. Half of the patients will receive MDMA-assisted therapy; the other half will receive placebo-assisted psychotherapy.

In other embodiments, an EAP other than MDMA-assisted therapy is used, with an entactogen other than MDMA. In other embodiments, a SUD other than AUD is treated. In other embodiments, a behavioral addiction is treated. Similar studies will be appreciated for such other SUDs, and for behavioral addictions, based on the teachings herein and the knowledge in the art.

In this study it will be shown that MDMA-assisted therapy is an effective treatment for patients with AUD, who have physical dependence. In other words, the study of this Example will show that MDMA-assisted therapy is superior to placebo dose-assisted psychotherapy and can reduce use of alcohol in patients with physical alcohol dependence—and, in some embodiments, especially those who require a course of detoxification before they undergo drug-assisted therapy—and improve their quality of life. In other examples of this study, an EAP other than MDMA-assisted therapy is used, with a disclosed entactogen other than MDMA.

Outcomes regarding abstinence from alcohol, concomitant drug use (the number and frequency of both prescribed medication use and recreational drug use, including any craving related to MDMA and subsequent use of illicit ecstasy), quality of life, and psychosocial functioning will be evaluated. The main outcome measure will be efficacy, which will be measured in weekly units of alcohol consumed, (wherein a standard drink contains about 14 g of pure alcohol, i.e., ethanol) at 3, 6, and 9 months post detox.

Here, as also in the Examples above, the study will adhere to the principles outlined in the Medicines for Human Use (Clinical Trials) Regulations 2004 (SI 2004/1031), amended regulations (SI 2006/1928) and the International Conference on Harmonisation Good Clinical Practice (ICH GCP) guidelines, the Data Protection Act, and such other regulatory requirements as appropriate and as would be understood to those of ordinary skill in the art.

A population of adult patients with a primary diagnosis of AUD, aged between 18 and 65, who have physical dependence (and therefore in some embodiments will require, and undergo, medical detoxification) and who meet the inclusion and exclusion criteria, will be recruited (in a number sufficient to generate statistically significant results). Patients will remain in the study for a duration of approximately 14 months.

As depicted in the table of FIG. 17 (but with such changes as will be readily appreciated, such as the replacement of “Pre-Therapy Prep-Group” with pre-detox group therapy, followed by detoxification, compare, e.g., TABLE 4 and TABLE 8), and as also further described in the other Examples herein, patients will first receive four weekly sessions of motivational or supportive group therapy pre-detox. Subsequently, alcohol detoxification will be achieved either by gradual reductions in daily alcohol use (which may begin from the start of the pre-detox group phase, with support from the group facilitators) and/or through a medical detox. Once abstinent, they will enter a 13-week course of MDMA-assisted therapy, comprising fifteen psychotherapy sessions (or, in an alternate aspect, an eight-week course as in the embodiment described above). Patients are randomized to either subtherapeutic/active control (placebo) or therapeutic dose. On three psychotherapy sessions (session 3, session 7 and session 13) patients will be given either active control (20 mg+10 mg subtherapeutic) dose of MDMA or active therapeutic (125 mg+62.5 mg) dose MDMA during the 6-8 hour assisted therapy sessions. Patients may optionally stay overnight, or go home after their MDMA session. Other sessions (1, 2, 4-6, 8-12, 14 and 15) will comprise non-drug-assisted 1-hour psychotherapy sessions.

The sessions in general will be as described above, and reference is thus made to the description in the above Examples. From Session 15 or if discontinued treatment, from 2 weeks post the final psychotherapy session to the final follow-up visit (9 months post baseline). Patients will be followed up at 3, 6, and 9 months from the date of baseline.

Results: Results are obtained as above, and through this Example, MDMA-assisted therapy is further demonstrated to be useful in treating AUD, and in particular AUD in patients who are physically dependent on alcohol, and to have such other benefits as claimed.

Assessing Comorbid SUDs: Disclosed treatment methods for exemplary SUD dependent use AUD, in some embodiments, will additionally be effective in treating one or more comorbid substance use disorders (SUDs). In such embodiments, the exemplary treatment methods will result in one or more of a reduction of substance use, a reduction of substance cravings, the promotion of abstinence from a substance, and improvement or a reduction in at least one symptom of an SUD. The substance use disorder may be any one or more of opioid use disorder, nicotine dependence and tobacco use disorder, sedative, hypnotic, and anxiolytic use disorder; and stimulant use disorder, including others known to one of skill in the art.

A reduction in substance use or abstinence therefrom may be determined according to observer- or subject-reported methods. In some examples, a reduction in substance use may be determined by evaluating biological specimens, e.g., saliva, blood, and urine, to determine the level of a marker of substance use. In other examples, a subject may self-report the frequency of substance use, including a reduction thereof, using a questionnaire or survey, e.g., the Addiction Severity Index (ASI). In some cases, these approaches may be combined to determine a reduction in substance use of abstinence from the same. Improvement of at least one symptom of an SUD, or a reduction in the severity of at least one diagnostic criterion may be determined by consulting available diagnostic criteria, e.g., the DSM-5 and other applicable measures.

Assessing Comorbid BAs: Disclosed treatment methods will additionally be effective in treating one or more comorbid behavioral addictions (BAs). In such embodiments, the exemplary treatment methods will result in any of a reduction of the behavior, abstinence from the behavior, and prevention of relapse into the behavior.

Methods of determining a reduction in behavior, relapse, and abstinence are known to one of skill in the art, and exemplary methods are described herein. The behavioral addiction may be any one or more of gambling disorder, compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction, including others known to one of skill in the art.

Assessing Comorbid Mental Health Conditions: The provided exemplary treatment methods will additionally be effective in treating one or more comorbid mental health disorders. In such embodiments, the exemplary treatment methods will result in an improvement, e.g., ameliorating or preventing at least one symptom of the mental health disorder and/or a reducing the severity of at least one diagnostic criterion for the mental health disorder. As non-limiting examples, the Montgomery-Asberg Depression Rating Scale (MADRS) may be used to evaluate depression, the General Anxiety Disorder-7 (GAD-7) may be used to assess anxiety, and the CAPS-5 may be used to evaluate PTSD.

In some embodiments, the mental health disorder is any of depression, major depressive disorder (MDD), treatment-resistant depression (TRD), atypical depression, postpartum depression, catatonic depression, a depressive disorder due to a medical condition, premenstrual dysphoric disorder, seasonal affective disorder, dysthymia, anxiety and phobia disorders, generalized anxiety disorder (GAD), agoraphobia, panic disorder, separation anxiety disorder, social anxiety disorder, post-traumatic stress disorder, adjustment disorders, feeding and eating disorders, including binge eating, bulimia, and anorexia nervosa, other binge behaviors, body dysmorphic syndromes, drug abuse or dependence disorders, disruptive behavior disorders, impulse control disorders, gaming disorders, gambling disorders, memory loss, dementia of aging, attention deficit hyperactivity disorder, personality disorders, including antisocial, avoidant, borderline, histrionic, narcissistic, obsessive compulsive, paranoid, schizoid and schizotypal personality disorders, attachment disorders, autism, social anxiety in an autistic subject, and dissociative disorders.

Data Collection for Comorbidities: Assessments of comorbidities will be made at screening, baseline, after the eight week MDMA-assisted therapy (or other EAP) course and at 3, 6, and 9-month follow-ups. Scores are expected to demonstrate improvements, e.g., reductions in symptom severity of substance use disorders, behavioral addictions, or mental health disorders, at follow-up time points, including 3 months, 6 months, and 9 months post-baseline.

A variety of additional data relating to subjective treatment efficacy and quality of life will also be collected, including, e.g., Clinical Global Impressions scales (CGI-I, CGI-S, CGI-C and CGI-E), QOL10 (see, e.g., Muller et al., BMC Med Res Methodol. 2016; 16: 60), changes to the quality of sleep and changes to compassion and empathy scales. Effects of the treatment are expected to improve overall quality of life.

Accordingly, in view of the above, and the other Examples herein, in some embodiments, the disclosed compositions (and their use in the disclosed methods) are used to improve the symptoms of a mental health disorder, wherein the mental health disorder is a substance abuse disorder. In some embodiments, that substance use disorder is AUD, and specifically physically dependent alcohol addiction. The symptoms of the mental health disorder to be treated (e.g., the symptoms of AUD or physically dependent alcohol addiction) shall be able to be determined by one of skill, by reference to the general understanding of the art regarding that disorder, for example by reference to the DSM-5). Accordingly, disclosed methods, and disclosed compositions when used in those methods, will be understood as improving any such symptoms.

In some embodiments, the pharmaceutical compositions of the invention (and their use in the disclosed methods) will produce a therapeutic effect, and such effect may be (1) an improvement in a symptom of a comorbid psychiatric disorder; (2) an increase in quality of life; (3) an increase in psychosocial functioning; (4) a decrease in use or frequency of prescription medication; (5) a decrease in use or frequency of recreational drugs, which can include illicit use of prescription drugs; (6) a decrease in units of alcohol consumed; (7) a reduction of cravings relating to alcohol; (8) a prevention of relapse.

### Example 10: EAP to Treat Subjects Having a Mental Health Disorder and Comorbidities

Purpose: To assess the effects of EAP in treating a subject having a mental health disorder and one or more comorbidities. For exemplary purposes, the mental health disorder is PTSD, which should not be construed as limiting. Comorbidities may be any one or more of an SUD, an additional BA, or a mental health disorder. Additionally, whether a subject's comorbidities have improved as a result of entactogen-assisted therapy is addressed.

Methods: Subjects are assessed for PTSD severity using the Clinician-Administered PTSD Scale for DSM-5 (CAPS-5). Prior to beginning an EAP regimen, as described herein, e.g., the methods of any of Examples 3 and 4, biological specimens are collected from each subject and/or each subject completes assessments and to determine baseline comorbidities. Comorbidities are coexisting conditions that may be any one or more of a substance use disorder, additional behavioral addiction, or mental health disorder.

Screening and baseline measures are collected onsite on Day 1, when the above self-report measures are conducted again. EEG measurements are collected. These measures are used to determine a baseline for each subject. The entactogen for administration to a subject as part of an EAP regimen is any of a substituted amphetamine, a substituted benzofuran, a substituted phenethylamine, or a substituted tryptamine. Exemplary dosing for MDMA is described in the previous Examples, and dosing a therapeutically amount for other entactogens may be determined according to one of skill in the art.

Results: Entactogen-assisted therapy is expected to treat the exemplary mental health disorder PTSD. Treatment of PTSD may be represented by a reduction in CAPS-5 score.

Assessing Comorbid SUDs: The disclosed EAP methods for treating the exemplary mental health disorder PTSD will be effective in treating one or more comorbid substance use disorders (SUDs). In such embodiments, the exemplary treatment methods will result in one or more of a reduction of substance use, a reduction of substance cravings, the promotion of abstinence from a substance, and improvement or a reduction in at least one symptom of an SUD. The substance use disorder may be any one or more of opioid use disorder, nicotine dependence and tobacco use disorder, sedative, hypnotic, and anxiolytic use disorder; and stimulant use disorder, including others known to one of skill in the art.

A reduction in substance use or abstinence therefrom may be determined according to observer- or subject-reported methods. In some examples, a reduction in substance use may be determined by evaluating biological specimens, e.g., saliva, blood, and urine, to determine the level of a marker of substance use. In other examples, a subject may self-report the frequency of substance use, including a reduction thereof, using a questionnaire or survey, e.g., the Addiction Severity Index (ASI). In some cases, these approaches may be combined to determine a reduction in substance use of abstinence from the same. Improvement of at least one symptom of an SUD, or a reduction in the severity of at least one diagnostic criterion may be determined by consulting available diagnostic criteria, e.g., the DSM-5 and other applicable measures.

Assessing Comorbid BAs: In some embodiments, the disclosed methods will additionally be effective in treating one or more comorbid behavioral addictions (BAs). In such embodiments, the exemplary treatment methods will result in any of a reduction of the behavior, abstinence from the behavior, and prevention of relapse into the behavior.

Methods of determining a reduction in behavior, relapse, and abstinence are known to one of skill in the art, and exemplary methods are described herein. The behavioral addiction may be any one or more of compulsive sexual behavior disorder, compulsive buying-shopping disorder, internet addiction, gaming disorder and internet gaming disorder, risk-taking addictions, kleptomania, pyromania, pornography addiction, technology addiction, binge eating disorder, workaholism, perfectionism, pathological skin picking, exercise addiction (including overtraining syndrome), excessive tanning, tattoo addiction, masturbation addiction, sex addiction, and love addiction, including others known to one of skill in the art.

Assessing Comorbid Mental Health Conditions: The provided exemplary treatment methods will additionally be effective in treating an additional comorbid mental health disorder. In such embodiments, the exemplary treatment methods will result in an improvement, e.g., ameliorating or preventing at least one symptom of the mental health disorder and/or a reducing the severity of at least one diagnostic criterion for the mental health disorder. As non-limiting examples, the Montgomery-Asberg Depression Rating Scale (MADRS) may be used to evaluate depression, the General Anxiety Disorder-7 (GAD-7) may be used to assess anxiety, and the CAPS-5 may be used to evaluate PTSD.

In some embodiments, the mental health disorder is selected from depression, major depressive disorder (MDD), treatment-resistant depression (TRD), atypical depression, postpartum depression, catatonic depression, a depressive disorder due to a medical condition, premenstrual dysphoric disorder, seasonal affective disorder, dysthymia, anxiety and phobia disorders, generalized anxiety disorder (GAD), agoraphobia, panic disorder, separation anxiety disorder, social anxiety disorder, post-traumatic stress disorder, adjustment disorders, feeding and eating disorders, including binge eating, bulimia, and anorexia nervosa, other binge behaviors, body dysmorphic syndromes, drug abuse or dependence disorders, disruptive behavior disorders, impulse control disorders, gaming disorders, gambling disorders, memory loss, dementia of aging, attention deficit hyperactivity disorder, personality disorders, including antisocial, avoidant, borderline, histrionic, narcissistic, obsessive compulsive, paranoid, schizoid and schizotypal personality disorders, attachment disorders, autism, social anxiety in an autistic subject, and dissociative disorders.

Data Collection for Comorbidities: Assessments of comorbidities will be made at screening, baseline, after the eight week EAP course and at 3, 6, and 9-month follow-ups. Scores are expected to demonstrate improvements, e.g., reductions in symptom severity of substance use disorders, behavioral addictions, or mental health disorders, at follow-up time points, including 3 months, 6 months, and 9 months post-baseline.

### Example 11: Exemplary EAP Protocol for Subjects with Behavioral Addictions

Purpose: Aims of this mechanistic study include to examine the impact of a single dose of an entactogen, which may be MDMA, in people with a BA on reward sensitivity and processing and on habitual responding, and to examine activation reward sensitivity and processing two hours following entactogen administration on a neuroimaging version of the reward processing task. Markers of effectiveness will be changes in reward processing, and attenuation of response on no-win trials, in particular response to “near-misses.” Other markers of craving, psychological wellbeing, and symptom severity are secondary outcomes. Preliminary explorations of the sub-acute impact of an entactogen on our procedural learning task would seek to examine whether the entactogen can disrupt striatal habitual responding.

Subjects and Design: Subjects meeting DSM-5 criteria for one or more BAs will be identified and randomised to receive either a dose of an entactogen, for example MDMA, or a placebo. Inclusion criteria will be over 18 years old; no serious mental health problems; and no contraindications for the entactogen.

Procedure: Following telephone screening by the research team, in person screening by a medical physician and psychologist, and informed consent, demographic data will be collected from subjects. Subjects will then undergo baseline measures of reward and procedural learning, alongside questionnaires of mood, symptom severity, trauma history and psychological well being. Entactogenic dosing will be via procedures well-known to those in the art.

Subjects will recline listening to music for the duration of the acute effect and then complete questionnaires of mood, wellbeing, symptoms, and mystical effects. The same cognitive tasks will be repeated and subjects will be moved to the MRI/PET unit to undergo an fMRI scan while completing a task characteristic of the behavioral addiction in the scanner (for example, a virtual slot machine task for a person diagnosed with gambling disorder).

Results: This study will show that a single dose of an entactogen, for instance MDMA, is an effective treatment for subjects with a behavioral addiction. The study of this Example will show that the entactogen is superior to placebo, as measured by any one or more of: (1) frequency of the compulsive or problematic behavior, (2) drive or urge to engage in the compulsive or problematic behavior, (3) abstinence from the compulsive or problematic behavior, (4) relapse into the compulsive or problematic behavior, (5) severity of symptoms of the compulsive or problematic behavior, (6) time spent on the compulsive or problematic behavior; (7) quality of life, including subjective sleep; and (8) psychosocial functioning.

E. THERAPEUTIC OUTCOMES

In some embodiments, the disclosed methods, such as in the eight-week or 13-week MDMA-assisted therapy course, or other embodiments disclosed herein, will provide one or more therapeutic effects for a patient or a subject diagnosed with or having an SUD, such as AUD, or symptoms thereof, or otherwise in need of such treatment. In some embodiments, the disclosed methods will result in one or more of: (a) a reduction of alcohol or other substance use (according to the SUD/SUDs being treated), (b) a reduction of alcohol or other substance cravings, (c) a promotion of alcohol or other substance abstinence, (d) a prevention of relapse into alcohol or other substance use, or (e) an improvement of at least one symptom of AUD or other SUD. Similar therapeutic effects, such as reductions in symptoms, will be understood for behavioral addictions.

In some embodiments, the disclosed methods will result in one or more of: (a) an increase in quality of life, (b) an increase in psychosocial functioning, (c) a decrease in use or frequency of a prescription medication, (d) a decrease in use or frequency of a recreational drug, (e) a decrease in obsessive compulsive thoughts, (f) a decrease in suicidality, (g) an increase in feelings of empathy, or (h) an increase in self-compassion. In some embodiments, the disclosed methods will result in an improvement of at least one symptom of a comorbid psychiatric disorder, such as antisocial personality disorder, borderline personality disorder, depression, anxiety, schizophrenia, attention deficit hyperactivity disorder, bipolar disorder, obsessive compulsive disorder, binge eating disorder, or PTSD.

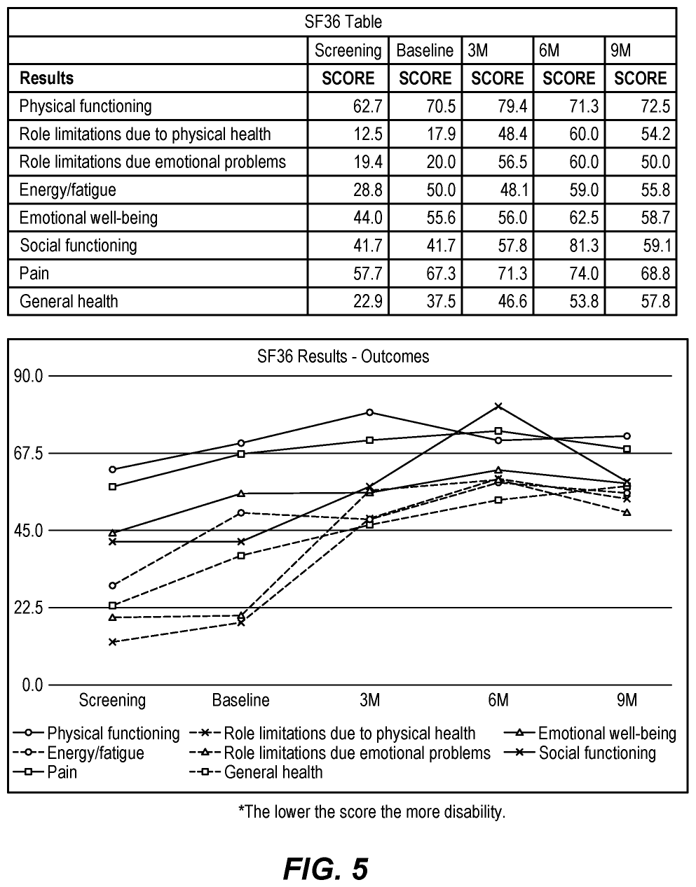

In some embodiments, the disclosed methods, such as in the eight-week or 13-week MDMA-assisted therapy (or other EAP) course, or other embodiments disclosed herein, will provide one or more therapeutic effects for a patient or a subject diagnosed with or having AUD, or symptoms thereof, or otherwise in need of such treatment, and will result in one or more of: (a) an increase in physical functioning; (b) a decrease in role limitations due to physical health; (c) an increase in emotional well-being; (d) a decrease in role limitations due to emotional problems; (e) an increase in energy; (f) a decrease in fatigue; (g) an increase in social functioning; (h) a decrease in pain; or (i) an increase in general health. In some embodiments the increase and/or the decrease will be demonstrated by a patient-reported measure on a Short Form (36) Health Survey, or as otherwise described in Sessa (2020) and shown here, for example, in FIG. 5 and accompanying description thereof.

In some embodiments, the measurement of such therapeutic effects may be performed by a therapist or other clinician (e.g., an interview), by a patient his or herself (e.g., a self-reported questionnaire), by a third-party human or by a medical or other device (e.g., a medical sensor or biosensor, a watch or fitness tracker); such measurements may be graded by a human decision-maker or an AI, by using machine learning, or by a computer algorithm.

In some embodiments, any one or more of such therapeutic effects will be durable effects, which will be shown when measured at a time following treatment by at least one month, at least three months, at least six months, at least nine months, at least one year, or greater than one year, including when measured at times in between. Such measurements “following treatment” may be measured, e.g., from the end of detoxification, if detoxification is completed, from baseline, from intake, or from the first psychotherapy session, and therefore “following treatment” may include a period measured from the start of treatment, or a point following the start of treatment, but before treatment is complete. In other embodiments, a measurement is from the conclusion of a therapy protocol, or after the conclusion of a certain or chosen phase of a therapy protocol. In other embodiments, a measurement is from the end of treatment, i.e., after treatment is complete, which in some embodiments will be from the completion of the MDMA-assisted therapy regimen.

In some embodiments, the disclosed methods will result in a reduction of alcohol or other substance use (according to the SUD or SUDs being treated). In some embodiments, a reduction of substance use may be measured by a reduction in the units of substance consumed per week (compared to the units of substance consumed per week prior to undergoing the disclosed methods). In some such embodiments, the amount of a substance of abuse consumed per patient is a durable effect, and will be shown at least 1 month, at least 3 months, at least 6 months, at least 9 months, or at least 1 year following treatment. In some embodiments, the amount of a substance of abuse consumed per patient is reduced by about 5% to about 50%, including about 5%, about 10%, about 15%, about 20%, about 25%, about 30%, about 35%, about 40%, about 45%, about 50%, and values in between. In some preferred embodiments, the amount of a substance of abuse consumed per patient is reduced by 50% or more. In some preferred embodiments, the amount of a substance of abuse consumed per patient is reduced by at least 50%, at least 55%, at least 60%, at least 65%, at least 70%, at least 75%, at least 80%, at least 85%, at least 90%, at least 95%, up to and including by 100% (i.e., a patient is no longer consuming the substance), and values in between.

In some embodiments, where the SUD is AUD, a reduction of substance use is a reduction of alcohol use, and such reduction in alcohol use may be measured by a reduction in the units of alcohol consumed per week (compared to the units of alcohol consumed per week prior to undergoing the disclosed methods), wherein a unit of alcohol refers to a standard drink, which contains about 14 g of pure alcohol (ethanol). For typical alcoholic beverages, this refers to 12 ounces of 5% alcohol beer, 5 ounces of 12% wine, and 1.5 ounces of 40% distilled spirits (NIH). In some such embodiments, the amount of alcohol consumed per patient is a durable effect, and will be shown at least 1 month, at least 3 months, at least 6 months, at least 9 months, or at least 1 year following treatment. In some embodiments, the amount of alcohol consumed per patient is reduced by about 5% to about 50%, including about 5%, about 10%, about 15%, about 20%, about 25%, about 30%, about 35%, about 40%, about 45%, about 50%, and values in between. In some preferred embodiments, the amount of alcohol consumed per patient is reduced by 50% or more. In some preferred embodiments, the amount of alcohol consumed per patient is reduced by at least 50%, at least 55%, at least 60%, at least 65%, at least 70%, at least 75%, at least 80%, at least 85%, at least 90%, at least 95%, up to and including by 100% (i.e., a patient is no longer consuming alcohol), and values in between.

In some embodiments, the disclosed methods will result in a reduction in substance cravings. In some embodiments, a reduction in substance cravings may be measured by subjective patient reports, as well as questionnaires, such as but not limited to the substance craving experience questionnaire. In some such embodiments, the reduction in substance cravings is a durable effect, and will be shown at least 1 month, at least 3 months, at least 6 months, at least 9 months, or at least 1 year following treatment. In some embodiments, substance cravings will be reduced by about 5% to about 100%. In some embodiments, substance cravings will be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including by 100% (i.e., the patient is no longer having substance cravings), and values in between.

In some embodiments, where the SUD is AUD, the disclosed methods will result in a reduction in alcohol cravings. In some embodiments, a reduction in alcohol cravings may be measured by subjective patient reports, as well as questionnaires, such as but not limited to the alcohol craving experience questionnaire. In some such embodiments, the reduction in alcohol cravings is a durable effect, and will be shown at least 1 month, at least 3 months, at least 6 months, at least 9 months, or at least 1 year following treatment. In some embodiments, alcohol cravings will be reduced by about 5% to about 100%. In some embodiments, alcohol cravings will be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including by 100% (i.e., the patient is no longer having alcohol cravings), and values in between.

In some embodiments, the disclosed methods will result in a promotion of abstinence, wherein abstinence refers to a prolonged period of time-such as at least 1 day, at least 2 days, at least 3, at least 4 days, at least 5, at least 6 days, at least 7 days, or more than 7 days—wherein the patient does not consume a substance of abuse, such as alcohol. In some embodiments, a promotion of abstinence may be measured by measuring the average duration of abstinence (e.g., the average time between “heavy drinking days,” as defined herein), before undergoing the disclosed methods, and after undergoing the disclosed methods. In some such embodiments, the promotion of abstinence is a durable effect, and will be shown at least 1 month, at least 3 months, at least 6 months, at least 9 months, or at least 1 year following treatment. In some embodiments, the promotion of abstinence will be illustrated by an average increase in time between “heavy drinking days,” as defined herein, by at least about 5%, preferably by at least 25%, more preferably by at least 50%, and most preferably by at least 100%. In some embodiments, the promotion of abstinence will be illustrated by an average increase in time between “heavy drinking days,” as defined herein, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%, and values in between. In some most preferred embodiments, the promotion of abstinence will be illustrated by an average increase in time between “heavy drinking days,” as defined herein, by at least 100%, such as at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, and, in some embodiments, the promotion of abstinence will be illustrated by an average increase in time between “heavy drinking days,” as defined herein, by more than about 1000%, as well as values in between.

In some embodiments, the disclosed methods will result in a prevention of relapse into substance use. In some embodiments, a prevention of relapse into substance use may be assessed by measuring the amount of the substance consumed after undergoing the disclosed methods. In some such embodiments, the prevention of relapse into substance use is a durable effect, and will be shown at least 1 month, at least 3 months, at least 6 months, at least 9 months, or at least 1 year following treatment. In embodiments, a prevention of relapse into substance use will be a reduction in the rate of relapse or the likelihood of relapse, for example, a reduction of about 5% to about 100%. In embodiments, a reduction in rate or likelihood of relapse will be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including 100% (i.e., complete prevention of relapse), and values in between.

In some embodiments, where the SUD is AUD, the disclosed methods will result in a prevention of relapse into alcohol use. In embodiments, a prevention of relapse into alcohol use is assessed by measuring the amount of alcohol consumed after undergoing the disclosed methods, and identifying whether or not the amount of alcohol consumed, and frequency with which it is consumed, is above the threshold to qualify the person as a heavy drinker, wherein a “heavy drinker” refers to an individual who “binge drinks” at least 5 or more days in a given month, and “binge drinking” refers to a male consuming 5 or more alcoholic drinks on the same occasion, or a female consuming 4 or more drinks on the same occasion. In some such embodiments, the prevention of relapse into alcohol use is a durable effect, and will be shown at least 1 month, at least 3 months, at least 6 months, at least 9 months, or at least 1 year following treatment.

In some embodiments, where a male patient has AUD, the patient will consume 5 or more drinks on the same occasion less than 5 times in a given month, for at least 1 month, at least 2 months, at least 3 months, at least 4 months, at least 5 months, at least 6 months, at least 7 months, at least 8 months, at least 9 months, at least 10 months, at least 11 months, or at least 12 months following the completion of the disclosed methods. In some embodiments, where a female patient has AUD, the patient will consume 4 or more drinks on the same occasion less than 5 times in a given month, for at least 1 month, at least 2 months, at least 3 months, at least 4 months, at least 5 months, at least 6 months, at least 7 months, at least 8 months, at least 9 months, at least 10 months, at least 11 months, or at least 12 months following the completion of the disclosed methods. In some embodiments, a prevention of relapse into alcohol use will be a reduction in the rate of relapse or the likelihood of relapse, for example, a reduction of about 5% to about 100%. In some embodiments, a reduction in rate or likelihood of relapse will be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including 100% (i.e., complete prevention of relapse), and values in between.

In some embodiments, the disclosed methods will result in an increase in quality of life. In some embodiments, an increase in quality of life may be measured by subjective reports from patients, questionnaires, such as, but not limited to, the Quality of Life Scale (QoLS), the Health Related Quality of Life (HRQoL) questionnaire, World Health Organization Quality of Life instruments, for example, WHOQOL-100 and its abbreviated version, WHOQOL-BREF, the Munich Quality-of-life Dimension List (MLDL), Medical Outcomes Study Short Form (SF-36), the Quality of Life Enjoyment and Satisfaction Questionnaire (Q-LES-Q), the EuroQoL-5D (EQ-5D), the Life Situation Survey (LSS), the Nottingham Health Profile (NHP), the Sleep Quality Scale (SQS), the Pittsburgh Sleep Quality Index (PSQI), and the Leeds Sleep Evaluation Questionnaire (LSEQ), as well as changes in mental health conditions, including but not limited to anxiety and depression (i.e., after completing the disclosed methods, confirming the severity of the patient's anxiety and/or depression has decreased compared to the severity of the same prior to undergoing the disclosed methods). In some embodiments, anxiety and/or depression are assessed using questionnaires, for example, the Patient Health Questionnaire (PHQ-9) or the Generalized Anxiety Disorder Assessment (GAD-7). In some such embodiments, the increase in the patient's quality of life is a durable effect, and will be shown at least 1 month, at least 3 months, at least 6 months, at least 9 months, or at least 1 year following treatment. In some embodiments, the patient's quality of life will be increased by between about 5%, to about 100%. In some embodiments, the patient's quality of life will be increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, and value in between. In some embodiments, wherein the patient's quality of life is determined by a reduction in depression, the patient's depression may decrease by between about 5%, to about 100%. In some embodiments, wherein the patient's quality of life is determined by a reduction in depression, the patient's depression may decrease by between about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, and values in between (i.e., the patient no longer has quantifiable depression). In some embodiments, wherein the patient's quality of life is determined by a reduction in anxiety, the patient's anxiety may decrease by between about 5%, to about 100%. In some embodiments, wherein the patient's quality of life is determined by a reduction in anxiety, the patient's anxiety may decrease by between about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including 100% (i.e., the patient no longer has quantifiable anxiety), and values in between.

As shown in FIG. 3, the disclosed methods are effective in reducing mild, moderate, moderately severe, and severe depression, which in embodiments may be a comorbid mental health disorder. Prior to undergoing the disclosed methods, specifically during the screening session, 12 patients were assessed for levels of depression. It was found that one patient suffered from mild depression, two suffered from moderate depression, four suffered from moderately severe depression, and five suffered from severe depression. Directly after going through the detoxification process (noted as “baseline” in FIG. 3), one patient had mild depression, two had moderate depression, zero had moderately severe depression, and two had severe depression (out of 11 patients remaining in the study). The patients were then reassessed at 3, 6, and 9 months post-baseline (i.e., after completing a detoxification course, and prior to beginning the MDMA-assisted therapy regimen), wherein it was discovered that, at 3 months, two had mild depression, one had moderate depression, three had moderately severe depression, and one had severe depression (out of 9 patients remaining in the study); at 6 months, no patients suffered from mild or moderate depression, two had moderate severe depression, and one had severe depression (out of 6 patients remaining in the study); finally, at 9 months, one patient had mild depression, one had moderate depression, and one had moderate severe depression, with none having severe depression. Thus, while 100% of the patients who began the study were diagnosed with depression, ranging from mild to severe, only 50% of patients remaining 9 months after completing the methods of invention were found to have depression, none of which were classified as “severe.” Thus, the disclosed methods successfully reduce both the incidence and severity of depression in patients diagnosed with AUD, and will similarly reduce the incidence and severity of depression where depression is comorbid to another disclosed condition.

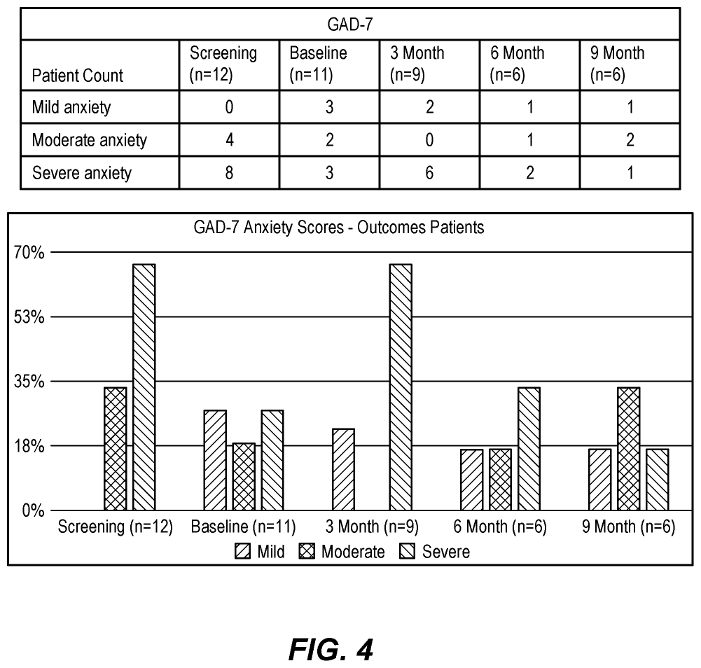

As shown in FIG. 4, the disclosed methods are effective in reducing mild, moderate, and severe anxiety, which in embodiments may be a comorbid mental health disorder. Prior to undergoing the disclosed methods, specifically during the screening session, 12 patients were assessed for levels of anxiety. It was found that four patients suffered from moderate anxiety, and eight patients suffered from severe anxiety. Directly after undergoing the detoxification process (noted as “baseline” in FIG. 4), three patients had mild anxiety, two patients had moderate anxiety, and three patients had severe anxiety (out of 11 patients remaining in the study). The patients were then reassessed at 3, 6, and 9 months post-baseline (i.e., after completing a detoxification course, and prior to beginning the MDMA-assisted therapy regimen), wherein it was discovered that, at three months, two patients had mild anxiety, zero patients had moderate anxiety, and 6 patients had severe anxiety (out of the 9 patients remaining in the study); at 6 months, one person had mild anxiety, one person had moderate anxiety, and two people had severe anxiety (out of the 6 patients remaining in the study); at 9 months, one person had mild anxiety, two people had moderate anxiety, and one person had severe anxiety (out of the 6 patients remaining in the study). Thus, while 100% of the patients who began the study were diagnosed with anxiety, ranging from mild to severe, only about 67% of the patients remaining 9 months after completing the disclosed methods were found to have anxiety, only one of which was classified as “severe.” Thus, the disclosed methods successfully reduce both the incidence and severity of anxiety in patients diagnosed with AUD, and will similarly reduce the incidence and severity of anxiety where anxiety is comorbid to another disclosed condition.

In some embodiments, the disclosed methods will result in an increase in psychosocial functioning, wherein “psychosocial functioning” refers to one's ability to perform the activities of daily life. In some embodiments, an increase in psychosocial functioning may be measured by obtaining subjective evidence from patients in the form of surveys, questionnaires, and/or other psychosocial functioning assessments, as would be apparent to one of skill. In some such embodiments, the increase in psychosocial functioning is a durable effect, and will be shown at least 1 month, at least 3 months, at least 6 months, at least 9 months, or at least 1 year following treatment. In some embodiments, the patient's psychosocial functioning will improve by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%, and values in between. In some most preferred embodiments, the patient's psychosocial functioning will improve by at least 100%, such as at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, and, in some embodiments, psychosocial functioning will improve will increase by more than about 1000%, as well as values in between.

In some embodiments, the disclosed methods will result in a decrease in use or frequency of a prescription medication. Meaning, in some embodiments, a patient decreases the frequency with which the patient takes a given prescription medication while, in some embodiments, the patient is prescribed a fewer number of medications than the patient was prescribed prior to undergoing the disclosed methods. Additionally, in some embodiments, the patient is both prescribed a fewer number of medications than the patient was prescribed prior to undergoing the disclosed methods, and decreases the frequency with which the patient takes a given prescription medication. As would be apparent to one of skill, in some embodiments, to ascertain whether a patient has decreased prescription medication use, a practitioner may ask the patient to provide information regarding the type, dosage, and frequency of administration of prescription medication. Alternatively, a practitioner may obtain current prescription records for the patient, or complete a prescription screen through urine or blood testing, as would be apparent to one of skill. In some such embodiments, the reduction in prescription medication use is a durable effect, and will be shown at least 1 month, at least 3 months, at least 6 months, at least 9 months, or at least 1 year following treatment. In some embodiments, the reduction in prescription medication use (both a decrease in use and frequency of use) persists for about 1 month, about 2 months, about 3 months, about 4 months, about 5 months, about 6 months, about 7 months, about 8 months, about 9 months, about 10 months, about 11 months, about 12 months, or more than 12 months following treatment (where “following treatment” means from the last session, including whether such session is a drug-assisted session, or a non-drug session).

In some embodiments, the disclosed methods will result in a decrease in use or frequency of at least one recreational drug. In some embodiments, a recreational drug is a prescription drug that is abused, misused, or used illicitly. Meaning, in some embodiments, a patient decreases the frequency with which the patient takes a given recreational drug, while, in some embodiments, the patient takes a fewer number of recreational drugs than the patient took prior to undergoing the disclosed methods. Additionally, in some embodiments, the patient both takes a fewer number of recreational drugs than the patient took prior to undergoing the disclosed methods, and decreases the frequency with which the patient takes a given recreational drug. In some embodiments, as would be apparent to one of skill, a decrease in use or frequency of a recreational drug may be measured by asking the patient to provide information regarding their usage of such substances. Alternatively, a practitioner may conduct a screening for recreational drugs via blood and urine testing, as would be apparent to one of skill. In some such embodiments, the reduction in recreational drug use is a durable effect, and will be shown at least 1 month, at least 3 months, at least 6 months, at least 9 months, or at least 1 year following treatment. In some embodiments, the reduction in recreational drug use (both a decrease in use and frequency of use) persists for about 1 month, about 2 months, about 3 months, about 4 months, about 5 months, about 6 months, about 7 months, about 8 months, about 9 months, about 10 months, about 11 months, about 12 months, or more than 12 months following treatment.

In some embodiments, the disclosed methods will result in a reduction in obsessive compulsive thoughts. In some embodiments, the reduction in obsessive compulsive thoughts may be measured by obtaining subjective reports from a patient, wherein the patient discusses the frequency and severity of obsessive compulsive thoughts; by administering to the patient at least one questionnaire or survey, wherein the at least one questionnaire or survey assesses the frequency and/or severity of the patient's obsessive compulsive thoughts; and/or having the patient log the frequency and severity of obsessive compulsive thoughts throughout the duration of the disclosed methods, so a practitioner may objectively determine whether or not the frequency and/or severity of such obsessive compulsive thoughts has reduced. In some such embodiments, the reduction in obsessive compulsive thoughts is a durable effect, and will be shown at least 1 month, at least 3 months, at least 6 months, at least 9 months, or at least 1 year following treatment. In some embodiments, the reduction in obsessive compulsive thoughts will be reduced by between about 5%, to about 100%. In some embodiments, the reduction in obsessive compulsive thoughts will be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including 100% (i.e., the patient has substantially no obsessive compulsive thoughts), and values in between.

In some embodiments, the disclosed methods will result in a decrease in suicidality. In some embodiments, a decrease in suicidality may be measured by obtaining subjective reports from a patient, wherein the patient discusses the frequency with which they experience suicidal thoughts; by administering to the patient at least one questionnaire and/or survey, wherein the at least one questionnaire and/or survey is capable of quantifying the suicidality of the patient; administering a suicide risk assessment test, suicide ideation risk assessment, or a suicide severity rating scale, such as the Columbia suicide severity rating scale (C-SSRS); and/or having the patient log the frequency of suicidal thoughts prior to, during, and after completing the disclosed methods, so that a practitioner may objectively determine weather or not the patient experienced a reduction in suicidality as a result of the disclosed methods. In some such embodiments, the reduction in suicidality is a durable effect, and will be shown at least 1 month, at least 3 months, at least 6 months, at least 9 months, or at least 1 year following treatment. In some embodiments, suicidality will be reduced by between about 5%, to about 100%. In some embodiments, suicidality will be reduced by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, up to and including 100% (i.e., the patient has substantially no suicidal thoughts), and values in between.

In some embodiments, the disclosed methods will result in an increase in feelings of empathy. In some embodiments, the increase in feelings of empathy may be measured by obtaining subjective reports from a patient, wherein the patient discusses the changes in empathetic feelings and actions they have experienced since completing the disclosed methods; and/or by utilizing questionnaires and/or surveys, wherein the questionnaires and/or surveys assess the subject's empathy through a series of questions. In such embodiments, a practitioner may assess the patient's level of empathy prior to, during, and after undergoing the disclosed methods, to determine whether the patient has experienced an increase in feelings of empathy as a result of the disclosed methods. In some such embodiments, the increase in feelings of empathy is a durable effect, and will be shown at least 1 month, at least 3 months, at least 6 months, at least 9 months, or at least 1 year following treatment. In some embodiments, feelings of empathy will increase by at least about 5%, preferably by at least 25%, more preferably by at least 50%, and most preferably by at least 100%. In some embodiments, feelings of empathy will increase by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%, and values in between. In some most preferred embodiments, feelings of empathy will increase by at least 100%, such as at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, and, in some embodiments, self-compassion will increase by more than about 1000%, as well as values in between.

In some embodiments, the disclosed methods will result in an increase in self-compassion. In some embodiments, the increase in self-compassion may be measured by obtaining subjective reports from a patient, wherein the patient discusses the changes in self-compassion that they have experienced since completing the disclosed methods; and/or by utilizing questionnaires and/or surveys, wherein the questionnaires and/or surveys assess the subject's self-compassion through a series of questions. In such embodiments, a practitioner may assess the patient's self-compassion prior to, during, and after undergoing the disclosed methods, to determine whether the patient has experienced an increase in self-compassion as a result of the disclosed methods. In some such embodiments, the increase in self-compassion is a durable effect, and will be shown at least 1 month, at least 3 months, at least 6 months, at least 9 months, or at least 1 year following treatment. In some embodiments, self-compassion will increase by at least about 5%, preferably by at least 25%, more preferably by at least 50%, and most preferably by at least 100%. In some embodiments, self-compassion will increase by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, and about 100%, and values in between. In some most preferred embodiments, self-compassion will increase by at least 100%, such as at least about 150%, at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 850%, at least about 900%, at least about 950%, at least about 1000%, and, in some embodiments, self-compassion will increase by more than about 1000%, as well as values in between.

In some embodiments, the disclosed methods will result in an improvement of at least one symptom of a comorbid psychiatric disorder, wherein the comorbid psychiatric disorder includes, but is not limited to, antisocial personality disorder, borderline personality disorder, depression, anxiety, schizophrenia, attention deficit hyperactivity disorder, bipolar disorder, obsessive compulsive disorder, binge eating disorder, and PTSD. In some embodiments, the improvement of at least one symptom of a comorbid psychiatric disorder may be measured by obtaining subjective reports from a patient, wherein the patient discusses any changes in the at least one symptom of a comorbid psychiatric disorder that they have experienced since completing the disclosed methods; by utilizing questionnaires and/or surveys, wherein the questionnaires and/or surveys assess the severity of the at least one symptom of a comorbid psychiatric disorder through a series of questions; and/or obtaining objective measurements, as disclosed herein. In some such embodiments, the improvement in at least one symptom of a comorbid psychiatric disorder is a durable effect, and will be shown at least 1 month, at least 3 months, at least 6 months, at least 9 months, or at least 1 year following treatment. In some embodiments, at least one symptom of a comorbid psychiatric disorder will improve by between about 5%, to about 100%. In some embodiments, at least one symptom of a comorbid psychiatric disorder will improve by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, and values in between.

a. Assessments of Quality of Life and Psychosocial Functioning

In some embodiments, quality of life and/or psychosocial functioning are evaluated, monitored, or rated to assess therapeutic outcomes in response to a treatment intervention, such as EAP or MDMA-assisted therapy. In some embodiments, quality of life and/or psychosocial functioning can be assessed at one or more points in time prior to, during, after completion of a treatment, such as EAP. In some embodiments, quality of life and/or psychosocial functioning can be assessed using subject-reported and/or interviewer-reported questionnaires. Such questionnaires include inquiries related to one or more aspects of a subject's physical, social, and emotional well-being. In some aspects, quality of life and psychosocial functioning assessments can share commonalities, especially when related to a subject's ability to perform the activities of daily living, a subject's engagement in relationships and satisfaction associated with the same, and the interactions of the subject with their workplace and/or community.

In some examples, quality of life and psychosocial functioning can be assessed by questions related to health or improvement of a condition, for example, an SUD such as AUD, or a behavioral addiction, or a mental health disorder. Examples of quality of life and psychosocial functioning questionnaires and their contents are described below for illustrative purposes and should not be construed as limiting. In some cases, the described questionnaires have been used in studies of subjects who display alcohol abuse and dependence (Ugochukwu, 2013). In embodiments, assessments and questionnaires such as any one or more of the below are used.

i. Quality of Life Scale (QoLS)

In some embodiments, the disclosed methods will result in an improvement on the QoLS.

Although its inquiries are general, e.g., a patient population, the Quality of Life Scale (QoLS) has been used to evaluate quality of life and psychosocial functioning of subjects with various disease states (Burckhardt, 2003). The QoLS may be self-administered or administered by an interviewer. If the interview format is chosen, subjects are provided with a copy of a scale to refer to when deciding on the most appropriate response. Updated versions of the QoLS employ a 7-point scale to rate subject satisfaction. On the 1 to 7 scale, 1 corresponds to “terrible,” 2 corresponds to “unhappy,” 3 corresponds to “mostly dissatisfied,” 4 corresponds to “mixed,” 5 corresponds to “mostly satisfied,” 6 corresponds to “pleased,” and 7 corresponds to “delighted” (Burckhardt, 2003).

Subjects are asked to rate various aspects of life, including material and physical well-being, health, relationships with parents, siblings and other relatives, having and raising children, relationship with spouse or significant other, relationships with friends, helping and encouraging others, participating in organizations and public affairs, intellectual development, understanding of self, occupational role, creativity/personal expression, socializing, passive and observational recreation, active and participatory recreation, and independence (doing for yourself). QOLS scores are added across domains, and a greater total score indicates a higher quality of life.

ii. Health Related Quality of Life (HRQoL)

In some embodiments, the disclosed methods will result in an improvement on the HRQoL.

The Health Related Quality of Life (HRQoL) questionnaire probes the impact of health on a subject's quality of life and psychosocial functioning. Subjects respond with graded, i.e. on a point scale, or non-graded responses to questions in three domains: healthy days, activity limitations, and healthy day symptoms (CDC, 2016). Examples of non-graded responses include providing the number of days physical health was not good, the number of days mental health was not good, and the number of days when health interfered with daily activities. Questions related to activity limitations evaluate the ability to fulfill personal care needs and the demands of a subject's routine. Healthy day symptoms include inquiries into pain, depression, anxiety, rest and sleep, and a general assessment of health and energy level (Stanford Sparqtools: Health-Related Quality of Life Scale).

iii. World Health Organization Quality of Life

In embodiments, the disclosed methods will result in an improvement on the WHOQOL.

World Health Organization Quality of Life instruments (WHOQOL) include, for example, the WHOQOL-100 and its abbreviated version, WHOQOL-BREF (26 items). The WHOQOL was developed by fifteen international field centres to develop a ross-culturally applicable quality of life assessment. The surveys may be self-administered or interviewer-administered. The WHOQOL-BREF prompts scoring of four domains related to quality of life: physical health, psychological, social relationships, and environment. It also includes overall quality of life and general health. Though abbreviated, results of the WHOQOL-BREF correlate highly to WHOQOL-100 domain scores, as calculated on a four domain structure (Development of the World Health Organization WHOQOL-BREF quality of life assessment, 1998).

In brief, scoring of WHOQOL-BREF entails examining two items separately (an individual's overall perception of quality of life and an individual's overall perception of their health) in addition to examining the four domains. The four domain scores denote an individual's perception of quality of life in each particular domain. Higher domain scores denote higher quality of life. The average score of items within each domain is used to calculate the domain score. If it is desired to compare domain results to those of the WHOQOL-100, the mean scores are then multiplied by 4 (WHO, 1996).

iv. Munich Quality-of-Life Dimension List (MLDL)

In some embodiments, the disclosed methods will result in an improvement on the MLDL.

The Munich Quality-of-life Dimension List (MLDL) allows a subject to self-assess the level of personal satisfaction in all basic areas of life. The MLDL includes 19 different items relevant to quality of life, including mental, physical, social and everyday-life dimensions during the last week on one overall scale and four subscales using Likert scales ranging from 0 (very unsatisfied/very unimportant) to 10 (very satisfied/very important).

Exemplary quality of life domains that may be assessed include “overall,” “physical aspects,” “psychological aspects,” “social life,” and “everyday life.” In addition to rating one's satisfaction, the scale also prompts the respondent to evaluate the personal relevance of every item using Likert scales from 0 (very unimportant) to 10 (very important). A calculation algorithm may be used to determine relevance-weighted satisfaction scores. In the absence of differences in relevance ratings in the group comparisons of interest, unweighted satisfaction scores have been used (Frischknecht, 2013).

v. Medical Outcomes Study Short Form (SF-36)

In some embodiments, the disclosed methods will result in an improvement on the SF-36.

The Medical Outcomes Study Short Form (SF-36) is an example of a questionnaire that can be used as a self-reported and an observer-reported survey. The survey was designed for self-administration by persons 14 years of age and older, and for administration by a trained interviewer in person or by telephone. The SF-36 evaluates eight health concepts on one multi-item scale: 1) limitations in physical activities because of health problems; 2) limitations in social activities because of physical or emotional problems; 3) limitations in usual role activities because of physical health problems; 4) bodily pain; 5) general mental health, e.g., psychological distress and well-being; 6) limitations in usual role activities because of emotional problems; 7) vitality, e.g., energy and fatigue; and 8) general health perceptions (Ware, 1992). Subsequent versions of the SF-36 have been developed that vary in length, e.g., the SF-6D and the SF-12, and rating scale, for example, reducing six-level response scales to a five-level scale.

A high or relatively higher SF-36 score indicates a better health state. After the survey has been completed, items and scales are scored in three steps: 1) item recoding, for the 10 items that require recoding; 2) computing scale scores by summing across items in the same scale (raw scale scores); and 3) transforming raw scale scores to a 0-100 scale (transformed scale scores). It is recommended that recoding and scale scoring be performed by a computer using the appropriate algorithms (Ware, 1993).

vi. Quality of Life Enjoyment and Satisfaction Questionnaire (Q-LES-Q)

In embodiments, the disclosed methods will result in an improvement on the Q-LES-Q.

The Quality of Life Enjoyment and Satisfaction Questionnaire (Q-LES-Q) prompts the respondent to rate satisfaction with various aspects of life in the past week, including mood, health, relationships, and economic status, among others. A numeric scale is correlated with qualitative measurements, for example, 1=Very Poor to 5=Very Good. Higher scores indicate greater satisfaction with different aspects of life.

vii. EuroQoL-5D (EQ-5D)

In some embodiments, the disclosed methods will result in an improvement on the EQ-5D.

The EuroQoL-5D (EQ-5D) assesses five domains: mobility, usual activities, self-care, pain or discomfort, and anxiety or depression. Responses in each domain are rated on a scale from 1 to 3, where 1 indicates no problems, 2 indicates moderate problems, and 3 indicates extreme problems. The ratings in each domain constitute the five-digit number collectively known as the EQ-5D self-reported health state (Gunther, 2007).

viii. Life Situation Survey (LSS)

In some embodiments, the disclosed methods will result in an improvement on the LSS.

The Life Situation Survey (LSS) is a generic scale that assesses an individual's current functioning using 20 QoL indicators, including work, leisure, nutrition, sleep, social nurturance, earnings, health, love/affection, environment, self-esteem, security, public support, stress, mobility, autonomy, energy level, social support, mood/affect, outlook, and egalitarianism. Variables are rated on a scale from 1 to 7, with total scores ranging from 20 to 140. Scores of less than 90 correspond to a poor quality of life, which can be defined as greater psychosocial impairment (Foster, 2000).

ix. Nottingham Health Profile (NHP)

In some embodiments, the disclosed methods will result in an improvement on the NHP.

The Nottingham Health Profile (NHP) is a self-evaluated questionnaire that measures a subject's perceived physical health and emotional and social problems. This scale evaluates the domains of energy, physical functioning/mobility, pain, sleep, affective control, and social isolation. Each section is rated with a score from 0 to 100, where 100 represents the most severe psychosocial/health-related dysfunction (Hunt, 1980).

x. Clinical Global Impression—Improvement Scale (CGI-I)

In some embodiments, the disclosed methods will result in an improvement on the CGI-I.

The Clinical Global Impression—Improvement Scale (CGI-I) facilitates an interviewer's or an observer's, e.g., a clinician's, rating of a subject's global functioning prior to and after initiating a treatment intervention. This facilitates tracking of clinical progress over time. All available information, including a knowledge of the patient's history, psychosocial circumstances, symptoms, behavior, and the impact of the symptoms on the subject's ability to function can be considered when rating using the CGI-I. The CGI-I comprises two companion one-item measures that evaluate the severity of psychopathology on a scale from 1 to 7 and changes from the initiation of treatment on a similar seven-point scale (Busner, 2007).

xi. Sleep Quality Scale (SQS)

In some embodiments, the disclosed methods will result in an improvement on the SQS.

The Sleep Quality Scale (SQS) consists of 28 items to self-assess six domains of sleep quality, including daytime symptoms, restoration after sleep, problems initiating and maintaining sleep, difficulty waking, and sleep satisfaction. Respondents use a four-point, Likert-type scale to indicate the frequency of certain sleep behaviors, i.e., 0=“few,” 1=“sometimes,” 2=“often,” and 3=“almost always”. Scores on items belonging to factors 2 and 5 (restoration after sleep and satisfaction with sleep) are reversed before being tallied. Total scores can range from 0 to 84, with higher scores indicative of acute sleep problems (Yi, 2006).

xii. Pittsburgh Sleep Quality Index (PSQI)

In some embodiments, the disclosed methods will result in an improvement on the PSQI.

The Pittsburgh Sleep Quality Index (PSQI) is a self-rated questionnaire, which is used to assess sleep quality, including sleep disturbances, over a 1-month time interval. Nineteen individual items generate seven “component” scores in the areas of subjective sleep quality, sleep latency, sleep duration, habitual sleep efficiency, sleep disturbances, use of sleeping medication, and daytime dysfunction. The scores across the seven components are added to yield one global score (Buysse, 1989).

xiii. Leeds Sleep Evaluation Questionnaire (LSEQ)

In some embodiments, the disclosed methods will result in an improvement on the LSEQ.

The Leeds Sleep Evaluation Questionnaire (LSEQ) uses ten 100 mm visual analogue scale items in four areas related to the ease of getting to sleep (GTS), the perceived quality of sleep (QOS), the ease of awakening from sleep (AFS), and the integrity of early-morning behavior following wakefulness (BFW). A subject is instructed to answer each question by placing a vertical mark on a line, i.e., the “scale.” If no change was experienced, a subject would place a mark in the middle of the line. If a change was experienced, then the position of the subject's mark indicates the nature and extent of the change, for example, large charges are represented as near the ends of the line and small changes near the middle of the line (Parrott, 1980).

b. Assessments of Alcohol and Drug Use and Cravings

In some embodiments, use and/or cravings of alcohol and/or drugs are evaluated, monitored, or rated to assess therapeutic outcomes in response to a treatment intervention, for example, EAP or MDMA-assisted therapy. In some embodiments, aspects of alcohol and drug use and cravings can be assessed at one or more points in time prior to, during, after completion of a treatment, such as MDMA-assisted therapy. In some embodiments, alcohol and drug use and cravings are assessed using subject-reported and/or interviewer-reported questionnaires. Alcohol- and drug-related inquiries include, but are not limited to, evaluations of alcohol use, alcohol cravings, use of prescribed medication(s), number and frequency of use of recreational drugs, cravings relating to MDMA or other entactogen, and use of illicit drug following the drug-assisted therapy. Examples of instruments for monitoring or rating alcohol and drug use and cravings and their contents are described below for illustrative purposes and should not be construed as limiting.

i. Alcohol Use and Cravings

In some embodiments, the disclosed methods will result in an improvement relating to alcohol use and/or cravings. In some embodiments, alcohol acquisition and consumption can be evaluated using a variety of mediums, including interviewing or asking a subject about drinking behavior, administering a self-assessment on drinking behavior, e.g., a questionnaire, or detecting a level of alcohol, e.g., blood alcohol content, such as from a breath, body fluid, or tissue specimen. In some embodiments, cravings for alcohol include, for example, experiencing anticipation and/or desire for consuming alcohol. In some embodiments, alcohol cravings manifest as alcohol-seeking behavior. In some embodiments, cravings may be prompted or triggered by a variety of stimuli including, for example, alcohol withdrawal, the sight or smell of alcohol, interactions with people who are drinking, or even reliving a memory where drinking was involved.

In some non-limiting examples, alcohol use and/or alcohol cravings can be evaluated using the Severity of Alcohol Dependence Questionnaire (SADQ) (Stockwell, 1983), Alcohol Dependence Syndrome section of the SCID-5-CT (Clinical Trials Version) (First, 2015), Alcohol Timeline Follow Back (TLFB) (Sobell, 2001), Clinical Institute Withdrawal Assessment for Alcohol-Revised Version (CIWA-Ar) (Saitz, 1994), Penn Alcohol Craving Scale (PACS) (Flannery, 1999), and Obsessive Compulsive Drinking Scale (OCDS) (Anton, 1995). In some embodiments, the disclosed methods will result in an improvement on the SADQ, SCID-5-CT, TLFB, CIWA-Ar, PACS, or OCDS, as well as any like assessment known to those of skill.

ii. Drug Use and Cravings

In some embodiments, the disclosed methods will result in an improvement related to drug use and cravings. In some embodiments, drug use, for example use of prescription or recreational drugs, including recreational entactogens or MDMA, can be evaluated using a variety of mediums, including interviewing or asking subject's prescribing physician about prescribed medications, interviewing or asking a subject about their drug use, administering a self-assessment on drug use, e.g., a questionnaire, or detecting a level of a drug and/or its metabolite(s) using a variety of methods known to one of skill in the art from, for example, a breath, body fluid, or tissue specimen.

In some embodiments, prescription drug use can be evaluated using a variety of mediums, including interviewing or asking the subject's prescribing physician about prescribed medications and/or interviewing or asking the subject about the same. In embodiments, use of drugs, such as prescription drugs, can be evaluated using measures of adherence and/or compliance. In embodiments, measures of adherence and/or compliance include, for example, use of electronic medication packaging (EMP) devices, pill counting, clinician assessments, and self-report (Lam, 2015). In embodiments, use of drugs, such as prescription drugs, is evaluated using self-reported or interviewer-reported questionnaires. For example, the Medication Adherence Questionnaire (MAQ) (Morisky, 1986) is used to identify and predict patterns of adherence (Culig, 2014). In some embodiments, the disclosed methods will result in an improvement on the MAQ.

In some embodiments, use of drugs, such as recreational drugs, is evaluated using self-reported or interviewer-reported questionnaires. For example, the Drug Abuse Screening Test (DAST-10 and DAST-20) is a self-reported 20-item questionnaire that assesses the extent of the problems related to drug misuse, using yes or no responses. The DAST total score is computed by summing all items (Villalobos-Gallegos, 2016). In some embodiments, the disclosed methods will result in an improvement on the DAST-10 or DAST-20.

In some embodiments, cravings for drugs, such as recreational drugs, include, for example, experiencing anticipation and/or desire for consuming such substances. In some embodiments, cravings for drugs, such as recreational drugs, manifest as drug-seeking behavior. In some embodiments, drug cravings are determined using self-reported questionnaires. In some embodiments, drug cravings can be identified or measured using reinforcement “proxies,” drug self-administration, psychophysiological responding, neurobiological responding, cognitive processing, and expressive behavior (Sayette, 2000).

In some embodiments, use and/or cravings for MDMA or ecstasy are determined using one or more of the methods described herein. In some embodiments, use and/or cravings for MDMA or ecstasy are determined by asking a subject, such as a subject who will, is, or has participated in an MDMA-assisted therapy treatment regimen, about their use of MDMA and cravings for the same. In some embodiments, use and/or cravings for MDMA or ecstasy are determined using a self-reported assessment. In some embodiments, use and/or cravings for MDMA or ecstasy are determined using other methods, for example, reinforcement “proxies,” drug self-administration, psychophysiological responding, neurobiological responding, cognitive processing, and expressive behavior (Sayette, 2000).

### ADDITIONAL EXEMPLARY DISCLOSED EMBODIMENTS

In some aspects are disclosed methods of treating alcohol use disorder (AUD) in a subject diagnosed therewith, comprising facilitating an EAP regimen, such as a MDMA-assisted therapy regimen, wherein the regimen comprises one or more entactogen-assisted or MDMA-assisted therapy sessions and one or more non-drug psychotherapy sessions.

In some embodiments, the method further comprises an alcohol detoxification course prior to the entactogen-assisted or MDMA-assisted therapy regimen. In some embodiments, the method further comprises a pre-detoxification motivational group therapy course prior to the alcohol detoxification course. In some embodiments, the method further comprises a group therapy preparation course prior to the entactogen-assisted or MDMA-assisted therapy regimen.

In some aspects are disclosed methods of maintaining reduced alcohol intake in a subject diagnosed with AUD, comprising facilitating an EAP regimen, such as a MDMA-assisted therapy regimen, wherein the regimen comprises one or more entactogen-assisted or MDMA-assisted therapy sessions and one or more non-drug sessions. In embodiments, the reduced alcohol intake is maintained for at least 1 month after the subject has completed the EAP or MDMA-assisted therapy regimen. In embodiments, the reduced alcohol intake is maintained for at least 3 months after the subject has completed the EAP or MDMA-assisted therapy regimen. In embodiments, the reduced alcohol intake is maintained for at least 6 months after the subject has completed the EAP or MDMA-assisted therapy regimen. In embodiments, the reduced alcohol intake is maintained for at least 9 months after the subject has completed the EAP or MDMA-assisted therapy regimen. In embodiments, the reduced alcohol intake is maintained for at least 12 months after the subject has completed the EAP or MDMA-assisted therapy regimen.

In some embodiments, alcohol intake is reduced by at least 50%. In some embodiments, alcohol intake is reduced by at least 55%, at least 60%, at least 65%, at least 70%, at least 75%, at least 80%, at least 85%, at least 90%, at least 95%, or at least 100%. In some embodiments, the subject remains completely abstinent from alcohol.

In some aspects are disclosed methods of preventing relapse into alcohol use in a subject diagnosed with AUD, comprising facilitating an EAP regimen, such as a MDMA-assisted therapy regimen, wherein the regimen comprises one or more entactogen-assisted or MDMA-assisted therapy sessions and one or more non-drug psychotherapy sessions. In some embodiments, the relapse into alcohol use is prevented for at least 1 month after the subject has completed the EAP or MDMA-assisted therapy regimen. In some embodiments, the relapse into alcohol use is prevented for at least 3 months after the subject has completed the EAP or MDMA-assisted therapy regimen. In some embodiments, the relapse into alcohol use is prevented for at least 6 months after the subject has completed the EAP or MDMA-assisted therapy regimen. In some embodiments, the relapse into alcohol use is prevented for at least 9 months after the subject has completed the EAP or MDMA-assisted therapy regimen. In embodiments, the relapse into alcohol use is prevented for at least 12 months after the subject has completed the EAP or MDMA-assisted therapy regimen.

For each of the embodiments herein, unless context indicates otherwise, reference to an “MDMA-assisted therapy regimen” and like terms and limitations shall be understood to also refer to EAP and entactogens broadly. Thus, and as examples, reference to an “MDMA-assisted” session or “MDMA-assisted” therapy shall refer to an entactogen-assisted session or entactogen-assisted therapy (EAP) broadly, and reference to “MDMA” shall refer to any disclosed entactogen.

In some aspects are disclosed methods of treating the symptoms of a comorbid psychiatric disorder in a subject diagnosed with AUD, comprising facilitating an MDMA-assisted therapy regimen, wherein the regimen comprises one or more MDMA-assisted therapy sessions and one or more non-drug psychotherapy sessions. In some embodiments, the comorbid psychiatric disorder is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, dissociative disorders, and substance-related disorders including substance use disorder (SUD).

In some embodiments, the comorbid psychiatric disorder is depression or anxiety. In some embodiments, the comorbid psychiatric disorder is depression, and the symptoms are treated as demonstrated by a reduction in a PHQ-9 depression score. In some embodiments, the comorbid psychiatric disorder is anxiety, and the symptoms are treated as demonstrated by a reduction in a GAD-7 anxiety score. In some embodiments, the comorbid psychiatric disorder is a behavioral addiction, including any of pathological gambling, kleptomania, pyromania, compulsive buying, compulsive sexual behavior, Internet addiction, pornography, and binge eating disorder.

In some embodiments, the subject has dependent use AUD. In some embodiments, the subject has harmful use AUD. In some embodiments, the subject has alcohol abuse, alcohol dependence, alcohol addiction, or alcoholism. In some embodiments, the subject has demonstrated motivation to reduce their alcohol intake. In embodiments, the subject has successfully completed the alcohol detoxification course prior to the MDMA-assisted therapy regimen. In embodiments, the subject has successfully completed the pre-detoxification motivational group therapy course prior to the alcohol detoxification course. In embodiments, the subject has successfully completed the group therapy preparation course prior to the MDMA-assisted therapy regimen.

In some embodiments, the subject is diagnosed with AUD as defined by a version of the Diagnostic and Statistical Manual of Mental Disorders (DSM), comprising scoring above the diagnostic threshold for AUD. In some embodiments, the severity of AUD is determined using one or more questionnaires. In some embodiments, the one or more questionnaire(s) comprise the Severity of Alcohol Questionnaire (SADQ) and/or the Short Inventory of Problems for Alcohol (SIP) questionnaire. In some embodiments, the one or more questionnaire(s) comprise the Severity of Alcohol Questionnaire (SADQ). In some embodiments, the severity of AUD is scored on the SADQ as mild (score of less than 16), moderate (score of 16-30), or severe (score of greater than or equal to 31). In some embodiments, the severity of AUD is mild (score of less than 16). In some embodiments, the severity of AUD is moderate (score of 16-30). In some embodiments, the severity of AUD is severe (score of greater than or equal to 31).

In some embodiments, the method results in one or more of a reduction of alcohol use, a reduction of alcohol cravings, a promotion of alcohol abstinence, a prevention of relapse into alcohol use, or an improvement of at least one symptom of alcohol use disorder.

In some embodiments, the method results in one or more of an increase in quality of life, an increase in psychosocial functioning, a decrease in use or frequency of a prescription medication, a decrease in use or frequency of a recreational drug, a decrease in obsessive compulsive thoughts, a decrease in suicidality, an increase in feelings of empathy, or an increase in self-compassion.

In some embodiments, the method results in one or more of an increase in physical functioning, a decrease in role limitations due to physical health, an increase in emotional well-being, a decrease in role limitations due to emotional problems, an increase in energy, a decrease in fatigue, an increase in social functioning, a decrease in pain, or an increase in general health. In some embodiments, the increase and/or the decrease is demonstrated by a patient-reported measure on a Short Form (36) Health Survey. In some embodiments, the results are measured at least 1 month, at least 3 months, at least 6 months, at least 9 months, or at least 1 year from the end of detoxification, if detoxification is completed, from baseline, from the first psychotherapy session, or from the completion of the MDMA-assisted therapy regimen.

In some embodiments, the MDMA-assisted therapy regimen comprises one or more non-drug psychotherapy sessions prior to and after administration of MDMA in conjunction with psychotherapy. In some embodiments, the MDMA-assisted therapy regimen comprises more than one non-drug psychotherapy session prior to and after the one or more MDMA-assisted therapy sessions. In some embodiments, the MDMA-assisted therapy regimen comprises administering one or more doses of MDMA during the one or more MDMA-assisted therapy sessions. In some embodiments, the MDMA-assisted therapy regimen comprises repeated MDMA-assisted therapy sessions. In some embodiments, the MDMA-assisted therapy regimen comprises an initial MDMA-assisted therapy session and a subsequent MDMA-assisted therapy session. In some embodiments, the MDMA-assisted therapy regimen comprises one, two, three, or four non-drug psychotherapy sessions prior to a first dose of MDMA in conjunction with psychotherapy. In some embodiments, the MDMA-assisted therapy regimen comprises two non-drug psychotherapy sessions prior to a first MDMA-assisted therapy session. In some embodiments, the MDMA-assisted therapy regimen comprises one or more non-drug psychotherapy session(s) after the first MDMA-assisted therapy session. In some embodiments, the MDMA-assisted therapy regimen comprises one, two, three, four, five, or six non-drug psychotherapy sessions after the first MDMA-assisted therapy session and prior to a subsequent MDMA-assisted therapy session. In some embodiments, the MDMA-assisted therapy regimen comprises three non-drug psychotherapy sessions after the first MDMA-assisted therapy session and prior to the subsequent MDMA-assisted therapy session. In embodiments, the subsequent MDMA-assisted therapy session is a second, a third, a fourth, or a fifth MDMA-assisted therapy session. In embodiments, the subsequent MDMA-assisted therapy session is a second MDMA-assisted therapy session. In embodiments, the MDMA-assisted therapy regimen comprises two MDMA-assisted therapy sessions and eight non-drug psychotherapy sessions. In embodiments, the MDMA-assisted therapy regimens comprises three MDMA-assisted therapy sessions and 11 psychotherapy non-drug sessions.

In some embodiments, the one or more MDMA-assisted therapy sessions comprise administering one single dose of MDMA. In some embodiments, the first and the subsequent MDMA-assisted therapy sessions each comprise administering one single dose of MDMA. In some embodiments, the one or more MDMA-assisted therapy sessions comprise administering two doses of MDMA, an initial dose and a booster dose. In some embodiments, the first and the subsequent MDMA-assisted therapy sessions each comprise two doses of MDMA, an initial dose and a booster dose. In embodiments, the booster dose is less than the initial dose. In embodiments, the booster dose is about 10% to about 90% of the amount of the initial dose. In embodiments, the booster dose is about 25%, about 50%, or about 75% of the amount of the initial dose. In embodiments, the booster dose is about 10%, 15%, 20%, 25%, 30%, 35%, 35%, 40%, 45%, 50%, 55%, 60%, 65%, 70%, 75%, 80%, 85%, or 90% of the amount of the initial dose. In embodiments, the booster dose is administered about 30 minutes, one hour, two hours, three hours, or four hours after the initial dose. In embodiments, the booster dose is administered two hours following the initial dose, or at such time as to extend peak subjective effects or to optimize the plasma concentration-time profile.

In some embodiments, the MDMA-assisted therapy regimen comprises between 5 and 20 psychotherapy sessions, inclusive, during which at least two of said psychotherapy sessions the subject is administered MDMA. In some embodiments, a total dose of about 40 mg to 200 mg of MDMA is administered, wherein the range is inclusive. In some embodiments, a total dose of about 40 mg to 100 mg, about 100 mg to 150 mg, about 150 to 200 mg, about 175 mg to 200 mg, about 180 mg to 190 mg, about 180 to 200 mg, or about 185 to 200 mg of MDMA is administered, wherein each range is inclusive. In some embodiments, a total dose of about 180 mg, 181 mg, 182 mg, 183 mg, 184 mg, 185 mg, 186 mg, 187 mg, 188 mg, 189 mg, or 190 mg of MDMA is administered. In some embodiments, a total dose of about 187.5 mg of MDMA is administered.

In some embodiments, MDMA is administered as a pharmaceutically acceptable salt, as a free base, or as a polymorph of MDMA. In some embodiments, MDMA is administered as a racemate. In some embodiments, MDMA is administered as an enantiomerically enriched mixture of R-MDMA and S-MDMA having an enantiomeric excess in any amount greater than 0%. In some embodiments, MDMA is administered as a pure or substantially pure individual enantiomer of R-MDMA or S-MDMA having an enantiomeric excess greater than 90%.

In some embodiments, the psychotherapy in MDMA-assisted and non-drug sessions comprises motivational interviewing. In some embodiments, the psychotherapy in MDMA-assisted and non-drug sessions comprises cognitive behavioral therapy. In embodiments, the psychotherapy in MDMA-assisted and non-drug sessions comprises motivational interviewing and cognitive behavioral therapy. In some embodiments, the cognitive behavioral therapy is a modified form of cognitive behavioral therapy comprising elements of Acceptance and Commitment Therapy (ACT).

In some embodiments, the MDMA-assisted and drug-free psychotherapy sessions comprise one or more of supporting subjects to maintain and/or improve a reduction in alcohol consumption, preventing relapse into heavy drinking, and developing skills to lead a fulfilling life without problematic drinking.

In some aspects are disclosed methods of selecting a subject for MDMA-assisted therapy, comprising identifying a subject who meets one or more of the following criteria: has an AUD diagnosis, wherein the severity of AUD is mild, moderate, or severe; has completed detoxification of alcohol prior to administration of MDMA-assisted therapy; and has tapered off of medications known to interact with or attenuate the effects of MDMA prior to administration of MDMA-assisted therapy; wherein the subject does not experience adverse events associated with administration of MDMA.

In some aspects are disclosed methods of minimizing or eliminating the risk of adverse events related to MDMA-assisted therapy in a subject having AUD comprising: selecting a subject who has been diagnosed with AUD, wherein the severity of AUD is mild, moderate, or severe; facilitating detoxification of alcohol prior to administration of MDMA-assisted therapy; and tapering off medications known to interact with or attenuate the effects of MDMA prior to administration of MDMA-assisted therapy; wherein the subject does not experience adverse events associated with administration of MDMA.

In some embodiments, detoxification comprises gradually cutting down alcohol consumption over many weeks and/or is a medically assisted detoxification regime. In some embodiments, the method further comprises facilitating a pre-detoxification motivational group therapy course prior to facilitating the detoxification of alcohol.

In some aspects are disclosed methods of selecting a subject for MDMA-assisted therapy, comprising identifying a subject who meets one or more of the following criteria: has an AUD diagnosis, wherein the severity of AUD is mild, moderate, or severe; has completed a group therapy preparation course prior to administration of MDMA-assisted therapy; and has tapered off of medications known to interact with or attenuate the effects of MDMA prior to administration of MDMA-assisted therapy; wherein the subject does not experience adverse events associated with administration of MDMA. In some embodiments, the methods comprise monitoring the subject having AUD for adverse events during administration of MDMA. In some embodiments, the adverse events during administration of MDMA comprise one or more of hyperthermia, hypertension, and elevated heart rate. In some embodiments, the subject diagnosed with AUD does not display adverse events during administration of MDMA.

In some embodiments, the methods comprise monitoring the subject having AUD for adverse events after administration of MDMA. In some embodiments, the adverse events after administration of MDMA comprise mood disturbances and/or depressed affect. In some embodiments, the subject does not report a disturbance in mood or a depressed affect after administration of MDMA-assisted therapy. In some embodiments, the subject does not report a disturbance in mood or a depressed affect within about one day, two days, three days, four days, five days, six days, seven days, eight days, nine days, or ten days after administration. In some embodiments, the subject diagnosed with AUD does not display adverse events after administration of MDMA-assisted therapy.

In embodiments, the methods comprise monitoring the subject having AUD for adverse events both during and after administration of MDMA. In embodiments, the adverse events during administration of MDMA comprise one or more of hyperthermia, hypertension, and elevated heart rate, and the adverse events after administration of MDMA comprise mood disturbances and/or depressed affect. In embodiments, the subject diagnosed with AUD does not display or report adverse events during or after administration of MDMA-assisted therapy. In embodiments, the subject does not display adverse events during administration of MDMA, or report adverse events after administration of MDMA, including within about one day, two days, three days, four days, five days, six days, seven days, eight days, nine days, or ten days after administration.

In some embodiments, the methods comprise evaluating the severity of anxiety and/or depression in a subject diagnosed with AUD prior to MDMA-assisted therapy. In some embodiments, the severity of anxiety and/or depression are assessed using questionnaires. In some embodiments, the severity of anxiety and/or depression are assessed using the Generalised Anxiety Disorder Assessment (GAD-7) and/or the Patient Health Questionnaire-9 (PHQ-9) questionnaire(s). In some embodiments, the severity of anxiety and/or depression are also assessed after MDMA-assisted therapy, and are reduced after the MDMA-assisted therapy in comparison to levels preceding said MDMA-assisted therapy.

In some embodiments, AUD is diagnosed as defined by a version of the Diagnostic and Statistical Manual of Mental Disorders (DSM), wherein diagnosis comprises scoring above the diagnostic threshold for AUD. In some embodiments, the subject has dependent use AUD. In some embodiments, the subject has harmful use AUD. In some embodiments, the subject has alcohol abuse, alcohol dependence, alcohol addiction, or alcoholism.

In some embodiments are disclosed a method of treating an alcohol use disorder (AUD) subject, the method comprising: confirming the subject has alcohol use disorder; conducting a screening visit; confirming the subject meets all inclusion criteria; confirming the subject does not meet any exclusion criteria; conducting a plurality of psychotherapy sessions as part of a treatment protocol, wherein at least three of the plurality of psychotherapy sessions include at least one dose of MDMA (MDMA-assisted therapy sessions); and evaluation of results at predetermined intervals after the subject completes the treatment protocol.

In some embodiments, the treatment protocol includes between 5 and 20 sessions or between 10 and 15 sessions. In some embodiments, the treatment protocol includes 14 sessions. In some embodiments, the treatment protocol includes 3 MDMA-assisted therapy sessions, and 11 non-drug psychotherapy sessions. In some embodiments, the MDMA-assisted therapy sessions comprise at least one MDMA administration. In some embodiments, the at least one MDMA administration comprises an initial dose and optionally a booster dose. In some embodiments, the initial dose is between about 100 mg to about 150 mg, and wherein the optional booster dose is half of the initial dose. In some embodiments, the optional booster dose is administered between about 90 minutes to about 120 minutes after the initial dose. In some embodiments, the third, seventh, and eleventh sessions are MDMA-assisted therapy sessions. In some embodiments, the MDMA-assisted therapy sessions last from between about 360 minutes to about 480 minutes. In some embodiments, the evaluation of results occurs at 3 months, 6 months, and 9 months after a subject successfully completes the treatment protocol.

In some embodiments, prior to conducting a plurality of psychotherapy sessions, the subject completes a group therapy preparation course. In some embodiments, the group therapy preparation course includes a plurality of sessions. In some embodiments, each of the plurality of sessions last for about 60 minutes. In some embodiments, each of the plurality of sessions includes a discussion surrounding a specific topic. In some embodiments, the plurality of sessions includes a first session, a second session, a third session, and a fourth session. In some embodiments, the specific topic of the first session is building confidence; the specific topic of the second session is resources, barriers, and goal setting; the specific topic of the third session is increasing rewarding activities and building support; the specific topic of the fourth session is building the foundations for recovery. In some embodiments, prior to conducting a plurality of psychotherapy sessions, the subject completes a detoxification course.

In some aspects are disclosed methods of verifiably reducing alcohol consumption by a subject diagnosed with alcohol use disorder (AUD), comprising the steps of: determining the amount of alcohol consumed by the subject prior to completing the method; conducting a treatment protocol comprising 14 psychotherapy sessions, wherein 3 of the 14 psychotherapy sessions are MDMA-assisted therapy sessions, and each of the MDMA-assisted therapy sessions includes an initial dose, and optionally a booster dose, wherein the initial dose is between about 100 mg to about 150 mg, and the optional booster dose is about half of the initial dose; measuring the amount of alcohol consumed of the subject three months, six months, and nine months after the final psychotherapy session; verifying that the amount of alcohol consumed by the subject nine months after the final psychotherapy session is less than the amount consumed by the subject as determined prior to completing the method.

In some embodiments, the amount of alcohol consumed by the subject nine months after the final psychotherapy session is at least 50% less than the amount consumed by the subject as determined prior to completing the method. In embodiments, the amount of alcohol consumed by the subject nine months after the final psychotherapy session is at least 55%, at least 60%, at least 65%, at least 70%, at least 75%, at least 80%, at least 85%, at least 90%, at least 95%, or 100% less than the amount consumed by the subject as determined prior to completing the method.

In some aspects are disclosed methods of decreasing the daily alcohol consumption in a subject diagnosed with alcohol use disorder (AUD), the method comprising: completing an 11 week psychotherapy regimen comprising 14 psychotherapy sessions, wherein 3 of the 14 psychotherapy sessions are about 360 minute to about 480 minute long MDMA-assisted therapy sessions comprising the administration of two doses of MDMA, the two doses including an initial dose and a booster dose, the initial dose comprising between about 100 mg to about 150 mg of MDMA, and the booster dose, administered between about 90 minutes to about 120 minutes after the initial dose, comprising about half of the initial dose, and wherein 11 of the 14 psychotherapy sessions are non-drug psychotherapy sessions lasting about 60 minutes. In some embodiments, the MDMA-assisted therapy sessions occur once every 3 weeks for 9 weeks of the psychotherapy regimen, and the non-drug psychotherapy sessions occur once every week for all 11 weeks of the psychotherapy regimen, so that a non-dosing psychotherapy session follows an MDMA dosing session on weeks where the MDMA dosing session occurs.

In some aspects are disclosed methods of treating alcohol use disorder (AUD) in a subject diagnosed therewith comprising a psychotherapy treatment protocol comprising the steps of: conducting a screening visit, wherein the screening visit confirms the subject meets all inclusion criteria and does not meet any exclusion criteria; completion of a plurality of psychotherapy sessions as part of a treatment protocol conducted under the supervision of a dyadic therapy team, the plurality of psychotherapy sessions including: a first session, wherein the dyadic therapy pair introduces the therapy program to the subject, prepares the subject for the treatment protocol, and discusses the subject's personal story; a second session, wherein the dyadic therapy team teaches and practices a relaxation technique with the subject and reviews the subject's current recovery plan; a third session, wherein the subject is administered at least one dose of MDMA, wherein the at least one dose of MDMA is between about 100 mg to about 150 mg; a fourth session, wherein the dyadic therapist team guides the subject through a mindfulness routine, and the subject optionally provides a narrative of their experience after being administered at least one dose of MDMA; a fifth session, wherein the dyadic therapy team discusses high risk situations that may lead to a relapse, and the workability of alcohol as a control strategy, with the subject; a sixth session, wherein the dyadic therapist team practices at least one defusion technique with the subject; a seventh session, wherein the subject is administered at least one dose of MDMA, wherein the at least one dose of MDMA is between about 100 mg to about 150 mg; an eighth session, wherein the dyadic therapist team guides the subject through a mindfulness routine, and the subject optionally provides a narrative of their experience after being administered at least one dose of MDMA; a ninth session, wherein the dyadic therapist team aids the subject in identifying problematic avoidance of internal experiences and practices at least one acceptance technique with the subject; a tenth session, wherein the dyadic therapist team aids the subject in identifying at least one thing important in their life, at least one skill they possess, and at least one barrier that may impact their success; an eleventh session, wherein the subject is administered at least one dose of MDMA, wherein the at least one dose of MDMA is between about 100 mg to about 150 mg; a twelfth session, wherein the dyadic therapist team guides the subject through a mindfulness routine, and the subject optionally provides a narrative of the MDMA administration; a thirteenth session, wherein the dyadic therapist team aids the subject in developing self-compassion, recognizing urges to consume alcohol, and practicing methods of establishing everyday mindfulness; and a fourteenth session, wherein the dyadic therapist team reviews the treatment protocol with the subject, and establishes an ongoing recovery plan with the subject.

In some aspects are disclosed MDMA for use in a method of treating alcohol use disorder (AUD) in a subject diagnosed therewith, comprising a psychotherapy treatment protocol comprising the steps of: conducting a screening visit, wherein the screening visit confirms the subject meets all inclusion criteria and does not meet at least one exclusion criteria; completion of a plurality of psychotherapy sessions as part of a treatment protocol conducted under the supervision of a dyadic therapy team, the plurality of psychotherapy sessions including: a first session, wherein the dyadic therapy pair introduces the therapy program to the subject, prepares the subject for the treatment protocol, and discusses the subject's personal story; a second session, wherein the dyadic therapy team teaches and practices a relaxation technique with the subject and reviews the subject's current recovery plan; a third session, wherein the subject is administered at least one dose of MDMA, wherein the at least one dose of MDMA is between about 100 mg to about 150 mg; a fourth session, wherein the dyadic therapist team guides the subject through a mindfulness routine, and the subject optionally provides a narrative of their experience after being administered at least one dose of MDMA; a fifth session, wherein the dyadic therapy team discusses high risk situations that may lead to a relapse, and the workability of alcohol as a control strategy, with the subject; a sixth session, wherein the dyadic therapist team practices at least one defusion technique with the subject; a seventh session, wherein the subject is administered at least one dose of MDMA, wherein the at least one dose of MDMA is between about 100 mg to about 150 mg; an eighth session, wherein the dyadic therapist team guides the subject through a mindfulness routine, and the subject optionally provides a narrative of their experience after being administered at least one dose of MDMA; a ninth session, wherein the dyadic therapist team aids the subject in identifying problematic avoidance of internal experiences and practices at least one acceptance technique with the subject; a tenth session, wherein the dyadic therapist team aids the subject in identifying at least one thing important in their life, at least one skill they possess, and at least one barrier that may impact their success; an eleventh session, wherein the subject is administered at least one dose of MDMA, wherein the at least one dose of MDMA is between about 100 mg to about 150 mg; a twelfth session, wherein the dyadic therapist team guides the subject through a mindfulness routine, and the subject optionally provides a narrative of the MDMA administration; a thirteenth session, wherein the dyadic therapist team aids the subject in developing self-compassion, recognizing urges to consume alcohol, and practicing methods of establishing everyday mindfulness; and a fourteenth session, wherein the dyadic therapist team reviews the treatment protocol with the subject, and establishes an ongoing recovery plan with the subject.

In some aspects are disclosed methods of reducing the units of alcohol consumed per week in a subject diagnosed with alcohol use disorder (AUD) by between about 70% to about 100% after completing the method, the method comprising: determining the amount of alcohol consumed by the subject in units per week prior to completing the method; conducting a treatment protocol comprising 14 psychotherapy sessions, wherein 3 of the 14 sessions are MDMA-assisted therapy sessions, and each of the MDMA-assisted therapy sessions includes an initial dose, and optionally a booster dose, wherein the initial dose is between about 100 mg to about 150 mg, and the optional booster dose is about half of the initial dose; measuring the amount of alcohol consumed of the subject in units per week three months, six months, and nine months after completing the 14 psychotherapy sessions; verifying that the amount of alcohol consumed by the subject nine months after completing the 14 psychotherapy sessions is between about 70% to about 100% less than the amount consumed by the subject as determined prior to completing the method.

In some embodiments, the subject has a genetic variation associated with a substance use disorder, such as alcohol use disorder, and including a genetic variation in mGluR5 or or FKBP5.

In embodiments, the subject is administered one or more therapeutic agents in addition to MDMA. In embodiments, the subject is also administered a therapeutically effective amount of an additional active agent selected from the group consisting of: an opioid antagonist (e.g., nalmefene, naltrexone), a CB1 antagonist (e.g., rimonabant), a CRH1 receptor antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an α7 nicotinic acetylcholine receptor agonist (e.g., varenicline), a 5-HT3 antagonist (e.g., ondansetron), a 5-HT1A receptor partial agonist (e.g., aripiprazole), a 5-HT2A receptor antagonist (e.g., quetiapine, olanzapine, mirtazapine), a 5-HT reuptake inhibitor (e.g., trazodone), a SERT inhibitor (e.g., duloxetine), an α1 adrenoreceptor antagonist (e.g., doxazosin, prazosin), a glucocorticoid receptor antagonist (e.g., mifepristone), an α1 adrenoreceptor agonist (e.g., guanfacine), an AChE inhibitor (e.g., citicoline), a dopamine D2 receptor antagonist (e.g., tiapride), an α2 adrenoreceptor agonist (e.g., clonidine), an NMDA receptor antagonist (e.g., acamprosate) and an aldehyde dehydrogenase inhibitor (e.g., disulfiram), and pharmaceutically acceptable salts thereof.

In some embodiments are pharmaceutical compositions of MDMA for use in the method of the invention, wherein said composition comprises a substantially pure MDMA compound having a chromatographic purity of greater than 95% as measured by HPLC. In some embodiments, the substantially pure MDMA is substantially free of impurities or adulterants.

In some embodiments are pharmaceutical compositions of MDMA for use in the method of the invention which is a pure or substantially pure individual enantiomer of R-MDMA or S-MDMA having an enantiomeric excess greater than 90%, or an enantiomerically enriched mixture having an enantiomeric excess in any amount greater than 0%.

In some embodiments are pharmaceutical compositions of MDMA for use in the method of the invention, wherein said MDMA is in unit dosage form. In some embodiments, the unit dosage form is an immediate release, controlled release, sustained release, extended release, or modified release formulation.

In some aspects are disclosed MDMA for use in a method of reducing the units of alcohol consumed per week in a subject diagnosed with alcohol use disorder (AUD) by between about 70% to about 100% after completing the method, the method comprising: determining the amount of alcohol consumed by the subject in units per week prior to completing the method; completion by the subject of a preparation group course; conducting a treatment protocol comprising 14 psychotherapy sessions, wherein 3 of the 14 psychotherapy sessions are MDMA-assisted therapy sessions, and each of the MDMA-assisted therapy sessions includes an initial dose, and optionally a booster dose, wherein the initial dose is between about 100 mg to about 150 mg, and the optional booster dose is about half of the initial dose; measuring the amount of alcohol consumed of the subject in units per week three months, six months, and nine months after completing the 14 psychotherapy sessions; verifying that the amount of alcohol consumed by the subject nine months after completing the 14 psychotherapy sessions is between about 70% to about 100% less than the amount consumed by the subject as determined prior to completing the method.

In some aspects are disclosed MDMA for use in a method of decreasing the daily alcohol consumption in a subject diagnosed with alcohol use disorder, the method comprising: completing an 11 week psychotherapy regimen comprising 14 psychotherapy sessions, wherein 3 of the 14 psychotherapy sessions are about 360 minute to about 480 minute long MDMA-assisted therapy sessions comprising the administration of two doses of MDMA, the two doses including an initial dose and a booster dose, the initial dose comprising between about 100 mg to about 150 mg of MDMA, and the booster dose, administered between about 90 minutes to about 120 minutes after the initial dose, comprising about half of the initial dose, and wherein 11 of the 14 psychotherapy sessions are non-drug psychotherapy sessions lasting about 60 minutes.

In some embodiments are uses of MDMA for the manufacture of a medicament for a treatment for an alcohol use disorder (AUD) subject, according to the disclosed methods. In some embodiments, the AUD subject has dependent use AUD, harmful use AUD, alcohol abuse, alcohol dependence, alcohol addiction, or alcoholism.

In some embodiments are uses of MDMA in conjunction with psychotherapy to treat an alcohol use disorder (AUD) subject, according to the disclosed methods. In some embodiments are uses of MDMA for the manufacture of a medicament for a treatment to reduce alcohol use by an AUD subject, according to the disclosed methods. In some embodiments are uses of MDMA for the manufacture of a medicament for a treatment to prevent relapse into alcohol use by an AUD subject, according to the disclosed methods. In some embodiments are uses of MDMA for the manufacture of a medicament for a treatment to promote alcohol abstinence by an AUD subject, according to the disclosed methods.

In some embodiments are uses of MDMA for the manufacture of a medicament for the treatment of the symptoms of depression or anxiety associated with AUD, according to the disclosed methods. In some embodiments are uses of MDMA for the manufacture of a medicament for the treatment of the symptoms of a psychiatric disorder comorbid with AUD, according to the disclosed methods. In some embodiments, the psychiatric disorder comorbid with AUD is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, dissociative disorders, or substance-related disorders including substance use disorder (SUD). In some embodiments, the psychiatric disorder comorbid with AUD is a behavioral addiction, including any of pathological gambling, kleptomania, pyromania, compulsive buying, compulsive sexual behavior, Internet addiction, pornography, and binge eating disorder.

In some embodiments, the use of MDMA comprises monitoring adverse events during MDMA-assisted therapy. In some embodiments, the adverse events comprise one or more of hyperthermia, hypertension, and elevated heart rate. In some embodiments, adverse events are not observed during MDMA-assisted therapy.

In some embodiments, the use of MDMA comprises monitoring for adverse events after administration of MDMA-assisted therapy. In some embodiments, the adverse events after MDMA-assisted therapy comprise mood disturbances and/or depressed affect. In some embodiments, a mood disturbance and/or depressed affect after administration of MDMA-assisted therapy are not observed and/or reported. In some embodiments, mood disturbances and/or depressed affect are not observed and/or reported within about one day, two days, three days, four days, five days, six days, seven days, eight days, nine days, or ten days after MDMA-assisted therapy. In some embodiments, adverse events after administration of MDMA-assisted therapy are not observed or reported.

In some embodiments, the use of MDMA comprises monitoring for adverse events both during and after administration of MDMA. In some embodiments, the adverse events during administration of MDMA comprise one or more of hyperthermia, hypertension, and elevated heart rate, and the adverse events after administration of MDMA comprise mood disturbances and/or depressed affect. In some embodiments, no adverse events are observed or reported during or after administration of MDMA-assisted therapy. In some embodiments, no adverse events are observed or reported during administration of MDMA, or reported after administration of MDMA, including within about one day, two days, three days, four days, five days, six days, seven days, eight days, nine days, or ten days after administration.

In some aspects are disclosed methods of treating one or more comorbidities in a subject diagnosed with a condition, comprising facilitating an entactogen-assisted psychotherapy (EAP) regimen, wherein the regimen comprises one or more EAP sessions with an entactogen, and one or more non-drug psychotherapy sessions. In some embodiments, the one or more comorbidities is selected from a substance use disorder, a behavioral addiction, and a mental health disorder. In some embodiments, the substance use disorder is one or more of alcohol use disorder, opioid use disorder, nicotine dependence or tobacco use disorder, sedative, hypnotic, and anxiolytic use disorder, and stimulant use disorder. In some embodiments, the behavioral addiction is one or more of gambling disorder, gaming disorder, compulsive sexual behavioral disorder, compulsive buying-shopping disorder, technology addiction, kleptomania, internet addiction, pornography addiction, technology addiction, binge eating disorder, workaholism, perfectionism, pathological skin picking, exercise addiction, excessive tanning, tattoo addiction, masturbation addiction, sex addiction, and love addiction.

In some embodiments, the subject's condition is a mental health condition, a neurodegenerative condition, a substance use disorder, or a behavioral addiction. In embodiments, the mental health condition is selected from, or is one or more of, depression, major depressive disorder (MDD), treatment-resistant depression (TRD), atypical depression, postpartum depression, catatonic depression, a depressive disorder due to a medical condition, premenstrual dysphoric disorder, seasonal affective disorder, dysthymia, anxiety and phobia disorders, generalized anxiety disorder (GAD), agoraphobia, panic disorder, separation anxiety disorder, social anxiety disorder, post-traumatic stress disorder, adjustment disorders, feeding and eating disorders, including binge eating, bulimia, and anorexia nervosa, other binge behaviors, body dysmorphic syndromes, drug abuse or dependence disorders, disruptive behavior disorders, impulse control disorders, gaming disorders, gambling disorders, memory loss, dementia of aging, attention deficit hyperactivity disorder, personality disorders, including antisocial, avoidant, borderline, histrionic, narcissistic, obsessive compulsive, paranoid, schizoid and schizotypal personality disorders, attachment disorders, autism, social anxiety in an autistic subject, and dissociative disorders.

In some embodiments, the neurodegenerative condition is selected from, or is one or more of, adrenoleukodystrophy, AIDS dementia complex, Alexander disease, Alper's disease, Alzheimer's disease, amyloid disease, amyotrophic lateral sclerosis (ALS), ataxia telangiectasia, Batten disease, bovine spongiform encephalopathy, Canavan disease, cerebral amyloid angiopathy, cerebellar ataxia, Cockayne syndrome, corticobasal degeneration, Creutzfeldt-Jakob disease, diffuse myelinoclastic sclerosis, fatal familial insomnia, Fazio-Londe disease, Friedreich's ataxia, frontotemporal dementia or lobar degeneration, hereditary spastic paraplegia, Huntington's disease, Kennedy's disease, Krabbe disease, Lewy body dementia, Lyme disease, Machado-Joseph disease, mild cognitive impairment (MCI), motor neuron disease, movement disorders, multiple sclerosis, Multiple systems atrophy, neuroacanthocytosis, Niemann-Pick disease, Parkinson's disease, Parkinson's disease dementia, Pelizaeus-Merzbacher Disease, Pick's disease, primary lateral sclerosis including its juvenile form, prion disease, progranulinopathy, progressive bulbar palsy, progressive supranuclear palsy, protein aggregation disease, Refsum's disease including its infantile form, Sandhoff disease, Schilder's disease, spinal muscular atrophy, spinocerebellar ataxia, Steele-Richardson-Olszewski disease, subacute combined degeneration of the spinal cord, survival motor neuron spinal muscular atrophy, synucleinopathy, Tabes dorsalis, tauopathy, Tay-Sachs disease, toxic encephalopathy, transmissible spongiform encephalopathy, vascular dementia, and X-linked spinal muscular atrophy. In some embodiments, the neurodegenerative condition is selected from, or is one or more of, dementia, Alzheimer's disease, Huntington's disease, multiple sclerosis, and Parkinson's disease.

In some embodiments, the substance use disorder is selected from, or is one or more of, opioid use disorder, nicotine dependence or tobacco use disorder, sedative, hypnotic, and anxiolytic use disorder, and stimulant use disorder. In some embodiments, the substance use disorder is opioid use disorder. In some embodiments, the substance use disorder is nicotine dependence or tobacco use disorder. In some embodiments, the substance use disorder is sedative, hypnotic, and anxiolytic use disorder. In some embodiments, the substance use disorder is stimulant use disorder.

In some embodiments, the behavioral addiction is selected from, or is one or more of, gambling disorder, gaming disorder, compulsive sexual behavioral disorder, compulsive buying-shopping disorder, technology addiction, kleptomania, internet addiction, pornography addiction, technology addiction, binge eating disorder, workaholism, perfectionism, pathological skin picking, exercise addiction, excessive tanning, tattoo addiction, masturbation addiction, sex addiction, and love addiction.

In some aspects are disclosed methods of treating one or more comorbidities in a subject diagnosed with AUD, comprising facilitating an EAP regimen, wherein the regimen comprises one or more EAP sessions and one or more non-drug psychotherapy sessions. In some embodiments, the one or more comorbidities is a substance use disorder and/or a behavioral addiction.

In some embodiments, the treatment of or treating the SUD comprises any one or more of (a) reducing use of the substance, (b) reducing cravings for the substance, (c) promoting abstinence from the substance, (d) preventing relapse into use of the substance, and (e) improving at least one symptom or reducing the severity of at least one diagnostic criterion of the substance use disorder.

In some aspects are disclosed methods of treating opioid use disorder, comprising facilitating an EAP regimen, wherein the regimen comprises one or more EAP sessions and one or more non-drug psychotherapy sessions (equivalently, non-drug therapy sessions), wherein the opioid use disorder is comorbid with a condition, such as AUD. In some aspects are disclosed methods of treating nicotine dependence or tobacco use disorder, comprising facilitating an EAP regimen, wherein the regimen comprises one or more EAP sessions and one or more non-drug psychotherapy sessions, wherein the opioid use disorder is comorbid with a condition, such as AUD. In some aspects are disclosed methods of treating sedative, hypnotic, and anxiolytic use disorder, comprising facilitating an EAP regimen, wherein the regimen comprises one or more EAP sessions and one or more non-drug psychotherapy sessions, wherein the sedative, hypnotic, and anxiolytic use disorder is comorbid with a condition, such as AUD. In some aspects are disclosed methods of treating stimulant use disorder, comprising facilitating an EAP regimen, wherein the regimen comprises one or more EAP sessions and one or more non-drug psychotherapy sessions, wherein the stimulant use disorder is comorbid with a condition, such as AUD. In some aspects are disclosed methods of treating a subject having one or more of opioid use disorder, nicotine dependence and tobacco use disorder, sedative, hypnotic, and anxiolytic use disorder, and stimulant use disorder, comprising facilitating an EAP regimen, wherein the regimen comprises one or more EAP sessions and one or more non-drug psychotherapy sessions, wherein the one or more disorders is comorbid with a condition, such as AUD.

In some embodiments, the subject is diagnosed with AUD as defined by a version of the Diagnostic and Statistical Manual of Mental Disorders (DSM), comprising scoring above the diagnostic threshold for AUD. In some embodiments, the subject has dependent use AUD or harmful use AUD. In some embodiments, the severity of AUD is determined using one or more questionnaires. In some embodiments, the severity of AUD is determined using the Severity of Alcohol Questionnaire (SADQ) and/or the Short Inventory of Problems for Alcohol (SIP) questionnaire. In some embodiments, the one or more questionnaire(s) comprise the Severity of Alcohol Questionnaire (SADQ). In some embodiments, the severity of AUD is scored on the SADQ as mild (score of less than 16), moderate (score of 16-30), or severe (score of greater than or equal to 31). In some embodiments, the severity of AUD is mild (score of less than 16). In some embodiments, the severity of AUD is moderate (score of 16-30). In some embodiments, the severity of AUD is severe (score of greater than or equal to 31).

In some embodiments, the methods further comprise assessing the one or more comorbidities at one or more points following administration of the entactogen-assisted or the EAP regimen. In some embodiments, the methods further comprise assessing the one or more comorbidities prior to the entactogen-assisted or the EAP regimen.

In some embodiments, the methods further comprise a substance detoxification course prior to the entactogen-assisted or the EAP regimen. In some embodiments, the substance detoxification course comprises reducing alcohol consumption and/or completing a medically-assisted detoxification regime prior to entactogen-assisted or the EAP regimen. In some embodiments, the methods further comprise a pre-detoxification motivational group therapy course prior to the substance detoxification course. In some embodiments, the methods further comprise a group therapy preparation course prior to the EAP regimen. In some embodiments, the EAP regimen comprises one or more non-drug psychotherapy sessions prior to and after the one or more EAP sessions. In some embodiments, the EAP regimen comprises administering one or more doses of an entactogen during the one or more EAP sessions. In some embodiments, the EAP regimen comprises repeated EAP sessions. In some embodiments, the EAP regimen comprises a first EAP session and a subsequent EAP session. In some embodiments, the EAP regimen comprises one, two, three, or four non-drug psychotherapy sessions prior to the first EAP session. In some embodiments, the EAP regimen comprises two non-drug psychotherapy sessions prior to the first EAP session. In some embodiments, the EAP regimen comprises one or more non-drug psychotherapy session(s) after the first EAP session. In some embodiments, the EAP regimen comprises one, two, three, four, five, or six non-drug psychotherapy sessions after the first EAP session and prior to a subsequent EAP session. In some embodiments, the EAP regimen comprises three non-drug psychotherapy sessions after the first EAP session and prior to the subsequent EAP session. In some embodiments, the subsequent EAP session is a second, a third, a fourth, or a fifth EAP session. In some embodiments, the subsequent EAP session is the second EAP session. In some embodiments, the first and the subsequent EAP sessions each comprise administering one single dose of an entactogen per session. In some embodiments, the EAP regimen comprises two EAP sessions and eight non-drug psychotherapy sessions. In some embodiments, the EAP regimens comprises three EAP sessions and 11 psychotherapy non-drug sessions. In some embodiments, the one or more EAP sessions comprise administering one single dose of an entactogen per session. In some embodiments, the one or more EAP sessions comprise administering two doses of an entactogen, an initial dose and a booster dose. In some embodiments, the first and the subsequent EAP sessions each comprise two doses of an entactogen, an initial dose and a booster dose. In some embodiments, the booster dose is less than the initial dose. In some embodiments, the booster dose is about 10% to about 90% of the amount of the initial dose. In some embodiments, the booster dose is about 25%, about 50%, or about 75% of the amount of the initial dose. In some embodiments, the booster dose is about 10%, 15%, 20%, 25%, 30%, 35%, 35%, 40%, 45%, 50%, 55%, 60%, 65%, 70%, 75%, 80%, 85%, or 90% of the amount of the initial dose. In some embodiments, the booster dose is administered about 30 minutes, one hour, two hours, three hours, or four hours after the initial dose. In some embodiments, the booster dose two hours following the initial dose, or at such time as to extend peak subjective effects or to optimize the plasma concentration-time profile.

In some embodiments, the EAP regimen comprises between 5-20 psychotherapy sessions, inclusive, during which at least two psychotherapy sessions the subject is administered an entactogen. In some embodiments, the one or more EAP sessions comprises administering a total dose of about 40 mg to 200 mg of an entactogen, wherein the range is inclusive. In some embodiments, the one or more EAP sessions comprises administering a total dose of about 40 mg to 100 mg, about 100 mg to 150 mg, about 150 to 200 mg, about 175 mg to 200 mg, about 180 mg to 190 mg, about 180 to 200 mg, or about 185 to 200 mg of an entactogen, wherein each range is inclusive. In some embodiments, the one or more EAP sessions comprises administering a total dose of about 180 mg, 181 mg, 182 mg, 183 mg, 184 mg, 185 mg, 186 mg, 187 mg, 188 mg, 189 mg, or 190 mg of an entactogen. In some embodiments, the one or more EAP sessions comprises administering a total dose of about 187.5 mg of an entactogen.

In some embodiments, the one or more EAP sessions comprises administering an entactogen as a pharmaceutically acceptable salt, as a free base, or as a polymorph of an entactogen. In some embodiments, the one or more EAP sessions comprises administering an entactogen as a racemate. In some embodiments, the one or more EAP sessions comprises administering an entactogen as a non-racemic mixture. In some embodiments, the one or more EAP sessions comprises administering an entactogen as a single enantiomer.

In some embodiments, the psychotherapy in any one or more of the entactogen-assisted session(s) and/or the non-drug session(s) comprises cognitive behavioral therapy (CBT). In some embodiments, the CBT is a modified form of CBT comprising elements of Acceptance and Commitment Therapy (ACT). In some embodiments, the psychotherapy in any one or more of the entactogen-assisted session(s) and/or the non-drug session(s) comprises motivational interviewing.

In embodiments, the entactogen-assisted psychotherapy (EAP) is MDMA-assisted therapy.

In some embodiments, the entactogen is any of MDMA, BDB, MBDB, MDA, MDEA, 3-MMC, 4-MMC, methylone, ethylone, butylone, αET, αMT, 5-IAI, MDAI, 5-APDI, 6-APB, 5-APB, 5-MAPB, MDAI, MDOH, and 4-FA. In some embodiments, the entactogen is MDMA.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents, by reference to which in view of the disclosure each of the above as well as other benefits, advantages, and improvements of the invention will be known.

### REFERENCES

1. Anton R F, Moak D H, Latham P. (1995) The Obsessive Compulsive Drinking Scale: a self-rated instrument for the quantification of thoughts about alcohol and drinking behavior. Alcohol Clin Exp Res. 1995 February; 19(1):92-9.
2. Anton, R. F., O'Malley, S. S., Ciraulo, D. A., Cisler, R. A., Couper, D., Donovan, D. M., et al. for the COMBINE Study Research Group (2006). Combined pharmacotherapies and behavioral Interventions for alcohol dependence. The COMBINE study: a randomized controlled trial. JAMA, 293, 2003-2017.
3. APA 2013. Diagnostic and statistical manual of mental disorders: DSM-5. Washington D.C.: American Psychiatric Association.
4. Arza et al. Formulation and evaluation of swellable and floating gastroretentive ciprofloxacin hydrochloride tablets, AAPS PharmSciTech., 10(1):220-226 (2009).
5. Asami, Y., Nomura, K., Shimada, H., Ohishi, H., & Ohishi, M. (2020). Validity and reliability of the Japanese version of the kleptomania symptom assessment scale: A comparison between individuals with kleptomania and prisoners in Japan. Comprehensive psychiatry, 96, 152133. https://doi.org/10.1016/j.comppsych.2019.152133
6. Atalayer, D. et al. (2013). Ghrelin and eating disorders. Progress in neuropsychopharmacology & biologicalpsychiatry, vol. 40: 70-82. doi:10.1016/j.pnpbp.2012.08.011
7. Back-Madruga C, Boone K B, Chang L et al. (2003) Neuropsychological effects of 3,4-methylenedioxymethamphetamine (MDMA or ecstasy) in recreational users. Clin Neuropsychol 2003; 17: 446-59.
8. Barrett, F. S., Johnson, M. W., & Griffiths, R. R. (2015). Validation of the revised Mystical Experience Questionnaire in experimental sessions with psilocybin. Journal of psychopharmacology (Oxford, England), 29(11), 1182-1190
9. Beck A T, Steer R A, Brown G K. Manual for The Beck Depression Inventory Second Edition (BDI-II). San Antonio: Psychological Corporation; 1996.
10. Berge et al. (1977) “Pharmaceutical Salts,” J. Pharm. Sci. 66:1-19.
11. Bhattachary S, Powell J H (2001) Recreational use of 3,4-methylenedioxymethamphetamine (MDMA) or “ecstasy”: evidence for cognitive impairment. Psychol Med 31: 647-658.
12. Bierer, L. M. et al., Intergenerational Effects of Maternal Holocaust Exposure on FKBP5 Methylation, American Journal of Psychiatry 2020 177:8, 744-753.
13. Bishehsari, F., Magno, E., Swanson, G., Desai, V, Voigt, R. M., Forsyth, C. B., & Keshavarzian, A. (2017). Alcohol and Gut-Derived Inflammation. Alcohol research: current reviews, 38(2), 163-171.
14. Bogenschutz M P, Forcehimes A A, Pommy J A, Wilcox C E, Barbosa P C, Strassman R J. (2015) Psilocybin-assisted treatment for alcohol dependence: a proof-of-concept study. J Psychopharmacol. 2015 March; 29(3):289-99.
15. Bolton D (2008). What is Mental Disorder?: An Essay in Philosophy, Science, and Values. OUP Oxford. p. 6. ISBN 978-0-19-856592-5.
16. Böthe, Beita & Tóth-Kirily, István & Zsila, Ágnes & Griffiths, Mark & Demetrovics, Zsolt & Gibor, Orosz. (2017). The Development of the Problematic Pornography Consumption Scale (PPCS). The Journal of Sex Research. 55. 10.1080/00224499.2017.1291798.
17. Bouju et al., Journal of Gambling Studies, 2014; 30(2):349-367.
18. Bowen, S., Chawla, N., Collins, S. E., Witkiewitz, K., Hsu, S., Grow, J., Clifasefi, S., Garner, M., Douglass, A., Larimer, M. E., & Marlatt, A. (2009). Mindfulness-based relapse prevention for substance use disorders: a pilot efficacy trial. Substance abuse, 30(4), 295-305. https://doi.org/10.1080/08897070903250084.
19. Brunner D, Hen R. (1997) Insights into the neuro-biology of impulsive behavior from serotonin receptor knockout mice. Ann NY Acad Sci 1997; 836:81-105.
20. Burckhardt, C. S., & Anderson, K. L. (2003). The Quality of Life Scale (QOLS): reliability, validity, and utilization. Health and quality of life outcomes, 1, 60
21. Busner, J., & Targum, S. D. (2007). The clinical global impressions scale: applying a research tool in clinical practice. Psychiatry (Edgmont (Pa.: Township)), 4(7), 28-37.
22. Buysse D J, Reynolds C F 3rd, Monk T H, Berman S R, Kupfer D J. (1989) The Pittsburgh Sleep Quality Index: a new instrument for psychiatric practice and research. Psychiatry Res. 1989 May; 28(2):193-213.
23. Calado F, Griffiths M. D. Problem gambling worldwide: an update and systematic review of empirical research (2000-2015). J Behav Addict. (2016) 5:592-613. 10.1556/2006.5.2016.073.
24. Carhart-Harris R L et al. (2013) The effect of MDMA on recollecting autobiographical memories: an fMRI study with implications for MDMA-assisted therapy. Int J Neuropsychopharmacology: Online: http://www.ncbi.nlm.nih.gov/pubmed/24345398
25. Carhart-Harris R L, Murphy K, Leech R, Erritzoe D, Wall M B, Ferguson B, Williams L T J, Roseman L, Brugger S, De Meer I, Tanner M, Tyacke R, Wolff K, Sethi A, Bloomfield M A P, Williams T M, Bolstridge M, Stewart L, Morgan C, Newbould R D, Feilding A, Curran H V, Nutt D J et al. (2015) The Effects of Acutely Administered 3,4-Methylenedioxymethamphetamine on Spontaneous Brain Function in Healthy Volunteers Measured with Arterial Spin Labeling and Blood Oxygen Level-Dependent Resting State Functional Connectivity, BIOLOGICAL PSYCHIATRY, Vol: 78, Pages: 554-562
26. Carhart-Harris R L, Wall M B, Erritzoe D, Kaelen M, Ferguson B, De Meer I, Tanner M3 Bloomfield M, Williams T M, Bolstridge M, Stewart L, Morgan C, Newbould R D, Feilding A, Curran H V, Nutt D J (2013) The effect of acutely administered MDMA on subjective and BOLD fMRI responses to favourite and worst autobiographical memories. Int J of Neuropsychopharmacology 17: 527-40
27. Cargill Z, Kattiparambil S, Hansi N, et al (2020) Severe alcohol-related liver disease admissions post-COVID-19 lockdown: canary in the coal mine? Frontline Gastroenterology Published Online First: 26 Oct. 2020. doi: 10.1136/flgastro-2020-101693.
28. Carvalho M, Pontes H, Remiio F, Bastos M L, Carvalho F. Mechanisms underlying the hepatotoxic effects of ecstasy. Curr Pharm Biotechnol. 2010 August; 11(5):476-95. doi: 10.2174/138920110791591535. PMID: 20420570.
29. Cash, H., Rae, C. D., Steel, A. H., & Winkler, A. (2012). Internet Addiction: A Brief Summary of Research and Practice. Current psychiatry reviews, 8(4), 292-298. https://doi.org/10.2174/157340012803520513
30. Castillo-Carniglia, Alvaro et al., Psychiatric comorbidities in alcohol use disorder, The Lancet Psychiatry, Vol. 6, Issue 12, 1068-1080.
31. Center for Drug Evaluation and Research. (2013, September 25). Drug interactions: What you should know. U.S. Food and Drug Administration. Retrieved Dec. 23, 2021, from https://www.fda.gov/drugs/resources-you-drugs/drug-interactions-what-you-should-know
32. Centers for Disease Control and Prevention. (2016). Health-related quality of life (HRQOL). Retrieved from: https://www.cdc.gov/hrqol/methods.htm.
33. Centers for Disease Control and Prevention. (2019, December 30). Binge drinking is a serious but preventable problem of excessive alcohol use. Centers for Disease Control and Prevention. Retrieved Dec. 23, 2021, www.cdc.gov/alcohol/fact-sheets/binge-drinking.htm
34. Chabrol H. & Oehen, P. (2013) MDMA assisted psychotherapy found to have a large effect for chronic post-traumatic stress disorder. J Psychopharmacol; s27(9):865-6.
35. Chamberlain, S. R., Lochner, C., Stein, D. J., Goudriaan, A. E., van Holst, R. J., Zohar, J., & Grant, J. E. (2016). Behavioural addiction—a rising tide?. European Neuropsychopharmacology, 26(5), 841-855.
36. Chen, L., Jiang, X., Wang, Q., Böthe, B., Potenza, M. N., & Wu, H. (2021). The Association between the Quantity and Severity of Pornography Use: A Meta-analysis. Journal of sex research, 1-16. Advance online publication. https://doi.org/10.1080/00224499.2021.1988500
37. Clay, J. M., Parker, M. O. (2020) Alcohol use and misuse during the COVID-19 pandemic: a potential public health crisis? The Lancet: Public Health, Correspondence, Volume 5, ISSUE 5, e259, May 1, 2020.
38. What is tapering? Coalition Recovery. (2019, October 2). Retrieved Dec. 23, 2021, from https://www.coalitionrecovery.com/rehab-blog/what-is-tapering/
39. Coleman, E., Swinburne Romine, R., Dickenson, J., & Miner, M. H. (in press). Compulsive sexual behaviour inventory—13. In R. R. Milhausen, J. K. Sakaluk, T. D. Fisher, C. M. Davis, W. L. Yarber (Eds.), Handbook of sexuality-related measures. New York, NY: Routledge.
40. Coles, T., Coon, C., DeMuro, C., McLeod, L., & Gnanasakthy, A. (2014). Psychometric evaluation of the Sheehan Disability Scale in adult patients with attention-deficit/hyperactivity disorder. Neuropsychiatric disease and treatment, 10, 887-895. https://doi.org/10.2147/NDT. S55220
41. Columb, D., Hussain, R., & O'Gara, C. (2020). Addiction psychiatry and COVID-19: impact on patients and service provision. Irish Journal of Psychological Medicine, 37(3), 164-168.
42. Cozzi N V, Sievert M K, Shulgin A T, et al. (1999) Inhibition of plasma membrane monoamine transporters by beta-ketoamphetamines. Eur J Pharmacol 1999; 381:63-9.
43. Cuncic, A. (2021, January 22). The mini-social phobia inventory screens for Social Anxiety Disorder. Verywell Mind. Retrieved May 24, 2022, from https://www.verywellmind.com/mini-social-phobia-inventory-mini-spin-3024440.
44. Culig J, Leppée M. From Morisky to Hill-bone; self-reports scales for measuring adherence to medication. Coll Antropol. 2014 March; 38(1):55-62. PMID: 24851597.
45. Curran L, Andrykowski M A, Studts, J L. (1995). Short Form of the Profile of Mood States (POMS-SF): Psychometric information. Psychological Assessment. 7 (1): 80-83
46. Currie C, Davies A, Blunt I, Ariti C, Bardsley M (2015) Alcohol-specific activity in hospitals in England. Research report. The Nuffield Trust.
47. Curry, D. W., Young, M. B., Tran, A. N., Daoud, G. E., & Howell, L. L. (2018). Separating the agony from ecstasy: R(−)-3,4-MDMA has prosocial and therapeutic-like effects without signs of neurotoxicity in mice. Neuropharmacology, 128, 196-206. https://doi.org/10.1016/j.neuropharm.2017.10.003.
48. Davis, A. K., Barrett, F. S., Griffiths, R. R. (2020). Psychological flexibility mediates the relations between acute psychedelic effects and subjective decreases in depression and anxiety. Journal of Contextual Behavioral Science. 15, 39-45.
49. Davis, M. H. (1980). A multidimensional approach to individual differences in empathy. JSAS Catalog of Selected Documents in Psychology, 10, 85.
50. de la Torre R, Farré M, Roset P N, Pizarro N, Abanades S, Segura M, Segura J, Cami J. Human pharmacology of MDMA: pharmacokinetics, metabolism, and disposition. Ther Drug Monit. 2004 April; 26(2):137-44. doi: 10.1097/00007691-200404000-00009. PMID: 15228154.
51. Development of the World Health Organization WHOQOL-BREF quality of life assessment. The WHOQOL Group. Psychol Med. 1998 May; 28(3):551-8. doi: 10.1017/s0033291798006667. PMID: 9626712.
52. Ding, W. X., & Yang, L. (2019). Alcohol and drug-induced liver injury: Metabolism, mechanisms, pathogenesis and potential therapies⋆. Liver research, 3(3-4), 129-131. https://doi.org/10.1016/j.livres.2019.11.006
53. Ditman, K. S., Bailey, J. J., 1967. Evaluating LSD as a psychotherapeutic agent. In: Abramson, H. (Ed.), The Use of LSD in Psychotherapy and Alcoholism. BobbsMerrill, New York, NY, pp. 74e80.
54. Dittrich A. (1998). The standardized psychometric assessment of altered states of consciousness (ASCs) in humans. Pharmacopsychiatry. 31, Suppl 2:80-4. doi: 10.1055/s-2007-979351. PMID: 9754838.
55. Doblin R1, Greer G, Holland J, Jerome L, Mithoefer M C, Sessa B. (2014) A reconsideration and response to Parrott A C (2013) “Human psychobiology of MDMA or ‘Ecstasy’: an overview of 25 years of empirical research”. Hum Psychopharmacol. 2014 March; 29(2):105-8.
56. Egerer G, Stickel F, Seitz H K, 45—Alcohol and the Gastrointestinal Tract, Comprehensive Handbook of Alcohol Related Pathology, Academic Press, 2005, Pages 557-570, ISBN 9780125643702, https://doi.org/10.1016/B978-012564370-2/50047-7. (https://www.sciencedirect.com/science/article/pii/B9780125643702500477)
57. Evren, C., Durkaya, M., Dalbudak, E., çelik, S., çetin, R., & çakmak, D. (2010). Factors related with relapse in male alcohol dependents: 12 months follow-up study. Düs̨ünen Adam: The Journal of Psychiatry and Neurological Sciences, 23(2), 92-99.
58. First M B, et al. (1997) Structured Clinical Interview for DSM-IV Personality Disorders, (SCID-II) Washington, DC: American Psychiatric Press; 1997.
59. First M B, Williams J B W, Karg R S, Spitzer R L. (2015) Structured Clinical Interview for DSM-5 Disorders, Clinical Trials Version (SCID-5-CT). Arlington, VA, American Psychiatric Association, 2015
60. Flannery B A, Volpicelli J R and Pettinati H M (1999) Psychometric properties of the Penn Alcohol Craving Scale. Alcohol Clin Exp Res 23: 1289-1295.
61. Foster J H, Peters T J, Marshall E J. Quality of life measures and outcome in alcohol-dependent men and women. Alcohol. 2000 August; 22(1):45-52. doi: 10.1016/s0741-8329(00)00102-6. PMID: 11109027.
62. Frischknecht U, Sabo T, Mann K. (2013) Improved Drinking Behavior Improves Quality of Life: A Follow-Up in Alcohol-Dependent Subjects 7 Years After Treatment, Alcohol and Alcoholism, 48(5):579-584.
63. Gamma A, Buck A, Berthold T, Liechti M E, Vollenweider F X. (2000) 3,4-Methylenedioxymethamphetamine (MDMA) modulates cortical and limbic brain activity as measured by [H(2)(15)O]-PET in healthy humans. Neuropsychopharmacology 23: 388-95.
64. Gouzoulis-Mayfrank E., Thimm B., Rezk M., Hensen G., Daumann J. (2003). Memory impairment suggests hippocampal dysfunction in abstinent ecstasy users. Prog. Neuropsychopharmacol. Biol. Psychiatry 27, 819-827
65. Green (1996) The Trauma History Questionnaire (THQ)
66. Greer G R, Tolbert R. (1986) Subjective Reports of the Effects of MDMA in a Clinical Setting. Journal of Psychoactive Drugs. Vol. 18(4) October-December, 1986
67. Greer G R, Tolbert R. (1998) A method of conducting therapeutic sessions with MDMA. 30: 371-379 Journal of Psychoactive Drugs.
68. Grof (2008) LSD Psychotherapy (Ben Lomond, CA: Multidisciplinary Association for Psychedelic Studies).
69. Günther O, Roick C, Angermeyer M C, König HH. The EQ-5D in alcohol dependent patients: relationships among health-related quality of life, psychopathology and social functioning. Drug Alcohol Depend. 2007 Jan. 12; 86(2-3):253-64. doi: 10.1016/j.drugalcdep.2006.07.001. Epub 2006 Aug. 22. PMID: 16930855.
70. Hallgren K A, Witkiewitz K. Missing data in alcohol clinical trials: a comparison of methods. Alcohol Clin Exp Res. 2013 December; 37(12):2152-60.
71. Halpern J H, Sherwood A R, Hudson J I, Gruber S, Kozin D, Pope H G, Jr. (2004) Residual neurocognitive features of long-term ecstasy users with minimal exposure to other drugs. Drug and Alcohol Dependence. 75, 135-147
72. Hamer, R. and Simpson, P., 2009. Last Observation Carried Forward Versus Mixed Models in the Analysis of Psychiatric Clinical Trials. American J. Psychiatry, 166(6):639-41
73. Hanson, K. L., & Luciana, M. (2010). Neurocognitive impairments in MDMA and other drug users: MDMA alone may not be a cognitive risk factor. Journal of Clinical and Experimental Neuropsychology, 32, 337-349.
74. Hanson, K. L., & Luciana, M. (2004). Neurocognitive function in users of MDMA: the importance of clinically significant patterns of use. Psychological Medi., 34, 229-246
75. Harris D S, Baggott M, Mendelson J H, Mendelson J E, Jones R T. (2002) Subjective and hormonal effects of 3,4-methylenedioxymethamphetamine (MDMA) in humans. Psychopharmacology (Berl); 162(4): 396-405.
76. Harris, R. (2009). ACT Made Simple: An Easy-To-Read Primer on Acceptance and Commitment Therapy. Oakland, CA: New Harbinger.
77. Hendershot, C. S., Witkiewitz, K., George, W. H., & Marlatt, G. A. (2011). Relapse prevention for addictive behaviors. Substance abuse treatment, prevention, and policy, 6, 17. https://doi.org/10.1186/1747-597X-6-17.
78. Holland, Julie; Metzner, Ralph; Adamson, Sophia (2001). Ecstasy: the complete guide; a comprehensive look at the risks and benefits of MDMA. Rochester, Vt., Park Street Press. ISBN 978-0-89281-857-0.
79. Holze, F., Vizeli, P., Müller, F., Ley, L., Duerig, R., Varghese, N., Eckert, A., Borgwardt, S., & Liechti, M. E. (2020). Distinct acute effects of LSD, MDMA, and D-amphetamine in healthy subjects. Neuropsychopharmacology: official publication of the American College of Neuropsychopharmacology, 45(3), 462-471. https://doi.org/10.1038/s41386-019-0569-3
80. HM Government (2012) The Government's Alcohol Strategy. Secretary of State for the Home Department H M Government. Page 3.
81. HSCIC (Health and Social Care Information Centre) (2014) Statistics on Alcohol—England, 2014. http://www.hscic.gov.uk/catalogue/PUB14184
82. Hsu S H, Grow J, Marlatt G A. (2008) Mindfulness and addiction. In: Galanter M, Kaskutas L A, editors. Recent Developments in Alcoholism. Vol. 18. pp. 229-250.
83. Hunt S M, McKenna S P, McEwen J, et al A quantitative approach to perceived health status: a validation study. Journal of Epidemiology & Community Health 1980; 34:281-286.
84. Husain, K., Ansari, R. A., & Ferder, L. (2014). Alcohol-induced hypertension: Mechanism and prevention. Worldjournal of cardiology, 6(5), 245-252. https://doi.org/10.4330/wjc.v6.i5.245
85. Hysek C M, Domes G, Liechti M E. (2012a) MDMA enhances “mind reading” of positive emotions and impairs “mind reading” of negative emotions. 222: 293-302. Psychopharmacology.
86. Hysek C M, et al. (2012b) Duloxetine inhibits effects of MDMA (“ecstasy”) in vitro and in humans in a randomized placebo-controlled laboratory study. PLoS One.; 7(5):
87. Hysek, C. M., et al. (2013) MDMA enhances emotional empathy and prosocial behavior. Soc Cogn Affect Neurosci. November; 9(11):1645-52.
88. Jaiswal et al., Nanoemulsion: an advanced mode of drug delivery system, Biotech 3(5):123-27 (2015).
89. Jerome, L, Schuster, S & Berra Yazar-Klosinski, B. (2013) Can MDMA Play a Role in the Treatment of Substance Abuse? Current Drug Abuse Reviews, 2013, 6, 000-000
90. Johnson, Richards, & Griffiths (2008). Human hallucinogen research: guidelines for safety. Journal of psychopharmacology, 22(6), 603-620.
91. Johnson M W, Garcia-Romeu A, Cosimano M P, Griffiths R R. (2014) Pilot study of the 5-HT2AR agonist psilocybin in the treatment of tobacco addiction.
92. Kabat-Zinn, J., Massion, A. O., Kristeller, J., Peterson, L. G., Fletcher, K. E., Pbert, L., Lenderking, W. R., & Santorelli, S. F. (1992). Effectiveness of a meditation-based stress reduction program in the treatment of anxiety disorders. The American journal of psychiatry, 149(7), 936-943. https://doi.org/10.1176/ajp.149.7.936.
93. Kabat-Zinn J. Wherever You Go, There You Are: Mindfulness Meditation in Everyday Life. New York: Hyperion; 1994.
94. Kaelen, M. et al. (2015) LSD enhances the emotional response to music. Psychopharmacology: 232 (19)
95. Kiluk, B. D., Dreifuss, J. A., Weiss, R. D., Morgenstern, J., & Carroll, K. M. (2013). The Short Inventory of Problems—Revised (SIP-R): Psychometric properties within a large, diverse sample of substance use disorder treatment seekers. Psychology of Addictive Behaviors: Journal of the Society of Psychologists in Addictive Behaviors, 27(1), 307-314.
96. Krampe, H., Stawicki, S., Wagner, T., Bartels, C., Aust, C., Ruther, E., Poser, W., Ehrenreich, H. (2006). “Follow-up of 180 Alcoholic Patients for up to 7 Years After Outpatient Treatment: Impact of Alcohol Deterrents on Outcome”. Alcoholism: Clinical and Experimental Research 30 (1): 86-95.
97. Kranzler H R, Soyka M. Diagnosis and Pharmacotherapy of Alcohol Use Disorder: A Review. JAMA. 2018; 320(8):815-824. doi:10.1001/jama.2018.11406
98. Kroenke K, Spitzer R L, Williams J B. (2001) The PHQ-9: validity of a brief depression severity measure. J Gen Intern Med. September; 16(9):606-13.
99. Kouimtsidis C (2017) Structured Preparation before Alcohol Detoxification; A Shift from the Current Treatment Paradigm. J Addict Res Ther 8:326. doi:10.4172/2155-6105.1000326
100. Krupitsky, E. M. & Grinenko, A. Y (1997) Ketamine psychedelic therapy (KPT): A review of the results of ten years of research. J. Psychoactive Drugs 29 (2), 165-183
101. Kurland, A. A., Unger, S., Shaffer, J. W, Savage, C., 1967. Psychedelic therapy utilizing LSD in the treatment of the alcoholic patient: a preliminary report. Am. J. Psychiatry 123 (10), 1202e1209.
102. Kurland, A. A., Savage, C., Pahnke, W. N., Grof, S., Olsson, J. E., 1971. LSD in the treatment of alcoholism. In: Vinar, O., Votava, Z., Bradley, P. B. (Eds.), Advances in Neuropsychopharmacology: Proceedings of the 7th Congress of the Collegium International Neuropsychopharma Cologicum. North-Holland, Amsterdam, pp. 361e372.
103. Kuypers K P, Ramaekers J G. (2007) Acute dose of MDMA (75 mg) impairs spatial memory for location but leaves contextual processing of visuospatial information unaffected. Psychopharmacology (Berl) 2007; 189(4): 557-63.
104. Lachman et al., Theory and Practice of Industrial Pharmacy (1986).
105. Lam, W. Y, & Fresco, P. (2015). Medication Adherence Measures: An Overview. BioMed research international, 2015, 217047. https://doi.org/10.1155/2015/217047
106. Lavelle A, Honner V, Docherty J R. (1999) Investigation of the prejunctional alpha2-adrenoceptor medi-ated actions of MDMA in rat atrium and vas deferens. Br J Pharmacol 1999; 128:975-80.
107. Liberman et al. Pharmaceutical Dosage Forms, 2 Ed., Vol. 1, pp. 209-214 (1990).
108. Liechti M E, Gamma A, Vollenweider F X. (2001) Gender differences in the subjective effects of MDMA. Psychopharmacology (Berl) 2001; 154(2): 161-8.
109. Lingford-hughes, A. R., Welch, S., Peters, L. and J Nutt, D. j. (2012) BAP updated guidelines: evidence-based guidelines for the pharmacological management of substance abuse, harmful use, addiction and comorbidity: recommendations from bap. J psychopharmacol. 2012 jul; 26(7):899-952.
110. Lowry, R. (2001-2013). Significance of the difference between two correlation coefficients. Retrieved from http://vassarstats.net/rdiff.html
111. Malcolm R, Roberts J S, Wang W, Myrick H, Anton R F. Multiple previous detoxifications are associated with less responsive treatment and heavier drinking during an index outpatient detoxification. Alcohol. 2000 November; 22(3):159-64. PubMed PMID: 11163123.
112. Marcus, M. T., & Zgierska, A. (2009). Mindfulness-Based Therapies for Substance Use Disorders: Part 1 (Editorial). Substance Abuse: Official Publication of the Association for Medical Education and Research in Substance Abuse, 30(4), 263.
113. Marlatt, G. A & Gordon, J. R. (1985). Relapse prevention: Maintenance strategies in the treatment of addictive behaviors. New York: Guilford Press.
114. Mas M, Farre M, de la Torre R, et al. (1999) Cardiovascular and neuroendocrine effects and pharmacokinetics of 3, 4-methylenedioxymethamphetamine in humans. J Pharmacol Exp Ther; 290(1): 136-45.
115. Mayo-Smith M F. (1997) Pharmacological management of alcohol withdrawal. A meta-analysis and evidence-based practice guideline. American Society of Addiction Medicine Working Group on Pharmacological Management of Alcohol Withdrawal. JAMA. 1997 Jul. 9; 278(2):144-51. PubMed PMID 9214531.
116. McCulloch, D. E., Grzywacz, M. Z., Madsen, M. K., Jensen, P. S., Ozenne, B., Armand, S., . . . Stenbok, D. S. (2021, December 14). A Quantitative and Qualitative Report of Psilocybin Induced Mystical-Type Experiences and Their Relation to Lasting Positive Effects. https:/doi org/10.3 1234/osf.io/xgzu2
117. McLean, J. R., MacDonald, D. C., Byrne, U. P, Hubbard, A. M., 1961. The use of LSD-25 in the treatment of alcoholism and other psychiatric problems. Q. J. Stud. Alcohol 22, 3445.
118. Metzner, Ralph (1993). Letter from Ralph Metzner. Newsletter of the Multidisciplinary Association for Psychedelic Studies MAPS. 4 (1). Retrieved 8 Jan. 2015.
119. Miller, W. R., and Rollnick, S. (1991). Motivational Interviewing: Preparing People To Change Addictive Behavior. New York: Guilford Press.
120. Miller W R, Wilbourne P L. (2002) Mesa Grande: a methodological analysis of clinical trials of treatments for alcohol use disorders. Addiction. 2002; 97:265-277
121. Mitchell, J. M., Bogenschutz, M., Lilienstein, A. et al. MDMA-assisted therapy for severe PTSD: a randomized, double-blind, placebo-controlled phase 3 study. Nat Med 27, 1025-1033 (2021). https://doi.org/10.1038/s41591-021-01336-3.
122. Mithoefer M C, Wagner M T, Mithoefer A T, Jerome L, Martin S F, Yazar-Klosinski B, Michel Y, Brewerton T D, Doblin R. (2013) Durability of improvement in post-traumatic stress disorder symptoms and absence of harmful effects or drug dependence after 3,4-methylenedioxymethamphetamine-assisted psychotherapy: a prospective long-term follow-up study. Journal of Psychopharmacology. 27(1):28-39.
123. Mithoefer M C, Wagner T M, Mithoefer A T, Jerome L, Doblin R (2010) The safety and efficacy of ±3,4-methylenedioxymethamphetamine-assisted psychotherapy in subjects with chronic, treatment-resistant posttraumatic stress disorder: the first randomized controlled pilot study. Journal of Psychopharmacology. 25(4): 439-452.
124. Mithoefer M C (2017). A Manual for MDMA-assisted therapy in the Treatment of Posttraumatic Stress Disorder. Ver. 8.1 https://maps.org/research-archive/mdma/MDMA-Assisted-Psychotherapy-Treatment-Manual-Version7-19Aug15-FINAL.pdf.
125. Mithoefer, M. C., Feduccia, A. A., Jerome, L. et al. MDMA-assisted therapy for treatment of PTSD: study design and rationale for phase 3 trials based on pooled analysis of six phase 2 randomized controlled trials. Psychopharmacology 236, 2735-2745 (2019). https://doi.org/i0.1007/s00213-019-05249-5.
126. Moos, R. H., & Moos, B. S. (2006). Rates and predictors of relapse after natural and treated remission from alcohol use disorders. Addiction (Abingdon, England), 101(2), 212-222. doi:10.1111/j.1360-0443.2006.01310.x).
127. Morisky D E, Green L W, Levine D M. Concurrent and predictive validity of a self-reported measure of medication adherence. Med Care. 1986 January; 24(1):67-74. doi: 10.1097/00005650-198601000-00007. PMID: 3945130.
128. Mundt, J C et al. (2013). “Prediction of suicidal behavior in clinical research by lifetime suicidal ideation and behavior ascertained by the electronic Columbia-Suicide Severity Rating Scale.”. The Journal of clinical psychiatry 74 (9): 887-93.
129. Multidisciplinary Association for Psychedelic Studies (MAPS) (2016) Investigators Brochure for MDMA-assisted therapy. Available for download at: https://maps.org/research/mdma/mdma-research-timeline/104-other-mdma-resources/5400-mdma-investigator-s-brochure-and-fda-annual-report&Itemid=485
130. Nair J B, Hakes L, Yazar-Klosinski B, Paisner K, Fully Validated, Multi-Kilogram cGMP Synthesis of MDMA, ACS Omega (IF3.512), 2021-12-20 10.1021/acsomega.1c05520.
131. Nash J F, Roth B L, Brodkin J D, et al. (1994) Effect of the R(−) and S(+) isomers of MDA and MDMA on phosphatidyl inositol turnover in cultured cells expressing 5-HT2A or 5-HT2C receptors. Neurosci Lett 1994; 177:111-5.
132. National Clinical Guideline Centre (2010). 2 Acute Alcohol Withdrawal. Alcohol Use Disorders: Diagnosis and Clinical Management of Alcohol-Related Physical Complications (No. 100 ed.). London: Royal College of Physicians (UK). Archived from the original on 31 Jan. 2014.
133. National Collaborating Centre for Mental Health (UK). Alcohol-Use Disorders: Diagnosis, Assessment and Management of Harmful Drinking and Alcohol Dependence. Leicester (UK): British Psychological Society; 2011. (NICE Clinical Guidelines, No. 115.) 2, ALCOHOL DEPENDENCE AND HARMFUL ALCOHOL USE. Available from: https://www.ncbi.nlm.nih.gov/books/NBK65500/
134. National Institute for Health and Care Excellence, NICE (June 2013) Alcohol-use disorder diagnosis and clinical management of alcohol-related physical complications CG100
135. Neff, KD. (2003). Development and validation of a scale to measure self-compassion. Self and Identity, 2, 223-250.
136. NICE (2011) Alcohol-use Disorders: Diagnosis, Assessment and Management of Harmful Drinking and Alcohol Dependence. NICE clinical guideline 115. Available at www.nice.org.uk/CG115 [“NICE Guidelines” ]
137. Nichols, D. E. (1986). “Differences Between the Mechanism of Action of MDMA, MBDB, and the Classic Hallucinogens. Identification of a New Therapeutic Class: Entactogens”. J. Psychoactive Drugs 18(4): 305-13.
138. Oehen, P, Traber, R., Widmer, V, & Schnyder, U. (2013). A randomized, controlled pilot study of MDMA (±3,4-Methylenedioxymethamphetamine)-assisted psychotherapy for treatment of resistant, chronic Post-Traumatic Stress Disorder (PTSD). Journal of psychopharmacology (Oxford, England), 27(1), 40-52. https://doi.org/10.1177/0269881112464827.
139. Oehen P, Traber, R., Widmer, V, Schnyder, U. (2013) Pilot study of MDMA-assisted therapy for treatment-resistant PTSD. J Psychopharmacol; 27(1): 40-52.
140. Oeri H. E. (2021). Beyond ecstasy: Alternative entactogens to MDMA with potential applications in psychotherapy. J psychopharmacol., 35(5), 512-536.
141. Office for National Statistics (ONS). (7 Dec. 2021). Alcohol-specific deaths in the UK: registered in 2020. Available at (last accessed 6 Jan. 2022): https://www.ons.gov.uk/peoplepopulationandcommunity/healthandsocialcare/causesofdeath/bullet ins/alcoholrelateddeathsintheunitedkingdom/registeredin2020
142. Osaji, J., Ojimba, C., & Ahmed, S. (2020). The Use of Acceptance and Commitment Therapy in Substance Use Disorders: A Review of Literature. Journal of clinical medicine research, 12(10), 629-633. https://doi.org/10.14740/jocmr4311.
143. Paille, F. and Martini, H. (2014). “Nalmefene: a new approach to the treatment of alcohol dependence”. Substance Abuse and Rehabilitation 5 (5): 87-94.
144. Parrott and Hindmarch, (1980). The Leeds Sleep Evaluation Questionnaire in Psychopharmacological Investigations—a Review Psychopharmacology 71, 173-179.
145. Passie (2012) Healing with Entactogens: Therapist and Patient Perspectives on MDMA-Assisted Group Psychotherapy (Ben Lomond, CA: Multidisciplinary Association for Psychedelic Studies).
146. Paterson et al. (2015) The Imperial College Cambridge Manchester (ICCAM) platform study: an experimental medicine platform for evaluating new drugs for relapse prevention in addiction. Part A: study description. J Psychopharmacol, September 2015, vol. 29, no. 9, 943-960
147. Piano M. R. (2017). Alcohol's Effects on the Cardiovascular System. Alcohol research: current reviews, 38(2), 219-241.
148. Pitts, E. G., Curry, D. W., Hampshire, K. N., Young, M. B., & Howell, L. L. (2018). (±)-MDMA and its enantiomers: potential therapeutic advantages of R(−)-vDMA. Psychopharmacology, 235(2), 377-392. https://doi.org/10.1007/s00213-017-4812-5.
149. Project MATCH Research Group (1998a). Matching alcoholism treatments to client heterogeneity: Project MATCH three year drinking outcomes. Alcoholism: Clinical and Experimental Research, 22, 1300-1311.
150. Rehm J. The risks associated with alcohol use and alcoholism. Alcohol Res Health. 2011; 34(2):135-43
151. Rezvani, A. H., Garges, P L., Miller, D. B. and Gordon C. J. (1992) Attenuation of Alcohol Consumption by MDMA (Ecstasy) in Two Strains of Alcohol-preferring Rats. Pharmacology, Biochemistry and Behavior, Vol 43, pp. 103-110.
152. Rogers G, Elston J, Garside R, et al. (2009) The harmful health effects of recreational ecstasy: a systematic review of observational evidence. Health Technol Assess 2009; 13(6): iii-iv, ix-xii, 1-315.
153. Rösner S, Hackl-Herrwerth A, Leucht S, Lehert P, Vecchi S, Soyka M (2010). Rosner, Susanne, ed. “Acamprosate for alcohol dependence”. Cochrane Database of Systematic Reviews (9): CD004332.doi:10.1002/14651858. CD004332.pub2.
154. Rydzynski, Z., Cwynar, S., Grzelak, L., 1968. Preliminary report on the experience with psychotomimetic drugs in the treatment of alcoholism. Act. Nerv. Super. (Praha) 10 (3), 273.
155. Saitz M, Mayo-Smith M F, Redmond H A, Bernard D R, Calkins D R. (1994) Individualized treatment for alcohol withdrawal. A randomized double-blind controlled trial. JAMA 1994; 272:519-23.
156. Saleemi, S., Pennybaker, S. J., Wooldridge, M., & Johnson, M. W. (2017). Who is ‘Molly’?MDMA adulterants by product name and the impact of harm-reduction services at raves. Journal of psychopharmacology (Oxford, England), 31(8), 1056-1060.
157. Sayette, M. A., Shiffman, S., Tiffany, S. T., Niaura, R. S., Martin, C. S., & Shadel, W. G. (2000). The measurement of drug craving. Addiction (Abingdon, England), 95 Suppl 2(Suppl 2), S189-S210. https://doi.org/10.1080/09652140050111762
158. Schmid, Gasser, Oehen, & Liechti (2020). Acute subjective effects in LSD- and MDMA-assisted therapy. Journal of psychopharmacology, 0269881120959604.
159. Selvaraj S, Hoshi R, Bhagwagar Z, et al. (2009) Brain serotonin transporter binding in former users of MDMA (‘ecstasy’). Br J Psychiatry; 194(4): 355-9.
160. Sessa, B. (2012) Could MDMA be useful in the treatment of PTSD? Progress in Neurology and Psychiatry. Volume 15, Issue 6, pages 4-7
161. Sessa & Fischer (2015), Underground MDMA-, LSD- and 2-CB-assisted individual and group psychotherapy in Zurich: Outcomes, implications and commentary. Drug Science, Policy and Law, 2, 2050324515578080.
162. Sessa B. (2017) Why MDMA therapy for alcohol use disorder? And why now? Neuropharmacol. 142:83-88. doi: 10.1016/j.neuropharm.2017.11.004. PMID: 29126911.
163. Sessa, B., Higbed, L. and Nutt, DJ (2019) A Review of 3,4-methylenedioxymethamphetamine (MDMA)-Assisted Psychotherapy. Frontiers Psychiatry 10:138. doi: 10.3389/fpsyt.2019.00138
164. Sessa, B., Sakal, C. M., O'Brien, S. & Nutt, D. J. (2019) First study of safety and tolerability of MDMA-assisted therapy in patients with alcohol use disorder: preliminary data on the first four participants. MDMA-AUD—BMJ Case Reports paper—July 2019(1) BMJ Case Reports. 2019; 12:e230109
165. Sessa, B. et al. (2020). How well are Patients doing Post-Alcohol Detox in Bristol? Results from the Outcomes Study. J. Alcohol Drug Depend. Subst. Abuse, 6:021
166. Sessa, B., Higbed, L., O'Brien, S., Durant, C., Sakal, C., Titheradge, D., Williams, T. M., Rose-Morris, A., Brew-Girard, E., Burrows, S., Wiseman, C., Wilson, S., Rickard, J., & Nutt, D. J. (2021). First study of safety and tolerability of 3,4-methylenedioxymethamphetamine-assisted psychotherapy in patients with alcohol use disorder. Journal of psychopharmacology (Oxford, England), 35(4), 375-383. https://doi.org/10.1177/0269881121991792
167. Shapiro S L, et al. Mechanisms of mindfulness. Journal of Clinical Psychology. 2006; 62(3):373-386.
168. Sheehan D V, Lecrubier Y, Sheehan K H, Amorim P, Janavs J, Weiller E, Hergueta T, Baker R, Dunbar G C. (1998) The Mini-International Neuropsychiatric Interview (M.I.N.I.): the development and validation of a structured diagnostic psychiatric interview for DSM-IV and ICD-10. J Clin Psychiatry; 59 Suppl 20:22-33; quiz 34-57.
169. Sheth et al. (1980). Compressed tablets, in Pharm. dosage forms, Vol. 1, Lieberman & Lachtman, eds., Dekker, NY
170. Shulgin, A. T. (1986). The Background and Chemistry of MDMA. Journal of Psychoactive Drugs, 18(4), 291-304. doi:10.1080/02791072.1986.1047.
171. Singh et al. Encyclopedia of Pharmaceutical Technology, 2nd Ed., pp. 751-753 (2002).
172. Sloshower, J., Guss, J., Krause, R., Wallace, R. M., Williams, M. T., Reed, S., Skinta., M. D. (2020). Psilocybin-assisted therapy of major depressive disorder using Acceptance and Commitment Therapy as a therapeutic frame. Journal of Contextual Behavioral Science, 15; 12-19.
173. Sobell, L. C., Agrawal, S., Annis, H. M., et al. (2001). Cross-cultural evaluation of two drinking related assessment instruments: Alcohol Timeline Follow Back and Inventory of Drinking Situations. Substance Use & Misuse, 36, 313-331.
174. Sobell L C, Sobell M B, 1996, Timeline FollowBack user's guide: A calendar method for assessing alcohol and drug use. Addiction Research Foundation, Toronto, Ontario, Canada; J. Anal. Toxicolo., 2002, 26: 393-400.
175. Sobell L C, Sobell M B, Maisto S A. Follow-up attrition in alcohol treatment studies: is ‘no news’ bad news, good news or no news? Drug Alcohol Depend. 1984 January; 13(1):1-7.
176. Soyka M, Horak M. Outpatient alcohol detoxification: implementation efficacy and outcome effectiveness of a model project. Eur Addict Res. 2004; 10(4):180-7.
177. Soyka M, Rosner S. (2008). “Opioid antagonists for pharmacological treatment of alcohol dependence—a critical review”. Curr Drug Abuse Rev 1 (3): 280-91.
178. Spitzer R. L., Kroenke K., Williams J. B., Löwe B. (2006) A brief measure for assessing generalized anxiety disorder: the GAD-7. Arch intern med. 22; 166(10):1092-7.
179. Stanford Sparqtools: Health-Related Quality of Life Scale. Retrieved Jan. 6, 2022 from sparqtools.org/mobility-measure/health-related-quality-of-life-scale/#all-survey-questions 180. Stockwell T, Murphy D, Hodgson R. (1983) The severity of alcohol dependence questionnaire: its use, reliability and validity. Bri. Journal of Addictions; 78:145-155.
181. Stolaroff M. (2004) The Secret Chief Revealed: Conversations with a pioneer of the underground therapy movement. Chapter 2, pages 45-49. Published by: Multidisciplinary Association for Psychedelic Studies, Sarasota, USA.
182. Sullivan J T, Sykora K, Schneiderman J, Naranjo C A, Sellers E M. Assessment of alcohol withdrawal: the revised clinical institute withdrawal assessment for alcohol scale (CIWA-Ar). Br J Addict. 1989 November; 84(11):1353-7.
183. Sumnall, H. R, Cole, J. and Jerome, L. (2006) The varieties of ecstatic experience: an exploration of the subjective experiences of ecstasy. Journal of Psychopharmacology. 20(5) (2006) 670-682.
184. Teasdale, J. D., Segal, Z. V, Williams, J. M., Ridgeway, V. A., Soulsby, J. M., & Lau, M. A. (2000). Prevention of relapse/recurrence in major depression by mindfulness-based cognitive therapy. Journal of consulting and clinical psychology, 68(4), 615-623. https://doi.org/10.1037//0022-006x.68.4.615.
185. Thompson M R, Callaghan P D, Hunt G E, Cornish J L, McGregor I S. (2007) A role for oxytocin and 5-HT(IA) receptors in the prosocial effects of 3,4 methylenedioxymethamphetamine (“ecstasy”). Neuroscience; 146(2): 509-14.
186. Ugochukwu, C, Bagot, KS; Delaloye, S, Pi, S, Vien, L; Garvey, T, Bolotaulo, Nestor I, Kumar, N, IsHak, WW (2013). The Importance of Quality of Life in Patients with Alcohol Abuse and Dependence, Harvard Review of Psychiatry: 21(1):1-17.
187. UKATT Research Team (2005). Effectiveness of treatment for alcohol problems: Findings of the randomised UK Alcohol Treatment Trial (UKATT). British Med. J., 311, 541-544.
188. U.S. Department of Health and Human Services. (2021). What is a standard drink? National Institute on Alcohol Abuse and Alcoholism. Retrieved Dec. 30, 2021, from niaaa.nih.gov/alcohols-effects-health/overview-alcohol-consumption/what-standard-drink
189. Villalobos-Gallegos, L., Pérez-López, A., Mendoza-Hassey, R., Graue-Moreno, J., & Marin-Navarrete, R. (2015). Psychometric and diagnostic properties of the Drug Abuse Screening Test (DAST): Comparing the DAST-20 vs. the DAST-10. Salud Mental, 38(2), 89-94. https://doi.org/10.17711/SM.0185-3325.2015.012
190. Ware, JE Jr, Sherbourne C D. (1992) The MOS 36-item short-form health survey (SF-36). I. Conceptual framework and item selection. Med Care. 30(6):473-83. PMID: 1593914.
191. Ware, JE. (1993) Health survey manual and interpretation guide. Section 6: Scoring the SF-36. The Health Institute, New England Medical Center. Nimrod Press, Boston, Massachusetts.
192. Watson, L. and Beck, J. (1991) New Age Seekers: MDMA as an Adjunct to Spiritual Pursuit. Journal of Psychoactive Drugs. Vol 23(3), July-September 1991.
193. Watts, R., & Luoma, J. (2020). The use of the psychological flexibility model to support psychedelic assisted therapy. Journal of contextual behavioral science, 15, 92-102.
194. The WHOQoL GROUP (1995) The World Health Organization Quality Of Life Assessment (WHOQoL): Position Paper From the World Health Organization. Soc Sci Med1014031409
195. WHO. (1996). WHOQOL-BREF: Introduction, administration, scoring and generic version of the assessment, Programme on Mental Health, World Health Organization, Geneva.
196. Witkiewitz K, et al., Recommendations for the Design and Analysis of Treatment Trials for Alcohol Use Disorders. Alcohol Clin Exp Res. 2015 September; 39(9):1557-70.
197. Witkiewitz, K., Litten, R. Z., & Leggio, L. (2019). Advances in the science and treatment of alcohol use disorder. Science Advances, 5(9). https://doi.org/10.1126/sciadv.aax4043
198. Yehuda, R., et al. (2016). Holocaust Exposure Induced Intergenerational Effects on FKBP5 Methylation. Biologicalpsychiatry, 80(5), 372-380.
199. Yi, H., Shin, K. and Shin, C. (2006), Development of the Sleep Quality Scale. Journal of Sleep Research, 15: 309-316.
200. Yin, S., Njai, R., Barker, L., Siegel, P. Z., & Liao, Y (2016). Summarizing health-related quality of life (HRQOL): development and testing of a one-factor model. Population health metrics, 14, 22. https://doi.org/10.1186/s12963-016-0091-3
201. Yubero-Lahoz, S., et al. Sex Differences in 3,4 methylenedioxymethamphetamine (MDMA; Ecstasy)-Induced Cytochrome P450 2D6 Inhibition in Humans. Clin Pharmacokinet, 2011. 50(5): p. 319-29.
202. Zanger, U. M., & Schwab, M. (2013). Cytochrome P450 enzymes in drug metabolism: regulation of gene expression, enzyme activities, and impact of genetic variation. Pharmacology & therapeutics, 138(1), 103-141. https://doi.org/10.1016/j.pharmthera.2012.12.007.

1. A method of treating a substance use disorder (SUD) in a patient diagnosed therewith, comprising providing an entactogen-assisted psychotherapy (EAP) regimen, wherein the EAP regimen comprises one or more drug-assisted therapy sessions with an entactogen, and one or more non-drug therapy sessions.

2. The method of claim 1, wherein the SUD is any of alcohol use disorder (AUD), opioid use disorder, nicotine dependence or tobacco use disorder, sedative, hypnotic, and anxiolytic use disorder, and stimulant use disorder.

3. The method of claim 1, wherein the SUD is AUD.

4. The method of claim 3, wherein the AUD is dependent use AUD.

5. The method of claim 4, further comprising providing an alcohol detoxification course prior to the EAP regimen.

6. The method of claim 5, further comprising providing a pre-detoxification motivational group therapy course prior to the alcohol detoxification course.

7. The method of claim 3, wherein the AUD is harmful use AUD.

8. The method of claim 7, further comprising providing a preparatory group therapy course prior to the EAP regimen.

9. The method of claim 1, wherein the method is effective to: (a) reduce use of the substance, (b) reduce cravings for the substance, (c) promote abstinence from the substance, (d) prevent relapse into use of the substance, (e) improve a symptom of the SUD, or (f) reduce the severity of a diagnostic criterion of the SUD.

10. The method of claim 9, wherein the method is effective for at least 12 months after the patient has completed the EAP regimen.

11. The method of claim 10, wherein the method is effective to reduce use of the substance for at least 12 months after the patient has completed the EAP regimen.

12. The method of claim 11, wherein the SUD is AUD, and the method is effective to reduce use of alcohol for at least 12 months after the patient has completed the EAP regimen.

13. The method of claim 1, wherein the EAP regimen comprises at least two drug-assisted therapy sessions with an entactogen.

14. The method of claim 13, wherein the entactogen is any of MDMA, BDB, MBDB, MDA, MDEA, 3-MMC, 4-MMC, methylone, ethylone, butylone, αET, αMT, 5-IAI, MDAI, 5-APDI, 6-APB, 5-APB, 5-MAPB, MDAI, MDOH, and 4-FA, including individual enantiomers and non-racemic mixtures thereof, and including pharmaceutically acceptable salts thereof.

15. The method of claim 14, wherein the entactogen is MDMA, including individual enantiomers and non-racemic mixtures thereof, and including pharmaceutically acceptable salts thereof.

16. The method of claim 1, further comprising administration of an additional active agent, wherein the additional active agent is any of an opioid antagonist, a CB1 antagonist, a CRH1 receptor antagonist, a NK1R antagonist, an OTR agonist, a GABA agent, a voltage-gated sodium channel inhibitor, a voltage-dependent calcium channel agonist, an α7 nicotinic acetylcholine receptor agonist, a 5-HT3 antagonist, a 5-HT1A receptor partial agonist, a 5-HT2A receptor antagonist, a 5-HT reuptake inhibitor, a SERT inhibitor, a serotonergic agent, an α1 adrenoreceptor antagonist, a glucocorticoid receptor antagonist, an α1 adrenoreceptor agonist, an AChE inhibitor, a dopamine D2 receptor antagonist, an α2 adrenoreceptor agonist, an NMDA modulator, an NMDA antagonist, an aldehyde dehydrogenase inhibitor, a phenethylamine, and a tryptamine, including pharmaceutically acceptable salts thereof.

17. A method of treating a behavioral addiction in a patient diagnosed therewith, comprising providing an entactogen-assisted psychotherapy (EAP) regimen, wherein the EAP regimen comprises one or more drug-assisted therapy sessions with an entactogen, and one or more non-drug therapy sessions.

18. The method of claim 17, wherein the behavioral addiction is any of gambling disorder, gaming disorder, compulsive sexual behavioral disorder, compulsive buying-shopping disorder, technology addiction, kleptomania, internet addiction, pornography addiction, technology addiction, binge eating disorder, workaholism, perfectionism, pathological skin picking, exercise addiction, excessive tanning, tattoo addiction, masturbation addiction, sex addiction, and love addiction.

19. A method of treating a comorbidity in a patient diagnosed with a condition, comprising providing an entactogen-assisted psychotherapy (EAP) regimen, wherein the EAP regimen comprises one or more drug-assisted therapy sessions with an entactogen, and one or more non-drug therapy sessions.

20. The method of claim 19, wherein the comorbidity is a comorbid mental health disorder in a patient diagnosed with AUD, and wherein the comorbid mental health disorder is any of post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, a phobia disorder, a binge disorder, body dysmorphic disorder, a mood disorder related to another health condition, a disruptive behavior disorder, an eating disorders, an impulse control disorder, obsessive compulsive disorder (OCD), attention deficit hyperactivity disorder (ADHD), a personality disorder, an attachment disorder, a dissociative disorder, a substance-related disorder including substance use disorder (SUD), and a behavioral addiction.
